SEQUENCE LISTING (194 sequences)

SEQ ID NO: 1 - DNA, 87662 bp, Artificial Sequence
  found in bacteriophage isolated from environment
acaaatagtgaagagataaaccaggttgagcaagaaaaatctttttattgtagtgcatatagatttattaagagggctagaacacatacgaagatagccttatcatcaatttatctaagaattataccaaaatttttagcaaaaagcaagaaaaataaaagcctgatctgctgagatcaggcttggttgattaatcctctggattaacatccaggaaatttttaatagcatcgtgcatagtttctttacgaagctgacgtccaatcaaaatatgaataggttcgactcgaccattaggtacttgatttgtgttctgttttgtggtagtcgtgtgcgcccgtttcattacagttttgccaaaaatatttttaaaattttttcttgactctttgaacgtggttatctagtatttgcatcaacggggagggaatgaccttccccactaaataaagtcctaaactggagattcaaaaatggcatatcgtgcacctaaattcatcaacaaagacaactttcgcaacgcactggagaagtcacttgatgaaaattttaaaggcaacattatcgttgttcacgcattcaattttaagtatgatgtaaacgggaatagaatcaaccattacacggcaaccatgttagatggtacgctatcaagtgaaaaagctgtactgaatgcacttgctggacgtggtaaaaacctgataaaatgtgataagagacgttatcagggtggcgcatatggttatgatgatgctgtttatcatcttgaaaatatgggctatcaagtagaaaaagctggagcgcctcaaataatcggtagtgatggttacgtaactatcttcaaaattaacaaataaagtactttacaaggggcttatagttcgataaagtaagccccagatagagagatttatcaaataaagcccttaaactaggagattctaaaatgaaattatcaaatcaagtgcgtgaaaatctgagcaaattactcaacaaaattgatttcattgaaacatcaaggaatagccatgatgttatggaatggttacacggcaaaataaaaggtcgtcgtgtagtgatttgcttatggaacagttgcagggagtataaaaaacctgtattgcaagtcaatatctatgatgacacttttaaaagtccagtaaagtctaaaagcgatttattagaatcctatgagataactgtaagtggcaaagtatcacgtaaagaggaaacaccatgttaatgactggtatcactgcaatcattgccattttcgcgctatataaggcatataaagcctataatctagcaaataaggcaatcacacagcaggttagcaataaccttgtagagagctttctagggcgtctcagtgatgagcaaataaagcggctagatatgagcttcagatataaagcgaaaacctatcagataagtgacatcttcaaagatgattttcagttagtcgatgattataattctctgataaatgctttaaatgtcagtgacttaaaagactactacgctgtaattgttcaagagttgagcaaacgaaaacaaaactgttgacactggtttttagatagattaaattacacatcaacggggagggaatgaccttccccactaaataaagtcctaaactggagattcaaaaaatgttttgtgcaggtgataaagttgtctggaatgatatagatgacggtttatgtacgaaagttgttgttatcttagaaattgattatatcactggtgttgcaactgtagcagataatgatataatctttgatgtcttaattagtgaactgtcaaaataaaatccttaaactaggagcttttaaaatgtatactactaacaacggtcgtactttaaatgtcacactacgtcactatgtgaacggtgtaatgcacttcgaggatttacaagctgaacagcacattttggattggcaagttgcaggactccaaaaaactgctacaggatacgggaaaaagattccaaccagttggaaagtatactacgaaggacgtttgcgcagaatttatcaagatgtgcaaagcaatagcgcatcgagttacattatagtaaaaggcaaaaaactgcatttaatgtaaagtactttacaaggggcttatagttcgataaagtaagccccagataaagagctttactactcaatcctaaattggagaacttaaaaatggctattaataaccgtgaattatcaattttaaaagcacgtttgaccgtcaacaggattaatgttctgacatcatcagcgcctgatgaaacattgcacaacattatcgggaagattcaaagtgttatcttagacgttgaaaatgtaaaaagttcattggctgacgttgcatcaggtatcacgctagacggcgcgcaatatgaaatgtcagacatgttaggcaaatctaaggtaatgaataaagagttagatttgaaaatgttccgatttgctgttaaagtgtggttatctgtcgagtatgatgctaactttgcaatcgctgactttttcgccacttggttgcagcgtaatttgtcaaatcacgattttcgtaatatctgcgatgcaatttatgcagaactctaaaaaattgttgacacttatttttagatagattaaattacacatcaacggggaggtattaaccttccccactaaataaagtccttaaactaggagattcaaaatgatgaattttttaaatatccctgatacttgccgactgttgacgcaaccagaaaatttaggcaaacctttcctgtaccatatgccagacaatacaccaaaaacatatagctacacagttaaccgctttacgggttacgctgcaaaaaacggttacaaggttactactcaaaaagtaacgctatttacaaatatcgaagagcttccaacaatcgctgtaaaactaatatttacgaagaagacgcaataaagtactttacaaggggcttaaaagttgatatcttaagccccagataaagaactttaccactaaatcctaaactggagattctaaaatggctattattaacggcgtatctatcgagactgtacacatcaaagacattaaagccggtgatacagttctttttcacggcgtagaaaaaactgtcactgcaaaggatattaaaggggattctttcatgggtaggactctcttcggtgattcttattgcttaggttatcttgcagttttaaaagttgtcaaaaataacaaataaagtactttacaaggggcttaaaagttgatatcttaagccccagataaagaactttactactcaatcctaaactggagattcaaaaatgaaatgttttcacggtactactcaagaaaatttcctcaacctgattaataacggtgataaaccatcaggcgcatggaattgttcggatatggacggcaatttttacgtgtatccagaaaataaaatctatggcgacgatgaggaagaaatagcatcagagggcattcagcaagctctaggtaatgccactatcaccgcagcttttcagatgaagactcaaaatattgttatcttagaactcgatattccagaggatgagctaaacgatgattattcttgtgataatatgtcgagtgtagcaagttttacagagtactttgatgtaaattggattaaaaaggtctatacaacagagtttaacgctatgtattcacctttttgtcttccttcattggataacccaaatttaaactatcttgatgagtctctggagcttcttgctaaaagtgttcaacagtcggacagtattcaggttttttgtgacatcatggatacactgacaggaaacattgcagaagaggatttaaagagctttttctaaaaacctatgcaatcaccttataaacactaatcagccccttgtaggggcttttagggggcttaaatcatgcttacaaccgtttaccttatcctttctatttgtaatggccattcatgtgattttaaagggcttgaggagttcacaggaagtaaagaaaatgctattcaagtttgccagatagcaaggcaagactatcccgcaagtgatgatattcagtgttactttaagacagaagatgacgacggaatctatttcgatagtgttgatggtcagtatgagattatcattgaaaaagactagacaagcctgataaaagtctgtaaattgtcaatcaacggggaggaaatgacctcccccactaaataaagtccttaaactaggagattcaaaaatggcttatgtaaccgtaattaccgataaagctggcgcatcttggtcaactcaagtaagtgataagatgtcacctatgcagtgcctaaaatactttgaacagtggaataaaggcgaggacacaagcccctttcaagtgatgcagattatccatgtagacaacgaagggaataaaacgacactgaatagtgagtactatgcaagccgttttgaaacgagaagtaaaacaatgaagcttttacgtgaatccggttatgcccatatcgccgcgttaatctgggatgacctgctaaaaagtcagcgtatcagctatgtaaaacctgaaaaaatattcatcagttaatcaataacttacaaaaaacttttaaaaaagtgttgacaataccccttgacattggtaactttgttcgagggggtttatctaaaagggattcacttaaaagactctttatagataagctcaaaaaattacttgcaatcagctatcaaaacaagtaaattactaatcaacgggagggatgacctcaccactaaataaaatccttaaactaggagagtcaaaatgaacaagtttaaagctatcaacttcatccgttcaaatgctgtaatgtctaagcctgtaaaagatacatacgaatttcgttgtaatggggtacactttgcaaccatcactaagtcagaaaacggggcttattatgtacatcgtcgtaatgttgcaaccgttgtagtctctcactttatgcaagctgtagcggaattattaccgttattcttagagatttacttagatgagtgtaaaaacgttcacaaccacgttaaaaaccttttaaacggttataaaatggcttatgaacgttctatcaagtccttaaacaagttctataaagcccctgaaaatactgtatcaatagcttatgatgtaagcggtgaaatggtcaacctctccaatgtagaacaaattggcctttatgacttacgtcctaacggttacgctaaaactctcggtgaaggtttaaccttcaatatcaagaacttagaaaaaactttaaaagatattgaaaaagatattgacgagacaatcaaaagtctgtaaattatcaatcaacggggagggaatgaccttccccactaaataaagtcctaaactggagttatcaagatggaaaacgtagaattaaccacactattgcagattgcaaccgacaaagaagatgtagcattatttcaccgtatcgaaagcgatttgttcaatggtgaaatcatcaaagaatcttatgaagttgtcggattcaaagatggtaaagcgtttggattctttgacctcgacaaaggcaacgctgttacaaagtatgatgttttgtcttgtgagattcttacaaaatacaacgaaaaaaggcttgcaacatacgccaatatgcagtaaattactaatcaacggggagggaatgaccttccccactaaataaagtccttaaactacttcctaaactggagaatctaaaaatggcaactatccgcgctactgttaacgagttcggtttttcctctctggatgttaaatttgacattgcaggcactgataacgcctttgaaatggtgaaaggtcttatcagtcagaccatcaaagattataatccttcggatgagttctgggaggaaacaaaacaagaagttctcaaggatggttatacttatcactattggggagaaaaaattgtctccctgtactacaacaaacaagattataactaatcgacgcagcggggctatttagccccagatacctgaacagcccctatttaaaggggcttttttacgtctatcgaatccttatacaatccctttccctcacctttctagaatctatccactgattcgatagaatctatctcataccttaaaagcaataactctatagtctattctatagccttttatcaaatcaacttaaatacaagcccataacccgaaagggaagggcaacccctaaaacgctctataaagctctataacagccttatcaacctaacttaagggattgcattaacctatcttaaaaagctctttacagggctttatagaggcttaaaagaatcttataagggtgtaagctttatcatctcgttagttatcttgtgaactactaaaaagggcttgttattgtcttaaaagtatgttataggggctataaagttactttgaagtagcttaaaagttacgtaaatgctagcccccttaacaatttcttaacaatccctacatagttatgcttttaagacacttgttagcaacttgttagccccttgttaaatctctgttaagaccacgtaaaagccttgtaagagtctttgacatagccttaaaagctcctgtgaagccctataaagagttatctgtacagttggggtgtaactactaggaagagacttaaaagagcttataaagagctatagagagcttttaagaggtatgtttaaggaatcactaagaaaggaaagggaacgataagaaagagatagtgaccatgttaatagctagttaaaaagattaaacacttgttagaaacttgttaattatcttaacaaaataactctttaaaattaacttgttagctcttgttagtcatctgttaagattctgtaaaacctatgtatcaactgttaagaaactgttacagccttgttagttctgttaagaatctgttaagacctttgaagagttgttaagaagatgtgagaactgttaagagattgttaaggatattttaaaattttgcaaagtacttgtaagggagctgttaagggctaccaagcagatatacatcctaatcagatatttttgagttccttcaaagaccctaaaaaatgacttgacagtcttattttataaaaattttaaaattctgaataattacctacacagtcttttaaaaatacaggtagaaacaggcttataagataccttgttagtaccgtgttagaccttgtgagataaggtctgtaaagatattcctgttaagtttgttaaaacagactatatagaggcttagtatttcctagtgaacacctatagagagtgtcataggtaacatagagattaagaggtgtataggagcttctaagaaggtgtagagagatgtttaaggtgattgtataggttgttaagaaggcttgttagaagagtgtttagatggctatttagaagggctatacagagggctattaagaccactagatagttactaagatactatatagtactatataactatatatagagagtaagattaagaaggttgttaagaaggatagatattttattcacatatgatggtgacccagaggttgggacaaccgataaaattttataaaaattaaaaagcccccgattaaggaggctcttaagatattttaaatcaggctttctaaaactgtcttcaatgatgacttagttgtctgataaaaatgagtgaatccattgttgttatcgaatatctgcatcataacagcatctcccttgattgggtacactcggatgtcatagtgaaatccattaaagacagcagaaccattacagaatacactaaaatcatccactgagatatactcttcatgacctgtgatttttatacagacacctttactcggtctcttagggttgatatcttccataatagacatactctgtccaccaagagataccatagtgaccactttacggttcttctgtttaaagtacattttaactttctgcaaagtgtcttcatagaacggataagacctttctacaaagattccttcaagtggctctttaccattttcaacttctctgttatgccgtacttcaaagataatttgttcgaaccagtcaagattttcttcggagagtgcttctcgaacatcactccagtcaagtactaggtagttaccttcaacttcttttaagttatctttttccatatctatcacaccattatcgtaaaacttgatattctggtactgcgaatttattactattgttcttgatgaggtcatcctctgaaactaaaaatgtaataccacataagtgtctgtaacgactatcatactcgaatatcttaaacagatatttaccatcatgataaacctgttgcacttcaccttctgcaacaatctcgcctgtaataaaattttttagtttatacacattaccaactttaggtactgttttcatctcaggctcttttagtgctacacctatttttgcatcaccgagtaaatgagaactctcaaaagctacgtgtttcatataagcttctgagcattcccagataaaacagtgtacaagaaccttagcagcatgtcttgaacagtttaacttatcacaaatttctgtgtacatctcatctttagtcgagttgtcaagcattgctctttgcactatctcttttactttcataagctctccgaataaccctattaagcatctctaaagtcagttttccagactctaaatgtggcataacaagctttaaagctttttcaacatcatgcacaacatctattataccttcttcataggcttctacaaggatatcttttacgttttgttggttatctttttgtggtattgctagacgcatattaaactcatttgcttgagggtggtcactaccacgccagtaaccatctttgttaaagctgatgtagtcaccgtggtaaaagtgtacaccattagctttaaacttctccagcacaaggcattgaattgggtactcagtattccactccctgactcctataaccttcactgttgcgatgtactcctgtgtttttgcatctatgatgtcaagttcttgaccagctatgatttgcatattcgctaccttctaagtttctacaaaggggctatcaagtaacccctcaagttattactcagacttttcagtcttcttgtcggatttcttactagtcttcttctcagctttctgctcaggctccttctcagaagttgccttaataaccatatcaagaagctctgataagtcactcttcagtaaagtaacaggctgctcattgtagaagtttttgtcaagataaactttaaactcttctatagaaggaaatccaaaaattggtacaggtgtcttaaccattacttagtctctccataagcatctttcaagattgccttgttaatcatttcagcgattgcaaaacgaacacatacagctataattgtaggttgcattctttacttacctcttcaagtttaacttcagagtccttaagtcgtttcttatgaactctgaatctcattagtgagatgatgataccaactaaagggacagcatacaagtaccaaacatcatatacctttgcagtaatctgtgtaatggtcaaatcttgagctatggaactgattgctgatactgccagtaatgttgccaagaaaaatgccagtaccattactgtactcaaacaacctctggcatggtgttcatgcttaatgcacttattcttttcgtcaacaactctgttgtagtcgtttgtaaagtctctgtagaatgctttcaagtattcttgagtgtagccttcaaagcaacctgtaccataccaataatcaccaccaaaatgtgctatgataccggtatctccatttggttttgttactcttagggagacatgatgaccagtcttctggtaatctatcttgtatctttcttcgtaaatcattttccaatccctctcaatcgtttgttccactcaaggtcaacaagtctttgtaggactttacctctaggtgttacaccttttaaatctgcgtcatagcaatggtaaaaatcaccttcagcgtgtctgccatagcatcttcttatactctgacttctacgatattgttctaatagagacagcttgtagtaatcttcaactgtagttagttttttcattctttcaccatagcatttctgaactgacgttgagtttctcttgtaatttcgtctggagtaaaccctgctttcagcatatctctgaaaagaacttctaagtgattcttttcaataatgtaaattgcattctgcacagatgttttaagttccttcaggttactgctaatttcttcgcacttgttgacaatctctgtatcaactttcttaaagactggcatggtatatctactaccaccattagcagcatccatgaaaaatttaaaagattcctcagagtcctttaaagcaatccctataaagttcttgtaagcacctgtgaaagctaaagattcaatctcagcagttgagtatacagttcctgcacacagtcttgcggaggtactaaatgtgtggtttggtgttatgtcaaagtctaagttcttcagtggtcgaaaataaactatcccattgtgtgagatgtgtgttacaacagcctcaacactctttgaaggattctgtttaacatacaaaataattttatcgtgttctttcactttagccatatctattctccagaaactaaaaagggaactacctctcgatagctcccatcataatcaatattgtatactcggtcaagagtattttttaggcagtaatgcactgcgcttgttactctcttcactcaacagtgcgataaattcatcacctttacccttccacggactaaatgtagggatgtgttctctgatagcatcaatcactgttttcagggcgttattttttaaccaccaatcagacactgctgatgctacactgctaataagcaccatcatctgtgaagattggctcagagatacatggtgaaatcatagtggtgatgatagagccattattcatgtaatatgttcttttttcaaccttatcaagaagcttatggaggtctgcaaagagactcacaacttctttatggtcaatatctttttcaagatttgctctgtatttcagcagtgcagcatcaacctctttatcattgaatttgaagtattggacttcttcattcacacatacactgatattgttggtgtttatcgtcaatttgcacagtttgactggagaattgatgttagtctgaaaaagtgctacacagccttctacatttccgaaatgttttactaatgtaacttctggttggatatgctcatgttcttctgacattacagtttcctcattaaatactttttaagttgtgtgttaggtttctcaaaaacttctatagaagatatcttaacagactcaccacgcataccagtatgtttgaatatgtagtactctagcatccctatgtactttgctggtaagtcatgcttgacatataccgtcacagaatcttcaaactcaacaacctttgtaccatctttgtagttaaactctttaggatggttgtagttgtctatatcgtacattaaaaagagtcttttacgttgtaagtcaagcttatttgttaaagactctttaatatctgagaagtttttgtacactatgacatggagcttactcatcgttaaactctctgacgaagaaatagttgactttgttattacctaccataaggtcaaagcaggtattttcaaaaggtttgttgtaggcatcgtatgaaatatatgggtcagctacttgcttagtgcttaacttcttacttgatgtatttaagaggccaccatacagcttcttgatagcctcttctacaacagaccagataatttcgtcagtaatttcttcgtacaggtcaagttcacgctctagtgagaagtcacaaatagggaaaacaacctgtactttactcattaacgaataacctgtgagacttgaattgcttcagttactgacaagtcacctactttctcttctttgataaaagtgaatgtgagttcaacaggttcttgctcttcttgctcaactggaatgccattaagctcatcgcggattgattgagttgtccagtcttctccaccatctggataatggatggcgtaaagagggaactccatatcataccagccaaatacagggcaaccatctgcatcatctcgcattctgcgaagtgtgctacctgagcctgtaaagcgaagactaatatttccattacactcaaaaatcactgcgtcggcaagtccagcatcttctcgaccatcatggccaaaagtgtcaacttttacttgaatctctttattcataaaaacctctctctgcttgttctatttttgctcttaaagcttcgcaatgttgtaaatgatatttagcttcttgttcatgtgttgctaactgcctttccagaacagatttatagtaacctgcaagttgctcttttgtcaatgggtgctctgaaataatatcaagtccactttcaccaagagggttgaattgtccatgttcgttgacattgtaagtctcaaacttgaacaggccattgttcacagactcaataggtgacaactcaacaagctctacagtaaaccgtgtatataccttgtcacctcttgaaaccacattgttcccgacaatgcgtacaagcgagccattacgagtgtacttaaaagttccaaagtcactagcccaaagtttcatagctttctccttagaaaataaaaaaggctcccgaaggagcctatgaagatattactttggatatacactgtcaagataaatgtcagcttccatcctacgtctgtttttcaggccgtttgaagtgaccttcttacctttgactgtaactttgttccaccactgcatagcttctgcacaacctaccttattaccatcgttgtggcgcttgataaatgttgagtcctgcatagctgtgataccgatgttgtatgtttcacttacaagtgcatcgaactcattctgagaagttggaaccttgatagctttgttcactgctgcaacgaacttttcaacatccgcgagaagatactgttcagcttgttcagcggtgattttcattcccatcttaacaggcttaccagcaatacggatagtcccatatccaattgtagggattccagcagaatcttcgtaagcctctagcttaagaccttcaaagaacttaatagcttctaaaccttttcttgagagttgcattatgcctcccctgctgggttaactgtaactgttgctgcattggatgttacagagccacctgcaccagtaactacacaggtgtaacttcctgcatcagcagtggttgcaccttctttggtgtaagttgcagtagttgcatcgaggatgtcttcctcacctttcttccattggtaacctgtcgcacctgttgccacaacattcaacgtcaaggtctcaccttcagtgatttcttgattggttggctgttgagtgattactggaagtgcatttacagtaactgtggctgcgttagatgtgacagagccacctgcacctgttacaacgcaagtgtatgaaccagcatctgttggcagagcaccagacttgctataggtagcatttgtcgctgaaggaatgtcagagccatctttcttccactggtaacccgttgcacccgttgcagtcacttccaagctgatgtttccaccctcattgatggtctgactagaaggttgctgtgtaatcacaggtagtgcgttaacagtaactgttgccgcatctgaggtaacacttgtcccagcctctccagatacaacacaagtgtataaaccagcatcagaaggtgctactgactgttttgtgtaagttgcagaattggcactagggatatcctcaccatccttcttccactgatatccagtggcgttagttgctagaatgcttagagtaagtgtgtcaccctcattgactgtttgacctgttggttgctgagtaattgctggtggcttgatagcatccttcagcttagcattcaacattgagaacggattaactcttgccaaccattcacagtaaatagtgtcaacatctttaccattgatgattgcatactgcaaatccatgaagaaatcagcagttctcatctgtgcaccaacactgtaaaggagttcatcactaaaaggaactttatagtctggtttatagtcaaacttctcaacttctgcaatatcagcttcaggccaatacgagctataggtaagtggtaaaatagcctcttgatatgttctaaaatcatacctcttaccagcctttacgtttgcaacgaagcccttcacaaactctttgaagtctgggtaagtctttgctttaatcatttcttttcagcctctaaatgtgcgtttagtctaaaataacctttgtgattcggagatacatcaccaacaacaatacctttaaccttataacctttaccaattacatactgaatatccataaggaatcttccaagaccaacaatatggaacattggatataatagattctcagagtctggaatattgatttctggtttgtaggtgcgttgttcgcttgttggtgtaatatcttcttcgggccatttactgagtctccatgtcaaaggtgttgggataacatcattaaggttgtacctttcaccattttgcacggaattgatataggattgtaggaagcaataaaagtcttttgcactcttagctttcattaaatatccttataaatagttaaaggggcatcagcccctattcacacttaagctttggtcttaagtaggttcttaagttttttaagattcatactctcatcaaacttaaaaccaaatccattgacataatcaatgaactcggatttcttactgagtgacaatgcgtaactcatatcaaagttcttacttgtttctgcgtctggactagagttcacatcaaatgaaatttctgtaggctcttgcttactttttctcatattatcaatcaacaaacatgtatagtttgctggatagaaatttacactaccatcaacgtagctgtacttattattatggataaggttggaaaataccttaaggaatgtttctgagtcgtggcaaacaagtttgaattggctgctctctccaagcgcaactaatgagtacggactttcactatcaaagtcataatttggattcgatgacgtcactgttacacgcatcaagttattagcaatagtcttctgtttaaaagaattttgaacatagtgaagacccctctcacactgattgactagctctttaagtaggtcatttgcatatggtgagttaattgatacagagccgtcactattgtaattaaaacttactgttaacatttttcagttctcctgcgatttataattttgctgcaatagagaaacgaagtaaactcactggaagaaaccactaagtgttgttcactagattgagccattgccctgaaatacattccagtcaaagcatcatagtgactattctttccaattgaggttatatagccatgactctttgcagtttgatataaatctataatatctgcgatttctaattgggaactacacatagataatcctcttgtcttctgtaattttgaaagaacaggtacagaccaatttcagaggatgaatagtagattaactctaccctttttgattccttacagtgattggtattcttacctttcttcggtttatttgattttctcattaagtctcctttgtagaacaaggcagacctacagctttaagtaagatggtacagtaatccatacaacaagtcaagaactattgcaagttaaaaaagttgttgacaaagtacttgacaaggtgtaggcttataaatacttagaagataacttaaaaggttcctctaaaaaggctatttaaacagatatctttctggatatctatcttttatcttaaaagccttttaagaggtcttataagaggcataccaatgaaaaacagaacaaacaaaggccagtttaaaaaaggccagtctggaaacccatctggaagaccaaaaggctctcgtaacaaaagttcactcgtaaaagctcaactgaccattgataattctgctgagtttgctgcaaagttgtttgaagcgattgttacaagggacgctgctaagcttgcagagtttgggttaaacacagacgacgtaaccatcaaaagcatgattgaggtcggtaaaactatcatgacccactcagcaggtgaaatgaaagcaattgcagcagacactaagaagactcctgataatggtggtcagtctcagacagacaacaaaccaacgttctctgctgttgcaacccttaaaaaataattttaaaagatgttgacagactctataaagttgctctaaagtctgtcacatcaaacaacaaatgagagaagagagtaaaatatgagcgaattatttaaacatgcgcaccttcatgcaggtcgaactgaaaatggtgctgtaaaccatacttcatcaatgtctgctcttgtagacttttacaaagctgctggttcaagccgtagcaatgtagaaatcttaccagacctgttctacaaagctttgcgtgaggatgttgatgttgcagttcgtattttgcttcatatgcgagatgtacgagaaggtatgggtgagcgtaaagccttcagaactgttttgcttcaagcaattgaagacaaagttttagagtctacgcaggttcttcgcattatggataagattgcagaacttggtcgttttgatgacttcaaaatcttcgtaggtactcgtttcgagacagatgccttcaaacatttagaagcagcattactagaccctgcaacagcaggtttggcggctaagtggttaccacgagtcaaaccacgtcataagcagtttgtaaaacgtttctgcaagtttgccaacttgagtgagaaagagtaccgcacactgttgtctgcactatctgatacagttgagcaaaaaatctctgctaatgagtttggtaagattgactacagcaagattccttcacttgctgctgcacgttaccaaaaactgtttaaccgtaaagatggagagcgctacaaagcttacattgagtccttatcaaagggcgaggctaagattaatgctggtgctgtttacccatacgatgtgattaaatctgttaagtatggtaatgcagatgttgctaatgagcagtggaaagcactaccaaactggatggcagaaggtgaaaacatcctatgtatgactgatgtttcaagctcaatgtcttgggtgaattttggttcaatcactgcacttgatattggtgtatcacttgccttgtatgtagcagaacgcaatacaggttgctttaaggatgagttgatggtttattcaacaaaccctcatttcattgaactgagtggtgatttacgaaaccgtcatcgtcaggtgatgcgacacgttgaatatggctcaaccaacttacaagcagcttttgaccgtattcttgagacaggtaaaagaaacaacttgactcagaaagatatgccaagtaagcttatcatattctctgatatggagttcaatcaggttgatggagctaacggtcgtacaaactttgaagcaattcagagcaagtacaaaaaagctggatatgagatgccacaactggtattctggtacttagcaaaccgtaatggtacttgcgaagtgtccgttaaggataacggtgtagcaatggtatctggtttctctccagccactttaaaagctctgcttggtggtgagaagtttgacccaatcagcgtaatgctcaaagcagtaatgattgaccgttatatctggtaaaaaagttttaaaaagggtattgacaatgtgtttaataccctttaatatgttctacatagaaacgaaatgagagcttttctaagatactgaaaaatattttaaaaaagttcttgacaaccactaaaaaataatgttaaagtggttacatagagtttgaaaagtttatctctgtttagctcagcttggtagagcgttccgtttggggcggtaaggccggaggttcaagtcctccaacagagaccaaattaatgttccagtagacaaaatggtaaagtcaccactctttcaaagtggatatctgagggttcaaatcccttctggaacgccagttttgacagaagaccaattacagcaaacttaatcttattcatctgaaggtaaatcggacaaagaagagatttggtctggtcattaagaattgcgggtatagagaaagggcgtctcacatgtctcattagcatggtatcggcaggttcgactcctgcacccgcctccaaatttacagaagaccgtttacagcaatatttattaaatttttagcggaaaaattgaaagatacggtctggtaatactaaacatggttaggaagcacataaggtatgtgcggtcgcctgttaagcgaatggcacagggttcgaatccctgactaaccgccaaatttaggagagaagcgattgctcaccgatttgtaaggtgcaagtccttagcctgaatagaaattaaatgtgtcgttatcccgtagatggtagcggtggggactgtaaatcccttgtcattgagactcggtaggttcgactcctacacggcacaccaataaaggttatgtagtttaatctggttaaaatactcccctgtcacgggagatgatgagggttcaaatcccttcgtaaccgccaattttgagagggctatttagcccctcccttaaagggttcttacgagtatcctttaaaggagcggaagaccaaatacagcaagtacgtatgatattttcaagccaatttaattttgaaaaatttaaaacttggtctggctcaacaaatttacagaagaccgtttacagcaaaacttaaacaatctatttctccggtaaagaaaaggtgaaggttcgattccttcactcggcagaggtcgagttggtgtaatggtagcacttaagatgataaacaacggtctggtaattatctttgtagataagttattaagcttacctgtgagtatgcgaccacaaagggtgaaagggtaaattctagatttaaacccaaaaaggcacatcgctacagaccttaagcaagtctgggtagcttatctacaaagataatttaatggaagtgtagcagaatggtgatgcggcagacttttaatctgacaggcgatgggttcgaatccctccacttctaccaatatggttcagtcgcagataaggtaatgcaggggtttcataagccctatgaatatgggttcgattcccatctgaacctccaattgtgggttagcataaatggtaatgcaaacggctgataaccgttagaagagggttcgataccctcacctacaaccaatttcaataaagttgttgacattgagatgtgactactttatagtaaccttagttttcgttgcgtagcgtctattttgcaaatttaaaataaatgcaaacgacaatgtttttctggcagtagcttgataggctaaacaccagtgaggtcttccaatccctcatcaaagaatttggcgtactcgcccacggtatgattaataaggtgggcatctttaagggctttctaagagagtctttaaagataattacggggcgtatctcagcggtcttctaaaccgccgttaaccgagtaagtggagtatgcgggttcgagtcctgccgcctcgaccaatttagttagcagattacttagacgacctaagcgggtcttcctgtagggaggtggtctgtatctcatgttttccagaacatgtaaataatctgagaaggggctttacagtgttgagataatacggtaagcttcgaattatagtatgggacgatgttaagactctaaggcatgagcaacggcctccaaaaccgtttcaaaggggttcaactcctccgtcccatgccaaagtcttattaggggtaggtagcggctaatggcagccaaacagtcttgaaaactgttgccactgtagagatacggtgagggttcgactcctttacttaccgcctaataagatttttaaagccaagcttcattcggatgttgctttggtatccctcgtgtattgtcgtacacactgataaaacacctagtaggtgtcacggagaagagataatatcaagctctccaaaggttctagtcaccggattaaacaagactatgcaaagaccttttaggtctttttagagggcttctaaggttattacggcttgttgtggaaccttgttgcttaggttcttagaagttctctaaaaagatttaatgggagattgacggtaattggtaaacctaccatccttagaagttggtgtttgagggttcgaatcccttgtctcctaccaaattaatgcaggtgtagcaaaatggttatgcggctgactcttaatcagtaagacgatgggttcaattccctccacctgtaccagacattggggtcatagtttatatggttaaaattcgagttttgcaaacttgggaactgagttcaattctcagtgactccaccaattagtgcatccatagtttaaacgggaaaattacagtcttccaaactgaggttgagggttcgattccctctggatgctccaaattaatgctgctttcgtataattggctattacacatcccttgtaaggatggagatgcgggttcaagtcctgtgagcagcaccaattcagaggtcaagtgaaagaccgctttgagtcaactgaagaccgtaacaaattccacggagttgagttagcggcacaacttcagacctctttcacactcgcttagtttatatggtaaaacatcacccttacaagatgaagaaaaaggttcaagtcctttagtgagtaccatgttccagtatcccaattggtagaggatgcaagctcaaaccttgtactagtgacggttcgaatccgtcttggaacaccaattttgcaggattagttcaaatggatagagcaacagtctacgaagctgttaataggggttcgaatcccttatcctgcgccaatttaaagaggctatttagatgagatacttaaagtatgcttcatggattttcttagctttgttagagccatttgcagaaatcttagcagttatcttagcaccttttgtagttccattctacagtgagaagaaaggccacttacctttcggatttagatggatggagacatatgacaacccaattgatggtgacgaaggtcatatcgaacgatgggctaagattagaaagattggtaagctgggtgtctacatgcagagagtcggatggctctggagaaacaaagcttataacttctcttaccatgtattaggaagagatgtaaaagatgttactaagtggaaaggtaatatcaacgtaagttctgaccctgaagataatcagacaggttatctcctaatgtggaacagtaatgcttggggattattcgcttttatcccatcaattaaagtctttggtaaacagttttactggagaatttatattggatggaaactaaaaagtgttgtcccagaagaaagagcattctcaaggggaagagttatgttggcattctttattcatccactaagaaagtaaagatttaaaggggattagtttacaaggttaaaacctcggtctttgaaatcgaagaagttggttcaattccaacatcccccgccaatgctccattactccaattggcagagaggccagacttaaaatctgtgttatgtatcggttcgaatccgatatggagtaccaaatttagcggtatagcataactggcaatgcaacagtctctgaagctgtcctattaaggttcaaatccttatgccgctgccacttctaagggttcttacgagagtccttaaatgtggccttatcataaatggtaatgacccatgctgtgaacatggtctatacgggttcaaatcccgtaggtcaccccaatttatagtccaagtagcttatatggttaaagcgcgtgtctgaaaaacatgagaagagggttcaaatcccactggactaccaatttcaaaggtgcttaatgaaagagatgacagaacaaggtaaggagatttttaatctcttaaaaaccggtaaagggttttctaacccacttattactggtgcagcagttctcggtggaaccgtagctgcttctacatcacttgtaagctctattagttctgtaacagaccctacaatgcaggagaaacttgttgctgctggacttacaacagtccttctaaacagctttacaacaagccttacaagcactacagcaactactaagaccctgacagactacggtcaaaaatctattgatgagttttcatcacgtatgcaggtagcaaaggggtattccaatgttatgggtgcagcaggagaacaagttggttgtacaccgttcagtggtattatgggtgttgctacagaatatggtcaaaaagctattgatacaattaacagtacactagagagtgttaacgatgtattaagtgacttacaagacgctattgatgagggtcttgatactatttctgatttagctaatcaggctgttagtaagattaatgaaggtatttcaaagattacagcttatgcagatgaagttgtgcagatgattgaagaagaagctgcccttattgcagagtaccttaaaacgaatatcaatgggttcttagcaggtatcttaccagactggtttgacgatgcctgtaaaactggtgtaattgacactattgcaacaccagaaatgaagaacgcattacagaaataaatggaagattaaccctaaaaggtaagggagcagtttgctaaactgccagtagctgagaaatcggtgtaccagttcaagtctggtatcttcctccaatttgaatccgtgacagaaatggctatgtgcctgtctgcaaaacaggtttataagggttcgagtcccttcggattctccaagttattttactctctctcaattaaaataataataataatgccctgctataagtattcttcctttcgctaccgaagagtatttttagacagggcttttttacagagttatactttataagaaggcttatacagtatgtctctgacgtacattgtggtttctccttgacggtctctgtgccgtcttttttaaggggaaacatggttaattatttggagaaaacataatgagtgagaccgtttacaaagaactttatgaagctaataaaaagctagaatttatgcagaacactatcatggcaattgcagatagattatctgtagcaacaggcattgatattaaagaagcatctatggatgcactccttgatgctgttgatgaaaagttcgaagttaagaaagaagagactgctacagactctgaataattctatttgcggaggcgcttaatggacttaaatgctgttaagcagaagcgagtggaagatgttaggaaagtcctagctggagagttggggctttctgatgaagtaaaagaaattattaaatcattcggtaaagacccctctaaattccttccaactcaaattctgactttattaagatacacaccagaccaagttcgcctcatctttaaattgatgactgataagaactatgtagcccctcaaccgggttctcaagaggtctttttaaatactaatgctgacttggttctctatggtggtgctgctggtgctggtaagactgccgcattgttaatggactctttaagatttattgaagaccctaactataatgctgtatatttccgtcgaaatacaacacagttacaaggtggtttatggcctgctgcaaagaaactatttggtaagtttggtgggattcctcatgagcagaaaatgactatcacattcccttctggggcaactatcaagtttacctacctagaacttgaaaagcacgctgaaggtcatcagggtattgaatactcagcaatctactttgacgaaggtacacacttctctgcttcacagatttcatacctacagactcgtctgcgttctggtgctgaaggtgattcatacatgaagatttccatgaacccagacagagaccactttatttatgattgggtagaaccattcttagatgaagaaggttatccagaccctgaaaagtgtggtcgtattcgttggtatgtaatgaatgatggtgtgatggtttctgattgggagagagacaagattcttgaaatgttccctcttgagattcctcaaacatacaccttcatctctggtacgattgatgataacccaattcttgacttcttagaacctaagtaccgtggtaagttggaaaacaacacccctgtaaacgttgcaagacttcgtttcggtaactggaaggctcgtgcagaaggttcaaactactggcaaagacaatggtgtgagattgttgattcactcccagaagatgtatttgatgttagagcatgggacttagcagcaactttaccatctgagattaaccctaatccagactggacagcaggtgttaagatgggtaaatctaaaaaagacggttgctattatatcattgatgtagtaagatttagagatagaccctctggagtcgaaacacaaattaatttgactgctgaaagtgacggtaagcgaactggtatttttatccctcaagacccaggcgctgctggtaaatcctacgcaacatccctcatcaggaaacttgccgagaaaggctatcgtgcaagagctaaaccaacaaataaagataaagttacccgctttgcgggattttctgctgcttctgaagctggacttgtaaaagtcttgagaggtagttggaacgaagcttactttcaagagcttgaaggcttttgtggtgatggcaaaactaaagatgaccaagtggatgctaccagtgatgctttcaacagtcttaacgaagttaaattattcaagccaccatcaatgggtgctcacacagacttagtgagaggaaacccatatgaggggcttagacgttgatagctaggtgagaagaatggcagatattacagaaacacaagaaagcttaccaccatttagaatgggtgaagtaggttctttgggtctgaaggttaagaatggaagaatctatgaagaacctcgtcaggcactaaggttccctgaaagtattaaaactttccaattaatgatgcgtgaccctgctgtagcagcatctgtaaatattattaagatgtttgtcagaaaagtcaactggagattcgtacctccaaagggaaaagagcaagaccctaaaatgcttgaaagagcagacttctttaattctttaatggatgacatggagcatgattgggcagattttattaattctgtaatgtcattctgcacttatgggttttgtgttaacgagaaagtttataagaagcgacagggtaaaaaaggaaagtaccagtcaaaatttgatgatggtctaattggatgggctaaattaccaatcagaaaccaatcaacacttgataagtggtattttgacgaagactttagaaaagttactggtgttagacagaatctgagaaatgtttcacatattgctggagcaattaatcttggagaaagaccactaacaagaaaacttccacgagctaaattcatgctgtttaagtatgatgatgaatatggaaacccagaaggtcgttcaccattacttaatgcctatgtaccgtggaagtataaagttcagattgaagagtacgaagctgttggtgtttcaagagacttggtaggtatgccaaagattggtttaccaccagattatctggatgaaaatgcagaacctgaaaagaaagctttcgtacaatactgcaaaactgttgttaatgatatgattgctaatgacagagcaggtttaatctggcctagatatatcgacccagatactaaagaggatatttttgagttctcattagtctctagacagggtgctaaagcatatgatacaggttctatcattgacagatattccaagcaaattatgatggcatttatgtcagatgttcttgctatgggtcagtcaaaatatggttcattctctcttgcagattctaagacaagcttattagcaatgtcagtagatattttgctgaagcaaattaagaacgtaattaaccgtgatttagttgcacagacttatgctcttaatatgtgggatgatgaagaacatgtacaaatcacatatgatgatatcgaaactccagaccttgaagcaattggttcttatattcagaagactgttgcagtaggtgctttggaagttgacaaagagctatctaacaaacttagagagcatattggtcttcctcctgctgatgagtctcaaccagtatctgaaaagctttctccaaatagccaaagccgctcaggagatggttataagaccgctggagaaggtactgcaaagacaccttcagcgaaagacccaagcacagcaaataaggcaaataaataatggctgaagttatctctatttcaaatgctacacgagtgcattcgtataggggtgtccttatcattactgataagttatctgtagaggctggctcaagggtcagcctttcaggttatgttagtgatggtggaatctctgacgttttcactatttgcaggttacttgatgcaccaatgagtggaaaaccgtttatttcaggaaattgtagtgaaattgttaaaattccatttgacagttcatgccttttgggtgtaaagttatataactgcgagaataaacgtatcaatgttaatagcattgaagccgctttcattaccctcgatactgcatttcaatctccaatgacagttaataaagaaacaaacagacttgaatacattttttcacaaaatgattacaaagtacttgtcaaaggtaaagtatatgatatgattgtaaatgtggtagatgaatctggtaaccattcaacagtccttaaacaaaaagtaaggtttaattaatgggaacattaactattgatggtaagaataaaatcctcgccacgctaaccccaacgactattattttacacaatgtagacccaacggcagaccctacagcaaataaggttactcagccagtggctatttatttttctgaaccggataatggcttaattgcctcagaagacacagttaatattactgtcccagcttctgcaacggtctcacactatagcttgtgggatgctaacgataaatgcgtggcaactggtgctcttagcaaacctcaattctttgctgaagaaggtatctatgttatctcttcagtctctgtagatttaaacaaatagggtgagtaaaaatgagttcgaatatcttcagacttgctgatagattattcaaccaacctttactagccactgaatcattagctcactcagcagcaacttatgtgaataacagattgctgggtgatgtccaagcagcagtaaactttgataaacccaaaggtgaagcaagaagtcttttaaaagtaaaagatgacattgctattatccctattatgggtggtttaacccatcgtatgacattcattgatgcaatgtgtacaggtggattaagctcttatgaaggtttgcgtagaggctttgacgaagctttagcagatgagtcaatcaagactattgttctgcacattgattctggtggtggtgaagcttcaggttgctttgaattagcacgtcacattatggcttcaagaggccaaaagaaaattattgcttatgtagatgagttcgcttgttccgctgcatacgctcttgcatcttctgctgaagaaattattgcatcaccagatgcagatgttggttctattggtgtaattatggttcatcaggaattaactaaggcatttgaaaagaatggcgtaacaattaacgtcatcaaagctggtgagtttaaaggtatgggttcaccattccaagcactctcagaagaaagcaaagaaagacttcaaaagagaattaatgatacctacgcaacctttacaggttttgtagccgaatctcgtaatctctctgaagaagctgtaaagaatactgaggcgaatgtttattctgctcaggaagctcttgaacttggtttaattaactcaatcatgtctcaagatgatttcttaaattacttacaaggttctgaagaggctcctgtaagtttaaacgttaacaattcaggtgaagaaatgactgaacaagaaaagcaagaactagaagctttgcgtcttcaggttgctcaaatgaaagctaaagaacaggaagctgctttgtcagatttgactaataagatttctgcttctgctgaagcttttggatttgatgcaaaagaagctgcaacgactattttaggtgctggtcttgataaccctctgagtgttctgtttatgaatgctatggaaggtgctaaccagaaacttaatgaaactatcgcgtcccatgcttctgcaatggaagaaaaagaatcagaaattaccaagctgaaagaaactgctggtgctgttcttgaacattccaacgctatggaagaaatgggtaatgacggcgaagctgatttggttgaagaagaaaaagaaccagctaagaatgcttccgaagacaccgctgaacaacgcaaactggctctccagaatgctctaaaatctcttatcaaataaggaacacaataatggcatatcaagattttactaagttaggtaaaagagaacctctgaatgatatcattctttgggaacaggttaccccaacaggccactctcgtaaagagtacactccacaggcatctacagaatatcgtgtaggtgaagttctgaaggaagacggtactaaagtcgcagcaggggaagaagctcaggctgattctgtatgtatcgttaacttttattcagacctgcaactgtcttaccacggtcagttgaaagttgttggtatttaccgtgacgcagaactgaaagacatgcttactcttgagtctggtgttaatgctgagaacgtcaagaaagctctggctgctaaaggtattgatttcgtaccaactggcctgtaataacaataataagacattctggagaattttacaatgttgactaattctgaaaaaagcagatttttccttgctgacctgactggtgaagtccagtctatcccaaatacttatgggtatatttccaacttaggtctgttccgttcagcaccaatcacccaaactactttccttatggacttgactgattgggatgttagcttgcttgatgcggtagaccgtgatagccgtaaagcagagactagcgcacctgagcgtgttcgtcaaatcagcttcccaatgatgtatttcaaagaagttgaaagcattactcctgatgagattcagggtgtacgtcagccaggcactgcaaacgaactgactactgaagctgtagtacgtgctaagaagctgatgaagattcgtaccaagttcgatattactcgtgagttcctgtttatgcaagctctgaagggtaaagttgttgatgctcgtggtactctgtatgctgacctgtacaagcagttcgacgttgagaagaaaactgtttacttcgaccttggcaaccctaatgctgacatcgacgcttctattgaagaactgcgtatgcacatggaagacgaagctaagactggcactgtaatcaacggtgaagaaattcacgtagttgttgaccgtgtattcttcagcaaactggttaagcatcctaagattcgtgacgcttatcttgcacagcagactccgctggcttggcaacagattactggttctctgagaactggtggtactgacggcgttcaggctcacatgaacaccttctactacggtggtgttaagtttgtccagtacaacggtaagttcaaagacaagcgtggtaaggttcatactctggtgagcattgatggtgctggtgcagaggttggtgtaggacacgctttccctaacgtttctatgcttggtgaagcaaacaacatcttcgaagtggcttatggtccatgtcctaagatgggttacgcaaatacacttggtcaggaactgtacgtattcgaatatgaaaaagaccgtgacgaaggtattgacttcgaagctcactcttacatgctgccatactgtactcgtcctcagttgctggtagatgttcgtgctgacgctaaagactaatattcttaaggagggttatgaatgtgttatacaggcgacccagccaataaccctcttgatagagtaagaatcctctgcacagacaccaataatgatgaaattcttattgaacagtctgtgctagagtggttctatctagaatctggaaaggatgaaaagaaagcagccatcaaagctcttaaatatttactctttcaagtagccaagatgggagatgagaaggttggtggtgtttatttacgtaactcttccagattcaaatctctgaaagctgtttatgacgaccttgttaaaagctctgtttcaggactaccctatgcaggtggtattaatcagtgtgacattgatatgcgtcgtcagaatccttgctctgtcaagaaatacacagaatatggtgatgctgccagatacgaaggcagagattactgcaaccgtgttaatggcgtatttattatcgagcgagatgaataatggttaaaagggttattcaccccgctagagcaaaattagtcggggctatgaagaacttgcaaacggctaatgctcaagttgggtattttcaagaacaaggtcaacatagctctggtttttcttatcctgctttaatgtatttacaagaagttattggggttccttcagcttctggtaaagtatatcgtaggttgtttgaaatcactatgatgctaaacagacagaccttgttagagcagactaagaagaatctatataagcaacttagcagtctcaacgtagacccctcagataccttagaagcatttgcaaagaatgctcaaaaggcaattaaaagaggttttggtaattctgctatcctccctcctaacgcaccttccacagtcaagaaaaaaggctttaatgcacctcttgttgagacaggtgacttaagggataaccttgcttataaaatttctactaagaagggtattaagaaatgagactcttaaacagacacagctttgtagtaaagcgtaaagtctctgaagacggttattataatgatgatggtgattgggtagcttcacaagatattgtagaggttaactgtaaaggtaatatccagccatatatcaaaggttctgtaaagaatggtacacagattgctttaccggaaggtattagactaaccgatacaagaatcctgtatactacatataaacttagaacttcagatgatgtagagtggaatgagtctgatattgttatgattgatggtcatgagtatgaagtatttatgactatggattggtcacagcaattagcccatacatcccattatgaatatatcattattagaagggataaaatgaatgcagttagaaacagcagaacttgaaaaaggtctagttagaaccttagtggatgttattggtcacagattagctcgtgataaaaataatagaccaaacgtaattagagcttacccttctgataactcaaatgacaaaggtttaaaacctgaccagccatttattaccgtatattgtcaagatgctgcaacaccttatggttgggttcttgataagtttgttgaagatgatgtagtttgttacagaattgcttttcagattcctgtattaattacggtaaatgggaaaggtgctcacagtattatgcttgagcttaaacaacgattagagatgagttcagtcagggatttaattcttgaagagacaggagctacagtgctagacactggagccatcccaaatgactacacttatctcaatacagatttcgaaaattctgcgcctcttgttgtaactcttgtaaaaaattcagtcctgaaagatgaacgcggaagtattattgagcgtgtcattgttgatggcgagttggtttatgaagaaggacaagagccaccagaatatactatccatctagatgtagactccaaaggggtaaaataaatgtggaatccaattgttaatgtagatattacattgaacaccgcaggaactacaagagaaggttttggtttgccactattcttagcttcaacagataactttgaagaaagagtgcgtggttatacttccttaactgaagttgctgaagatttcgatgagaatactgctgcatataaggctgctaaacaactttggagtcagactcctaaagtaactcagctttatattggtagatgcgctatgcagtacactgtatcaattcctgatgaggttactgagagcacggactactcaattactgtagctgtcggtgggggaatgtcacaaccattccagtacacctcacaaggtgaagatactgccgagaatgtattacaacagtttaaaacacagattgaagctgaccaatcaattaaagataaagtttcagtcaatgttactggtagcaacggttctgccacaatggtcatctccaaagctggtgacaatgatttcgtgaaggtaaccactacagcacaaactgtgtctattgcaagcacaactgctgatacagcgtcaactgctctggcagctattgaagcttattctaccgactggtatttcattgcagcagaagacagaactcaacagtttgttttggcaatggcttctgagattcaggctcgtaagaaaatcttcttcacagctaactctgatgtagcagcactgcaaggtacagaattaaatagtgcaaatgatgttccagcacagcttgctaagagtatgtatactcgcacagtttgcttgtggcatcacacagcggcagaagattatccagagatggcatatattgcttatggcgctccatacgatgcagggtcaattgcttggggtaatgctcagttgactggtgtagctgcttctttacagccagctaataagagacctctgacaagcattcagaagtcagctttagatgtacgtcactgtaactttatcgaccttgatggtggtgttccagtggttcgtagagggattacttctggtggggaatggattgatatcgtccgtggtgttgactggttagaatcagacctgaaaacttctctgagagacttgctaattaaccagaagggtggtaagattacttatgatgatactggtattacccgtattcgtcaagtcattgaaacctctctacaaagagcagtcaacagaaacttcctgtcatcttacacagttaatgttcctaaagcctctcaagttgctttggcagacaagaaagctcgtatcctgaaagatgttaccttcgcaggtatcttagcaggggctatcttggatgttgacttgaaaggtacagtggcttacgaataatagaggtaaattggaatggctatgtatcagcaatattcccctaaagacgttgtatgtagctggaatggtattgccattgaaggctttgccccagactcatttttacgtctacagagaacatcaccacttatcacaccagttgtaggtgcaggtggtcaggttgctctgacaagaaatgcagacaagacaggtactattgagattgagctaatgcagacttctctctctaaccagatgctttctgcaattcaagctaaacaagacaacatggaacttgaagaagatatctcttctaacttcgtaatctacgacccatcaggctctgttctggcaactggtattaatgcttggttgcaggaattaccacagattgaacttgggcgtgaccagaactctaaaacttggatttttggttgtgagaagctagactacacttctacaattccagcgtcaagtgtttaataaatcctataagggggagactttaaaaggtcttccccttttttgtttcttttaaaagtattaaggaatcacaatgaaaacagaatctagagtaattaatggtaagaaagtaaatattgttctgcttggggcaagagatgggattaagatgtctatgaagctgggtaaaattgttgttccaacatttgcacagatgctatcaagtctgactgataaagataagaaagaagctccaatggttccatttaaagaccttgttgaagcttgttttgacagaattgaagaaattaaccttgaagaaatggctaccctgttatttcaaggggcaactgttgatgacttcccactcaatattgatacatacttccaagcaaactacggtgagtttattgattacttagcatttgcgctggaggcaaacttcggaagttttttcgaagcaagcattttcaaaagcctaacttctcagtaaacatgggtaatactctacagacaccactaactgatgctgctgtagaggcaacctatgaagaagcagacgagatgaaatttgtgcttgctatttatggtatggaagggtgtaaagaaacacttgaccaactctttgctatgacattctctgatttattatcattgagacaatttcttgaaattcagaggtcgtataaagaggaaattgcttacaacgaacttagaagagcaggaaaaatgtaatggcacaatatacagttgatagcttcattgtggaacttggtttcagtgaaaaggttgttaagggcttgcaaagagttgagaaaatgtctatgcaagctgctcagcgtattgagcgaaatatcaataaagcctttgatgtgaaacctaataaaagttctcaggaagcacttaatagaattgtaaaaaatgctcagtctgcttcgggcagaatcaataaagcactcaacagttccttgaaccttgattcacaaggtgttaaatctcttaagaaacttgaaactcaggcaaaaaagactgcaaaaggtattaacaagtcattaagagatgctatgaaggttgacggtaaaatcactatcaagacagggagagggagaggtggacagagtatccctcctgttggtggtggagcacccagaggtccgagagtagatgttgctcagagacaaatggaaagaatgtttaacaacaacttctattcagggttaacccgtagactggaaacaatcggtggtcaaggtaaccaaatggcagcctctttcagaggaagcttgcaaaatatctataacagatataaaggtactggtaaggttggtgagtatgagatggaagttaaaaagctcatcgacgtaaccaaacgttgggttattgcagaaaatgctagactaaaatcagttaaagaatcagcttggctacaggatagagctaacgcatcattacgtcaattagttggtggatttgtttcagcttatgctttactggaactctcacaaaaaactattgaagctggtgtaaaaagacaatctgcacaattagcctctacagctatctttggagcagacactcagcaagctagaatgttcgctgcatcattcgcacaccaaattggtcagaactacacagataccatgaagcagtactcaaactttgctgctggtgctcaaccaacacttggctttcagggtactcaagagttctataagaatgctgcaatgtttgcccgtatcaggggtgctactgatgaagacttgaaaggtatcatggttgcattccagcagatggcgtcaaaaggtaaagttcaggctgaagaacttcgtggacagttaggtgaccgtttagcaggtgctgtgcagctattcgctgatgctattggtaagaccccacaagaacttgataagatgatgcaagacggtaaacttcttgctcaagacgttcttcctaaagtatctgaaagaatggctgaacttgtcaagcaagcaggtggtatgaatgctgtgtccaagcagactgctacatcaatgggtcaagcgaaggctatgtgggataacacacttgtagcactgtttaacaattctagtgagggcatttcacagttatctaactccgttgcaatgttcttgcaaggttctatgggtagcacgcaggctttaggtcttgtgattgggaacctgttaaaaggcgctagtaatctacttgacttcattacagacttcatgtacagaacatctgcactatactactatgcaagagcttggtataaagaccttgacaacagccagcaaaagcttatcaaaagtgctggtgaatttctaggaacagttgtcacaattggaggcgcagttgctgtagtatcaaaagcagtcaagctcctaagtggtttggttggtggtggtatctttggtaaaatcttacaaagacttggtgttagtgcagcaggtacagcagcagccggagaagcagccgcagcagcaggtggagttacagcaacgagaatggcacttggtactgttggttcagcattaatgctaagaggctctacagacccaaatgctgctaaaaactacagtgaagttacattaccaaaaccatttgaaaatgctgttgcaaatattacaaacccaaaaaggccaatgttctttgatgagaatggtcaacttcagtttgcacagtacactcaagacgttgaaggcaacagaaagttaattgataatggtctatctaattgggagattatcatggagaagctatcaacatctattgataattttgccaataagtttaaccagacaccaatgatgatgacaccttctggtttacctatgcagactaaacaaaccctgaatgttactttcaatcttgatggtaaacaaattgctactaagatggtggatattactgacaagaatcaagaagacattcttctaagttcaagctatccagaggaagaataatgttatgggattctaatatgcaaatcaaatatagtggcaaagacggcatctatttccacttaagagataatgtagatgccttcttaaccttatcagcaactgaaaatatggaatttgacagccctatgcaggtaactacacagaacatgcaatcagggcaaaccgtcacagataatgtgcaaagagcacccagaacaatcactattagtggtgtcgttgtagttggctatgaaggaagcttattattaactcgtcagggtcaattagtagaaaacttcatcgacactcttgaaagctggcgtgaccagaagcaaattatttcagtcatctgtaaagacggaattaaaattgatgattccattatcacaagttttaaagcctctaaagatgttggtatttcaaatggtctaagaatccagctaacttttcaggaaattaactttaaagctatcgtgggacaaactgatatttctgctgcaactggaaagaccgctaccacgaatgatggtggtgctaccagtaaaaagaatacagggaataccacaacaagtctaggtaatgggaaattaaactgtcagttattattcgaccttgatgccaatggagtaagggaacttactagtgctgaagataatgcccttgcaaaatgttccatgtctgcaaaaaccagaaaaggtgttactacattcagtgaagaggctgagaaaaatgccggagcagcattaaacagaactgctggagatggaaaagcattacaaaagcattcagtgaatccgaataaaaaggggacttattaatgtcacaatatattcctgttcctgatacaggatggtctacacaaactgtaactctggatggcactgtctttgtaattgaattaaagtataaagagaggcttgacagatggttcttgacgctatctgatgttgatggtaatgtattattatctgaaaagaaatgccttgcagaccaatcaatcacaggacgctatgtaattccttcattagctggagagctttttgttgaacgaatgtatggtactgatttacagcctaccagaaataacttcggaagagaaaaggcatttgagcttaattattacactcaagaagatatgagattaatggagaacttgtaatgtctgtaaaagatagcactgctggggcttcttttaggtgctatcaattggctgtaggtagtgaaactacagcctttaatgataaacctacaagccatgctaaagattctatgcaaatggactattttgacaacttacaatttacttgtaatgtgtcttatacgtctcagaagaataaagtaacctctgatgataccacttttgaaatctacaaccttaataaagagatgagagccaaattcaaaaccgttggtgcaacagttatgcttagagcaggttacactactggttttaaaagggacgcaaatggcgaccttattattgaatatgataacctcccattaatctatctaggcactattgaatatgcttatacatataagcgtggtgttgatatgattacaaaggttatctgttccaatgataaaatggaaagaaccacgattaagacatcaatttcttataaagcagggacaacgcgtaaaagtgtgattagagatttagtcaacagactaggtttctcacttattgatgaagacctttcaagtattgatagttacacttacaagaatggttttagtgtttggggaagtgttgcagaggcacttacaaaggtttgtgaagaaagtagtctacgttggtatacatttaacaagcaaatccgagtagttccttttaacgctaaggctagacaactttcttgggaaatttacccatataacgttattgattctttgcaaggctactacagaagaactagaaaggttctgaaaaaagagaataagacagttattaaagttaaaactggagttcgttgcaaaatccatttagatgggagaattaagatgggtgataacatcactattagggaaagtgaagattttgaaggtcagtatcgagtaaaaggtctttctcacaatcttgactttactggtggtgcttggacaactgaactagatttagagaaggtggaataatgaagtcaccagttactagaatgtctgggtatgtttcagaatgtcttgatgaatttagaaaagaactgtatactggattaccagccattattcagtctttcgattcaaagactcaaacagccactgttaagccactttactctattaacggtttacctatgccagagattactggtgtacctgttcaatttccaagtggtggtggggcatctttaacattccccgtaaaaactaatgacagatgctggttagctttctcaatgttacctttagatgacttcgttgtcaatgacaagaatgttcagatggaaacaaacatgagaaggacacatgatatttcagactgtgtagctttcgtaggcatctgcacaagaacacagaattttaaaccagacccaacagcagttagacttcatttcggtgactctgtgttaagagttactgatgatggtaacttctactttgaaggtgatgtacacatttctaaaaacttgtacgtaacagaagaagtacacggctcagattttatcagtgacacgactggtgtgagctttaatgaacacacgcaccattattactggacagaccctgctggtgaggctgatactacagaggcacaataatgaaaacagactttgcattaaatctaggtggtgactatgttgccactttaggttcagattcagtgtatgtggctcatggagatttaaagattactggtaaccaaattagaattatcccagaagatgataaagctactcaggttgctcaaagacttcatattagatgccttttaagggctggtgaagttttctttaatacatctgctgggttcccatatttacaacttgccaaatttaaacagaaaacttctatctttgataattacatgaaagcctaccttgttgaaacaagagatgtatctaacatctataactattcttcttcaatggataatgctcagagaaaagtaactgttaattttgatgcaactactacaacagatattttaacagacattacgcaagaggttaatatctaatggcaggattaactacaacaggattacaaactctaagatatcaggaaatttttgataatatcaaatcaaggcttcttagagatatttcaccaaaccttgacgtttctgaggatagtcagttaggtctctttctagcttcaattgcaaggtctttagcagacactcatgaaattctatcagaaatctatgatggtggaacgattgataaagctgaaggttttaaccttgatgatattacagctttaaacgctgtgtatagatatgttgctcaagctacaaggggtcaagtagagtttactggaacaacaggtgcaacaattccgtctacaaccagattaagaagtactgctgggaatatcttctatccagtttcaaacattacattaaccccttcatactgtgttgaggctattcttgaagttaactctttacgaactgatgcaaattatgttattattattgataacgttattttctcttatcagccaaaatcttcagacacaatcacagtactattaactgagcttgctaatgcaattaatggtggtattgtagcaaaagctgaagtcatcaatgatggttcagcattacgagtttataaagatgaaggcgacattattgcaagaaccaaccctatggtagtgactgctaccacattcctcacattcacaaaaattacaactatccatgatgttgttgctgaggaagttggtgcaattcctgcattagctgggacacttatcgaaattgaaacaactgtagatggtcttgatagcgtatataaccgttatggcctgacaacaggtagaaatgaagagaccgatacagaacttcgtcagagatatttagaatccctgtcagttactggtgtaggtactctggatgcaatcgtagctgctgttaagagggttcagggcgtatcagatgcttcaggtgttgaaaatgatactgaagagacaagctcagaaggactcccaccaaaatctttcaagattgttgtagttggtggtcaaaatgataatgttgctcaagcaatctgggacactaaacctgctggtattagagcttatggggctatctttgggacagcttatgacttaggtggactatctcataatatttacttcagtaggccaacacctaagtatgcttttgtaaaagtatcatactctctatataatgaagaaagcttgacaattccagaagaagatatcagagacagtattattcaaggtattaacgcttatggtagaactttgaaagttggtaatgatgttatccctaatagaatctatgggtatatttataatgtcattaaaggtatcgagattaatgaaatcaaagtagcactatccaatagccagtcagtaccacctagtgatggacagtacactacagcaagaattactgttgatggtgaccaatacactgtatgggaaagtagcaagtacaccattactaaggagtgataatgtttcagaaaattgatgatgtttattacaagactcttgatgaaaggactgttacacagtttaaagagaaattcatctatacgagtttgctgaaagctatcactgatgagctacaaacattggaagatgtttcatggcagatgcacactgaaaggaatatcaggaaggctattggacagcaacttgacaacatcggtgcattaattaaagtaccacgaccacttggtgctgatgatgaaacatatagagcaattctttatattcagatttttctaagacgttctgatacaaccccaacatttttacagaatgctatcatgactctgtatgatgcaacattttcacagctttttgaacatattacacctatgactgctggtattgtggttagagttaacacaagaaataacgttgttgacacagcatacacattagcaaaaattgctgcaacaactattggttcagcagtcattctaagagatgtaaccctgaatggtactgcttggacacctgtagaggttgctgactctgctttagcaattgttgatgataaagataactggcttgttacagatgctaacaagggtctggttaccaacaatacaggaggttctttagagaagaacttactagggagtttagcagatgctggtgtaagagatgcttatttcaagattgatagaacggctaatagtggctcagtagattacttgaaggttaataaaaattataatgctacagacaactacatcgttggtaaagaaactgtagcaggtggtgattatggtgttatggctgaagtagctcaaatcatcaaaggtagaaaagataaatcacagcaagaaggaagttcttaatggcatttttaaattggtctacagatgaagtagatgctgatggtaaccagctaaaagtattaccaccaccagaaattcaggcgactggtttattaagaggtgaaccaatgggtcgccaatggtttaattatatcttaaactatctcctgaagaaagcaaatggtactgttggtgaggtaaggtcttttgccactgcgcaaccagatttggtggctaatggctggaagcttattaagaccgaaacaggtactgcatcaacaagtacaaaaaatctttatacttatgaatttgtaggagcttaataatggcagtaggtgaaattcaaattagtgccttgcctcaagcatccttaccaattgaccttagtgatatcttccatcttaagcagggtattgaggataagaggtgtactcttgagcaattacttgctccacacgcaagcctaagaaataaccctcatggtgttactaaaacacaaattggtttagataatgttattaatgctcttcagttagtcgctgcaaataacttatcagatattgttaatgtagctgaggcaagagcaaatctacagattatgtcttcagaagaggttaataaccttgtccagcaacatatcaaggacaagagtaacccacacaatacaactaaggcacaggttggtttaagtaatgttcagaactggacaacatctaatctgtataacgaagatgcagataagtatgctacagcaagagcagtaaataacttgtacaaggctgttcaggcttcttatccagtaggtactatccatctttctatgaactctgcaaacccttccacatacttaatttgtggaggtacttgggagttagtttcaaaaggcagggcattggttggctatgactctgatacaagaccagcaggttctgtatttggcgcttcaactgtcactttgtcagttaataacatgcctcagcactcccactctgtgtctcttagcggtggtggtcacactcataatgcaactgtgtcaatttcagcttttgattatgggactaagactacaaactcttttgactatgggacaaaagctactaacacaactggtaaccatacacacagtgtgtcaggtaacactagcaccaatggtactcacaaccatgatgtgtatcctagagaaaatgatggcactggtaatgctgtggctgactctgatggtggtggtgcaagaaggacatcaaggacttctagtgatggtgcgcactcacacagctttaacgtcacatctggtgcatcaggtgaccacagccacacggttggtattggtgctcacagtcatacagttggtattggttcacatacgcatagtggcactgtaacagtttcttcttctgaacatacacatagtggcaatacaaatagtgttggtggaggtcaggcagttaatattgaacagccatccttcgtactgtatgtatggcagagaacagcttaatttctttacaggggctttatagccccttttaagaggaaaatttataatggcagattacaaattgagtgagctaaactcaatcgacacaatccgttcagatgaccttcttcatgtcagagttaaaaagagacctaaaatgctgggtgatgaagaccgtcgaatgacctatcaagatttcttagcatcttttaagcttgaaagatttgttcagattgctggtagcactatgacgggtgacttaggaattgttaagctactttacggtggcaaggctgtattcgaccccacaggctcttctgagattactgttggtgatgttttaaagacttttaaaatcaacgcaaacggccttaaactaactattgcagacgcttcaaggtcggcaactgtttatcatactttgaataagccaagccctaatgaacttgggatgagaactaatgaagagaatgacgcaaggtatgcaagactagccgtgaataatacctttagaggtactcaagctatcctctctgacaatgaagcgctcattattaaaaatatcactcaaggtgctccactctatcttcgtggtcaagatgcagatggtactaacagatggtatctaggtaatgataataggggtacaaataatcttgtattgaagaatgtaaagtctgacgtttcaattgctattttagagaatttggtgacagtcaacaaaacagtcagaatcactggacaggtgcaaccttctgattggactaatattgattctcgatacattccagcagctactttgagtaatcttgctaaactaagtgatgtaaacactttcaggtctgcacagattattaatcaaaatggcttattgttacagctaaagaatactacacaagatagccagttatactttgctggctttaatgctgataatgttagaaggtggtacatcggtaacggggaacaaaaaccagaaagtttcgttatccacaatgataggactgctactaccattttcatggtgaatgacttcttactgaacaaaacagtcagaatcactggacaggtgcaaccttctgacttctctaacttagatgctagatactatacccaatctgccgcaaacgcaagatatatgcttgcaagcatcactggttcggcttctgaagcagatggtggtgatggtgttgcatgggatgcaaaaacaggtctttataaggttactaacaccactggacagtcttcttcacttgtatatcacatgtttttagggactggttctacaccatctgcacaattaaagttttcgtttaaaaatggtgggatgtggtatagaacatcaagagatgcctatggcttcgaagttaattggtctaaagtttacacggaagcacaaaaaccaaccccttcagagattggtgcatacactaaagctgaaactgaccagaaaattgcaacagcaattagtgactctacagaccttaataaaatctatccagtaggtattgtaacgtggtttaacagtaatgttgaccctaatacagcacttcctgggttaacttggacgtacctgaacaatggtgttggtagaactatcagaattgcagcagcaaacggttcagatgttgctacaactggtggttcagattctgtgacgttgtctgttggtaacttgccatcacacactcatagtttctctgctaccacttcatcttttgattatggtacgaagggaactaacagtactggtggtcattctcactctgtaagtggtacaacaagtgctgctggcaaccacgctcaccacttaggtctgttattagttaatggtggtgatgctcagtatggttacacaactgttagcaacagcaagacaagaactcttgactggcttgatagaagagataacaagttccctaacactaacacaactggtaaccatacacacacgtggtctggtactacaagcggtgctggtgaccattcccactctgtaggtattggtgctcatagccacacagttagtggtaacactggtagcacaggttctggttcggcatttagtgttactaaccagttctacaagttaatggcttgggtaagaactgcttaatcccttgttgactgattgttaagatggtgttaatattctttatgggtattctcaccatcttggctgatgaggtgattaaatgcctacaatcctagcaattcttctaaaaaatctaggtagcttcttctggaaactcattttatcccttctaagtgaatacatgattgagaaagtgttctttaagcttgcaagataccttgcgagtaaaacagacacacctattgatgatgagttcgtagataacttagaaaaagcttttaagggggagaaataaatgaagtggctagaggaagcttttaaaaataatattggtgcaattgtagttggtatttttagtgttattgggatgtttaccacgatgcaggtttcaagtggtaaacaagaagtgtctgtcacaacaaagttacagcagctagataattattccaaaagtaactattcagctattcgtgatttacagtctgacatgagactgattcagctagggatggagaaccagaaagttcagttagagaacgttaagggtgagaatgcaaaacttaccaaaactcttgataaattctctgacagtgtgaataatctggctcaatcagtatcagccttgcaagctattactgaaaagaacacaaagaatactgaaaaataaagtttaaggctcctttttacgggagccttttttgttttacttgcctactgacagaggtgcttcaatcttccctgcatgttggtagttgttaattccaccaataaagtcacttgcagtaagatgttttaagtcacttaaagtgttaagctgtacaccaatctcaaaagtaggtgcatggaactcttcattgttcatcagttcataaacctgcttcatatggttctgataaatctgagtatctccaaagacacctatcaagtaccgtggagtgtatccagtcatcttacaaagaacttccagaataaaaccataagacgcaatattaaatggaagccctaaaaaggtgtctacagaacgctgataccattgcaagtcaacttcaccttcgttggtgatataaatctgaaacagaacatgacaaggtgctaaagccattgagtttgctgcaatatctgctgcattccaagcattaacaagcatgtaacggtctgtgatatcacctttcatcttcgttactaaggtttctaactggtctacaacacaaccattatgaccttcaaagcttctccactgaaccccgtagattctcccacctgcatcttctaaccagtcttgctcagaagaatagttagagcttaaccaacgtttaaaatcatctgaccagatagtccaacgttctccatcattttcaccccaagtgcgataacggagttcaccaagtttattctcaccattcaggaaccataaagcttccccaatgacttgccgtgtaaacacctgtttagatgttaagagcgggaaaccagttcgcatatcaaaacgaaactgaggtggagcaaatgcagagataacatctccagttcgtgtagtacgcagttcaccaacggataaaacatggttcaggatatttttgtaacttgaatctgcttgtgacatatcaaaagtgtccttttacttgtggaacataaatttcaaaagttgctttaccatcctctgatggttttgactgtacctttgtaaatacacggttatcatacagcttctcaaagaagttttcaaaaggtaaatgcacagtggcctcttcagtaactttatggaagactgtatggaacaccacaccagcatacggtaaagcattgacaaggacacctgcaccaccaattacaaagacatcttcatcaatagagctatcaagatacttcaaaaatgctccaaatgactctttactggctttagcatacattacatcatctttgccaaagtctatgcctaagtatggtacagaatttgttaagattaggcttgcacgttctggtagaggtttactacccagagtcttaaaagtctcattacccatgactactaagttatttttagtgagtctcttaaactcttgcatgtcctgtttatgtcgaggccacggcataccagttggagtaccaaactcaccattttcaccacttgcaaagattaatttaatcattttgtaaatcctttcttaatgaaccagttgataacactcttcacaactttactacgatggtagataaaatgtggagagggttggtatgaaccataagcccagaaagcgataatttcattacaccctgtgcatcgggtactttcttcagcaatatgaccgttgactatttcacttacctcaccttgtaagtctgtacacccgcaatgtggacaacggataggtgttccatcagactcatagcagtgttcaattttcatctatttaacctcaaaaactggttcaagaaccaatataaagtctacacagtcatcttcttcatccagaataccatcaaagttcttaggtacaaagtcgaagaggctcatagaatatcggtcagaagtaaccctgatagtgttccctatattctctacagttaatattgtacggctttctgttacctcacttagaagattactcacaaagtcatctaagaagtgtttcatcagtgacttgaagcctttagggtaatctgtaccagagtatgtttcaatctgaggaacaagcttaacatttatactagtctttgttaagaacgttttagagaacatgctatcaggtagcttacaaacagtctttccagtaccttcatgttcaatctcaacaaaaccagcttcatcgccacatgtaactattttaaaagatgcttcattctcatactgtgaagccataaactttttaagcatcgttttaaggaaatctgcatgggcatcttgtaagaatatatcaatcatagttaatcacctattatgggaacagacacttgatgtcatcgtttttgaaaacaaaacttgtgtagttatgaccatcaacatccagccaaacagcctgaacatctacaccatcaagattgtggtaaagctctttgatgaagtggtctttcacaatctgactgctagttgtttccagcttaatgttcgagattgctgaacctacagccatatagcccatgacacgtttcttctcagtatgcttaatcttaccatctttgtaagccataatgagattggagagaatcttcacaccataatatttttcaaggtgagtaggtacgccataaaagcctaatgcgaagttttcagagtggataatgttttcacgtttcatattaatctgccaaccttaaattgaaattaacaccaaatttttcacatacttccaacatatactcaagggagacgttaccagtgagatggattatgttggatacacgagcctgagagataccacaaattttagcaacttgctcttgagaaagacctctggatttaatctctctcttaaaacgatgtgcaacgaagtctctcatttcgtctacatccattggacaaatataactctccatttcagcctctctatcagcctcccaatcatcttgtggggcataagggtcaaaaacctcattcatctttcttcttccgtttgcgtgaagtgtcaattaatgagtctatgtttgtagccctaaccttttcacgttcatcgttaagataatcgtcaagagccttttcaatctgtgaatcatcttgacagataactgtaaagactgtaaagccgtctgtatcgtcaattatcttaccataaccgcttctgggatgtaactttttaagagccatatcaaatacctgtgcagcttctgttttctaagaaatactcaacaccatacatagctttttgcatcaaatcttctttaaactggatacgatgattgagcatagcctgtacatcaatatcatcagggtgcttctctttaatatttgcagattgcaggaaatactctgcaagaacacgatactgatagtcagttaccttatcagtcatctctgttaaagactcttcagagattggtttaagtagttcacctttagatagggcaacacctttaacaatctgacaagtgtcttctggaacacctgataagtccacatttggaagttctgttgcattgactggtaaattcattgccataaccattgcagcagctaataaaatctttttcataatgacctcaaagttatattgaattttgtattacatttagtataaaaataagctataacacctgacttgtcaacagattttatagcttattttggcagattttttataaggctactgccatccttccataccttaccttaccacacctaaccatacctaaccaaactacaccatacaataccttgttaaaagaccctcttaaagaccttttaaaaaggagcctccgaagaggctacctagtcaacttagaaaggttttctgcgttgcttgttaatgttagctttcatagcagctaagtggttttgctgttcaattagtcgtgaacgttcttggtggctaagtgccatagtgttcacattctcaattgcttgagtagcttgctttaaagcacgaccaattttaccagacattttacgttttgcaatagttgcttgatgcttaggttcaaccacacgataaccaacacccattgaagcaaccagataaatcttctcttctttaagaagcttctcaataaacttctcaagtcggtttagacgtgtaagagcataagccttcatttcatcttcgtacttaacaatgtcaccttgataagttggcttcacaatacctaagaaggtgtccatctcagagtgtgatacagtgtcaccataactaaactttgctgctaatgcttttgtattcataattaaacgttaccttctgaaataacttctacagaaaaacgaccaaagcgtggacgccaatcacctacaccacaaaggttgccagcattttccagaaccatcaggagttcttctttggtgatttgctcttcatcgaacatcaaaccaaactctacagaccagtcacggaagattggacggtagctcataactcgtgctgtgccaatcttaacagatttagcatagataaagtctggattcttagcaagttcttgtgggtttgctgggcagttcttgatagtcattgggaagactacatctgtcagcataatggctcggtcaatcaccttaccaagtttgttgagttttgcaccagacttgatacaagcctcaatcatctcaccgttcataacaaaacctagctgctcatcgtagtaacaagaggttaccagttgactttctgctaagagcgcatagtcttcgtcagtctttttacgtttactagacagagacttgtgatattttgtcagagggtttaatgggtctgacagtgtatcagcgtggctcaggaaagggcgagtaccagtgattttgatatttaacagtttcatagcatttagctccacactttagttgattgttaagttgtttgtaaagaaccctcaacagaagactctttagaaacttggtagatttttaacgtggctactaccatccacccataccttacctaaccaaaccgaacctcacacaaccagaccgagccgcaccccgccttattaaaagaccctcttcaaagaccttttaaaaaggtaagtctcattttaatgtagctgagactaactacccatactgtaccgcacctgacccgacctgaccagaccagacctaacattaccgtgccacaccgtaccgaacagaacactgccatacctcacatcacttaaaaaccctcttagaagactcttaagtgatgctggtgagtacccgttggtttatgctgctcagtatactcaccaaattttctatgtcaacaactttttatataaaagtcctgatagctatatgtgttacaaaacagaagattgtgaacaaaacacccattgcaaaagatgtgtccaaaagcttacttgcaatgtatgacaatacaaagacaaccagtataaacagaatgactgcactgaacatagtttactcataatccctaatagctagcccgacagggaactgtaaagaacccttacgagtcatcttctggaactgaaccgttaaaggcttcccgataaactcttcaggatgctcagcaagatactgtttcttctcatgagtagtcttccatgagacatctacaaagacgttaggaagagtctctacaacgaacttgccatgaccacgcttatcagtctttacaccagtcactttaaactcttcagtgtgcatcttcttgtgcttaatcaagaagtatgaacggtgacaacactcatagaaggagtcttcagagattgaacggtacattgcaccttcaaacttagcttcaacccacttatcgtgagcttcatcaaactcttcccaagaatttacacgacgagacttaacaggaacaactttacagccgtctctaaagtcattcaatggtgaagtctcaataatatctctacgctcaggccacgtcttagtactgtcacagatatcgtaccagtaaaacttgagcaagtgtctgtcaggattgtcttcatttttaatcatggagacgatatcttctaagtcccacccatgagcataaatctctccatcaaattcatcaacatctttgtggagccttaacattgccataatgtcaggtattagttcagtagggattttgtaaacagtattctcacgagaatatgctgtaaatacactgccaatactcaagtgccttgaaatcctacaacggacaccatcaagtttcggttgagcatcagcagggaacttcagatactttgcatggctaacctttgcagcatcatgagcaagctgtacacctaccttctcagtattctgtgcagaatcttttgtgtaagcataacctttgcggtcaacttgctttttatacttagcagcaacttcaaagagagcttgctgttcagcattacgctcattctttttaccgatgttcttaggctctgctgtgtactcttcaaacatcatcttgccattttctttaccataggttgtgataactttgtcacctacagcaacacatgaccagacgttgaaggaaccatctttattttgtttgtacagaattgtcatttttaacctctgagtgataccaagtgttgcaggcattacatgcgtaaagctctaagtcgtttttatctggtttatggaaacagtttgacccacaacgacaccggaataacttaccatcaacacgaagaatgaatgtttcaagctcaccatcttctgtaagaactgcgttctgaaatttattatctttacccatttgtactctcgcctgtgtcagattttatataagacctctcactacgtcttttaatgaaatgtacaacgcattttggtgtattttttacatctttaatgtgcaacctttcgaaactgtgtactgtgaagattgcatctgtcattgtttgcttagatttcattttctcttaccaccaatttccacgtcgataacttgaacatcaccagcaacatcaaacttatgaatgatgctttccacagtgtaagtccaaccctcaatgcgaaccaattcaccttctcgcggtacgattgggttacgttgggccacggttgcagtggaataaacaacatcgtagcagtagatatagtgtactaaaacttgcatattagtctcatccatcaataacagtagtgtgtgattgaccatttttgccatatctataagatgttgttgtttgagagtttgggcatctgacaacaacaatatccctgcttatagcatccccattgagtttgtagattttgcagtcttgcatctctggagggaggctgtaagaggcatctttgttttcataagatgggttacagccagccagaaaaattactccaagtgcaaccattgctttcacacaactcttaaccttattgttcacgataatctacctctctgtatgtttttaaatagtgagtgatgagttctggtaattcactacgtttaacgtttgaacgaagaagtgtccagtcaaaacctttaccaaccctcccccagagactgtacattttcttcatagcaactccagaatatcaatacgttcacctttgagtactccagtacggtcttttgcaatcttcccttcaacaataagaatgtgtgcagaccttaaagtgtcactgttaaaggcttttacgaacttgctaggcatcactacaatacacatctcctgaatcaactctggagtatagcagttgtcgtgaagttgctgaataacgtctgcaagtgtcatttttgatgcctccaatactcaatatcaataagttcttgtgcaatgcttttaaggttacctttcaagaaaaccttctcagaacctgaaagagatgaattgtcaattaacctgttcatcctctcaaacaacttctgcttacgattcttgatagtctcttttgaagcacctttcatcatcgctgcatgggttcttccacggtggctcataagccctcctatataagatatagtataaggtactcttccatgagcaccttgtcaacttagttttgcaaactctccgtggagtttaatagcagcttcacagtaagctttatgggcttcttcaggtgtgtcaaacataccaagatgatagtttttaccttgtaaacgtatcattgctcgaaaccttttacttatcttctgtacaccttttggcaacccagatggggactctcttgaccgattgcaagagttttgtgaagctgtacaaacccttaaattcaagtacctattgtcccaaccgtctccattcttatggtcaacctgtaaggttttatcaggtagatatccatccatatacaagataactaaacgatttgcttggtaaccccttccatcaatcagcaactgaactgacttgtgtccatcagtatttgttctgtagtttccagccctcttacctccaaatctagagttaaagtttgcattaccttctctaaacttccaagtaaagacccctgtttcggggtcgtagtccaatagctccttaagacgaatttctgtaatcatttcaacaccatcaatgtacttccaaccagctcttaccaaccttgcctgtgccagttacaagcgtctcactcttcaaacctaaattcttagatgcctgaccatacatccagtcagttatggctttaacttcttgagtcatactttctgggcattcccaactgttttcatcgtggtatgccagcaaaagcctagcaccaattgctggttttcgtccatttaattttgtcaacccttcttcacatgctcttctacaagcaaggttaattgcctcattttgaatctgagcctctgaacccatgagcaaataattcaacagcttatgcggagatttacaccacaaccaagctccagcaacctttacaaaaccccctttagagattgcagcagtcttaccaaacacctcttcaagtgcctttttagttgctttgtagtcagcttccaagtcatctagcagctttttaattttaggcaaacgaataaagtaagtctgtttagtcagcgcaccttcctgagtagttgcagctttaatcgtcttagcaaacttctcatcacctgcaccgaacagcagtgcatagataccattcttagctttcttacgacccttgctaatctcatgaagaagattctcatcctgagtagctcgacaagtcaagatgtcctgctcatcgttcaagttaaagtaaatactgttcagtgtatgtgcatcagtccctgaatagacaacgtacaggtcattctcagagtcatagcgaaggtacttatcaatctctgggttgaggtactcatcaaagtgtttacagtaatagcgtccatcttcttgacgaatgaactcaatttcttctttaccttgcgttaccgctttggtgaagtctttatcacccataaagttacaaagaagaactagctgggctgagttcatgtctacagagacaacgttagtaccttcctcacaaatccacactgccctcattggttccccatagacagcagcaccagacggtacgtttacaataccatactgtgtcatacgtccagttgaagtaccaaacaccattgcaccagcactaaggcgaccatcaggacgaatctggttcaaccaacctttttcatcatcctttgagttctcaatagttctgcgtcggtgcatcaaagtgtagtatttagcaatcttctgtccaagctcaccttcaatcgtatcgtaagatgattcagtaagttttggtgaagtacggattaagcaaggctcaagcaaatctgtgtacttcttcacagaccagttatgctcaatgtactggacgccttcgtgttcaacataactcaaaccacaccgctcaaccatttcctgccacttaggatgctttgtaatcatctttttattgtctttgaaacgacaaactttgacagggcgaccatcagagtctttcttgtagttccagtcatctggaatccaaccaactgatttcaagtagtctttaacaactgctacctgagtcatacgagaaacttcaaactcaattggtgtatatggtgcatcaatcaaacctgtatagcgacttgactcaagttcaaagtggttaacaacatgactgttgtagtacttcacagttttgactgtttttactggcttccagtctttgcatttcttaccaatctttgcattcaactcgttgcaaattgcacgagcatccttcattgctacaaaaccttccttgtactcttcaccagttacagagttggttggtgtatagcaatttctcttttcaatgttaaaaatctttgtagttggcttaccaaatggcttgatttcatatgtctgcatctcaccgttacgtacctgctgacggtactttgtcttagggtacttggtaattctcttcagtccatctgcatgaccaaatgtttcaacatactcattccaagcttttgcaaactcttctccagtgactttacctttggtcttaatagttggaggtagatgcggttcaacctctgaagcaagctcattagtcaacttgtcaagttcctttacatggaactccataagctctttatcagctttccaaccattgatagcttgttgactcatccagaaagatgtttctttagctcgcatgtaggtttcataagtgtctatgccacacttcttaagcttgagatactcattatcaagtgcccgtttagttttggcgttaatacggatatcttctacaacacgagtgaagatttctgcattccacacaccccaatgttcaatctctggtttacgaacaccaacacgagcaccccatgcagccaagccatgagcacctttataaccctttggagttggtctatccatccactgaacacgagactggataagagaatcctgaaagaaattactccacggcttacactttggattatcaaagttccataaatccggtgcaatgtgattgaaaacccaccagtcataacctagaccattatgaatgcaaagacgttttgcctttaacgcaaactcaataccttcacgtaagccacccttaatgtacttggtatacttgtgaccgaggataggttcatctgtaaagacccatacaggaggctcttcatcatcagatttatagtctgcaaaagccattacgtgaactttagtgaactcaaggagtaaaccatcagtttctgtatcaccaaccaaatgtaagtttttaaaatctacgttttccatttttactccctctaaaatcattaacctcatacattactgcaaaaaattataaaaagcaagttgacaatgcacttgacaaggtgttaatctttgcgaagtgggtttttacttaaaaggttacttaacagtttaactatacagatactttaaaagtcttaaaagattatttaatagcttttaaaaagctttaaagtataacgtatagaatacgttaagataaaagattaaaacttaatagttacttaaaagaggcttacatgcagaaaatgtttgttcacccagacatagctgactttgttagagctttgcaaaagttggaagaacttgacattgctgctcaacgggaatatgctcaccatcacaggagaatggttgatatacaacatgaaatagagttgtctgaaaactatgaagatgacttaaaatgctctgtatttgagcatatgaaagatgttgctacagcgagacgtaaagctaaagacacagttgctttactagattctcttaaaaagaggttgcaaagtggtacagacctgtgtaatctagcgtcactgattaacgatgttgaaacttcttgggataggcactattatccacgttctgaaaagacacttgacttttcttcatccgaaaacttaaaatgttctagaaagaaacttaaccaactgagagagaaataatatgaatatcgatgtagaaaagatgcttgaagagaaaggtattgacgaagcatttttaatcaatgccgtagaaagcatggctatgattcttcgtggtaacaactacacagaacagtttatgcctgcaagctataacttgcctgaagacaagtctgataacggtgcttttgcagaacaggttaacatgatgctttgcgacaaaacacttgctttagcatctatcgttgtaggtgttgatgcccttgcagagttcattttttatgagatgcagaaaaatgattatattgccatgaccactgaagaaaaagacttgctactactcgcaggtaacgatgaactaattgacctgttaatttcatcaactagtgctgttatggctgctcttgagaagcgcatggaagaaaaactttcacgatatgaaaaacttgattggggtgtagaagaattaaacccacgaatcaaagaaattatccagcaaactaaagagtctttggaagaagctgttgaaaatgactctgtagctgatatagaacctcttcaggtgaaagttgcactgatgattccagctatctctacgatgatgaatatcgttgcactaatccaactttcacagatgatgggtgtaagtattgagttcattgaagagatgactggtggcgttagtatgcaagcaatcaatgatgttttggtgaacattggtgcttcaatcattgaagagaaactacgcattaacttcggtgatgattttgtgaagcaaatctcagctatggctgaaaaagagtatgctgagcaactgcactaagtctttataaatcttacaagcctccttagttgggggctttttagtaattaatggagataaaatgagtaatattatcgcatttactggaaaggcacgttctggaaaagacacctcatgttctattgtgaagaacatcttagaggatgagcacggctacaatgttgctgtaatggcctttgcagacaaccttaaactgtctgcatcaaagatatttgacctgacttggaatgacctgtatggtgaaactaaagagactccacaggtatttgacttgtcatatcctgaactgatgtttaaagttacggaagctatggagttcaccttcagagatgaacgttaccatatggacttcaagcttatgtcagagttaaccggacgattaatcatggagcttaagaaggttgctaaaccaactctattgacacgtctggggtttagcaaaaagtataaattctcatcaagacaaattcaacagatttggggcactgaagttgtccgtaaagtaatgggagacaagttctggtctaaagaccttgaaaaaagaatggttagattctatgaaagttgctcacttagaaatcaatatggttttgtcttaatcagtgatttaagatttgactctgaagctgagtggctgagtagatttgcacaccaaactattgaagtaaaaagagacaatgtggataagatttcatcacacgtttcagaaaatgggctatccaccaaatatgagcgtgacattattcataataatggtactcttacagaccttgagaacaagctaagagccatcattaaagtttaaaagagagaatgaagatgagagtaaaagacaattttaaagttattgaccaccgtttagtggaactctcatctctttccaatgaggtaatgattgaacgtcttaagagagttgaatcacgaaggaaagagattgcagatgaaattcatgaactggacaagattgaaaatggattgaaggcagaactacaacgaagaggtgctaatgtctaaaggtcgtaaattgaaagaggctggtcagtttgttggtcattgtgcatgtccacgttgtggttcatcagtggctggttcaatctatcatcatgacgatgattcttattcgatgacttgctttagttgtaacaaaggtttcccagagtgggattttgataaaggacaaatcgtgagcacttattctactggttcagacaataaaaaccgtactttccgtgggatggatttagacgatgtaaaagacaaccttgaagcaatggacttaaaggacaggaagattcctgcaaaagttcttgagcgtttgggtatcaaggttgacattgatagtgacggtgaaattgatgcgcatttctacccaacttacaaacgcaatgaagatggcaagctagagcatgttggctaccgtgttcgtcaccgttacccagaagaccatccaaaagagcacctacgcggtaagttaaaagacttttctggtggtgttggagatattaaaggtgaactggcaatgttcggttcatggattgctccagaaggtggtaaccgcctattcatctgggaaggtgagatggaatgtgcgacagcaatctatatgacttctcttgcgattaaggacaagtctcgtcgtaagaattactgtcacgtatctgttccatcaggtgcaaacatcaagtctatcaaagataactatcagtacattacatcatttgatgaaatttacttgtgctttgataacgatgaagcaggtgctaaagccactaaagaggctgctggtatcctacctattgagaaggttcgtttattccagtatccagaaggtgtaaaagaccttaatgaatggtggacaaagttctataaagagaaagatacagttctggaaggatttaagcagcgtatctacaatgcacctcgttactgccctgctggtatcaagaactttgcagacggttttgaggcaatgaagaatcgtggtcagattccattgattccttttccagaatctttcggagacttgaacaggctaacttacggtggttacggtttaggtgagattacgactattgcagcaccatcttcagtaggtaagtcagcttacactcgtgagatgatttattcagcttggaaagaaactgattataatatcggtgtaattcctgtagaagatacctatgaagagttgatggaaatgctctgtgcaatccacctgagcaaacagatttctgagattccttatgatgaacgggattgggatgaattaaagggagcacacgcagagctttcaaaaggtcgtcgcatccatatcgttgaccatcaaggggcgattgaccaagataacctgctggagtttgttgactatcttgttaacagtttagactgtaagattattattcttgaccctattacattggctctgtcacgttctgatacggatgaagaggaagtcttgtctgaactattgcgtcgttgcaaacgctaccagtatgcacaggtaaacgtttgtcacgttcgtaagagtgcaggtggtcagaaagctaactctgaaggtggagatatctctgaagaggatattaagggttctggtgcgtatttccagatttccatgaacaacattctgttaatgcgtaacaaggttgacccagaccctgttaagaaaaacttgacaaaaatcaagttaactaagtgtcgtcgtcatggtaagtcaacaggtattgctggtcatacttggtacaatccagacacaggacgtcttatcaaagcttctggatgtggtgttgacattgatggtgcagcagaaaatattcgtcaacagtttggtattggtgaagctgaagaccattatgacgactctttacctcattatgaggatgaagtgtttgaccgtgagactggtgaagtctatactgaagaacagcgtcaaagctcaacgattccacctgtattaagtgaggatgcagatgactgcccgtttgaaactgagtgatagttacaaaagagaggggttcatacccttctttgaagaaaaaagtttaaatgaatttcaggaagagtgtttgacaaaggttgataggttctatagaatgctctacatgaaatgtgatggaggcgttgaggtagaagaaaagttccatcacagtatgataacagtcactattactatacctcgacataatattggttggctgtttataattaaagaaaatgtgtttgaatatcaagtatatcggagaatatcatgaaacactctaaagcatttgaacaagtttttggggattccttaaaagccactgctggtaaaccagcaaaatactatgaagagaagcgtgtaagaactggaaaaactgcacgtaaagcagcttctaaagataagcacaacttccagtaattaatgtttgacaatagagtatcaacaaattagaattggtactcttcataaattgagatagaggtttaaataatgtctaaagttgttaaaatgaaagctccggtagagaagtacaatggtactgaacgtcaaactctgcgttaccttctgaaagatgtttggttttattacctgaacacttcaccacgtccgggaaaaggtaaatccattgataagaaattcccaggcaaagactgcaactacagtgtttcaattctggcagaagatggtaacaagctgtttaaagagtttactaagtctaagaaaaacccagaaggttgggataaagttactactgaagcagttgatgcagatgacttcgaagagaagtttggttgtaaaccaccttttgaagcagacacttactacatcttgaaagtaagtcgtgcagcagcttataaagatggtgctgtatggacagctaaacagtcattccctgtaatgctgattgaagaagtaaatggtaagcgtgtagctgttaaacagccgatgaagaaaatcaaagctcaagcatccgataagcatgaagatgacaagaactatgatgtaattcatccagatattgcagttggtaacggttcttttggtagtgtgattctttctactcacttctacacttttgagaacaatgttctgacaaaacctattcaggaacagtttatcattgatactcttgtaccttacactggtggtaacggtgctaacggtgaacctgaactggatgaagacgaactggctatgcttggtcttgatggtgttgaagataatggtgaaatcactgaagaagacgcaacagaccacaaaccttcgaacaattctgatgatggtgacgatgaagacttgccagacccagatgacgaagaagatgaagacttcgatacagaagactaatctctaaaagttactttaaagccctgtacttagtatggggctttttcatatggagagtcacaatggagaagtatacactaacaaaacttcccgattcagttacacatgtctttattgactctgatagcattgcctataaaggtgcttgtgtagttgagaaagcaaaatataaatatgtcaataaactcacagcagaagaatctgaaccatttgataatgcaaaagatgctgcaagatggttagcagaccagagaatccttgtggaagagcttggcctgacatttgatgaagatgaatgggaaagacagacttggaaagaagctaagagtgaaaaagaagctatcatggctactcagcaggttcttcaggaatggcttaaggttgttggtaaagaaagaacttgggtaggctacttaacagagaagggtgtacataaacataaagacattaaaggtcttgagcaccaatatcaaggtaaccgtaaagatgctgtcacaccaacacacttagttgcttgtcgtgaatatcttctgtcaagaccagagttcaagctcttaaagggaggcttcgaagccgattccatcgtaattgctaaagctgaaaagatgggtaagaaggctgctcttatgagtattgacaaagaccttcgacaagctgaagggacttattgtattgatatgacttatgaaaagtcaccactaatttttattgctgataataatgttggtgatatctgggattgtcctatcaaatcaaaaccaaaagctgcaaaaacagttggtgtaggttttaaatttttgtgttatcaggctgttgctggagataatgcagacaactacttcggccttaaaggtgctggcaaagtagctgtgatgaatgctttagatggtaaagagacctataaagagtgtcttgatgctatctatgaactctatgctaaaaaagagtcttacacctatgtgtcatgggatggacagactgttaccaaaacaccattagagttaatggaacaacatttctgggtagcatatcaagagagaaatccaaaagataatttctcttttaagaaatataattgggagccgtgtatgaatgaagattaaaggatttggcaattgccccacctatggtcattgggtatctttgtgtggggaagttgaccccactaaacattttggctttgtctatttagtatactgtaagaaaaccggacaatattatatcggaaagaaacagcttaatagtgtgaccaaaagaaaagttgctggcaagactcggaagaaggtagtcactaaggaaagtgattggatgacttatgagacttcttctgagtatattaaaaaagatattgagagctttggaaaagaattttttgacttttatattattcaaacctaccacacaaaaggtggtctagtttatggtgaagcaaaccttcaacataaattcgatgtaatgacaaaaaggattgactctaagctcagactcttctacaatgccaatattgcagcaattaagtttattactaaagaaacttatgaagatgctgaaaaaagaatccataaggtaatgaaagcaaactgtgcttgataactattgagagagaataaaatgtttaacaaaagtaaagctgtgagtcatgtagcaaaagttgatagcaagattgaagaacttgagcgcattcttgcaaatgccagagagtcaataattaaagaggttgaagctgttgaatctcaaatgcagcacctaatgcttaaacgtcaagagctacgtgagcatctggaatatattgaaacacgagagctaaaggttaacagatattttaaggagtcagaatgtttaactcaagagaatcagtaaaaaactggaatcttcgttgtggaaacactcaaaagcaaccctacagcaatgagtattgggaatctttgaaatctcaatctctatgtatgcttgaagaagcaaaagagcttgtaaaagcaattgaagagaaagaccccattgagacactggatgctcaggcagatttgcaatatgttcttgacggtttgatttacctgtcacaacatgaccataatggtgctatggaagctgtttgccataacaatgacctaaaatacacagacaactatgaagaagctttaaaacgtcttgcagatattgagaagcgtactggtcaagagtgtatcatcagaatgtcagtagttgatggcaaagaatggtatgcaattgttcgtgcagccgatgggaagattatgaagcaatctaatctccctaaagttcagcttggtgaatacattgtagaacttgaaagccaagaactttttgtggtagtatctgagacatgcgttatctgcaaaggtattgtatgtagcttaaaggatttgggtgtagatggttttgtagaagttaatccaattacctctaaagcagataaagatttctgtaaagagaatggactatggattgcagatattgtctactatgatggtgagcagttccatgtaacctcatacccaaaactgaattatgatgctaataacctgaagtgctggttaaaaggggttggttataatggattcacagaacattaataaagaaggtgtggctcagaagagccacttctcaaactacaatatctctatgacggtgtttatgaatgacccgttacttgagaagtatggtgagactccagatacacttctggataatgaacaagttttaaaagcagttctgtacaaatacgggattgatattgagaaagagtattcttttgaaatctgtcaacacaggaatacttttggtaaagttgtaatggctccactcttcatgggtgtagaaagaactgactatggttggttatatctaaaaagaaacttggagaaataccgtgtctaaagcaaaaaagctatcttatgatgacattatctcaggtgctaaattaggtgttgatagtatcggacaagatgttaagcacggggacacagtcatgtactgtgacgacagaagaggcagaagtgcaatcttgtttggaagagttgtgtgcaagatgcgaggtaattacgttgttgaagacatgaatgtggatgtcacaaaacagcttgaaataattatgaatagtgatatatcatggttcgtgaattgtatgcacacctcttcagtcacaaaggtaagtgataagttttacgatatgtggcagaatgagcaaattttcaagatttaaactagggagcctcttcggaggctcttttcatctgtaggattcaaaaatgattaagacaattaaaaaatcaaacggtacagtagtaagctttgacccagaaagactgaataagtgggcatcatgggcagataagcgtggaatcatctggtcagaagtcactatggaagccatgaaacgtgtctatggaggttgcactacaaaagagatgcaccaagctatgattgatgtttgtgttgataaacaaactcaagagtactcagacatggctggacggctacttctaggaattatctacaaagaagcctttggaggctttactaaggttcctacgctggttaccttcgttaaaaatatggagagagcaggactttgggagaagatggattactcacaggaagagcttgaatacctgcaaggttacatcgtgcactcaaaagatatctcttacggttatgcagtcttgaaacagttcagagacaagtatggtatccgtgatattaaaacgggaagactttttgagtcaccacaatttatgtttatgggtatggctatgaaagcctttgagaagcaaccaaagcaccgtagactgcaagatgttatcaagctgtacacttacctatctgacctgaagattaacgctcctacgccttatttaaacggtttgagggcaactaaatcaggttatgcgtcatgctgcttgattaaggcaaacgatactgctgaatcacttggtattgctgcaaaggttgcttatgatatgactacaaagcaagctggtatcggtatgctgatggagactcgcactattggtgatggtatccgtcaaaacactatcgaacacatgggtaaactgccttattacaagcttgtacgttcatctgtagaggcaaacaaacagaagagtcgtggtggttcagctaataacttctacactgctctagacccgcagattgaagatttactgcgtttgaagcaccctacaacggttccttctaaacgtattaatgagatggactactcattcggcacaaacgattatttctggcagtgtgttcagtatgatacagattggttgctattctcttacaaagatgcaccaaaactctatgacatgttctacacagcatctgctgatgagtttgctatggcagttggtcacgcagtacattcaggagttaagcacagacgagtcaaggctcgtgaaattgctaagctgtttattcaacagcgttatgctacaggtcgtgtgtatccattcttcactaacaacgcaaacacacatacaccatttaaagaaccattgaagatgtcaaatctttgtatggaaattgtgttgccagtgtatggttttgagaaagagacagacctttacagagacgatgctgtgaaagaggatggtgaggtggctctttgcttcctagctagtttggttgcagggagaatttcagaagatgaatacgctgacgttgcttattatgctcttgcaatggttgactccgttattgaccttatggattatccgtatccatcgatgcgcaaccatgttcagaagcgtcgttctgttgggattggccttacaaatgtggctcattaccttgcgaagaactacgtgaactactcttcacgagcaggtaaaacgaaacttcatgaactggcagagatgcactcttactatctgcatgaagcctctctgagacttgctaaagagcgtggcgttccagagtacatgaagttcactaagtatcctgaaggctgggttcctccaaagacagctaacaagaagattgatgaaaagcatgatgcaaaactacgatatgattgggatgacttagcacaacgtatcaaagaaaatggtggaatccgaaactctgtattagaagcttacatgcctaatgagagttcttcactggcaactaatacgacaaatggcttgtacccaattcgtgactttattttaactaaaaagtctgcaactggtaacgtgctgtttattgttccagattatgaagagttgaagtatgtatatgaaattgcttggggtattgacacctttgacctgattgattgttatgcaattgttcaaaagttcactggtcaagctatctcttcagatttctatgttgactacgcaaaagctaagaaggtatcattggctcaagctttgaagtacatgatttatgccaactcggtaggtatgaaaaccatgtactaccttaacagtcgcattggtgtaggtaaatctgcactgcaagatgcttattgcgagggttgtggtgtttagttttaataactatgagggtcgtagaagaccctctaaaaataactttggagagaccatgaaagatttaatagaaaaacatgagcgaccaatatacttgctctacaaaccccgcaaaacattgtactatgtgagcagcacaggtatcatgataaagcagaatgatgacttatgggtcactggagcctcttacatctctacagcagatggtaaaatctacgcaagaccttatgagatgtttaacaaagagaattgggaagttttagacagaaaacaagccttagaaatgataaagaaaggagaaatcacgctatgattaaccagcacccaatctttttaggtggtgagagaaaaacttttgactcactcaataagcactacccaaaaatctttgaactttataaacaacaaaaagcacaagactggtcagaagatgagttcccttttgaacaatcacgtcttgattttgagagtgtcccagcatcaatgtcaggtgtaatgcttgagattcttaagtggcagtgggaagcagacactcaagttgctaagagcttggcatttgcctttgcaccctttatctctgatgacatctatgcaactgcaattatgaagcagtctgagattgaaaacctacatgctcttacttactcagagattgtaagacaatgtattaaaaaccctgaaacaatcttagatgagattaaccagaatgttgctgtacaagaccgattaaaaactgtgaatcgtgttcttgaagaattactggatgaaggtataaactatcgtctgagttatgtgcgtgactcacttctggacaaagaccctttacacttccataaagtgattctaaaagggttgtttgcagtgactgcgcttgaaggtatatcttttatggcatcctttgcatgtacttttgcacttgatgctcaagataaatttcagggtattgctcaagctgtccagaaaattatgcttgacgaaatccttcacactaaaattgatattgaagttttaaaagaaactttaagagatgatgagtggcagaaagcttttcaacaaattcttccagagattaaagtaatcttagatgaagtagttgaaagtgaagagaaatggtcgtattatatcttctccgaaggacgtgctgtagttggcttaaatacaaagcttcttcatgagtgggtttactataatgctgccccactgtatgatatgtttggcattcccagagattttgtagctcctaaagaaccacctttgaagtatatgattaagaagatggaaattgataaagagcagaatgctaatcaggagcaacagaacggtgcgtacctattgaatactgttgtagatgatttgaatagtggatttttagaggttccttaatgacttatgtaatttactccaaaactggatgccctcagtgtgagactgcaaagaattttgcaaaagctcgtggtattgaccatgttgtgagaatgttagggcaggactatgaactatcagacctgatggagattgcacagatgccagttcgtcagatgccattcatcatgaaaactgatggacaaaacctaaaacctgttgggacgctacagaattttatggcagaggtgaataatgcttaaacgcatttgggaaggtttggttgttgatgcaccagcgattgtgattggtatgttgattgttaacctatttactgactttgaacaaggttcattgtttggagccatgttactatgggttatcttcgaaatattagagatacaattaggtatcactgaaaaactaagagaactctttgcgaagttttctaaaaagatttaaaatgaaagggtctcttcggagacctttttagcatgtaaaggggcaataatgaacaaagtacagattattaaaaagaatggctcactcgaagaacctgatatcaaaaaagttttagcagcagtcacaaagtcagctaacagggttggatataaagaactgccaccagatgttacccaagctcttgagtcagcatttatgaggattctggtaaagtccactaaacagaataatttactaatttcggtaaatgatattcacagtattgttgagggtgctttggcagaagtcaatcacgaaatctatgagtcttactcaacatatagaaattaccgtaaagaagttgctcaaaattgggatgagctttaccagaagaccaaagatacactcttcttaggtgacagagagaatgctaactttgacagtagtttaatctctacaaaaggttcaattattcgtggttatctgactaaagaaatctttaaacagtaccatttaacaccggaggaacttgaagccattgaaaaagggtttatctatatccatgatttaagagacctaatttttggtggtatcaactgttgcctgttcgacattggtaaagtcctgaaaggtggttttgaaatgtctggcatcgaatattgtgaaccaaagtctgtactgtcagccttacaggttattggtgatgtagttctttcagcaactgcacagcaatttggtggctttactttagcagagattgataaggtacttgtaccgtatgctaagaagtctctacgttaccatgctgaaaaagcagcatcttacggtattcctaaagaacattaccacaattatgtcatggagcagctacagattgaattaactcaaggttttcagtctcttgaaatgaagctcaatactgtaccttgtagccgtggggattttgcattcacaactttgacattcgggttacttgactcagatatgtctaatgaagataaccgactgcaatacatgattgcaagtactatcctagatgttcgtatgaatggacaaggtaagagcaagaagcctgttgtattccctaaactggtttatatttatgaccagaaaagacataatgagaatatctgtcaaggccacctgtacagtaaagctattgagtgttgctctaaagcgatgtatccagatttcttaagtgtatctggtcatggtgctgtagcagaagcttttgagcgttctggcaaggtaatttcgccgatgggttgtagagccttcttatctccatatcttaatgaggatggggaagagttctacgttggtcgtgctaacattggtgctgtatctttgaacttaccaatgatttaccagtattctaaagagaatggtttagatttctggaaagagcttgataagtacctagaaatgattcgcagcttccacaagaaacgttacgaaatgattgctaatatgccagcaagttctaaccctcttgcatttacacaaggtggtttgtataaaggaactaagaagcctactgacagggttggttgggatattgtgaagtccttcacagcttcttttggggttactgcgcttgatgaactatctgttcttgctgaaggtaaacgacttcatgaagttggaagctgtagttttgcatacgatgttctggaatacatcaacatgaaaactgaagagtttaaaaatgaagatggtttcttatatgcagtctatggcactccagcagaatcactttgtggaactcagctaaaacagttcagagagatgtttggcgttagcaaaggtgtttctgataaggaatactttacaaatagtttccatatgaatgttgcagcagacatcacaccatttgagaagcaggacttagaagaacctttcttccatatctgtagaggtggaagaatccagtatgtaagggtagctaatccagagaacttatcagcacttaagagttgtattacaagagggatgttgaaaggtttctatcagggacttaactttgacttagcaatctgtgaacattgtggtaacagaccaaaggctgatgttgaagaatgtgaggtttgccattcacatgatatctctgtgattaacagggtgtgcgggtatcttggatggactaagattaaaggtgaatcccgaatgaacgatgcaaaaatcgctgagattcgtgacaggatttctatgtaaaacttgacaaggtgatgtgggtcttgataggcttacatcacctttttttttttattggatgttagatatggcagatagtattattgggttattcataggttctgtcttactagggttctttttaggactttcttattgtgaattgagagataagttaaaatgtttaagatatcaaaaacgatgatatacatactccacattctagtcttcttctttggggttagtgttatggtatggggtttttcagacccacaatggagtttctcatatcatggtcagatggatttatggtcatgcttcaaaccatttttagggttagctatagcattcagtgcattaccaactaaggtgaaattatgattaagttgagccaaaaacagttagagtggattaaggattacgcctcagagtgtggctcttgtgaaaagaatcacgtaaggtactcaacattccacacggtcttcacattgtatgttggcgacaacgttctaagtgattccgtagtggatggtgtaccgttacctaatgagttacttgacaaattagctgtagttactggaacgtggagtgaagaagatggtcacgacctatctaacgatgttgtcttctacagtcttgaaaatattatgaatccagagtacattatgctaatgacttgtgcacaagactgtgttccattacaaaacttcattaaagagcactgtgaagagtttattactaaacaggttccttgtcaggtagtgtttgaatgagtaaaacaattaaatagtattggtgaggtaactagatgaattacatggagattcgaccatttgacacagctaatggtgaaggggttcgggtaagcctcttcgtagctggctgtaaacatcactgtgaaggctgctttaacagggagtcttggaagtttaatgctggtaaagagtttacttatgcaaacctctacggcatcattaagttaatggatgataaggccattagtggactgtcaatacttggtggggagcctctggatgatagaaacattcaagaggtcaccaacatatgcaagcgtattaaaactgtttatccagaaaagtctatatggctttggacaggttttcaactgcacgaaaaaatccacttagatgtgatgaaatatgttgacgtggtgattgatggtaagtatgattcatctaaaccaacagttaaaccatatcgtggttctgataaccaaaacctctggagaaaagagtatgggtggcaaggtgattgtcagtggagagccgagtaagtctcgtcgtaaagtaggttatcggtaaaggccactttacaatgtgctacagaacaaacttaaccgttggtgtcactgaagaagaacttaaagcattttgtgaaaagattgagagagatacgaaatgggaatctggaacagttttaaatcaaaagtaaaagcagcactggcatcactaggtatgctcactggtgttgatgtgactcatctcatgcagcacttcaagatggatgagaaactagctaatgagattatgaaaagtatctatgtaggccgtggtaaaggtggtaagaaacaagcacatcgaccaactggtgcagcagctattaaacgggcagctaagaaagctcgtaaccgtaaacgtaataagaaggctaaataatcatgagcaaagtttataacactcgtaaattacagattttcgtactgtgccagtttatggctaaagagtataactgctactactgtggcacagggttcatcagtgacaatgacggctcatatctaccgttcaaagaggctgtaaaactcttcaatgaagaagtaagctctaagaaagatatcgaaaaggttaagctaacctacagtaagaaagataagaagattatctgcctagataactttgtaaaagttactggagaagcaaaagagtttatggatgagagtgagatttcattcacaagcatcttgaaagtagctcagtaaacagatactataagggaactgcaaaggttccctttttatttttggagataacatgtatctgtcaaatctgaaacgttcggctgcaatgtcagttctaagactcagctttgaggaacgtcaagagttcattgactcccaccaatacgacccttctaactctaaccgcatgattctctggaatcgtgatagtactcgtgaaagggcactattccgctattacccacattacaccatagataacctgtatgaatgttttgttgtgaagaacactatcacaattctcaacaaactttgcagatacacaggtaatcaatccttcacattaggtcaccacaagcctgttacaaaaggtggtgaacatcactgtgcaaactggtttatccagactaaacttgataaccagaagcaaggcgatagtcttttaagcacccctaagatgacctatgaagagcaagagaaatatatcaaaaataatatgccagatgtgcttgacaacagctatacagatttggcaatatctctcctgttgaagttcgagacagtttatagggcaacttacgatggctagaacagattgggaagtaaaagaacttgtaagtgatgctggaaatggctgtgaggtgtatatgattcgtggccatgtgccagacccattagctattgagatgattgaccatcacacaagtggattctacaaagaactcggtgaaccaagtgttgaaagaacttggacaaaaccagtaccagatagcacaggtaactgttcagtaatctatcacattgtgaatccacaaaaatgcaaaacagctatggctgtgacattcgtaacttttgattgagagagaaaattatgaaaactattattgaagccgttgtcaccgcacaagagaaattcttccacacagaaaaatttaatatcatccaatttccatctggagaaattggtggaaacttctctgaagactttgttaagtttactgagcgtaatgctggtaagattgacaatgttatattgactgtgcagggatatgataaagatacactgtttgcattagctttggctaaggatgccgttgacagtcttgttccatacaaatcagcgatgaaaaccgttgttttcgggttcttaccaaatgctcgatatgaccgccatatgttcaaaggtgattcagcagcactgaaagcttttgccaacctcgttaatgcaatgggctttgatgcagtgtgtgctcttgaccctcacagtaatgttgcagaaaatttatttaagtgctttcagagcatgaagcaaaaagatgtagcagcacactttgcaagtgacccacgcattgatttcttggtagccccagatgcaggtgcagctaagaagactgaagagactgctaaagaggtagataagccatatattacaatgtctaaagtacgcaaccttaagactggtgaaattactggtatgcgaatccttgatactgttgatttgaccgataaaactgtcatgattcttgatgatatttgtgatggaggccgaaccttcatagaagcagctaaacatcttcgtgaagcaggtgcaaaacgtgtggaactctatgtaactcatggcattttctctaaaggtgttgaaaacttacttgacaatggtgttgaccatatctacactacaaactctttaggggaagctaaagaccgtggcttaacacattatacccaagttactgtagtaaccattgattgaaactttataggggcttcaaagccccattttgagagagattaaaagatgactaaatcactttatgcagtaccagcaggtttaaacgcagatgcttacaaatctggtcacatttaccagtaccctagtgcaacagaatacttgatgttcaatctgacaccacgtagtgataagtggttcaacagccccttagcaattgatggtgtagtcgcttttggtattcaacgttttgttaaagattacctgatagaccactggaacgccaccttcttcgaacgtgacaaaaaagaagcaattgacgaaatcttagaagttatgaacggtgttctgggtaaagatgctattggtcgagaacattgggaagcactccacgacttaggttatctaccagttgaagtctacgctgtagaagaaggcacagttgttcctatgcgtgtaccaatgattgtcttccagaacactgtttcaggtttccattgggtagcgggatatctggaagatgctttctctgctgagatttggaaggcttgtaccattgcaactattgcattgcactacaaacgtatctgtaagaagtggtctgaccttacttgtgacaacgacttacatttgccttatcagtgccatgactttgctatgcgtggtatgtccggctttactgatgacgcatttaacgctgtaggtcacttaacaagctttaaagggactgatagcttccctgcggtatatacagctaaacgcatctatggacagtcctatccaatctctgatattggtagctctgtaccagccactgaacactctgtaatgtgtgcaaacattgcttgggaaggtggtaatgagttgattgaagaagaaagacgcttcaaaggtgagttacaaaccttccgtcgcttcttaacagaaacttacccaactggtattgcaagtgttgtttcagacacttataatttctggagaactgtatcagtaatactaccagaactccgtaaagagattatggaacgtgatggcaaactggtaattcgtcctgactctggagaccctgtacatattgtcacaggatataaagcaattcacttagagtgtgctaaaaaggcttattacgaacacctaagcaagcagaatgccagtgacacaatgttgagcaccgtcctgaacatgaagcttgaagatattaactatgttatggctgggtggttacgtgcagaaggctatgaaatggtggttgacagagaagactttgaagtagccgatacagtgttgctgaaagatgcttacatcaacggctctgcaaacgttgtaacacgtcctgtagcggagattgatggagctattaagacactgtacaacatctttgggggaactattaactctaaagggtttaaggtactggatgagcacatcggccttatctatggtgactctatcacgctggaacgtgcaaacgaaatcctgaagcgtctgtatgaaatgggttttgcaagctctaacgtagtgtttggtgtaggctcttacacttaccagtacatgactcgtgatacctttgcatttgctgtcaaagcaactcttgcaagcattggtggtaaagagattatgcttgcaaaagaccctaaaacagatagtggtgttaagaaatctgcttttggtggtgtagcacccatgtgggatggcgaaaagctgaaagctgtagatggctatgggttccagagctttgcagatgtacttgaccacccagcttgtgctttacgtctagtctttagtgactctgagcagttcggctacacaactcttggtgatattcgaagtaatattgacaagcagctttaaaagtatatgataagaggctcctacgggagcctttttaatttctggagaatattatgaaaatcaaagagatgaacatcaacattgtcttagaagaacgttgggagaacatcaagaagcctgaagatggtcataagttccttaacaaaatcttagtagcagctaaagaggaactgactggtaagattgcagcagcaatcacaataaaggtttgtgtaaaaggtcttccagacaaccaccaatttgcacttgacgagtttaaagaaagcttctacaatccaaacaaacagatgcttgaaagtaactttgcagtgtctacaagtatcgtccatgacagaagctttatcctttacaagaatatgagaggtgagtcatgcaagcatattggatagaaattttactgtcacttggcagtgtagcagtctttgtttaccttctttgcaagtactatgcagagcacaagaagtgtgattactgcaatggggaagggtatacaagagcaggttgttgccctatgtgtggcggttctggtaaaatgtttaataagtaattcaacagtaaactaagaggaaagtattatgcgcatggtaaacgaccacgcagaagtgattaagagttcaacttctttggagacatctcaagcacagattacaatgacacctgaaatgttcagccttctgagttctggtgtatacacatttaaagaaagggcagtgattcgtgaactgtcatgtaacgcagtagatgctcagaaagaagctggaagagagaacatcccgttccatgtgcatttacctactcgttttgagccttactttgaagttcgtgattttggaactggattgactcatgataaagttatgagtttgtacctaaactacggggcttctacgaagaatgactctaatgattacattggtgcaatgggtatcggctcaaaatctccatttgcaattgctcagtcattcacagtatctagctatgttgacggtgttgttaataagtactctgtttatcttgagaatggtatccctcaagtaactaagctgacaactaacccaacaaaagaacctaatggtttagctgtacgtgtggcagttgctgaccaccgtatctcaaagttctttgaagaagctggtaatgtatactcatactttgctgtaaaaccagaaagtaatattgtgtacgacgatgtattagcagatatgaacgtcattgcccgtgaagagggtgtttatgatgccatgattcataagcaaagttggcgttctagtggtaacaggacagagtttaatgtggtgatgggtaacattgcctaccctgttaacatggaagcattacttggtgatgacttcttcaaagtattgccagaatttttccgtaggagcgtagaccttgtaaacatctacatgcctattggttcagttgctattgcagcttctcgtgaagcattgcagatgaatgacacgacaaaaaatgttatcattgaggctactaaaaagataactgaagccattacaaaggatgttatcaagaaagttaatagccaacctacactcatggatgctgcacaggcttatgctgagttacgtttgaactcacgagagatgtttaatgctgtgtgtccaaagctagagtggggaggcgttaagcttgattctctggaagaagaattgctaaacattcgtcgtgggattatctacgcagaagacggctcagtcatttatgaacgtgatggtaaaggtaatattaaggttgacagtaaaggaaacaacattcctaaagtggactacctatacaatccagttgcttatgtcaagttcaactctttagagagtaagattcgtgcaacagcactttcctacactcaagaggcaagtatgttcaatatctttggtgcaatgcgtaaaagccaaattgaacagtttttgttcgtgattaatgaccgtcgcaataaaaacggtactgagaagactgtaggacgcaatcaaattttgcgtggtgcatgtcgagactacgccagtgagtcaaatctgtttcacagatacaatggtattgtctttgtattctcgactgaaaaagagttagatgatttaatcaatctacacaaacttgataaaagcttgttaaagattgtgaagatgtctgataaggaacaccactatcagcgtaaagaagctgtaaggggtgttgtaaaactctggaaagctgttccagcagaaactaaagcttcatacaaagaggtgtcagaagacttagacacaattgaagagcctcagctttatattaaggcagtaggtgacacagttgatggtagatgtttttgctcatctccagaggatgtagctaagtcagttgctaatgttacaggcaagacagtctatgttttccgaaaagcaaattggaaaaagataccagaagactggattgaagtagatgagaagctcttgaacgacagcttaactgatgttcattggattaaccataacaggtatatgacacgcatctacatggatggtgtcctcgaccttacaagcagttggattattgccagaaactttacgttcaataacaggaagatttctcgcgggtattgttattcaagggatactattaagacaatcttcttagaagggaatgaagatgctgttgaggcaatgttcggtaagattcagtatgtggctgcaccgtttgcatacacttacactatcagtgttcttcaggatttgaagtcatgccttgataatgacacaaaactgtacaaaaagattaaaaaagcaggtgaccgcatggttatcaagctgacaaactatctttcaaaaagaaaacaagaaaacttcttgctttcacacttagattggaataaagtatcacctatcgaagtgagtaagtttttaggctttaatgtgaaatgtgttccagaaggtacaacaatttacgattaaagtgtttgacaaggggctttgaagccccttagaatagcctcacaaattgattgattaactaacaagagagagtaataagatgactactaaaactaaagcacagattgacgcagaaatttacaaactggttaaagaaggtaagctgactaaaacagctattgcacagaagttcaatacttcaacccgttcagttggtcgtgctgtagagcgtcatgaagcaaccttaaaaggcaagaagaaggcaacttctacaccagctacgaagactttgaaacaagttgctaagacttttaaaaagaaagctggcaaaccagttgaaaaggtcgttaaagacactgtacagaaagctccagtaaataaactgcacgaagctatgcagaaagatgacaagattgagtacatgattactggtgattctgtaattatgacttatggttcagaatctgaaattgttgagtctactcatccgaactatcaagagattgtagttcatgttgtgaaaggtgagttcaagaaagcatttgaactgatgaacattcgtaagtctatcgaaaacttcactcagggtgctatcacaatcaaaggcgacaagctattctacggtgctgttgagatgcgttctactctggttgaccgtattcttcacatgatgaaaactggcgataaaggttttgaacgacttgtaatgttcttcgaaaaactgatggaaaacccatctaaagattctgtagaacaactttggggatttgtatctcaccttgatgttgaaattgatgaagaaggctacatcattggttggaagaaagtctctactcgtgaaggcaagctggttgactctcacacctacaaagtgcctaatgatttgggtaacattgtagaaatgccacgttggatggttgataataaccgtaacgtaacttgctctcaaggtcttcatgttggtgcttgggattatgttcgttgcttctcaggtgatacaatcctgaaggttcgcgtacatccacgagatgttgtatctgttccaactgattacaatgatatgaagatgcgttcctctcgttatgaagttgcagcaatcgttgataatcaacgtaaagtgctggaagcatgggacggtaagactgaagctttgcatgtcatcgttggcactgctggtgaactcatctctcaacgtaaacgtgaaatctaataagtaatttcttaaaaggctgcttcggcggccttttttatttgtattttgtgcagagtgctgtataattgatgtcacgataaactaaacaggagaaccaatgaagaaattgattctaggtttgtgcttaatgtttacagcacacttatcttatgcagttgactgcccagagctatcaattagtcaaaaagtaaacatgttaaaagcttaccagtatggtgagaataatatggggaaaggctggggtattactctagcagctatagccttacaagagtcagagttaggtctgaaggtagagaataaaaagacccacgactatggtattttccagaatcacttgaagactgttgtaaagcgtaacaaaattagtcctaatgtggctaaaaagaaactcttaaaagactttgactatgcagcaagagagacacataaagagcttgagttttggactaaggtacatggtcacccaaagtcaaagaagactttacaaaaagttttagcatcatataatgctgggtattcgtacaaaatccctaaagctaagaaatactctcaagatgtctataacaatatgaaagttatcgctcaatgtgaatttgcaacaaacatttctaaggtaaatcatgaaaaaattaagaaagtctgatgaagtcctgtgccatgcttacgacttgcaccctcatgagttaggtcttgattcctgtgtatggactccagaacagtgcagggattttgaagatactgcaagagaagttgtatgttcacttgaagagttccacacatcagaaccaattgtgagcgttgtggacaaagagactggacaaactgtaggtgtaaggcgtgacagcttagtcatagtcaataaagaccttgtagaaaaaggtaaccttatcttagcagatattgatggtgttcttacaaattttaatcacgaagattgctcaacggaattgactgatgggtcattctcacagtacactaatcttcttgactctgtaagagcaaaaccaacatatgtttttagtattattgatgcaattgctaatcatgctgccattggactcttgacagcgagaggtgaatctcagagaatacctactgagatgtttttaaggcataacatagagcatgattacatgctttttatgcgtggttttggaactaactctataagtgcagaaagtttaaaagtgaggatgattcagtcttgcattcttccttattttaatatagtatgctttatagaagatacagagaagaatgttcagaaggtaaacagaatccttccacacattaaaaccatgttagttaaacattgagagaaaaacttatgaacaatatcattacggtagcactggacgatacggcaaacaaatctgaagtagttcgtaaaatgattcaaggtaactttgaaggtaaaattttccgtgctgttaatgtaaaagctgatggcagtattcgtgaataccgtgcattgttgaatgttaaaaagcacgtaaaaggtgctggttcaacgactgcacacaaagaaaacctgatgactatctatgatatgggtaaggcttcagagttaggtgctgaaggtatcgttaaagaaggtgctccgtatcgctctttcaatctggaaactactcttatgctttcctttacgagtggttctaaaacaatcacttatctctttactgatgctgcaacggtatctgctattaaggatagcactgtcaaagctggcgtagttgctgctgcaaaagcttcttcgatggctgcgaatatccttgctaaagtcctcggttaaggttaagatacaggctccttcgggagcctttttcatttcaggagattgttaaatgacatttaaagagttctgtcaagctactttcataattgttttcttagttggtgcaggtgtctggggaggatactcttacagagactatcaagttgctgaaactgagctaaataatgagaaactaataagtgttgctaaagatgcttatcaggaaggtttagccacactcagcaccaattacaaaaatgatttgaaagatgtgcttgctaagaataagcatacaaaagaggtactaacatatgaaaaaactaagccagagttttataatgtttgtgctactgataactatgtcagggtgttcaacgaacaaagtgaacagtacattcaaaaactcccaagtaagtgagagtgataagtacactcaagaagaatcacggtatatcatcaaaggcaacacaggcagtgatgtagcagcagcccttgagttctatcgtgacggcttttaccagtgcacaatcaaagctaacaaccttattgacatgattttattagggaataagcagcaatgacagagaatcaagatactttctatgtagaaggttacttactgttgccacgaccaaaagaaacttatatgcgaattgatttcttaccaaccattatggataatgtgatgtgccatatctttatgcaaggtgtcacagcacagcttaagcatgttggtaaagagtgcaaaataaaggttgacactcatccagaaatcaaagagaatcactacacatggttcttaccagactctaaagaaatcttagcagttcttaaaacgaggaagtaactatgcagattaatggaagagactttgtagccgtttactacgagaaaggtaaagaagttggcgtagcacaagtgtcttatggcaatggtaagtggctatatggaacaatcgcagtagttggaacaagaagtgatacaaaaacttttaaagattgtgttgaccttcttgaagaatccattcagaatcattggtgtctgatatggatgactgacaatgaagtgatagaacgcttcaaaaagattgatatcaacattgatagtattgagcatgttgatttatatgagctaactgagaaggtaaactatgagagtaattctagccagagataaaaagacacggaaactcatccggtcagcagtattcattggaagaaatgaagtcatcccgtttacagcaaacgatgtgatagcctacaggaaaaacttagtatttagtgattgcttgcgtggcttcatctcccaaccgttggaagactttaaaagccgttgtggtaaaggtatcatcattcaagaggcaattatcaatggctgacttctgcaaagactgttctatagaaatgtttggacgtgatacaggtgacttaaaaggtcttatcactgaagatgactttaaagctggctacgcaatgccagtaatctgtgaaggttgtgggtgcatctgggtagaccacgaaggacagcgtgtaaaaccttcagaagataaagaatcttgggagagatgttaaatgggtattgtaaaaatcatcaaaggtgatatctttgatgcctttgataaaggcaaatttgatatcattggtcatggttgcaactgtatgaatttcatgggtgcaggtattgcagcaactatttctaagcgttatccaaaagtgtatgatactgacacagaagtttacctgtttgcaggtggcattcgtcacaaaccctgtgaaaatttactaggtaatttctctgtagcacgtttaaaacaaggccgtatagctaacctttacacccaacttcatactggtaaagatgcccgatacagtgctttagagtcatctttgaaacagcttaacagatactgtgaagttaaccagttgaagaaagttggtctacctatgattggtgcaggtatcggtgggcttgaccctcaagctgtcacagttatcattaatcaggtgatgaagagtgtagatgtttatctgtatgtctatgaagcaaaaatgtataatgagttacactcaggttggaaaaactactgtgaaccagaatactttgcaggtgtagttgtgttcacagataacgaagttaccctcttcagacgaagaaaaggtaagatacatcaaagtaaccctccggttgaaaagatgtctctgagtaacgctctagttacccacctgtcgaagagcaatcacaggattgcagtaacatttggcagtgatgcagatacttatatctatgcaagaactgatgaggatatcgagacagttttctcttcgccagaagttaccttcttagacgcaaagaactaagaaacttcgtaggatattcacaatagactaagcctttcttatctggtaaatttttcaggtaaggagggctttttcatttcagaattgtgcaggtatatttctatagagaaatctgtagaggaaattttgaactccaaagagaaaattcattttcaaactgtgcaggtaatctccaaagggttttttcattttcaaaacgtgcagagaaaaatcaggtcgggcaagaaaaatcattttagatttgtgcaggtaggtttttatagagagccagaccaacccctctccccctcttactccccctctccccaaaagtcaagagaaaaatttgtagcggattgaaaaataattcttgatttgtgttctgttttgtggtagtcgtgtgcgcccgtttcattacagttttgccaaaaatatttttaaaattttttcttgactctttgaacgtggttatctagtatttgcatcaacggggagggaatgaccttccccactaaataaagtcctaaactggagattcaaaaatggcatatcgtgcacctaaattcatcaacaaagacaactttcgcaacgcactggagaagtcacttgatgaaaattttaaaggcaacattatcgttgttcacgcattcaattttaagtatgatgtaaacgggaatagaatcaaccatta

SEQ ID NO: 2 - DNA, 87662 bp, Artificial
  found in bacteriophage isolated from environment
taatggttgattctattcccgtttacatcatacttaaaattgaatgcgtgaacaacgataatgttgcctttaaaattttcatcaagtgacttctccagtgcgttgcgaaagttgtctttgttgatgaatttaggtgcacgatatgccatttttgaatctccagtttaggactttatttagtggggaaggtcattccctccccgttgatgcaaatactagataaccacgttcaaagagtcaagaaaaaattttaaaaatatttttggcaaaactgtaatgaaacgggcgcacacgactaccacaaaacagaacacaaatcaagaattatttttcaatccgctacaaatttttctcttgacttttggggagagggggagtaagagggggagaggggttggtctggctctctataaaaacctacctgcacaaatctaaaatgatttttcttgcccgacctgatttttctctgcacgttttgaaaatgaaaaaaccctttggagattacctgcacagtttgaaaatgaattttctctttggagttcaaaatttcctctacagatttctctatagaaatatacctgcacaattctgaaatgaaaaagccctccttacctgaaaaatttaccagataagaaaggcttagtctattgtgaatatcctacgaagtttcttagttctttgcgtctaagaaggtaacttctggcgaagagaaaactgtctcgatatcctcatcagttcttgcatagatataagtatctgcatcactgccaaatgttactgcaatcctgtgattgctcttcgacaggtgggtaactagagcgttactcagagacatcttttcaaccggagggttactttgatgtatcttaccttttcttcgtctgaagagggtaacttcgttatctgtgaacacaactacacctgcaaagtattctggttcacagtagtttttccaacctgagtgtaactcattatacatttttgcttcatagacatacagataaacatctacactcttcatcacctgattaatgataactgtgacagcttgagggtcaagcccaccgatacctgcaccaatcataggtagaccaactttcttcaactggttaacttcacagtatctgttaagctgtttcaaagatgactctaaagcactgtatcgggcatctttaccagtatgaagttgggtgtaaaggttagctatacggccttgttttaaacgtgctacagagaaattacctagtaaattttcacagggtttgtgacgaatgccacctgcaaacaggtaaacttctgtgtcagtatcatacacttttggataacgcttagaaatagttgctgcaatacctgcacccatgaaattcatacagttgcaaccatgaccaatgatatcaaatttgcctttatcaaaggcatcaaagatatcacctttgatgatttttacaatacccatttaacatctctcccaagattctttatcttctgaaggttttacacgctgtccttcgtggtctacccagatgcacccacaaccttcacagattactggcattgcgtagccagctttaaagtcatcttcagtgataagaccttttaagtcacctgtatcacgtccaaacatttctatagaacagtctttgcagaagtcagccattgataattgcctcttgaatgatgatacctttaccacaacggcttttaaagtcttccaacggttgggagatgaagccacgcaagcaatcactaaatactaagtttttcctgtaggctatcacatcgtttgctgtaaacgggatgacttcatttcttccaatgaatactgctgaccggatgagtttccgtgtctttttatctctggctagaattactctcatagtttaccttctcagttagctcatataaatcaacatgctcaatactatcaatgttgatatcaatctttttgaagcgttctatcacttcattgtcagtcatccatatcagacaccaatgattctgaatggattcttcaagaaggtcaacacaatctttaaaagtttttgtatcacttcttgttccaactactgcgattgttccatatagccacttaccattgccataagacacttgtgctacgccaacttctttacctttctcgtagtaaacggctacaaagtctcttccattaatctgcatagttacttcctcgttttaagaactgctaagatttctttagagtctggtaagaaccatgtgtagtgattctctttgatttctggatgagtgtcaacctttattttgcactctttaccaacatgcttaagctgtgctgtgacaccttgcataaagatatggcacatcacattatccataatggttggtaagaaatcaattcgcatataagtttcttttggtcgtggcaacagtaagtaaccttctacatagaaagtatcttgattctctgtcattgctgcttattccctaataaaatcatgtcaataaggttgttagctttgattgtgcactggtaaaagccgtcacgatagaactcaagggctgctgctacatcactgcctgtgttgcctttgatgatataccgtgattcttcttgagtgtacttatcactctcacttacttgggagtttttgaatgtactgttcactttgttcgttgaacaccctgacatagttatcagtagcacaaacattataaaactctggcttagttttttcatatgttagtacctcttttgtatgcttattcttagcaagcacatctttcaaatcatttttgtaattggtgctgagtgtggctaaaccttcctgataagcatctttagcaacacttattagtttctcattatttagctcagtttcagcaacttgatagtctctgtaagagtatcctccccagacacctgcaccaactaagaaaacaattatgaaagtagcttgacagaactctttaaatgtcatttaacaatctcctgaaatgaaaaaggctcccgaaggagcctgtatcttaaccttaaccgaggactttagcaaggatattcgcagccatcgaagaagcttttgcagcagcaactacgccagctttgacagtgctatccttaatagcagataccgttgcagcatcagtaaagagataagtgattgttttagaaccactcgtaaaggaaagcataagagtagtttccagattgaaagagcgatacggagcaccttctttaacgataccttcagcacctaactctgaagccttacccatatcatagatagtcatcaggttttctttgtgtgcagtcgttgaaccagcaccttttacgtgctttttaacattcaacaatgcacggtattcacgaatactgccatcagcttttacattaacagcacggaaaattttaccttcaaagttaccttgaatcattttacgaactacttcagatttgtttgccgtatcgtccagtgctaccgtaatgatattgttcataagtttttctctcaatgtttaactaacatggttttaatgtgtggaaggattctgtttaccttctgaacattcttctctgtatcttctataaagcatactatattaaaataaggaagaatgcaagactgaatcatcctcacttttaaactttctgcacttatagagttagttccaaaaccacgcataaaaagcatgtaatcatgctctatgttatgccttaaaaacatctcagtaggtattctctgagattcacctctcgctgtcaagagtccaatggcagcatgattagcaattgcatcaataatactaaaaacatatgttggttttgctcttacagagtcaagaagattagtgtactgtgagaatgacccatcagtcaattccgttgagcaatcttcgtgattaaaatttgtaagaacaccatcaatatctgctaagataaggttacctttttctacaaggtctttattgactatgactaagctgtcacgccttacacctacagtttgtccagtctctttgtccacaacgctcacaattggttctgatgtgtggaactcttcaagtgaacatacaacttctcttgcagtatcttcaaaatccctgcactgttctggagtccatacacaggaatcaagacctaactcatgagggtgcaagtcgtaagcatggcacaggacttcatcagactttcttaattttttcatgatttaccttagaaatgtttgttgcaaattcacattgagcgataactttcatattgttatagacatcttgagagtatttcttagctttagggattttgtacgaatacccagcattatatgatgctaaaactttttgtaaagtcttctttgactttgggtgaccatgtaccttagtccaaaactcaagctctttatgtgtctctcttgctgcatagtcaaagtcttttaagagtttctttttagccacattaggactaattttgttacgctttacaacagtcttcaagtgattctggaaaataccatagtcgtgggtctttttattctctaccttcagacctaactctgactcttgtaaggctatagctgctagagtaataccccagcctttccccatattattctcaccatactggtaagcttttaacatgtttactttttgactaattgatagctctgggcagtcaactgcataagataagtgtgctgtaaacattaagcacaaacctagaatcaatttcttcattggttctcctgtttagtttatcgtgacatcaattatacagcactctgcacaaaatacaaataaaaaaggccgccgaagcagccttttaagaaattacttattagatttcacgtttacgttgagagatgagttcaccagcagtgccaacgatgacatgcaaagcttcagtcttaccgtcccatgcttccagcactttacgttgattatcaacgattgctgcaacttcataacgagaggaacgcatcttcatatcattgtaatcagttggaacagatacaacatctcgtggatgtacgcgaaccttcaggattgtatcacctgagaagcaacgaacataatcccaagcaccaacatgaagaccttgagagcaagttacgttacggttattatcaaccatccaacgtggcatttctacaatgttacccaaatcattaggcactttgtaggtgtgagagtcaaccagcttgccttcacgagtagagactttcttccaaccaatgatgtagccttcttcatcaatttcaacatcaaggtgagatacaaatccccaaagttgttctacagaatctttagatgggttttccatcagtttttcgaagaacattacaagtcgttcaaaacctttatcgccagttttcatcatgtgaagaatacggtcaaccagagtagaacgcatctcaacagcaccgtagaatagcttgtcgcctttgattgtgatagcaccctgagtgaagttttcgatagacttacgaatgttcatcagttcaaatgctttcttgaactcacctttcacaacatgaactacaatctcttgatagttcggatgagtagactcaacaatttcagattctgaaccataagtcataattacagaatcaccagtaatcatgtactcaatcttgtcatctttctgcatagcttcgtgcagtttatttactggagctttctgtacagtgtctttaacgaccttttcaactggtttgccagctttctttttaaaagtcttagcaacttgtttcaaagtcttcgtagctggtgtagaagttgccttcttcttgccttttaaggttgcttcatgacgctctacagcacgaccaactgaacgggttgaagtattgaacttctgtgcaatagctgttttagtcagcttaccttctttaaccagtttgtaaatttctgcgtcaatctgtgctttagttttagtagtcatcttattactctctcttgttagttaatcaatcaatttgtgaggctattctaaggggcttcaaagccccttgtcaaacactttaatcgtaaattgttgtaccttctggaacacatttcacattaaagcctaaaaacttactcacttcgataggtgatactttattccaatctaagtgtgaaagcaagaagttttcttgttttctttttgaaagatagtttgtcagcttgataaccatgcggtcacctgcttttttaatctttttgtacagttttgtgtcattatcaaggcatgacttcaaatcctgaagaacactgatagtgtaagtgtatgcaaacggtgcagccacatactgaatcttaccgaacattgcctcaacagcatcttcattcccttctaagaagattgtcttaatagtatcccttgaataacaatacccgcgagaaatcttcctgttattgaacgtaaagtttctggcaataatccaactgcttgtaaggtcgaggacaccatccatgtagatgcgtgtcatatacctgttatggttaatccaatgaacatcagttaagctgtcgttcaagagcttctcatctacttcaatccagtcttctggtatctttttccaatttgcttttcggaaaacatagactgtcttgcctgtaacattagcaactgacttagctacatcctctggagatgagcaaaaacatctaccatcaactgtgtcacctactgccttaatataaagctgaggctcttcaattgtgtctaagtcttctgacacctctttgtatgaagctttagtttctgctggaacagctttccagagttttacaacaccccttacagcttctttacgctgatagtggtgttccttatcagacatcttcacaatctttaacaagcttttatcaagtttgtgtagattgattaaatcatctaactctttttcagtcgagaatacaaagacaataccattgtatctgtgaaacagatttgactcactggcgtagtctcgacatgcaccacgcaaaatttgattgcgtcctacagtcttctcagtaccgtttttattgcgacggtcattaatcacgaacaaaaactgttcaatttggcttttacgcattgcaccaaagatattgaacatacttgcctcttgagtgtaggaaagtgctgttgcacgaatcttactctctaaagagttgaacttgacataagcaactggattgtataggtagtccactttaggaatgttgtttcctttactgtcaaccttaatattacctttaccatcacgttcataaatgactgagccgtcttctgcgtagataatcccacgacgaatgtttagcaattcttcttccagagaatcaagcttaacgcctccccactctagctttggacacacagcattaaacatctctcgtgagttcaaacgtaactcagcataagcctgtgcagcatccatgagtgtaggttggctattaactttcttgataacatcctttgtaatggcttcagttatctttttagtagcctcaatgataacattttttgtcgtgtcattcatctgcaatgcttcacgagaagctgcaatagcaactgaaccaataggcatgtagatgtttacaaggtctacgctcctacggaaaaattctggcaatactttgaagaagtcatcaccaagtaatgcttccatgttaacagggtaggcaatgttacccatcaccacattaaactctgtcctgttaccactagaacgccaactttgcttatgaatcatggcatcataaacaccctcttcacgggcaatgacgttcatatctgctaatacatcgtcgtacacaatattactttctggttttacagcaaagtatgagtatacattaccagcttcttcaaagaactttgagatacggtggtcagcaactgccacacgtacagctaaaccattaggttcttttgttgggttagttgtcagcttagttacttgagggataccattctcaagataaacagagtacttattaacaacaccgtcaacatagctagatactgtgaatgactgagcaattgcaaatggagattttgagccgatacccattgcaccaatgtaatcattagagtcattcttcgtagaagccccgtagtttaggtacaaactcataactttatcatgagtcaatccagttccaaaatcacgaacttcaaagtaaggctcaaaacgagtaggtaaatgcacatggaacgggatgttctctcttccagcttctttctgagcatctactgcgttacatgacagttcacgaatcactgccctttctttaaatgtgtatacaccagaactcagaaggctgaacatttcaggtgtcattgtaatctgtgcttgagatgtctccaaagaagttgaactcttaatcacttctgcgtggtcgtttaccatgcgcataatactttcctcttagtttactgttgaattacttattaaacattttaccagaaccgccacacatagggcaacaacctgctcttgtatacccttccccattgcagtaatcacacttcttgtgctctgcatagtacttgcaaagaaggtaaacaaagactgctacactgccaagtgacagtaaaatttctatccaatatgcttgcatgactcacctctcatattcttgtaaaggataaagcttctgtcatggacgatacttgtagacactgcaaagttactttcaagcatctgtttgtttggattgtagaagctttctttaaactcgtcaagtgcaaattggtggttgtctggaagaccttttacacaaacctttattgtgattgctgctgcaatcttaccagtcagttcctctttagctgctactaagattttgttaaggaacttatgaccatcttcaggcttcttgatgttctcccaacgttcttctaagacaatgttgatgttcatctctttgattttcataatattctccagaaattaaaaaggctcccgtaggagcctcttatcatatacttttaaagctgcttgtcaatattacttcgaatatcaccaagagttgtgtagccgaactgctcagagtcactaaagactagacgtaaagcacaagctgggtggtcaagtacatctgcaaagctctggaacccatagccatctacagctttcagcttttcgccatcccacatgggtgctacaccaccaaaagcagatttcttaacaccactatctgttttagggtcttttgcaagcataatctctttaccaccaatgcttgcaagagttgctttgacagcaaatgcaaaggtatcacgagtcatgtactggtaagtgtaagagcctacaccaaacactacgttagagcttgcaaaacccatttcatacagacgcttcaggatttcgtttgcacgttccagcgtgatagagtcaccatagataaggccgatgtgctcatccagtaccttaaaccctttagagttaatagttcccccaaagatgttgtacagtgtcttaatagctccatcaatctccgctacaggacgtgttacaacgtttgcagagccgttgatgtaagcatctttcagcaacactgtatcggctacttcaaagtcttctctgtcaaccaccatttcatagccttctgcacgtaaccacccagccataacatagttaatatcttcaagcttcatgttcaggacggtgctcaacattgtgtcactggcattctgcttgcttaggtgttcgtaataagcctttttagcacactctaagtgaattgctttatatcctgtgacaatatgtacagggtctccagagtcaggacgaattaccagtttgccatcacgttccataatctctttacggagttctggtagtattactgatacagttctccagaaattataagtgtctgaaacaacacttgcaataccagttgggtaagtttctgttaagaagcgacggaaggtttgtaactcacctttgaagcgtctttcttcttcaatcaactcattaccaccttcccaagcaatgtttgcacacattacagagtgttcagtggctggtacagagctaccaatatcagagattggataggactgtccatagatgcgtttagctgtatataccgcagggaagctatcagtccctttaaagcttgttaagtgacctacagcgttaaatgcgtcatcagtaaagccggacataccacgcatagcaaagtcatggcactgataaggcaaatgtaagtcgttgtcacaagtaaggtcagaccacttcttacagatacgtttgtagtgcaatgcaatagttgcaatggtacaagccttccaaatctcagcagagaaagcatcttccagatatcccgctacccaatggaaacctgaaacagtgttctggaagacaatcattggtacacgcataggaacaactgtgccttcttctacagcgtagacttcaactggtagataacctaagtcgtggagtgcttcccaatgttctcgaccaatagcatctttacccagaacaccgttcataacttctaagatttcgtcaattgcttcttttttgtcacgttcgaagaaggtggcgttccagtggtctatcaggtaatctttaacaaaacgttgaataccaaaagcgactacaccatcaattgctaaggggctgttgaaccacttatcactacgtggtgtcagattgaacatcaagtattctgttgcactagggtactggtaaatgtgaccagatttgtaagcatctgcgtttaaacctgctggtactgcataaagtgatttagtcatcttttaatctctctcaaaatggggctttgaagcccctataaagtttcaatcaatggttactacagtaacttgggtataatgtgttaagccacggtctttagcttcccctaaagagtttgtagtgtagatatggtcaacaccattgtcaagtaagttttcaacacctttagagaaaatgccatgagttacatagagttccacacgttttgcacctgcttcacgaagatgtttagctgcttctatgaaggttcggcctccatcacaaatatcatcaagaatcatgacagttttatcggtcaaatcaacagtatcaaggattcgcataccagtaatttcaccagtcttaaggttgcgtactttagacattgtaatatatggcttatctacctctttagcagtctcttcagtcttcttagctgcacctgcatctggggctaccaagaaatcaatgcgtgggtcacttgcaaagtgtgctgctacatctttttgcttcatgctctgaaagcacttaaataaattttctgcaacattactgtgagggtcaagagcacacactgcatcaaagcccattgcattaacgaggttggcaaaagctttcagtgctgctgaatcacctttgaacatatggcggtcatatcgagcatttggtaagaacccgaaaacaacggttttcatcgctgatttgtatggaacaagactgtcaacggcatccttagccaaagctaatgcaaacagtgtatctttatcatatccctgcacagtcaatataacattgtcaatcttaccagcattacgctcagtaaacttaacaaagtcttcagagaagtttccaccaatttctccagatggaaattggatgatattaaatttttctgtgtggaagaatttctcttgtgcggtgacaacggcttcaataatagttttcataattttctctctcaatcaaaagttacgaatgtcacagccatagctgttttgcatttttgtggattcacaatgtgatagattactgaacagttacctgtgctatctggtactggttttgtccaagttctttcaacacttggttcaccgagttctttgtagaatccacttgtgtgatggtcaatcatctcaatagctaatgggtctggcacatggccacgaatcatatacacctcacagccatttccagcatcacttacaagttcttttacttcccaatctgttctagccatcgtaagttgccctataaactgtctcgaacttcaacaggagagatattgccaaatctgtatagctgttgtcaagcacatctggcatattatttttgatatatttctcttgctcttcataggtcatcttaggggtgcttaaaagactatcgccttgcttctggttatcaagtttagtctggataaaccagtttgcacagtgatgttcaccaccttttgtaacaggcttgtggtgacctaatgtgaaggattgattacctgtgtatctgcaaagtttgttgagaattgtgatagtgttcttcacaacaaaacattcatacaggttatctatggtgtaatgtgggtaatagcggaatagtgccctttcacgagtactatcacgattccagagaatcatgcggttagagttagaagggtcgtattggtgggagtcaatgaactcttgacgttcctcaaagctgagtcttagaactgacattgcagccgaacgtttcagatttgacagatacatgttatctccaaaaataaaaagggaacctttgcagttcccttatagtatctgtttactgagctactttcaagatgcttgtgaatgaaatctcactctcatccataaactcttttgcttctccagtaacttttacaaagttatctaggcagataatcttcttatctttcttactgtaggttagcttaaccttttcgatatctttcttagagcttacttcttcattgaagagttttacagcctctttgaacggtagatatgagccgtcattgtcactgatgaaccctgtgccacagtagtagcagttatactctttagccataaactggcacagtacgaaaatctgtaatttacgagtgttataaactttgctcatgattatttagccttcttattacgtttacggttacgagctttcttagctgcccgtttaatagctgctgcaccagttggtcgatgtgcttgtttcttaccacctttaccacggcctacatagatacttttcataatctcattagctagtttctcatccatcttgaagtgctgcatgagatgagtcacatcaacaccagtgagcatacctagtgatgccagtgctgcttttacttttgatttaaaactgttccagattcccatttcgtatctctctcaatcttttcacaaaatgctttaagttcttcttcagtgacaccaacggttaagtttgttctgtagcacattgtaaagtggcctttaccgataacctactttacgacgagacttactcggctctccactgacaatcaccttgccacccatactcttttctccagaggttttggttatcagaaccacgatatggtttaactgttggtttagatgaatcatacttaccatcaatcaccacgtcaacatatttcatcacatctaagtggattttttcgtgcagttgaaaacctgtccaaagccatatagacttttctggataaacagttttaatacgcttgcatatgttggtgacctcttgaatgtttctatcatccagaggctccccaccaagtattgacagtccactaatggccttatcatccattaacttaatgatgccgtagaggtttgcataagtaaactctttaccagcattaaacttccaagactccctgttaaagcagccttcacagtgatgtttacagccagctacgaagaggcttacccgaaccccttcaccattagctgtgtcaaatggtcgaatctccatgtaattcatctagttacctcaccaatactatttaattgttttactcattcaaacactacctgacaaggaacctgtttagtaataaactcttcacagtgctctttaatgaagttttgtaatggaacacagtcttgtgcacaagtcattagcataatgtactctggattcataatattttcaagactgtagaagacaacatcgttagataggtcgtgaccatcttcttcactccacgttccagtaactacagctaatttgtcaagtaactcattaggtaacggtacaccatccactacggaatcacttagaacgttgtcgccaacatacaatgtgaagaccgtgtggaatgttgagtaccttacgtgattcttttcacaagagccacactctgaggcgtaatccttaatccactctaactgtttttggctcaacttaatcataatttcaccttagttggtaatgcactgaatgctatagctaaccctaaaaatggtttgaagcatgaccataaatccatctgaccatgatatgagaaactccattgtgggtctgaaaaaccccataccataacactaaccccaaagaagaagactagaatgtggagtatgtatatcatcgtttttgatatcttaaacattttaacttatctctcaattcacaataagaaagtcctaaaaagaaccctagtaagacagaacctatgaataacccaataatactatctgccatatctaacatccaataaaaaaaaaaaggtgatgtaagcctatcaagacccacatcaccttgtcaagttttacatagaaatcctgtcacgaatctcagcgatttttgcatcgttcattcgggattcacctttaatcttagtccatccaagatacccgcacaccctgttaatcacagagatatcatgtgaatggcaaacctcacattcttcaacatcagcctttggtctgttaccacaatgttcacagattgctaagtcaaagttaagtccctgatagaaacctttcaacatccctcttgtaatacaactcttaagtgctgataagttctctggattagctacccttacatactggattcttccacctctacagatatggaagaaaggttcttctaagtcctgcttctcaaatggtgtgatgtctgctgcaacattcatatggaaactatttgtaaagtattccttatcagaaacacctttgctaacgccaaacatctctctgaactgttttagctgagttccacaaagtgattctgctggagtgccatagactgcatataagaaaccatcttcatttttaaactcttcagttttcatgttgatgtattccagaacatcgtatgcaaaactacagcttccaacttcatgaagtcgtttaccttcagcaagaacagatagttcatcaagcgcagtaaccccaaaagaagctgtgaaggacttcacaatatcccaaccaaccctgtcagtaggcttcttagttcctttatacaaaccaccttgtgtaaatgcaagagggttagaacttgctggcatattagcaatcatttcgtaacgtttcttgtggaagctgcgaatcatttctaggtacttatcaagctctttccagaaatctaaaccattctctttagaatactggtaaatcattggtaagttcaaagatacagcaccaatgttagcacgaccaacgtagaactcttccccatcctcattaagatatggagataagaaggctctacaacccatcggcgaaattaccttgccagaacgctcaaaagcttctgctacagcaccatgaccagatacacttaagaaatctggatacatcgctttagagcaacactcaatagctttactgtacaggtggccttgacagatattctcattatgtcttttctggtcataaatataaaccagtttagggaatacaacaggcttcttgctcttaccttgtccattcatacgaacatctaggatagtacttgcaatcatgtattgcagtcggttatcttcattagacatatctgagtcaagtaacccgaatgtcaaagttgtgaatgcaaaatccccacggctacaaggtacagtattgagcttcatttcaagagactgaaaaccttgagttaattcaatctgtagctgctccatgacataattgtggtaatgttctttaggaataccgtaagatgctgctttttcagcatggtaacgtagagacttcttagcatacggtacaagtaccttatcaatctctgctaaagtaaagccaccaaattgctgtgcagttgctgaaagaactacatcaccaataacctgtaaggctgacagtacagactttggttcacaatattcgatgccagacatttcaaaaccacctttcaggactttaccaatgtcgaacaggcaacagttgataccaccaaaaattaggtctcttaaatcatggatatagataaaccctttttcaatggcttcaagttcctccggtgttaaatggtactgtttaaagatttctttagtcagataaccacgaataattgaaccttttgtagagattaaactactgtcaaagttagcattctctctgtcacctaagaagagtgtatctttggtcttctggtaaagctcatcccaattttgagcaacttctttacggtaatttctatatgttgagtaagactcatagatttcgtgattgacttctgccaaagcaccctcaacaatactgtgaatatcatttaccgaaattagtaaattattctgtttagtggactttaccagaatcctcataaatgctgactcaagagcttgggtaacatctggtggcagttctttatatccaaccctgttagctgactttgtgactgctgctaaaacttttttgatatcaggttcttcgagtgagccattctttttaataatctgtactttgttcattattgcccctttacatgctaaaaaggtctccgaagagaccctttcattttaaatctttttagaaaacttcgcaaagagttctcttagtttttcagtgatacctaattgtatctctaatatttcgaagataacccatagtaacatggctccaaacaatgaaccttgttcaaagtcagtaaataggttaacaatcaacataccaatcacaatcgctggtgcatcaacaaccaaaccttcccaaatgcgtttaagcattattcacctctgccataaaattctgtagcgtcccaacaggttttaggttttgtccatcagttttcatgatgaatggcatctgacgaactggcatctgtgcaatctccatcaggtctgatagttcatagtcctgccctaacattctcacaacatggtcaataccacgagcttttgcaaaattctttgcagtctcacactgagggcatccagttttggagtaaattacataagtcattaaggaacctctaaaaatccactattcaaatcatctacaacagtattcaataggtacgcaccgttctgttgctcctgattagcattctgctctttatcaatttccatcttcttaatcatatacttcaaaggtggttctttaggagctacaaaatctctgggaatgccaaacatatcatacagtggggcagcattatagtaaacccactcatgaagaagctttgtatttaagccaactacagcacgtccttcggagaagatataatacgaccatttctcttcactttcaactacttcatctaagattactttaatctctggaagaatttgttgaaaagctttctgccactcatcatctcttaaagtttcttttaaaacttcaatatcaattttagtgtgaaggatttcgtcaagcataattttctggacagcttgagcaataccctgaaatttatcttgagcatcaagtgcaaaagtacatgcaaaggatgccataaaagatataccttcaagcgcagtcactgcaaacaacccttttagaatcactttatggaagtgtaaagggtctttgtccagaagtgagtcacgcacataactcagacgatagtttataccttcatccagtaattcttcaagaacacgattcacagtttttaatcggtcttgtacagcaacattctggttaatctcatctaagattgtttcagggtttttaatacattgtcttacaatctctgagtaagtaagagcatgtaggttttcaatctcagactgcttcataattgcagttgcatagatgtcatcagagataaagggtgcaaaggcaaatgccaagctcttagcaacttgagtgtctgcttcccactgccacttaagaatctcaagcattacacctgacattgatgctgggacactctcaaaatcaagacgtgattgttcaaaagggaactcatcttctgaccagtcttgtgctttttgttgtttataaagttcaaagatttttgggtagtgcttattgagtgagtcaaaagtttttctctcaccacctaaaaagattgggtgctggttaatcatagcgtgatttctcctttctttatcatttctaaggcttgttttctgtctaaaacttcccaattctctttgttaaacatctcataaggtcttgcgtagattttaccatctgctgtagagatgtaagaggctccagtgacccataagtcatcattctgctttatcatgatacctgtgctgctcacatagtacaatgttttgcggggtttgtagagcaagtatattggtcgctcatgtttttctattaaatctttcatggtctctccaaagttatttttagagggtcttctacgaccctcatagttattaaaactaaacaccacaaccctcgcaataagcatcttgcagtgcagatttacctacaccaatgcgactgttaaggtagtacatggttttcatacctaccgagttggcataaatcatgtacttcaaagcttgagccaatgataccttcttagcttttgcgtagtcaacatagaaatctgaagagatagcttgaccagtgaacttttgaacaattgcataacaatcaatcaggtcaaaggtgtcaataccccaagcaatttcatatacatacttcaactcttcataatctggaacaataaacagcacgttaccagttgcagactttttagttaaaataaagtcacgaattgggtacaagccatttgtcgtattagttgccagtgaagaactctcattaggcatgtaagcttctaatacagagtttcggattccaccattttctttgatacgttgtgctaagtcatcccaatcatatcgtagttttgcatcatgcttttcatcaatcttcttgttagctgtctttggaggaacccagccttcaggatacttagtgaacttcatgtactctggaacgccacgctctttagcaagtctcagagaggcttcatgcagatagtaagagtgcatctctgccagttcatgaagtttcgttttacctgctcgtgaagagtagttcacgtagttcttcgcaaggtaatgagccacatttgtaaggccaatcccaacagaacgacgcttctgaacatggttgcgcatcgatggatacggataatccataaggtcaataacggagtcaaccattgcaagagcataataagcaacgtcagcgtattcatcttctgaaattctccctgcaaccaaactagctaggaagcaaagagccacctcaccatcctctttcacagcatcgtctctgtaaaggtctgtctctttctcaaaaccatacactggcaacacaatttccatacaaagatttgacatcttcaatggttctttaaatggtgtatgtgtgtttgcgttgttagtgaagaatggatacacacgacctgtagcataacgctgttgaataaacagcttagcaatttcacgagccttgactcgtctgtgcttaactcctgaatgtactgcgtgaccaactgccatagcaaactcatcagcagatgctgtgtagaacatgtcatagagttttggtgcatctttgtaagagaatagcaaccaatctgtatcatactgaacacactgccagaaataatcgtttgtgccgaatgagtagtccatctcattaatacgtttagaaggaaccgttgtagggtgcttcaaacgcagtaaatcttcaatctgcgggtctagagcagtgtagaagttattagctgaaccaccacgactcttctgtttgtttgcctctacagatgaacgtacaagcttgtaataaggcagtttacccatgtgttcgatagtgttttgacggataccatcaccaatagtgcgagtctccatcagcataccgataccagcttgctttgtagtcatatcataagcaacctttgcagcaataccaagtgattcagcagtatcgtttgccttaatcaagcagcatgacgcataacctgatttagttgccctcaaaccgtttaaataaggcgtaggagcgttaatcttcaggtcagataggtaagtgtacagcttgataacatcttgcagtctacggtgctttggttgcttctcaaaggctttcatagccatacccataaacataaattgtggtgactcaaaaagtcttcccgttttaatatcacggataccatacttgtctctgaactgtttcaagactgcataaccgtaagagatatcttttgagtgcacgatgtaaccttgcaggtattcaagctcttcctgtgagtaatccatcttctcccaaagtcctgctctctccatatttttaacgaaggtaaccagcgtaggaaccttagtaaagcctccaaaggcttctttgtagataattcctagaagtagccgtccagccatgtctgagtactcttgagtttgtttatcaacacaaacatcaatcatagcttggtgcatctcttttgtagtgcaacctccatagacacgtttcatggcttccatagtgacttctgaccagatgattccacgcttatctgcccatgatgcccacttattcagtctttctgggtcaaagcttactactgtaccgtttgattttttaattgtcttaatcatttttgaatcctacagatgaaaagagcctccgaagaggctccctagtttaaatcttgaaaatttgctcattctgccacatatcgtaaaacttatcacttacctttgtgactgaagaggtgtgcatacaattcacgaaccatgatatatcactattcataattatttcaagctgttttgtgacatccacattcatgtcttcaacaacgtaattacctcgcatcttgcacacaactcttccaaacaagattgcacttctgcctcttctgtcgtcacagtacatgactgtgtccccgtgcttaacatcttgtccgatactatcaacacctaatttagcacctgagataatgtcatcataagatagcttttttgctttagacacggtatttctccaagtttctttttagatataaccaaccatagtcagttctttctacacccatgaagagtggagccattacaactttaccaaaagtattcctgtgttgacagatttcaaaagaatactctttctcaatatcaatcccgtatttgtacagaactgcttttaaaacttgttcattatccagaagtgtatctggagtctcaccatacttctcaagtaacgggtcattcataaacaccgtcatagagatattgtagtttgagaagtggctcttctgagccacaccttctttattaatgttctgtgaatccattataaccaaccccttttaaccagcacttcaggttattagcatcataattcagttttgggtatgaggttacatggaactgctcaccatcatagtagacaatatctgcaatccatagtccattctctttacagaaatctttatctgctttagaggtaattggattaacttctacaaaaccatctacacccaaatcctttaagctacatacaatacctttgcagataacgcatgtctcagatactaccacaaaaagttcttggctttcaagttctacaatgtattcaccaagctgaactttagggagattagattgcttcataatcttcccatcggctgcacgaacaattgcataccattctttgccatcaactactgacattctgatgatacactcttgaccagtacgcttctcaatatctgcaagacgttttaaagcttcttcatagttgtctgtgtattttaggtcattgttatggcaaacagcttccatagcaccattatggtcatgttgtgacaggtaaatcaaaccgtcaagaacatattgcaaatctgcctgagcatccagtgtctcaatggggtctttctcttcaattgcttttacaagctcttttgcttcttcaagcatacatagagattgagatttcaaagattcccaatactcattgctgtagggttgcttttgagtgtttccacaacgaagattccagttttttactgattctcttgagttaaacattctgactccttaaaatatctgttaacctttagctctcgtgtttcaatatattccagatgctcacgtagctcttgacgtttaagcattaggtgctgcatttgagattcaacagcttcaacctctttaattattgactctctggcatttgcaagaatgcgctcaagttcttcaatcttgctatcaacttttgctacatgactcacagctttacttttgttaaacattttattctctctcaatagttatcaagcacagtttgctttcattaccttatggattcttttttcagcatcttcataagtttctttagtaataaacttaattgctgcaatattggcattgtagaagagtctgagcttagagtcaatcctttttgtcattacatcgaatttatgttgaaggtttgcttcaccataaactagaccaccttttgtgtggtaggtttgaataatataaaagtcaaaaaattcttttccaaagctctcaatatcttttttaatatactcagaagaagtctcataagtcatccaatcactttccttagtgactaccttcttccgagtcttgccagcaacttttcttttggtcacactattaagctgtttctttccgatataatattgtccggttttcttacagtatactaaatagacaaagccaaaatgtttagtggggtcaacttccccacacaaagatacccaatgaccataggtggggcaattgccaaatcctttaatcttcattcatacacggctcccaattatatttcttaaaagagaaattatcttttggatttctctcttgatatgctacccagaaatgttgttccattaactctaatggtgttttggtaacagtctgtccatcccatgacacataggtgtaagactcttttttagcatagagttcatagatagcatcaagacactctttataggtctctttaccatctaaagcattcatcacagctactttgccagcacctttaaggccgaagtagttgtctgcattatctccagcaacagcctgataacacaaaaatttaaaacctacaccaactgtttttgcagcttttggttttgatttgataggacaatcccagatatcaccaacattattatcagcaataaaaattagtggtgacttttcataagtcatatcaatacaataagtcccttcagcttgtcgaaggtctttgtcaatactcataagagcagccttcttacccatcttttcagctttagcaattacgatggaatcggcttcgaagcctccctttaagagcttgaactctggtcttgacagaagatattcacgacaagcaactaagtgtgttggtgtgacagcatctttacggttaccttgatattggtgctcaagacctttaatgtctttatgtttatgtacacccttctctgttaagtagcctacccaagttctttctttaccaacaaccttaagccattcctgaagaacctgctgagtagccatgatagcttctttttcactcttagcttctttccaagtctgtctttcccattcatcttcatcaaatgtcaggccaagctcttccacaaggattctctggtctgctaaccatcttgcagcatcttttgcattatcaaatggttcagattcttctgctgtgagtttattgacatatttatattttgctttctcaactacacaagcacctttataggcaatgctatcagagtcaataaagacatgtgtaactgaatcgggaagttttgttagtgtatacttctccattgtgactctccatatgaaaaagccccatactaagtacagggctttaaagtaacttttagagattagtcttctgtatcgaagtcttcatcttcttcgtcatctgggtctggcaagtcttcatcgtcaccatcatcagaattgttcgaaggtttgtggtctgttgcgtcttcttcagtgatttcaccattatcttcaacaccatcaagaccaagcatagccagttcgtcttcatccagttcaggttcaccgttagcaccgttaccaccagtgtaaggtacaagagtatcaatgataaactgttcctgaataggttttgtcagaacattgttctcaaaagtgtagaagtgagtagaaagaatcacactaccaaaagaaccgttaccaactgcaatatctggatgaattacatcatagttcttgtcatcttcatgcttatcggatgcttgagctttgattttcttcatcggctgtttaacagctacacgcttaccatttacttcttcaatcagcattacagggaatgactgtttagctgtccatacagcaccatctttataagctgctgcacgacttactttcaagatgtagtaagtgtctgcttcaaaaggtggtttacaaccaaacttctcttcgaagtcatctgcatcaactgcttcagtagtaactttatcccaaccttctgggtttttcttagacttagtaaactctttaaacagcttgttaccatcttctgccagaattgaaacactgtagttgcagtctttgcctgggaatttcttatcaatggatttaccttttcccggacgtggtgaagtgttcaggtaataaaaccaaacatctttcagaaggtaacgcagagtttgacgttcagtaccattgtacttctctaccggagctttcattttaacaactttagacattatttaaacctctatctcaatttatgaagagtaccaattctaatttgttgatactctattgtcaaacattaattactggaagttgtgcttatctttagaagctgctttacgtgcagtttttccagttcttacacgcttctcttcatagtattttgctggtttaccagcagtggcttttaaggaatccccaaaaacttgttcaaatgctttagagtgtttcatgatattctccgatatacttgatattcaaacacattttctttaattataaacagccaaccaatattatgtcgaggtatagtaatagtgactgttatcatactgtgatggaacttttcttctacctcaacgcctccatcacatttcatgtagagcattctatagaacctatcaacctttgtcaaacactcttcctgaaattcatttaaacttttttcttcaaagaagggtatgaacccctctcttttgtaactatcactcagtttcaaacgggcagtcatctgcatcctcacttaatacaggtggaatcgttgagctttgacgctgttcttcagtatagacttcaccagtctcacggtcaaacacttcatcctcataatgaggtaaagagtcgtcataatggtcttcagcttcaccaataccaaactgttgacgaatattttctgctgcaccatcaatgtcaacaccacatccagaagctttgataagacgtcctgtgtctggattgtaccaagtatgaccagcaatacctgttgacttaccatgacgacgacacttagttaacttgatttttgtcaagtttttcttaacagggtctgggtcaaccttgttacgcattaacagaatgttgttcatggaaatctggaaatacgcaccagaacccttaatatcctcttcagagatatctccaccttcagagttagctttctgaccacctgcactcttacgaacgtgacaaacgtttacctgtgcatactggtagcgtttgcaacgacgcaatagttcagacaagacttcctcttcatccgtatcagaacgtgacagagccaatgtaatagggtcaagaataataatcttacagtctaaactgttaacaagatagtcaacaaactccagcaggttatcttggtcaatcgccccttgatggtcaacgatatggatgcgacgaccttttgaaagctctgcgtgtgctccctttaattcatcccaatcccgttcatcataaggaatctcagaaatctgtttgctcaggtggattgcacagagcatttccatcaactcttcataggtatcttctacaggaattacaccgatattataatcagtttctttccaagctgaataaatcatctcacgagtgtaagctgacttacctactgaagatggtgctgcaatagtcgtaatctcacctaaaccgtaaccaccgtaagttagcctgttcaagtctccgaaagattctggaaaaggaatcaatggaatctgaccacgattcttcattgcctcaaaaccgtctgcaaagttcttgataccagcagggcagtaacgaggtgcattgtagatacgctgcttaaatccttccagaactgtatctttctctttatagaactttgtccaccattcattaaggtcttttacaccttctggatactggaataaacgaaccttctcaataggtaggataccagcagcctctttagtggctttagcacctgcttcatcgttatcaaagcacaagtaaatttcatcaaatgatgtaatgtactgatagttatctttgatagacttgatgtttgcacctgatggaacagatacgtgacagtaattcttacgacgagacttgtccttaatcgcaagagaagtcatatagattgctgtcgcacattccatctcaccttcccagatgaataggcggttaccaccttctggagcaatccatgaaccgaacattgccagttcacctttaatatctccaacaccaccagaaaagtcttttaacttaccgcgtaggtgctcttttggatggtcttctgggtaacggtgacgaacacggtagccaacatgctctagcttgccatcttcattgcgtttgtaagttgggtagaaatgcgcatcaatttcaccgtcactatcaatgtcaaccttgatacccaaacgctcaagaacttttgcaggaatcttcctgtcctttaagtccattgcttcaaggttgtcttttacatcgtctaaatccatcccacggaaagtacggtttttattgtctgaaccagtagaataagtgctcacgatttgtcctttatcaaaatcccactctgggaaacctttgttacaactaaagcaagtcatcgaataagaatcatcgtcatgatgatagattgaaccagccactgatgaaccacaacgtggacatgcacaatgaccaacaaactgaccagcctctttcaatttacgacctttagacattagcacctcttcgttgtagttctgccttcaatccattttcaatcttgtccagttcatgaatttcatctgcaatctctttccttcgtgattcaactctcttaagacgttcaatcattacctcattggaaagagatgagagttccactaaacggtggtcaataactttaaaattgtcttttactctcatcttcattctctcttttaaactttaatgatggctcttagcttgttctcaaggtctgtaagagtaccattattatgaataatgtcacgctcatatttggtggatagcccattttctgaaacgtgtgatgaaatcttatccacattgtctctttttacttcaatagtttggtgtgcaaatctactcagccactcagcttcagagtcaaatcttaaatcactgattaagacaaaaccatattgatttctaagtgagcaactttcatagaatctaaccattcttttttcaaggtctttagaccagaacttgtctcccattactttacggacaacttcagtgccccaaatctgttgaatttgtcttgatgagaatttatactttttgctaaaccccagacgtgtcaatagagttggtttagcaaccttcttaagctccatgattaatcgtccggttaactctgacataagcttgaagtccatatggtaacgttcatctctgaaggtgaactccatagcttccgtaactttaaacatcagttcaggatatgacaagtcaaatacctgtggagtctctttagtttcaccatacaggtcattccaagtcaggtcaaatatctttgatgcagacagtttaaggttgtctgcaaaggccattacagcaacattgtagccgtgctcatcctctaagatgttcttcacaatagaacatgaggtgtcttttccagaacgtgcctttccagtaaatgcgataatattactcattttatctccattaattactaaaaagcccccaactaaggaggcttgtaagatttataaagacttagtgcagttgctcagcatactctttttcagccatagctgagatttgcttcacaaaatcatcaccgaagttaatgcgtagtttctcttcaatgattgaagcaccaatgttcaccaaaacatcattgattgcttgcatactaacgccaccagtcatctcttcaatgaactcaatacttacacccatcatctgtgaaagttggattagtgcaacgatattcatcatcgtagagatagctggaatcatcagtgcaactttcacctgaagaggttctatatcagctacagagtcattttcaacagcttcttccaaagactctttagtttgctggataatttctttgattcgtgggtttaattcttctacaccccaatcaagtttttcatatcgtgaaagtttttcttccatgcgcttctcaagagcagccataacagcactagttgatgaaattaacaggtcaattagttcatcgttacctgcgagtagtagcaagtctttttcttcagtggtcatggcaatataatcatttttctgcatctcataaaaaatgaactctgcaagggcatcaacacctacaacgatagatgctaaagcaagtgttttgtcgcaaagcatcatgttaacctgttctgcaaaagcaccgttatcagacttgtcttcaggcaagttatagcttgcaggcataaactgttctgtgtagttgttaccacgaagaatcatagccatgctttctacggcattgattaaaaatgcttcgtcaatacctttctcttcaagcatcttttctacatcgatattcatattatttctctctcagttggttaagtttctttctagaacattttaagttttcggatgaagaaaagtcaagtgtcttttcagaacgtggataatagtgcctatcccaagaagtttcaacatcgttaatcagtgacgctagattacacaggtctgtaccactttgcaacctctttttaagagaatctagtaaagcaactgtgtctttagctttacgtctcgctgtagcaacatctttcatatgctcaaatacagagcattttaagtcatcttcatagttttcagacaactctatttcatgttgtatatcaaccattctcctgtgatggtgagcatattcccgttgagcagcaatgtcaagttcttccaacttttgcaaagctctaacaaagtcagctatgtctgggtgaacaaacattttctgcatgtaagcctcttttaagtaactattaagttttaatcttttatcttaacgtattctatacgttatactttaaagctttttaaaagctattaaataatcttttaagacttttaaagtatctgtatagttaaactgttaagtaaccttttaagtaaaaacccacttcgcaaagattaacaccttgtcaagtgcattgtcaacttgctttttataattttttgcagtaatgtatgaggttaatgattttagagggagtaaaaatggaaaacgtagattttaaaaacttacatttggttggtgatacagaaactgatggtttactccttgagttcactaaagttcacgtaatggcttttgcagactataaatctgatgatgaagagcctcctgtatgggtctttacagatgaacctatcctcggtcacaagtataccaagtacattaagggtggcttacgtgaaggtattgagtttgcgttaaaggcaaaacgtctttgcattcataatggtctaggttatgactggtgggttttcaatcacattgcaccggatttatggaactttgataatccaaagtgtaagccgtggagtaatttctttcaggattctcttatccagtctcgtgttcagtggatggatagaccaactccaaagggttataaaggtgctcatggcttggctgcatggggtgctcgtgttggtgttcgtaaaccagagattgaacattggggtgtgtggaatgcagaaatcttcactcgtgttgtagaagatatccgtattaacgccaaaactaaacgggcacttgataatgagtatctcaagcttaagaagtgtggcatagacacttatgaaacctacatgcgagctaaagaaacatctttctggatgagtcaacaagctatcaatggttggaaagctgataaagagcttatggagttccatgtaaaggaacttgacaagttgactaatgagcttgcttcagaggttgaaccgcatctacctccaactattaagaccaaaggtaaagtcactggagaagagtttgcaaaagcttggaatgagtatgttgaaacatttggtcatgcagatggactgaagagaattaccaagtaccctaagacaaagtaccgtcagcaggtacgtaacggtgagatgcagacatatgaaatcaagccatttggtaagccaactacaaagatttttaacattgaaaagagaaattgctatacaccaaccaactctgtaactggtgaagagtacaaggaaggttttgtagcaatgaaggatgctcgtgcaatttgcaacgagttgaatgcaaagattggtaagaaatgcaaagactggaagccagtaaaaacagtcaaaactgtgaagtactacaacagtcatgttgttaaccactttgaacttgagtcaagtcgctatacaggtttgattgatgcaccatatacaccaattgagtttgaagtttctcgtatgactcaggtagcagttgttaaagactacttgaaatcagttggttggattccagatgactggaactacaagaaagactctgatggtcgccctgtcaaagtttgtcgtttcaaagacaataaaaagatgattacaaagcatcctaagtggcaggaaatggttgagcggtgtggtttgagttatgttgaacacgaaggcgtccagtacattgagcataactggtctgtgaagaagtacacagatttgcttgagccttgcttaatccgtacttcaccaaaacttactgaatcatcttacgatacgattgaaggtgagcttggacagaagattgctaaatactacactttgatgcaccgacgcagaactattgagaactcaaaggatgatgaaaaaggttggttgaaccagattcgtcctgatggtcgccttagtgctggtgcaatggtgtttggtacttcaactggacgtatgacacagtatggtattgtaaacgtaccgtctggtgctgctgtctatggggaaccaatgagggcagtgtggatttgtgaggaaggtactaacgttgtctctgtagacatgaactcagcccagctagttcttctttgtaactttatgggtgataaagacttcaccaaagcggtaacgcaaggtaaagaagaaattgagttcattcgtcaagaagatggacgctattactgtaaacactttgatgagtacctcaacccagagattgataagtaccttcgctatgactctgagaatgacctgtacgttgtctattcagggactgatgcacatacactgaacagtatttactttaacttgaacgatgagcaggacatcttgacttgtcgagctactcaggatgagaatcttcttcatgagattagcaagggtcgtaagaaagctaagaatggtatctatgcactgctgttcggtgcaggtgatgagaagtttgctaagacgattaaagctgcaactactcaggaaggtgcgctgactaaacagacttactttattcgtttgcctaaaattaaaaagctgctagatgacttggaagctgactacaaagcaactaaaaaggcacttgaagaggtgtttggtaagactgctgcaatctctaaagggggttttgtaaaggttgctggagcttggttgtggtgtaaatctccgcataagctgttgaattatttgctcatgggttcagaggctcagattcaaaatgaggcaattaaccttgcttgtagaagagcatgtgaagaagggttgacaaaattaaatggacgaaaaccagcaattggtgctaggcttttgctggcataccacgatgaaaacagttgggaatgcccagaaagtatgactcaagaagttaaagccataactgactggatgtatggtcaggcatctaagaatttaggtttgaagagtgagacgcttgtaactggcacaggcaaggttggtaagagctggttggaagtacattgatggtgttgaaatgattacagaaattcgtcttaaggagctattggactacgaccccgaaacaggggtctttacttggaagtttagagaaggtaatgcaaactttaactctagatttggaggtaagagggctggaaactacagaacaaatactgatggacacaagtcagttcagttgctgattgatggaaggggttaccaagcaaatcgtttagttatcttgtatatggatggatatctacctgataaaaccttacaggttgaccataagaatggagacggttgggacaataggtacttgaatttaagggtttgtacagcttcacaaaactcttgcaatcggtcaagagagtccccatctgggttgccaaaaggtgtacagaagataagtaaaaggtttcgagcaatgatacgtttacaaggtaaaaactatcatcttggtatgtttgacacacctgaagaagcccataaagcttactgtgaagctgctattaaactccacggagagtttgcaaaactaagttgacaaggtgctcatggaagagtaccttatactatatcttatataggagggcttatgagccaccgtggaagaacccatgcagcgatgatgaaaggtgcttcaaaagagactatcaagaatcgtaagcagaagttgtttgagaggatgaacaggttaattgacaattcatctctttcaggttctgagaaggttttcttgaaaggtaaccttaaaagcattgcacaagaacttattgatattgagtattggaggcatcaaaaatgacacttgcagacgttattcagcaacttcacgacaactgctatactccagagttgattcaggagatgtgtattgtagtgatgcctagcaagttcgtaaaagcctttaacagtgacactttaaggtctgcacacattcttattgttgaagggaagattgcaaaagaccgtactggagtactcaaaggtgaacgtattgatattctggagttgctatgaagaaaatgtacagtctctgggggagggttggtaaaggttttgactggacacttcttcgttcaaacgttaaacgtagtgaattaccagaactcatcactcactatttaaaaacatacagagaggtagattatcgtgaacaataaggttaagagttgtgtgaaagcaatggttgcacttggagtaatttttctggctggctgtaacccatcttatgaaaacaaagatgcctcttacagcctccctccagagatgcaagactgcaaaatctacaaactcaatggggatgctataagcagggatattgttgttgtcagatgcccaaactctcaaacaacaacatcttatagatatggcaaaaatggtcaatcacacactactgttattgatggatgagactaatatgcaagttttagtacactatatctactgctacgatgttgtttattccactgcaaccgtggcccaacgtaacccaatcgtaccgcgagaaggtgaattggttcgcattgagggttggacttacactgtggaaagcatcattcataagtttgatgttgctggtgatgttcaagttatcgacgtggaaattggtggtaagagaaaatgaaatctaagcaaacaatgacagatgcaatcttcacagtacacagtttcgaaaggttgcacattaaagatgtaaaaaatacaccaaaatgcgttgtacatttcattaaaagacgtagtgagaggtcttatataaaatctgacacaggcgagagtacaaatgggtaaagataataaatttcagaacgcagttcttacagaagatggtgagcttgaaacattcattcttcgtgttgatggtaagttattccggtgtcgttgtgggtcaaactgtttccataaaccagataaaaacgacttagagctttacgcatgtaatgcctgcaacacttggtatcactcagaggttaaaaatgacaattctgtacaaacaaaataaagatggttccttcaacgtctggtcatgtgttgctgtaggtgacaaagttatcacaacctatggtaaagaaaatggcaagatgatgtttgaagagtacacagcagagcctaagaacatcggtaaaaagaatgagcgtaatgctgaacagcaagctctctttgaagttgctgctaagtataaaaagcaagttgaccgcaaaggttatgcttacacaaaagattctgcacagaatactgagaaggtaggtgtacagcttgctcatgatgctgcaaaggttagccatgcaaagtatctgaagttccctgctgatgctcaaccgaaacttgatggtgtccgttgtaggatttcaaggcacttgagtattggcagtgtatttacagcatattctcgtgagaatactgtttacaaaatccctactgaactaatacctgacattatggcaatgttaaggctccacaaagatgttgatgaatttgatggagagatttatgctcatgggtgggacttagaagatatcgtctccatgattaaaaatgaagacaatcctgacagacacttgctcaagttttactggtacgatatctgtgacagtactaagacgtggcctgagcgtagagatattattgagacttcaccattgaatgactttagagacggctgtaaagttgttcctgttaagtctcgtcgtgtaaattcttgggaagagtttgatgaagctcacgataagtgggttgaagctaagtttgaaggtgcaatgtaccgttcaatctctgaagactccttctatgagtgttgtcaccgttcatacttcttgattaagcacaagaagatgcacactgaagagtttaaagtgactggtgtaaagactgataagcgtggtcatggcaagttcgttgtagagactcttcctaacgtctttgtagatgtctcatggaagactactcatgagaagaaacagtatcttgctgagcatcctgaagagtttatcgggaagcctttaacggttcagttccagaagatgactcgtaagggttctttacagttccctgtcgggctagctattagggattatgagtaaactatgttcagtgcagtcattctgtttatactggttgtctttgtattgtcatacattgcaagtaagcttttggacacatcttttgcaatgggtgttttgttcacaatcttctgttttgtaacacatatagctatcaggacttttatataaaaagttgttgacatagaaaatttggtgagtatactgagcagcataaaccaacgggtactcaccagcatcacttaagagtcttctaagagggtttttaagtgatgtgaggtatggcagtgttctgttcggtacggtgtggcacggtaatgttaggtctggtctggtcaggtcgggtcaggtgcggtacagtatgggtagttagtctcagctacattaaaatgagacttacctttttaaaaggtctttgaagagggtcttttaataaggcggggtgcggctcggtctggttgtgtgaggttcggtttggttaggtaaggtatgggtggatggtagtagccacgttaaaaatctaccaagtttctaaagagtcttctgttgagggttctttacaaacaacttaacaatcaactaaagtgtggagctaaatgctatgaaactgttaaatatcaaaatcactggtactcgccctttcctgagccacgctgatacactgtcagacccattaaaccctctgacaaaatatcacaagtctctgtctagtaaacgtaaaaagactgacgaagactatgcgctcttagcagaaagtcaactggtaacctcttgttactacgatgagcagctaggttttgttatgaacggtgagatgattgaggcttgtatcaagtctggtgcaaaactcaacaaacttggtaaggtgattgaccgagccattatgctgacagatgtagtcttcccaatgactatcaagaactgcccagcaaacccacaagaacttgctaagaatccagactttatctatgctaaatctgttaagattggcacagcacgagttatgagctaccgtccaatcttccgtgactggtctgtagagtttggtttgatgttcgatgaagagcaaatcaccaaagaagaactcctgatggttctggaaaatgctggcaacctttgtggtgtaggtgattggcgtccacgctttggtcgtttttctgtagaagttatttcagaaggtaacgtttaattatgaatacaaaagcattagcagcaaagtttagttatggtgacactgtatcacactctgagatggacaccttcttaggtattgtgaagccaacttatcaaggtgacattgttaagtacgaagatgaaatgaaggcttatgctcttacacgtctaaaccgacttgagaagtttattgagaagcttcttaaagaagagaagatttatctggttgcttcaatgggtgttggttatcgtgtggttgaacctaagcatcaagcaactattgcaaaacgtaaaatgtctggtaaaattggtcgtgctttaaagcaagctactcaagcaattgagaatgtgaacactatggcacttagccaccaagaacgttcacgactaattgaacagcaaaaccacttagctgctatgaaagctaacattaacaagcaacgcagaaaacctttctaagttgactaggtagcctcttcggaggctcctttttaaaaggtctttaagagggtcttttaacaaggtattgtatggtgtagtttggttaggtatggttaggtgtggtaaggtaaggtatggaaggatggcagtagccttataaaaaatctgccaaaataagctataaaatctgttgacaagtcaggtgttatagcttatttttatactaaatgtaatacaaaattcaatataactttgaggtcattatgaaaaagattttattagctgctgcaatggttatggcaatgaatttaccagtcaatgcaacagaacttccaaatgtggacttatcaggtgttccagaagacacttgtcagattgttaaaggtgttgccctatctaaaggtgaactacttaaaccaatctctgaagagtctttaacagagatgactgataaggtaactgactatcagtatcgtgttcttgcagagtatttcctgcaatctgcaaatattaaagagaagcaccctgatgatattgatgtacaggctatgctcaatcatcgtatccagtttaaagaagatttgatgcaaaaagctatgtatggtgttgagtatttcttagaaaacagaagctgcacaggtatttgatatggctcttaaaaagttacatcccagaagcggttatggtaagataattgacgatacagacggctttacagtctttacagttatctgtcaagatgattcacagattgaaaaggctcttgacgattatcttaacgatgaacgtgaaaaggttagggctacaaacatagactcattaattgacacttcacgcaaacggaagaagaaagatgaatgaggtttttgacccttatgccccacaagatgattgggaggctgatagagaggctgaaatggagagttatatttgtccaatggatgtagacgaaatgagagacttcgttgcacatcgttttaagagagagattaaatccagaggtctttctcaagagcaagttgctaaaatttgtggtatctctcaggctcgtgtatccaacataatccatctcactggtaacgtctcccttgagtatatgttggaagtatgtgaaaaatttggtgttaatttcaatttaaggttggcagattaatatgaaacgtgaaaacattatccactctgaaaacttcgcattaggcttttatggcgtacctactcaccttgaaaaatattatggtgtgaagattctctccaatctcattatggcttacaaagatggtaagattaagcatactgagaagaaacgtgtcatgggctatatggctgtaggttcagcaatctcgaacattaagctggaaacaactagcagtcagattgtgaaagaccacttcatcaaagagctttaccacaatcttgatggtgtagatgttcaggctgtttggctggatgttgatggtcataactacacaagttttgttttcaaaaacgatgacatcaagtgtctgttcccataataggtgattaactatgattgatatattcttacaagatgcccatgcagatttccttaaaacgatgcttaaaaagtttatggcttcacagtatgagaatgaagcatcttttaaaatagttacatgtggcgatgaagctggttttgttgagattgaacatgaaggtactggaaagactgtttgtaagctacctgatagcatgttctctaaaacgttcttaacaaagactagtataaatgttaagcttgttcctcagattgaaacatactctggtacagattaccctaaaggcttcaagtcactgatgaaacacttcttagatgactttgtgagtaatcttctaagtgaggtaacagaaagccgtacaatattaactgtagagaatatagggaacactatcagggttacttctgaccgatattctatgagcctcttcgactttgtacctaagaactttgatggtattctggatgaagaagatgactgtgtagactttatattggttcttgaaccagtttttgaggttaaatagatgaaaattgaacactgctatgagtctgatggaacacctatccgttgtccacattgcgggtgtacagacttacaaggtgaggtaagtgaaatagtcaacggtcatattgctgaagaaagtacccgatgcacagggtgtaatgaaattatcgctttctgggcttatggttcataccaaccctctccacattttatctaccatcgtagtaaagttgtgaagagtgttatcaactggttcattaagaaaggatttacaaaatgattaaattaatctttgcaagtggtgaaaatggtgagtttggtactccaactggtatgccgtggcctcgacataaacaggacatgcaagagtttaagagactcactaaaaataacttagtagtcatgggtaatgagacttttaagactctgggtagtaaacctctaccagaacgtgcaagcctaatcttaacaaattctgtaccatacttaggcatagactttggcaaagatgatgtaatgtatgctaaagccagtaaagagtcatttggagcatttttgaagtatcttgatagctctattgatgaagatgtctttgtaattggtggtgcaggtgtccttgtcaatgctttaccgtatgctggtgtggtgttccatacagtcttccataaagttactgaagaggccactgtgcatttaccttttgaaaacttctttgagaagctgtatgataaccgtgtatttacaaaggtacagtcaaaaccatcagaggatggtaaagcaacttttgaaatttatgttccacaagtaaaaggacacttttgatatgtcacaagcagattcaagttacaaaaatatcctgaaccatgttttatccgttggtgaactgcgtactacacgaactggagatgttatctctgcatttgctccacctcagtttcgttttgatatgcgaactggtttcccgctcttaacatctaaacaggtgtttacacggcaagtcattggggaagctttatggttcctgaatggtgagaataaacttggtgaactccgttatcgcacttggggtgaaaatgatggagaacgttggactatctggtcagatgattttaaacgttggttaagctctaactattcttctgagcaagactggttagaagatgcaggtgggagaatctacggggttcagtggagaagctttgaaggtcataatggttgtgttgtagaccagttagaaaccttagtaacgaagatgaaaggtgatatcacagaccgttacatgcttgttaatgcttggaatgcagcagatattgcagcaaactcaatggctttagcaccttgtcatgttctgtttcagatttatatcaccaacgaaggtgaagttgacttgcaatggtatcagcgttctgtagacacctttttagggcttccatttaatattgcgtcttatggttttattctggaagttctttgtaagatgactggatacactccacggtacttgataggtgtctttggagatactcagatttatcagaaccatatgaagcaggtttatgaactgatgaacaatgaagagttccatgcacctacttttgagattggtgtacagcttaacactttaagtgacttaaaacatcttactgcaagtgactttattggtggaattaacaactaccaacatgcagggaagattgaagcacctctgtcagtaggcaagtaaaacaaaaaaggctcccgtaaaaaggagccttaaactttatttttcagtattctttgtgttcttttcagtaatagcttgcaaggctgatactgattgagccagattattcacactgtcagagaatttatcaagagttttggtaagttttgcattctcacccttaacgttctctaactgaactttctggttctccatccctagctgaatcagtctcatgtcagactgtaaatcacgaatagctgaatagttacttttggaataattatctagctgctgtaactttgttgtgacagacacttcttgtttaccacttgaaacctgcatcgtggtaaacatcccaataacactaaaaataccaactacaattgcaccaatattatttttaaaagcttcctctagccacttcatttatttctcccccttaaaagctttttctaagttatctacgaactcatcatcaataggtgtgtctgttttactcgcaaggtatcttgcaagcttaaagaacactttctcaatcatgtattcacttagaagggataaaatgagtttccagaagaagctacctagattttttagaagaattgctaggattgtaggcatttaatcacctcatcagccaagatggtgagaatacccataaagaatattaacaccatcttaacaatcagtcaacaagggattaagcagttcttacccaagccattaacttgtagaactggttagtaacactaaatgccgaaccagaacctgtgctaccagtgttaccactaactgtgtggctatgagcaccaatacctacagagtgggaatggtcaccagcaccgcttgtagtaccagaccacgtgtgtgtatggttaccagttgtgttagtgttagggaacttgttatctcttctatcaagccagtcaagagttcttgtcttgctgttgctaacagttgtgtaaccatactgagcatcaccaccattaactaataacagacctaagtggtgagcgtggttgccagcagcacttgttgtaccacttacagagtgagaatgaccaccagtactgttagttcccttcgtaccataatcaaaagatgaagtggtagcagagaaactatgagtgtgtgatggcaagttaccaacagacaacgtcacagaatctgaaccaccagttgtagcaacatctgaaccgtttgctgctgcaattctgatagttctaccaacaccattgttcaggtacgtccaagttaacccaggaagtgctgtattagggtcaacattactgttaaaccacgttacaatacctactggatagattttattaaggtctgtagagtcactaattgctgttgcaattttctggtcagtttcagctttagtgtatgcaccaatctctgaaggggttggtttttgtgcttccgtgtaaactttagaccaattaacttcgaagccataggcatctcttgatgttctataccacatcccaccatttttaaacgaaaactttaattgtgcagatggtgtagaaccagtccctaaaaacatgtgatatacaagtgaagaagactgtccagtggtgttagtaaccttataaagacctgtttttgcatcccatgcaacaccatcaccaccatctgcttcagaagccgaaccagtgatgcttgcaagcatatatcttgcgtttgcggcagattgggtatagtatctagcatctaagttagagaagtcagaaggttgcacctgtccagtgattctgactgttttgttcagtaagaagtcattcaccatgaaaatggtagtagcagtcctatcattgtggataacgaaactttctggtttttgttccccgttaccgatgtaccaccttctaacattatcagcattaaagccagcaaagtataactggctatcttgtgtagtattctttagctgtaacaataagccattttgattaataatctgtgcagacctgaaagtgtttacatcacttagtttagcaagattactcaaagtagctgctggaatgtatcgagaatcaatattagtccaatcagaaggttgcacctgtccagtgattctgactgttttgttgactgtcaccaaattctctaaaatagcaattgaaacgtcagactttacattcttcaatacaagattatttgtacccctattatcattacctagataccatctgttagtaccatctgcatcttgaccacgaagatagagtggagcaccttgagtgatatttttaataatgagcgcttcattgtcagagaggatagcttgagtacctctaaaggtattattcacggctagtcttgcataccttgcgtcattctcttcattagttctcatcccaagttcattagggcttggcttattcaaagtatgataaacagttgccgaccttgaagcgtctgcaatagttagtttaaggccgtttgcgttgattttaaaagtctttaaaacatcaccaacagtaatctcagaagagcctgtggggtcgaatacagccttgccaccgtaaagtagcttaacaattcctaagtcacccgtcatagtgctaccagcaatctgaacaaatctttcaagcttaaaagatgctaagaaatcttgataggtcattcgacggtcttcatcacccagcattttaggtctctttttaactctgacatgaagaaggtcatctgaacggattgtgtcgattgagtttagctcactcaatttgtaatctgccattataaattttcctcttaaaaggggctataaagcccctgtaaagaaattaagctgttctctgccatacatacagtacgaaggatggctgttcaatattaactgcctgacctccaccaacactatttgtattgccactatgtgtatgttcagaagaagaaactgttacagtgccactatgcgtatgtgaaccaataccaactgtatgactgtgagcaccaataccaaccgtgtggctgtggtcacctgatgcaccagatgtgacgttaaagctgtgtgagtgcgcaccatcactagaagtccttgatgtccttcttgcaccaccaccatcagagtcagccacagcattaccagtgccatcattttctctaggatacacatcatggttgtgagtaccattggtgctagtgttacctgacacactgtgtgtatggttaccagttgtgttagtagcttttgtcccatagtcaaaagagtttgtagtcttagtcccataatcaaaagctgaaattgacacagttgcattatgagtgtgaccaccaccgctaagagacacagagtgggagtgctgaggcatgttattaactgacaaagtgacagttgaagcgccaaatacagaacctgctggtcttgtatcagagtcatagccaaccaatgccctgccttttgaaactaactcccaagtacctccacaaattaagtatgtggaagggtttgcagagttcatagaaagatggatagtacctactggataagaagcctgaacagccttgtacaagttatttactgctcttgctgtagcatacttatctgcatcttcgttatacagattagatgttgtccagttctgaacattacttaaaccaacctgtgccttagttgtattgtgtgggttactcttgtccttgatatgttgctggacaaggttattaacctcttctgaagacataatctgtagatttgctcttgcctcagctacattaacaatatctgataagttatttgcagcgactaactgaagagcattaataacattatctaaaccaatttgtgttttagtaacaccatgagggttatttcttaggcttgcgtgtggagcaagtaattgctcaagagtacacctcttatcctcaataccctgcttaagatggaagatatcactaaggtcaattggtaaggatgcttgaggcaaggcactaatttgaatttcacctactgccattattaagctcctacaaattcataagtataaagattttttgtacttgttgatgcagtacctgtttcggtcttaataagcttccagccattagccaccaaatctggttgcgcagtggcaaaagaccttacctcaccaacagtaccatttgctttcttcaggagatagtttaagatataattaaaccattggcgacccattggttcacctcttaataaaccagtcgcctgaatttctggtggtggtaatacttttagctggttaccatcagcatctacttcatctgtagaccaatttaaaaatgccattaagaacttccttcttgctgtgatttatcttttctacctttgatgatttgagctacttcagccataacaccataatcaccacctgctacagtttctttaccaacgatgtagttgtctgtagcattataatttttattaaccttcaagtaatctactgagccactattagccgttctatcaatcttgaaataagcatctcttacaccagcatctgctaaactccctagtaagttcttctctaaagaacctcctgtattgttggtaaccagacccttgttagcatctgtaacaagccagttatctttatcatcaacaattgctaaagcagagtcagcaacctctacaggtgtccaagcagtaccattcagggttacatctcttagaatgactgctgaaccaatagttgttgcagcaatttttgctaatgtgtatgctgtgtcaacaacgttatttcttgtgttaactctaaccacaataccagcagtcataggtgtaatatgttcaaaaagctgtgaaaatgttgcatcatacagagtcatgatagcattctgtaaaaatgttggggttgtatcagaacgtcttagaaaaatctgaatataaagaattgctctatatgtttcatcatcagcaccaagtggtcgtggtactttaattaatgcaccgatgttgtcaagttgctgtccaatagccttcctgatattcctttcagtgtgcatctgccatgaaacatcttccaatgtttgtagctcatcagtgatagctttcagcaaactcgtatagatgaatttctctttaaactgtgtaacagtcctttcatcaagagtcttgtaataaacatcatcaattttctgaaacattatcactccttagtaatggtgtacttgctactttcccatacagtgtattggtcaccatcaacagtaattcttgctgtagtgtactgtccatcactaggtggtactgactggctattggatagtgctactttgatttcattaatctcgatacctttaatgacattataaatatacccatagattctattagggataacatcattaccaactttcaaagttctaccataagcgttaataccttgaataatactgtctctgatatcttcttctggaattgtcaagctttcttcattatatagagagtatgatacttttacaaaagcatacttaggtgttggcctactgaagtaaatattatgagatagtccacctaagtcataagctgtcccaaagatagccccataagctctaataccagcaggtttagtgtcccagattgcttgagcaacattatcattttgaccaccaactacaacaatcttgaaagattttggtgggagtccttctgagcttgtctcttcagtatcattttcaacacctgaagcatctgatacgccctgaaccctcttaacagcagctacgattgcatccagagtacctacaccagtaactgacagggattctaaatatctctgacgaagttctgtatcggtctcttcatttctacctgttgtcaggccataacggttatatacgctatcaagaccatctacagttgtttcaatttcgataagtgtcccagctaatgcaggaattgcaccaacttcctcagcaacaacatcatggatagttgtaatttttgtgaatgtgaggaatgtggtagcagtcactaccatagggttggttcttgcaataatgtcgccttcatctttataaactcgtaatgctgaaccatcattgatgacttcagcttttgctacaataccaccattaattgcattagcaagctcagttaatagtactgtgattgtgtctgaagattttggctgataagagaaaataacgttatcaataataataacataatttgcatcagttcgtaaagagttaacttcaagaatagcctcaacacagtatgaaggggttaatgtaatgtttgaaactggatagaagatattcccagcagtacttcttaatctggttgtagacggaattgttgcacctgttgttccagtaaactctacttgaccccttgtagcttgagcaacatatctatacacagcgtttaaagctgtaatatcatcaaggttaaaaccttcagctttatcaatcgttccaccatcatagatttctgatagaatttcatgagtgtctgctaaagaccttgcaattgaagctagaaagagacctaactgactatcctcagaaacgtcaaggtttggtgaaatatctctaagaagccttgatttgatattatcaaaaatttcctgatatcttagagtttgtaatcctgttgtagttaatcctgccattagatattaacctcttgcgtaatgtctgttaaaatatctgttgtagtagttgcatcaaaattaacagttacttttctctgagcattatccattgaagaagaatagttatagatgttagatacatctcttgtttcaacaaggtaggctttcatgtaattatcaaagatagaagttttctgtttaaatttggcaagttgtaaatatgggaacccagcagatgtattaaagaaaacttcaccagcccttaaaaggcatctaatatgaagtctttgagcaacctgagtagctttatcatcttctgggataattctaatttggttaccagtaatctttaaatctccatgagccacatacactgaatctgaacctaaagtggcaacatagtcaccacctagatttaatgcaaagtctgttttcattattgtgcctctgtagtatcagcctcaccagcagggtctgtccagtaataatggtgcgtgtgttcattaaagctcacaccagtcgtgtcactgataaaatctgagccgtgtacttcttctgttacgtacaagtttttagaaatgtgtacatcaccttcaaagtagaagttaccatcatcagtaactcttaacacagagtcaccgaaatgaagtctaactgctgttgggtctggtttaaaattctgtgttcttgtgcagatgcctacgaaagctacacagtctgaaatatcatgtgtccttctcatgtttgtttccatctgaacattcttgtcattgacaacgaagtcatctaaaggtaacattgagaaagctaaccagcatctgtcattagtttttacggggaatgttaaagatgccccaccaccacttggaaattgaacaggtacaccagtaatctctggcataggtaaaccgttaatagagtaaagtggcttaacagtggctgtttgagtctttgaatcgaaagactgaataatggctggtaatccagtatacagttcttttctaaattcatcaagacattctgaaacatacccagacattctagtaactggtgacttcattattccaccttctctaaatctagttcagttgtccaagcaccaccagtaaagtcaagattgtgagaaagaccttttactcgatactgaccttcaaaatcttcactttccctaatagtgatgttatcacccatcttaattctcccatctaaatggattttgcaacgaactccagttttaactttaataactgtcttattctcttttttcagaacctttctagttcttctgtagtagccttgcaaagaatcaataacgttatatgggtaaatttcccaagaaagttgtctagccttagcgttaaaaggaactactcggatttgcttgttaaatgtataccaacgtagactactttcttcacaaacctttgtaagtgcctctgcaacacttccccaaacactaaaaccattcttgtaagtgtaactatcaatacttgaaaggtcttcatcaataagtgagaaacctagtctgttgactaaatctctaatcacacttttacgcgttgtccctgctttataagaaattgatgtcttaatcgtggttctttccattttatcattggaacagataacctttgtaatcatatcaacaccacgcttatatgtataagcatattcaatagtgcctagatagattaatgggaggttatcatattcaataataaggtcgccatttgcgtcccttttaaaaccagtagtgtaacctgctctaagcataactgttgcaccaacggttttgaatttggctctcatctctttattaaggttgtagatttcaaaagtggtatcatcagaggttactttattcttctgagacgtataagacacattacaagtaaattgtaagttgtcaaaatagtccatttgcatagaatctttagcatggcttgtaggtttatcattaaaggctgtagtttcactacctacagccaattgatagcacctaaaagaagccccagcagtgctatcttttacagacattacaagttctccattaatctcatatcttcttgagtgtaataattaagctcaaatgccttttctcttccgaagttatttctggtaggctgtaaatcagtaccatacattcgttcaacaaaaagctctccagctaatgaaggaattacatagcgtcctgtgattgattggtctgcaaggcatttcttttcagataataatacattaccatcaacatcagatagcgtcaagaaccatctgtcaagcctctctttatactttaattcaattacaaagacagtgccatccagagttacagtttgtgtagaccatcctgtatcaggaacaggaatatattgtgacattaataagtcccctttttattcggattcactgaatgcttttgtaatgcttttccatctccagcagttctgtttaatgctgctccggcatttttctcagcctcttcactgaatgtagtaacaccttttctggtttttgcagacatggaacattttgcaagggcattatcttcagcactagtaagttcccttactccattggcatcaaggtcgaataataactgacagtttaatttcccattacctagacttgttgtggtattccctgtattctttttactggtagcaccaccatcattcgtggtagcggtctttccagttgcagcagaaatatcagtttgtcccacgatagctttaaagttaatttcctgaaaagttagctggattcttagaccatttgaaataccaacatctttagaggctttaaaacttgtgataatggaatcatcaattttaattccgtctttacagatgactgaaataatttgcttctggtcacgccagctttcaagagtgtcgatgaagttttctactaattgaccctgacgagttaataataagcttccttcatagccaactacaacgacaccactaatagtgattgttctgggtgctctttgcacattatctgtgacggtttgccctgattgcatgttctgtgtagttacctgcatagggctgtcaaattccatattttcagttgctgataaggttaagaaggcatctacattatctcttaagtggaaatagatgccgtctttgccactatatttgatttgcatattagaatcccataacattattcttcctctggatagcttgaacttagaagaatgtcttcttgattcttgtcagtaatatccaccatcttagtagcaatttgtttaccatcaagattgaaagtaacattcagggtttgtttagtctgcataggtaaaccagaaggtgtcatcatcattggtgtctggttaaacttattggcaaaattatcaatagatgttgatagcttctccatgataatctcccaattagatagaccattatcaattaactttctgttgccttcaacgtcttgagtgtactgtgcaaactgaagttgaccattctcatcaaagaacattggcctttttgggtttgtaatatttgcaacagcattttcaaatggttttggtaatgtaacttcactgtagtttttagcagcatttgggtctgtagagcctcttagcattaatgctgaaccaacagtaccaagtgccattctcgttgctgtaactccacctgctgctgcggctgcttctccggctgctgctgtacctgctgcactaacaccaagtctttgtaagattttaccaaagataccaccaccaaccaaaccacttaggagcttgactgcttttgatactacagcaactgcgcctccaattgtgacaactgttcctagaaattcaccagcacttttgataagcttttgctggctgttgtcaaggtctttataccaagctcttgcatagtagtatagtgcagatgttctgtacatgaagtctgtaatgaagtcaagtagattactagcgccttttaacaggttcccaatcacaagacctaaagcctgcgtgctacccatagaaccttgcaagaacattgcaacggagttagataactgtgaaatgccctcactagaattgttaaacagtgctacaagtgtgttatcccacatagccttcgcttgacccattgatgtagcagtctgcttggacacagcattcataccacctgcttgcttgacaagttcagccattctttcagatactttaggaagaacgtcttgagcaagaagtttaccgtcttgcatcatcttatcaagttcttgtggggtcttaccaatagcatcagcgaatagctgcacagcacctgctaaacggtcacctaactgtccacgaagttcttcagcctgaactttaccttttgacgccatctgctggaatgcaaccatgatacctttcaagtcttcatcagtagcacccctgatacgggcaaacattgcagcattcttatagaactcttgagtaccctgaaagccaagtgttggttgagcaccagcagcaaagtttgagtactgcttcatggtatctgtgtagttctgaccaatttggtgtgcgaatgatgcagcgaacattctagcttgctgagtgtctgctccaaagatagctgtagaggctaattgtgcagattgtctttttacaccagcttcaatagttttttgtgagagttccagtaaagcataagctgaaacaaatccaccaactaattgacgtaatgatgcgttagctctatcctgtagccaagctgattctttaactgattttagtctagcattttctgcaataacccaacgtttggttacgtcgatgagctttttaacttccatctcatactcaccaaccttaccagtacctttatatctgttatagatattttgcaagcttcctctgaaagaggctgccatttggttaccttgaccaccgattgtttccagtctacgggttaaccctgaatagaagttgttgttaaacattctttccatttgtctctgagcaacatctactctcggacctctgggtgctccaccaccaacaggagggatactctgtccacctctccctctccctgtcttgatagtgattttaccgtcaaccttcatagcatctcttaatgacttgttaataccttttgcagtcttttttgcctgagtttcaagtttcttaagagatttaacaccttgtgaatcaaggttcaaggaactgttgagtgctttattgattctgcccgaagcagactgagcattttttacaattctattaagtgcttcctgagaacttttattaggtttcacatcaaaggctttattgatatttcgctcaatacgctgagcagcttgcatagacattttctcaactctttgcaagcccttaacaaccttttcactgaaaccaagttccacaatgaagctatcaactgtatattgtgccattacatttttcctgctcttctaagttcgttgtaagcaatttcctctttatacgacctctgaatttcaagaaattgtctcaatgataataaatcagagaatgtcatagcaaagagttggtcaagtgtttctttacacccttccataccataaatagcaagcacaaatttcatctcgtctgcttcttcataggttgcctctacagcagcatcagttagtggtgtctgtagagtattacccatgtttactgagaagttaggcttttgaaaatgcttgcttcgaaaaaacttccgaagtttgcctccagcgcaaatgctaagtaatcaataaactcaccgtagtttgcttggaagtatgtatcaatattgagtgggaagtcatcaacagttgccccttgaaataacagggtagccatttcttcaaggttaatttcttcaattctgtcaaaacaagcttcaacaaggtctttaaatggaaccattggagcttctttcttatctttatcagtcagacttgatagcatctgtgcaaatgttggaacaacaattttacccagcttcatagacatcttaatcccatctcttgccccaagcagaacaatatttactttcttaccattaattactctagattctgttttcattgtgattccttaatacttttaaaagaaacaaaaaaggggaagaccttttaaagtctcccccttataggatttattaaacacttgacgctggaattgtagaagtgtagtctagcttctcacaaccaaaaatccaagttttagagttctggtcacgcccaagttcaatctgtggtaattcctgcaaccaagcattaataccagttgccagaacagagcctgatgggtcgtagattacgaagttagaagagatatcttcttcaagttccatgttgtcttgtttagcttgaattgcagaaagcatctggttagagagagaagtctgcattagctcaatctcaatagtacctgtcttgtctgcatttcttgtcagagcaacctgaccacctgcacctacaactggtgtgataagtggtgatgttctctgtagacgtaaaaatgagtctggggcaaagccttcaatggcaataccattccagctacatacaacgtctttaggggaatattgctgatacatagccattccaatttacctctattattcgtaagccactgtacctttcaagtcaacatccaagatagcccctgctaagatacctgcgaaggtaacatctttcaggatacgagctttcttgtctgccaaagcaacttgagaggctttaggaacattaactgtgtaagatgacaggaagtttctgttgactgctctttgtagagaggtttcaatgacttgacgaatacgggtaataccagtatcatcataagtaatcttaccacccttctggttaattagcaagtctctcagagaagttttcaggtctgattctaaccagtcaacaccacggacgatatcaatccattccccaccagaagtaatccctctacgaaccactggaacaccaccatcaaggtcgataaagttacagtgacgtacatctaaagctgacttctgaatgcttgtcagaggtctcttattagctggctgtaaagaagcagctacaccagtcaactgagcattaccccaagcaattgaccctgcatcgtatggagcgccataagcaatatatgccatctctggataatcttctgccgctgtgtgatgccacaagcaaactgtgcgagtatacatactcttagcaagctgtgctggaacatcatttgcactatttaattctgtaccttgcagtgctgctacatcagagttagctgtgaagaagattttcttacgagcctgaatctcagaagccattgccaaaacaaactgttgagttctgtcttctgctgcaatgaaataccagtcggtagaataagcttcaatagctgccagagcagttgacgctgtatcagcagttgtgcttgcaatagacacagtttgtgctgtagtggttaccttcacgaaatcattgtcaccagctttggagatgaccattgtggcagaaccgttgctaccagtaacattgactgaaactttatctttaattgattggtcagcttcaatctgtgttttaaactgttgtaatacattctcggcagtatcttcaccttgtgaggtgtactggaatggttgtgacattcccccaccgacagctacagtaattgagtagtccgtgctctcagtaacctcatcaggaattgatacagtgtactgcatagcgcatctaccaatataaagctgagttactttaggagtctgactccaaagttgtttagcagccttatatgcagcagtattctcatcgaaatcttcagcaacttcagttaaggaagtataaccacgcactctttcttcaaagttatctgttgaagctaagaatagtggcaaaccaaaaccttctcttgtagttcctgcggtgttcaatgtaatatctacattaacaattggattccacatttattttacccctttggagtctacatctagatggatagtatattctggtggctcttgtccttcttcataaaccaactcgccatcaacaatgacacgctcaataatacttccgcgttcatctttcaggactgaattttttacaagagttacaacaagaggcgcagaattttcgaaatctgtattgagataagtgtagtcatttgggatggctccagtgtctagcactgtagctcctgtctcttcaagaattaaatccctgactgaactcatctctaatcgttgtttaagctcaagcataatactgtgagcacctttcccatttaccgtaattaatacaggaatctgaaaagcaattctgtaacaaactacatcatcttcaacaaacttatcaagaacccaaccataaggtgttgcagcatcttgacaatatacggtaataaatggctggtcaggttttaaacctttgtcatttgagttatcagaagggtaagctctaattacgtttggtctattatttttatcacgagctaatctgtgaccaataacatccactaaggttctaactagacctttttcaagttctgctgtttctaactgcattcattttatcccttctaataatgatatattcataatgggatgtatgggctaattgctgtgaccaatccatagtcataaatacttcatactcatgaccatcaatcataacaatatcagactcattccactctacatcatctgaagttctaagtttatatgtagtatacaggattcttgtatcggttagtctaataccttccggtaaagcaatctgtgtaccattctttacagaacctttgatatatggctggatattacctttacagttaacctctacaatatcttgtgaagctacccaatcaccatcatcattataataaccgtcttcagagactttacgctttactacaaagctgtgtctgtttaagagtctcatttcttaatacccttcttagtagaaattttataagcaaggttatcccttaagtcacctgtctcaacaagaggtgcattaaagccttttttcttgactgtggaaggtgcgttaggagggaggatagcagaattaccaaaacctcttttaattgccttttgagcattctttgcaaatgcttctaaggtatctgaggggtctacgttgagactgctaagttgcttatatagattcttcttagtctgctctaacaaggtctgtctgtttagcatcatagtgatttcaaacaacctacgatatactttaccagaagctgaaggaaccccaataacttcttgtaaatacattaaagcaggataagaaaaaccagagctatgttgaccttgttcttgaaaatacccaacttgagcattagccgtttgcaagttcttcatagccccgactaattttgctctagcggggtgaataacccttttaaccattattcatctcgctcgataataaatacgccattaacacggttgcagtaatctctgccttcgtatctggcagcatcaccatattctgtgtatttcttgacagagcaaggattctgacgacgcatatcaatgtcacactgattaataccacctgcatagggtagtcctgaaacagagcttttaacaaggtcgtcataaacagctttcagagatttgaatctggaagagttacgtaaataaacaccaccaaccttctcatctcccatcttggctacttgaaagagtaaatatttaagagctttgatggctgctttcttttcatcctttccagattctagatagaaccactctagcacagactgttcaataagaatttcatcattattggtgtctgtgcagaggattcttactctatcaagagggttattggctgggtcgcctgtataacacattcataaccctccttaagaatattagtctttagcgtcagcacgaacatctaccagcaactgaggacgagtacagtatggcagcatgtaagagtgagcttcgaagtcaataccttcgtcacggtctttttcatattcgaatacgtacagttcctgaccaagtgtatttgcgtaacccatcttaggacatggaccataagccacttcgaagatgttgtttgcttcaccaagcatagaaacgttagggaaagcgtgtcctacaccaacctctgcaccagcaccatcaatgctcaccagagtatgaaccttaccacgcttgtctttgaacttaccgttgtactggacaaacttaacaccaccgtagtagaaggtgttcatgtgagcctgaacgccgtcagtaccaccagttctcagagaaccagtaatctgttgccaagccagcggagtctgctgtgcaagataagcgtcacgaatcttaggatgcttaaccagtttgctgaagaatacacggtcaacaactacgtgaatttcttcaccgttgattacagtgccagtcttagcttcgtcttccatgtgcatacgcagttcttcaatagaagcgtcgatgtcagcattagggttgccaaggtcgaagtaaacagttttcttctcaacgtcgaactgcttgtacaggtcagcatacagagtaccacgagcatcaacaactttacccttcagagcttgcataaacaggaactcacgagtaatatcgaacttggtacgaatcttcatcagcttcttagcacgtactacagcttcagtagtcagttcgtttgcagtgcctggctgacgtacaccctgaatctcatcaggagtaatgctttcaacttctttgaaatacatcattgggaagctgatttgacgaacacgctcaggtgcgctagtctctgctttacggctatcacggtctaccgcatcaagcaagctaacatcccaatcagtcaagtccataaggaaagtagtttgggtgattggtgctgaacggaacagacctaagttggaaatatacccataagtatttgggatagactggacttcaccagtcaggtcagcaaggaaaaatctgcttttttcagaattagtcaacattgtaaaattctccagaatgtcttattattgttattacaggccagttggtacgaaatcaatacctttagcagccagagctttcttgacgttctcagcattaacaccagactcaagagtaagcatgtctttcagttctgcgtcacggtaaataccaacaactttcaactgaccgtggtaagacagttgcaggtctgaataaaagttaacgatacatacagaatcagcctgagcttcttcccctgctgcgactttagtaccgtcttccttcagaacttcacctacacgatattctgtagatgcctgtggagtgtactctttacgagagtggcctgttggggtaacctgttcccaaagaatgatatcattcagaggttctcttttacctaacttagtaaaatcttgatatgccattattgtgttccttatttgataagagattttagagcattctggagagccagtttgcgttgttcagcggtgtcttcggaagcattcttagctggttctttttcttcttcaaccaaatcagcttcgccgtcattacccatttcttccatagcgttggaatgttcaagaacagcaccagcagtttctttcagcttggtaatttctgattctttttcttccattgcagaagcatgggacgcgatagtttcattaagtttctggttagcaccttccatagcattcataaacagaacactcagagggttatcaagaccagcacctaaaatagtcgttgcagcttcttttgcatcaaatccaaaagcttcagcagaagcagaaatcttattagtcaaatctgacaaagcagcttcctgttctttagctttcatttgagcaacctgaagacgcaaagcttctagttcttgcttttcttgttcagtcatttcttcacctgaattgttaacgtttaaacttacaggagcctcttcagaaccttgtaagtaatttaagaaatcatcttgagacatgattgagttaattaaaccaagttcaagagcttcctgagcagaataaacattcgcctcagtattctttacagcttcttcagagagattacgagattcggctacaaaacctgtaaaggttgcgtaggtatcattaattctcttttgaagtctttctttgctttcttctgagagtgcttggaatggtgaacccatacctttaaactcaccagctttgatgacgttaattgttacgccattcttttcaaatgccttagttaattcctgatgaaccataattacaccaatagaaccaacatctgcatctggtgatgcaataatttcttcagcagaagatgcaagagcgtatgcagcggaacaagcgaactcatctacataagcaataattttcttttggcctcttgaagccataatgtgacgtgctaattcaaagcaacctgaagcttcaccaccaccagaatcaatgtgcagaacaatagtcttgattgactcatctgctaaagcttcgtcaaagcctctacgcaaaccttcataagagcttaatccacctgtacacattgcatcaatgaatgtcatacgatgggttaaaccacccataatagggataatagcaatgtcatcttttacttttaaaagacttcttgcttcacctttgggtttatcaaagtttactgctgcttggacatcacccagcaatctgttattcacataagttgctgctgagtgagctaatgattcagtggctagtaaaggttggttgaataatctatcagcaagtctgaagatattcgaactcatttttactcaccctatttgtttaaatctacagagactgaagagataacatagataccttcttcagcaaagaattgaggtttgctaagagcaccagttgccacgcatttatcgttagcatcccacaagctatagtgtgagaccgttgcagaagctgggacagtaatattaactgtgtcttctgaggcaattaagccattatccggttcagaaaaataaatagccactggctgagtaaccttatttgctgtagggtctgccgttgggtctacattgtgtaaaataatagtcgttggggttagcgtggcgaggattttattcttaccatcaatagttaatgttcccattaattaaaccttactttttgtttaaggactgttgaatggttaccagattcatctaccacatttacaatcatatcatatactttacctttgacaagtactttgtaatcattttgtgaaaaaatgtattcaagtctgtttgtttctttattaactgtcattggagattgaaatgcagtatcgagggtaatgaaagcggcttcaatgctattaacattgatacgtttattctcgcagttatataactttacacccaaaaggcatgaactgtcaaatggaattttaacaatttcactacaatttcctgaaataaacggttttccactcattggtgcatcaagtaacctgcaaatagtgaaaacgtcagagattccaccatcactaacataacctgaaaggctgacccttgagccagcctctacagataacttatcagtaatgataaggacacccctatacgaatgcactcgtgtagcatttgaaatagagataacttcagccattatttatttgccttatttgctgtgcttgggtctttcgctgaaggtgtctttgcagtaccttctccagcggtcttataaccatctcctgagcggctttggctatttggagaaagcttttcagatactggttgagactcatcagcaggaggaagaccaatatgctctctaagtttgttagatagctctttgtcaacttccaaagcacctactgcaacagtcttctgaatataagaaccaattgcttcaaggtctggagtttcgatatcatcatatgtgatttgtacatgttcttcatcatcccacatattaagagcataagtctgtgcaactaaatcacggttaattacgttcttaatttgcttcagcaaaatatctactgacattgctaataagcttgtcttagaatctgcaagagagaatgaaccatattttgactgacccatagcaagaacatctgacataaatgccatcataatttgcttggaatatctgtcaatgatagaacctgtatcatatgctttagcaccctgtctagagactaatgagaactcaaaaatatcctctttagtatctgggtcgatatatctaggccagattaaacctgctctgtcattagcaatcatatcattaacaacagttttgcagtattgtacgaaagctttcttttcaggttctgcattttcatccagataatctggtggtaaaccaatctttggcatacctaccaagtctcttgaaacaccaacagcttcgtactcttcaatctgaactttatacttccacggtacataggcattaagtaatggtgaacgaccttctgggtttccatattcatcatcatacttaaacagcatgaatttagctcgtggaagttttcttgttagtggtctttctccaagattaattgctccagcaatatgtgaaacatttctcagattctgtctaacaccagtaacttttctaaagtcttcgtcaaaataccacttatcaagtgttgattggtttctgattggtaatttagcccatccaattagaccatcatcaaattttgactggtactttccttttttaccctgtcgcttcttataaactttctcgttaacacaaaacccataagtgcagaatgacattacagaattaataaaatctgcccaatcatgctccatgtcatccattaaagaattaaagaagtctgctctttcaagcattttagggtcttgctcttttccctttggaggtacgaatctccagttgacttttctgacaaacatcttaataatatttacagatgctgctacagcagggtcacgcatcattaattggaaagttttaatactttcagggaaccttagtgcctgacgaggttcttcatagattcttccattcttaaccttcagacccaaagaacctacttcacccattctaaatggtggtaagctttcttgtgtttctgtaatatctgccattcttctcacctagctatcaacgtctaagcccctcatatgggtttcctctcactaagtctgtgtgagcacccattgatggtggcttgaataatttaacttcgttaagactgttgaaagcatcactggtagcatccacttggtcatctttagttttgccatcaccacaaaagccttcaagctcttgaaagtaagcttcgttccaactacctctcaagacttttacaagtccagcttcagaagcagcagaaaatcccgcaaagcgggtaactttatctttatttgttggtttagctcttgcacgatagcctttctcggcaagtttcctgatgagggatgttgcgtaggatttaccagcagcgcctgggtcttgagggataaaaataccagttcgcttaccgtcactttcagcagtcaaattaatttgtgtttcgactccagagggtctatctctaaatcttactacatcaatgatataatagcaaccgtcttttttagatttacccatcttaacacctgctgtccagtctggattagggttaatctcagatggtaaagttgctgctaagtcccatgctctaacatcaaatacatcttctgggagtgaatcaacaatctcacaccattgtctttgccagtagtttgaaccttctgcacgagccttccagttaccgaaacgaagtcttgcaacgtttacaggggtgttgttttccaacttaccacggtacttaggttctaagaagtcaagaattgggttatcatcaatcgtaccagagatgaaggtgtatgtttgaggaatctcaagagggaacatttcaagaatcttgtctctctcccaatcagaaaccatcacaccatcattcattacataccaacgaatacgaccacacttttcagggtctggataaccttcttcatctaagaatggttctacccaatcataaataaagtggtctctgtctgggttcatggaaatcttcatgtatgaatcaccttcagcaccagaacgcagacgagtctgtaggtatgaaatctgtgaagcagagaagtgtgtaccttcgtcaaagtagattgctgagtattcaataccctgatgaccttcagcgtgcttttcaagttctaggtaggtaaacttgatagttgccccagaagggaatgtgatagtcattttctgctcatgaggaatcccaccaaacttaccaaatagtttctttgcagcaggccataaaccaccttgtaactgtgttgtatttcgacggaaatatacagcattatagttagggtcttcaataaatcttaaagagtccattaacaatgcggcagtcttaccagcaccagcagcaccaccatagagaaccaagtcagcattagtatttaaaaagacctcttgagaacccggttgaggggctacatagttcttatcagtcatcaatttaaagatgaggcgaacttggtctggtgtgtatcttaataaagtcagaatttgagttggaaggaatttagaggggtctttaccgaatgatttaataatttcttttacttcatcagaaagccccaactctccagctaggactttcctaacatcttccactcgcttctgcttaacagcatttaagtccattaagcgcctccgcaaatagaattattcagagtctgtagcagtctcttctttcttaacttcgaacttttcatcaacagcatcaaggagtgcatccatagatgcttctttaatatcaatgcctgttgctacagataatctatctgcaattgccatgatagtgttctgcataaattctagctttttattagcttcataaagttctttgtaaacggtctcactcattatgttttctccaaataattaaccatgtttccccttaaaaaagacggcacagagaccgtcaaggagaaaccacaatgtacgtcagagacatactgtataagccttcttataaagtataactctgtaaaaaagccctgtctaaaaatactcttcggtagcgaaaggaagaatacttatagcagggcattattattattattttaattgagagagagtaaaataacttggagaatccgaagggactcgaacccttataaacctgttttgcagacaggcacatagccatttctgtcacggattcaaattggaggaagataccagacttgaactggtacaccgatttctcagctactggcagtttagcaaactgctcccttaccttttagggttaatcttccatttatttctgtaatgcgttcttcatttctggtgttgcaatagtgtcaattacaccagttttacaggcatcgtcaaaccagtctggtaagatacctgctaagaacccattgatattcgttttaaggtactctgcaataagggcagcttcttcttcaatcatctgcacaacttcatctgcataagctgtaatctttgaaataccttcattaatcttactaacagcctgattagctaaatcagaaatagtatcaagaccctcatcaatagcgtcttgtaagtcacttaatacatcgttaacactctctagtgtactgttaattgtatcaatagctttttgaccatattctgtagcaacacccataataccactgaacggtgtacaaccaacttgttctcctgctgcacccataacattggaatacccctttgctacctgcatacgtgatgaaaactcatcaatagatttttgaccgtagtctgtcagggtcttagtagttgctgtagtgcttgtaaggcttgttgtaaagctgtttagaaggactgttgtaagtccagcagcaacaagtttctcctgcattgtagggtctgttacagaactaatagagcttacaagtgatgtagaagcagctacggttccaccgagaactgctgcaccagtaataagtgggttagaaaaccctttaccggtttttaagagattaaaaatctccttaccttgttctgtcatctctttcattaagcacctttgaaattggtagtccagtgggatttgaaccctcttctcatgtttttcagacacgcgctttaaccatataagctacttggactataaattggggtgacctacgggatttgaacccgtatagaccatgttcacagcatgggtcattaccatttatgataaggccacatttaaggactctcgtaagaacccttagaagtggcagcggcataaggatttgaaccttaataggacagcttcagagactgttgcattgccagttatgctataccgctaaatttggtactccatatcggattcgaaccgatacataacacagattttaagtctggcctctctgccaattggagtaatggagcattggcgggggatgttggaattgaaccaacttcttcgatttcaaagaccgaggttttaaccttgtaaactaatcccctttaaatctttactttcttagtggatgaataaagaatgccaacataactcttccccttgagaatgctctttcttctgggacaacactttttagtttccatccaatataaattctccagtaaaactgtttaccaaagactttaattgatgggataaaagcgaataatccccaagcattactgttccacattaggagataacctgtctgattatcttcagggtcagaacttacgttgatattacctttccacttagtaacatcttttacatctcttcctaatacatggtaagagaagttataagctttgtttctccagagccatccgactctctgcatgtagacacccagcttaccaatctttctaatcttagcccatcgttcgatatgaccttcgtcaccatcaattgggttgtcatatgtctccatccatctaaatccgaaaggtaagtggcctttcttctcactgtagaatggaactacaaaaggtgctaagataactgctaagatttctgcaaatggctctaacaaagctaagaaaatccatgaagcatactttaagtatctcatctaaatagcctctttaaattggcgcaggataagggattcgaacccctattaacagcttcgtagactgttgctctatccatttgaactaatcctgcaaaattggtgttccaagacggattcgaaccgtcactagtacaaggtttgagcttgcatcctctaccaattgggatactggaacatggtactcactaaaggacttgaacctttttcttcatcttgtaagggtgatgttttaccatataaactaagcgagtgtgaaagaggtctgaagttgtgccgctaactcaactccgtggaatttgttacggtcttcagttgactcaaagcggtctttcacttgacctctgaattggtgctgctcacaggacttgaacccgcatctccatccttacaagggatgtgtaatagccaattatacgaaagcagcattaatttggagcatccagagggaatcgaaccctcaacctcagtttggaagactgtaattttcccgtttaaactatggatgcactaattggtggagtcactgagaattgaactcagttcccaagtttgcaaaactcgaattttaaccatataaactatgaccccaatgtctggtacaggtggagggaattgaacccatcgtcttactgattaagagtcagccgcataaccattttgctacacctgcattaatttggtaggagacaagggattcgaaccctcaaacaccaacttctaaggatggtaggtttaccaattaccgtcaatctcccattaaatctttttagagaacttctaagaacctaagcaacaaggttccacaacaagccgtaataaccttagaagccctctaaaaagacctaaaaggtctttgcatagtcttgtttaatccggtgactagaacctttggagagcttgatattatctcttctccgtgacacctactaggtgttttatcagtgtgtacgacaatacacgagggataccaaagcaacatccgaatgaagcttggctttaaaaatcttattaggcggtaagtaaaggagtcgaaccctcaccgtatctctacagtggcaacagttttcaagactgtttggctgccattagccgctacctacccctaataagactttggcatgggacggaggagttgaacccctttgaaacggttttggaggccgttgctcatgccttagagtcttaacatcgtcccatactataattcgaagcttaccgtattatctcaacactgtaaagccccttctcagattatttacatgttctggaaaacatgagatacagaccacctccctacaggaagacccgcttaggtcgtctaagtaatctgctaactaaattggtcgaggcggcaggactcgaacccgcatactccacttactcggttaacggcggtttagaagaccgctgagatacgccccgtaattatctttaaagactctcttagaaagcccttaaagatgcccaccttattaatcataccgtgggcgagtacgccaaattctttgatgagggattggaagacctcactggtgtttagcctatcaagctactgccagaaaaacattgtcgtttgcatttattttaaatttgcaaaatagacgctacgcaacgaaaactaaggttactataaagtagtcacatctcaatgtcaacaactttattgaaattggttgtaggtgagggtatcgaaccctcttctaacggttatcagccgtttgcattaccatttatgctaacccacaattggaggttcagatgggaatcgaacccatattcatagggcttatgaaacccctgcattaccttatctgcgactgaaccatattggtagaagtggagggattcgaacccatcgcctgtcagattaaaagtctgccgcatcaccattctgctacacttccattaaattatctttgtagataagctacccagacttgcttaaggtctgtagcgatgtgcctttttgggtttaaatctagaatttaccctttcaccctttgtggtcgcatactcacaggtaagcttaataacttatctacaaagataattaccagaccgttgtttatcatcttaagtgctaccattacaccaactcgacctctgccgagtgaaggaatcgaaccttcaccttttctttaccggagaaatagattgtttaagttttgctgtaaacggtcttctgtaaatttgttgagccagaccaagttttaaatttttcaaaattaaattggcttgaaaatatcatacgtacttgctgtatttggtcttccgctcctttaaaggatactcgtaagaaccctttaagggaggggctaaatagccctctcaaaattggcggttacgaagggatttgaaccctcatcatctcccgtgacaggggagtattttaaccagattaaactacataacctttattggtgtgccgtgtaggagtcgaacctaccgagtctcaatgacaagggatttacagtccccaccgctaccatctacgggataacgacacatttaatttctattcaggctaaggacttgcaccttacaaatcggtgagcaatcgcttctctcctaaatttggcggttagtcagggattcgaaccctgtgccattcgcttaacaggcgaccgcacataccttatgtgcttcctaaccatgtttagtattaccagaccgtatctttcaatttttccgctaaaaatttaataaatattgctgtaaacggtcttctgtaaatttggaggcgggtgcaggagtcgaacctgccgataccatgctaatgagacatgtgagacgccctttctctatacccgcaattcttaatgaccagaccaaatctcttctttgtccgatttaccttcagatgaataagattaagtttgctgtaattggtcttctgtcaaaactggcgttccagaagggatttgaaccctcagatatccactttgaaagagtggtgactttaccattttgtctactggaacattaatttggtctctgttggaggacttgaacctccggccttaccgccccaaacggaacgctctaccaagctgagctaaacagagataaacttttcaaactctatgtaaccactttaacattattttttagtggttgtcaagaacttttttaaaatatttttcagtatcttagaaaagctctcatttcgtttctatgtagaacatattaaagggtattaaacacattgtcaataccctttttaaaacttttttaccagatataacggtcaatcattactgctttgagcattacgctgattgggtcaaacttctcaccaccaagcagagcttttaaagtggctggagagaaaccagataccattgctacaccgttatccttaacggacacttcgcaagtaccattacggtttgctaagtaccagaataccagttgtggcatctcatatccagcttttttgtacttgctctgaattgcttcaaagtttgtacgaccgttagctccatcaacctgattgaactccatatcagagaatatgataagcttacttggcatatctttctgagtcaagttgtttcttttacctgtctcaagaatacggtcaaaagctgcttgtaagttggttgagccatattcaacgtgtcgcatcacctgacgatgacggtttcgtaaatcaccactcagttcaatgaaatgagggtttgttgaataaaccatcaactcatccttaaagcaacctgtattgcgttctgctacatacaaggcaagtgatacaccaatatcaagtgcagtgattgaaccaaaattcacccaagacattgagcttgaaacatcagtcatacataggatgttttcaccttctgccatccagtttggtagtgctttccactgctcattagcaacatctgcattaccatacttaacagatttaatcacatcgtatgggtaaacagcaccagcattaatcttagcctcgccctttgataaggactcaatgtaagctttgtagcgctctccatctttacggttaaacagtttttggtaacgtgcagcagcaagtgaaggaatcttgctgtagtcaatcttaccaaactcattagcagagattttttgctcaactgtatcagatagtgcagacaacagtgtgcggtactctttctcactcaagttggcaaacttgcagaaacgttttacaaactgcttatgacgtggtttgactcgtggtaaccacttagccgccaaacctgctgttgcagggtctagtaatgctgcttctaaatgtttgaaggcatctgtctcgaaacgagtacctacgaagattttgaagtcatcaaaacgaccaagttctgcaatcttatccataatgcgaagaacctgcgtagactctaaaactttgtcttcaattgcttgaagcaaaacagttctgaaggctttacgctcacccataccttctcgtacatctcgcatatgaagcaaaatacgaactgcaacatcaacatcctcacgcaaagctttgtagaacaggtctggtaagatttctacattgctacggcttgaaccagcagctttgtaaaagtctacaagagcagacattgatgaagtatggtttacagcaccattttcagttcgacctgcatgaaggtgcgcatgtttaaataattcgctcatattttactctcttctctcatttgttgtttgatgtgacagactttagagcaactttatagagtctgtcaacatcttttaaaattattttttaagggttgcaacagcagagaacgttggtttgttgtctgtctgagactgaccaccattatcaggagtcttcttagtgtctgctgcaattgctttcatttcacctgctgagtgggtcatgatagttttaccgacctcaatcatgcttttgatggttacgtcgtctgtgtttaacccaaactctgcaagcttagcagcgtcccttgtaacaatcgcttcaaacaactttgcagcaaactcagcagaattatcaatggtcagttgagcttttacgagtgaacttttgttacgagagccttttggtcttccagatgggtttccagactggccttttttaaactggcctttgtttgttctgtttttcattggtatgcctcttataagacctcttaaaaggcttttaagataaaagatagatatccagaaagatatctgtttaaatagcctttttagaggaaccttttaagttatcttctaagtatttataagcctacaccttgtcaagtactttgtcaacaacttttttaacttgcaatagttcttgacttgttgtatggattactgtaccatcttacttaaagctgtaggtctgccttgttctacaaaggagacttaatgagaaaatcaaataaaccgaagaaaggtaagaataccaatcactgtaaggaatcaaaaagggtagagttaatctactattcatcctctgaaattggtctgtacctgttctttcaaaattacagaagacaagaggattatctatgtgtagttcccaattagaaatcgcagatattatagatttatatcaaactgcaaagagtcatggctatataacctcaattggaaagaatagtcactatgatgctttgactggaatgtatttcagggcaatggctcaatctagtgaacaacacttagtggtttcttccagtgagtttacttcgtttctctattgcagcaaaattataaatcgcaggagaactgaaaaatgttaacagtaagttttaattacaatagtgacggctctgtatcaattaactcaccatatgcaaatgacctacttaaagagctagtcaatcagtgtgagaggggtcttcactatgttcaaaattcttttaaacagaagactattgctaataacttgatgcgtgtaacagtgacgtcatcgaatccaaattatgactttgatagtgaaagtccgtactcattagttgcgcttggagagagcagccaattcaaacttgtttgccacgactcagaaacattccttaaggtattttccaaccttatccataataataagtacagctacgttgatggtagtgtaaatttctatccagcaaactatacatgtttgttgattgataatatgagaaaaagtaagcaagagcctacagaaatttcatttgatgtgaactctagtccagacgcagaaacaagtaagaactttgatatgagttacgcattgtcactcagtaagaaatccgagttcattgattatgtcaatggatttggttttaagtttgatgagagtatgaatcttaaaaaacttaagaacctacttaagaccaaagcttaagtgtgaataggggctgatgcccctttaactatttataaggatatttaatgaaagctaagagtgcaaaagacttttattgcttcctacaatcctatatcaattccgtgcaaaatggtgaaaggtacaaccttaatgatgttatcccaacacctttgacatggagactcagtaaatggcccgaagaagatattacaccaacaagcgaacaacgcacctacaaaccagaaatcaatattccagactctgagaatctattatatccaatgttccatattgttggtcttggaagattccttatggatattcagtatgtaattggtaaaggttataaggttaaaggtattgttgttggtgatgtatctccgaatcacaaaggttattttagactaaacgcacatttagaggctgaaaagaaatgattaaagcaaagacttacccagacttcaaagagtttgtgaagggcttcgttgcaaacgtaaaggctggtaagaggtatgattttagaacatatcaagaggctattttaccacttacctatagctcgtattggcctgaagctgatattgcagaagttgagaagtttgactataaaccagactataaagttccttttagtgatgaactcctttacagtgttggtgcacagatgagaactgctgatttcttcatggatttgcagtatgcaatcatcaatggtaaagatgttgacactatttactgtgaatggttggcaagagttaatccgttctcaatgttgaatgctaagctgaaggatgctatcaagccaccagcaattactcagcaaccaacaggtcaaacagtcaatgagggtgacacacttactctaagcattctagcaactaacgccactggatatcagtggaagaaggatggtgaggatatccctagtgccaattctgcaacttacacaaaacagtcagtagcaccttctgatgctggtttatacacttgtgttgtatctggagaggctgggacaagtgttacctcagatgcggcaacagttactgttaacgcactacctgtgattacacagcaaccttctagtcagaccatcaatgagggtggaaacatcagcttggaagtgactgcaacgggtgcaacgggttaccagtggaagaaagatggctctgacattccttcagcgacaaatgctacctatagcaagtctggtgctctgccaacagatgctggttcatacacttgcgttgtaacaggtgcaggtggctctgtcacatctaacgcagccacagttactgtaaatgcacttccagtaatcactcaacagccaaccaatcaagaaatcactgaaggtgagaccttgacgttgaatgttgtggcaacaggtgcgacaggttaccaatggaagaaaggtgaggaagacatcctcgatgcaactactgcaacttacaccaaagaaggtgcaaccactgctgatgcaggaagttacacctgtgtagttactggtgcaggtggctctgtaacatccaatgcagcaacagttacagttaacccagcaggggaggcataatgcaactctcaagaaaaggtttagaagctattaagttctttgaaggtcttaagctagaggcttacgaagattctgctggaatccctacaattggatatgggactatccgtattgctggtaagcctgttaagatgggaatgaaaatcaccgctgaacaagctgaacagtatcttctcgcggatgttgaaaagttcgttgcagcagtgaacaaagctatcaaggttccaacttctcagaatgagttcgatgcacttgtaagtgaaacatacaacatcggtatcacagctatgcaggactcaacatttatcaagcgccacaacgatggtaataaggtaggttgtgcagaagctatgcagtggtggaacaaagttacagtcaaaggtaagaaggtcacttcaaacggcctgaaaaacagacgtaggatggaagctgacatttatcttgacagtgtatatccaaagtaatatcttcataggctccttcgggagccttttttattttctaaggagaaagctatgaaactttgggctagtgactttggaacttttaagtacactcgtaatggctcgcttgtacgcattgtcgggaacaatgtggtttcaagaggtgacaaggtatatacacggtttactgtagagcttgttgagttgtcacctattgagtctgtgaacaatggcctgttcaagtttgagacttacaatgtcaacgaacatggacaattcaaccctcttggtgaaagtggacttgatattatttcagagcacccattgacaaaagagcaacttgcaggttactataaatctgttctggaaaggcagttagcaacacatgaacaagaagctaaatatcatttacaacattgcgaagctttaagagcaaaaatagaacaagcagagagaggtttttatgaataaagagattcaagtaaaagttgacacttttggccatgatggtcgagaagatgctggacttgccgacgcagtgatttttgagtgtaatggaaatattagtcttcgctttacaggctcaggtagcacacttcgcagaatgcgagatgatgcagatggttgccctgtatttggctggtatgatatggagttccctctttacgccatccattatccagatggtggagaagactggacaactcaatcaatccgcgatgagcttaatggcattccagttgagcaagaagagcaagaacctgttgaactcacattcacttttatcaaagaagagaaagtaggtgacttgtcagtaactgaagcaattcaagtctcacaggttattcgttaatgagtaaagtacaggttgttttccctatttgtgacttctcactagagcgtgaacttgacctgtacgaagaaattactgacgaaattatctggtctgttgtagaagaggctatcaagaagctgtatggtggcctcttaaatacatcaagtaagaagttaagcactaagcaagtagctgacccatatatttcatacgatgcctacaacaaaccttttgaaaatacctgctttgaccttatggtaggtaataacaaagtcaactatttcttcgtcagagagtttaacgatgagtaagctccatgtcatagtgtacaaaaacttctcagatattaaagagtctttaacaaataagcttgacttacaacgtaaaagactctttttaatgtacgatatagacaactacaaccatcctaaagagtttaactacaaagatggtacaaaggttgttgagtttgaagattctgtgacggtatatgtcaagcatgacttaccagcaaagtacatagggatgctagagtactacatattcaaacatactggtatgcgtggtgagtctgttaagatatcttctatagaagtttttgagaaacctaacacacaacttaaaaagtatttaatgaggaaactgtaatgtcagaagaacatgagcatatccaaccagaagttacattagtaaaacatttcggaaatgtagaaggctgtgtagcactttttcagactaacatcaattctccagtcaaactgtgcaaattgacgataaacaccaacaatatcagtgtatgtgtgaatgaagaagtccaatacttcaaattcaatgataaagaggttgatgctgcactgctgaaatacagagcaaatcttgaaaaagatattgaccataaagaagttgtgagtctctttgcagacctccataagcttcttgataaggttgaaaaaagaacatattacatgaataatggctctatcatcaccactatgatttcaccatgtatctctgagccaatcttcacagatgatggtgcttattagcagtgtagcatcagcagtgtctgattggtggttaaaaaataacgccctgaaaacagtgattgatgctatcagagaacacatccctacatttagtccgtggaagggtaaaggtgatgaatttatcgcactgttgagtgaagagagtaacaagcgcagtgcattactgcctaaaaaatactcttgaccgagtatacaatattgattatgatgggagctatcgagaggtagttccctttttagtttctggagaatagatatggctaaagtgaaagaacacgataaaattattttgtatgttaaacagaatccttcaaagagtgttgaggctgttgtaacacacatctcacacaatgggatagtttattttcgaccactgaagaacttagactttgacataacaccaaaccacacatttagtacctccgcaagactgtgtgcaggaactgtatactcaactgctgagattgaatctttagctttcacaggtgcttacaagaactttatagggattgctttaaaggactctgaggaatcttttaaatttttcatggatgctgctaatggtggtagtagatataccatgccagtctttaagaaagttgatacagagattgtcaacaagtgcgaagaaattagcagtaacctgaaggaacttaaaacatctgtgcagaatgcaatttacattattgaaaagaatcacttagaagttcttttcagagatatgctgaaagcagggtttactccagacgaaattacaagagaaactcaacgtcagttcagaaatgctatggtgaaagaatgaaaaaactaactacagttgaagattactacaagctgtctctattagaacaatatcgtagaagtcagagtataagaagatgctatggcagacacgctgaaggtgatttttaccattgctatgacgcagatttaaaaggtgtaacacctagaggtaaagtcctacaaagacttgttgaccttgagtggaacaaacgattgagagggattggaaaatgatttacgaagaaagatacaagatagattaccagaagactggtcatcatgtctccctaagagtaacaaaaccaaatggagataccggtatcatagcacattttggtggtgattattggtatggtacaggttgctttgaaggctacactcaagaatacttgaaagcattctacagagactttacaaacgactacaacagagttgttgacgaaaagaataagtgcattaagcatgaacaccatgccagaggttgtttgagtacagtaatggtactggcatttttcttggcaacattactggcagtatcagcaatcagttccatagctcaagatttgaccattacacagattactgcaaaggtatatgatgtttggtacttgtatgctgtccctttagttggtatcatcatctcactaatgagattcagagttcataagaaacgacttaaggactctgaagttaaacttgaagaggtaagtaaagaatgcaacctacaattatagctgtatgtgttcgttttgcaatcgctgaaatgattaacaaggcaatcttgaaagatgcttatggagagactaagtaatggttaagacacctgtaccaatttttggatttccttctatagaagagtttaaagtttatcttgacaaaaacttctacaatgagcagcctgttactttactgaagagtgacttatcagagcttcttgatatggttattaaggcaacttctgagaaggagcctgagcagaaagctgagaagaagactagtaagaaatccgacaagaagactgaaaagtctgagtaataacttgaggggttacttgatagcccctttgtagaaacttagaaggtagcgaatatgcaaatcatagctggtcaagaacttgacatcatagatgcaaaaacacaggagtacatcgcaacagtgaaggttataggagtcagggagtggaatactgagtacccaattcaatgccttgtgctggagaagtttaaagctaatggtgtacacttttaccacggtgactacatcagctttaacaaagatggttactggcgtggtagtgaccaccctcaagcaaatgagtttaatatgcgtctagcaataccacaaaaagataaccaacaaaacgtaaaagatatccttgtagaagcctatgaagaaggtataatagatgttgtgcatgatgttgaaaaagctttaaagcttgttatgccacatttagagtctggaaaactgactttagagatgcttaatagggttattcggagagcttatgaaagtaaaagagatagtgcaaagagcaatgcttgacaactcgactaaagatgagatgtacacagaaatttgtgataagttaaactgttcaagacatgctgctaaggttcttgtacactgttttatctgggaatgctcagaagcttatatgaaacacgtagcttttgagagttctcatttactcggtgatgcaaaaataggtgtagcactaaaagagcctgagatgaaaacagtacctaaagttggtaatgtgtataaactaaaaaattttattacaggcgagattgttgcagaaggtgaagtgcaacaggtttatcatgatggtaaatatctgtttaagatattcgagtatgatagtcgttacagacacttatgtggtattacatttttagtttcagaggatgacctcatcaagaacaatagtaataaattcgcagtaccagaatatcaagttttacgataatggtgtgatagatatggaaaaagataacttaaaagaagttgaaggtaactacctagtacttgactggagtgatgttcgagaagcactctccgaagaaaatcttgactggttcgaacaaattatctttgaagtacggcataacagagaagttgaaaatggtaaagagccacttgaaggaatctttgtagaaaggtcttatccgttctatgaagacactttgcagaaagttaaaatgtactttaaacagaagaaccgtaaagtggtcactatggtatctcttggtggacagagtatgtctattatggaagatatcaaccctaagagaccgagtaaaggtgtctgtataaaaatcacaggtcatgaagagtatatctcagtggatgattttagtgtattctgtaatggttctgctgtctttaatggatttcactatgacatccgagtgtacccaatcaagggagatgctgttatgatgcagatattcgataacaacaatggattcactcatttttatcagacaactaagtcatcattgaagacagttttagaaagcctgatttaaaatatcttaagagcctccttaatcgggggctttttaatttttataaaattttatcggttgtcccaacctctgggtcaccatcatatgtgaataaaatatctatccttcttaacaaccttcttaatcttactctctatatatagttatatagtactatatagtatcttagtaactatctagtggtcttaatagccctctgtatagcccttctaaatagccatctaaacactcttctaacaagccttcttaacaacctatacaatcaccttaaacatctctctacaccttcttagaagctcctatacacctcttaatctctatgttacctatgacactctctataggtgttcactaggaaatactaagcctctatatagtctgttttaacaaacttaacaggaatatctttacagaccttatctcacaaggtctaacacggtactaacaaggtatcttataagcctgtttctacctgtatttttaaaagactgtgtaggtaattattcagaattttaaaatttttataaaataagactgtcaagtcattttttagggtctttgaaggaactcaaaaatatctgattaggatgtatatctgcttggtagcccttaacagctcccttacaagtactttgcaaaattttaaaatatccttaacaatctcttaacagttctcacatcttcttaacaactcttcaaaggtcttaacagattcttaacagaactaacaaggctgtaacagtttcttaacagttgatacataggttttacagaatcttaacagatgactaacaagagctaacaagttaattttaaagagttattttgttaagataattaacaagtttctaacaagtgtttaatctttttaactagctattaacatggtcactatctctttcttatcgttccctttcctttcttagtgattccttaaacatacctcttaaaagctctctatagctctttataagctcttttaagtctcttcctagtagttacaccccaactgtacagataactctttatagggcttcacaggagcttttaaggctatgtcaaagactcttacaaggcttttacgtggtcttaacagagatttaacaaggggctaacaagttgctaacaagtgtcttaaaagcataactatgtagggattgttaagaaattgttaagggggctagcatttacgtaacttttaagctacttcaaagtaactttatagcccctataacatacttttaagacaataacaagccctttttagtagttcacaagataactaacgagatgataaagcttacacccttataagattcttttaagcctctataaagccctgtaaagagctttttaagataggttaatgcaatcccttaagttaggttgataaggctgttatagagctttatagagcgttttaggggttgcccttccctttcgggttatgggcttgtatttaagttgatttgataaaaggctatagaatagactatagagttattgcttttaaggtatgagatagattctatcgaatcagtggatagattctagaaaggtgagggaaagggattgtataaggattcgatagacgtaaaaaagcccctttaaataggggctgttcaggtatctggggctaaatagccccgctgcgtcgattagttataatcttgtttgttgtagtacagggagacaattttttctccccaatagtgataagtataaccatccttgagaacttcttgttttgtttcctcccagaactcatccgaaggattataatctttgatggtctgactgataagacctttcaccatttcaaaggcgttatcagtgcctgcaatgtcaaatttaacatccagagaggaaaaaccgaactcgttaacagtagcgcggatagttgccatttttagattctccagtttaggaagtagtttaaggactttatttagtggggaaggtcattccctccccgttgattagtaatttactgcatattggcgtatgttgcaagccttttttcgttgtattttgtaagaatctcacaagacaaaacatcatactttgtaacagcgttgcctttgtcgaggtcaaagaatccaaacgctttaccatctttgaatccgacaacttcataagattctttgatgatttcaccattgaacaaatcgctttcgatacggtgaaataatgctacatcttctttgtcggttgcaatctgcaatagtgtggttaattctacgttttccatcttgataactccagtttaggactttatttagtggggaaggtcattccctccccgttgattgataatttacagacttttgattgtctcgtcaatatctttttcaatatcttttaaagttttttctaagttcttgatattgaaggttaaaccttcaccgagagttttagcgtaaccgttaggacgtaagtcataaaggccaatttgttctacattggagaggttgaccatttcaccgcttacatcataagctattgatacagtattttcaggggctttatagaacttgtttaaggacttgatagaacgttcataagccattttataaccgtttaaaaggtttttaacgtggttgtgaacgtttttacactcatctaagtaaatctctaagaataacggtaataattccgctacagcttgcataaagtgagagactacaacggttgcaacattacgacgatgtacataataagccccgttttctgacttagtgatggttgcaaagtgtaccccattacaacgaaattcgtatgtatcttttacaggcttagacattacagcatttgaacggatgaagttgatagctttaaacttgttcattttgactctcctagtttaaggattttatttagtggtgaggtcatccctcccgttgattagtaatttacttgttttgatagctgattgcaagtaattttttgagcttatctataaagagtcttttaagtgaatcccttttagataaaccccctcgaacaaagttaccaatgtcaaggggtattgtcaacacttttttaaaagttttttgtaagttattgattaactgatgaatattttttcaggttttacatagctgatacgctgactttttagcaggtcatcccagattaacgcggcgatatgggcataaccggattcacgtaaaagcttcattgttttacttctcgtttcaaaacggcttgcatagtactcactattcagtgtcgttttattcccttcgttgtctacatggataatctgcatcacttgaaaggggcttgtgtcctcgcctttattccactgttcaaagtattttaggcactgcataggtgacatcttatcacttacttgagttgaccaagatgcgccagctttatcggtaattacggttacataagccatttttgaatctcctagtttaaggactttatttagtgggggaggtcatttcctccccgttgattgacaatttacagacttttatcaggcttgtctagtctttttcaatgataatctcatactgaccatcaacactatcgaaatagattccgtcgtcatcttctgtcttaaagtaacactgaatatcatcacttgcgggatagtcttgccttgctatctggcaaacttgaatagcattttctttacttcctgtgaactcctcaagccctttaaaatcacatgaatggccattacaaatagaaaggataaggtaaacggttgtaagcatgatttaagccccctaaaagcccctacaaggggctgattagtgtttataaggtgattgcataggtttttagaaaaagctctttaaatcctcttctgcaatgtttcctgtcagtgtatccatgatgtcacaaaaaacctgaatactgtccgactgttgaacacttttagcaagaagctccagagactcatcaagatagtttaaatttgggttatccaatgaaggaagacaaaaaggtgaatacatagcgttaaactctgttgtatagacctttttaatccaatttacatcaaagtactctgtaaaacttgctacactcgacatattatcacaagaataatcatcgtttagctcatcctctggaatatcgagttctaagataacaatattttgagtcttcatctgaaaagctgcggtgatagtggcattacctagagcttgctgaatgccctctgatgctatttcttcctcatcgtcgccatagattttattttctggatacacgtaaaaattgccgtccatatccgaacaattccatgcgcctgatggtttatcaccgttattaatcaggttgaggaaattttcttgagtagtaccgtgaaaacatttcatttttgaatctccagtttaggattgagtagtaaagttctttatctggggcttaagatatcaacttttaagccccttgtaaagtactttatttgttatttttgacaacttttaaaactgcaagataacctaagcaataagaatcaccgaagagagtcctacccatgaaagaatcccctttaatatcctttgcagtgacagttttttctacgccgtgaaaaagaactgtatcaccggctttaatgtctttgatgtgtacagtctcgatagatacgccgttaataatagccattttagaatctccagtttaggatttagtggtaaagttctttatctggggcttaagatatcaacttttaagccccttgtaaagtactttattgcgtcttcttcgtaaatattagttttacagcgattgttggaagctcttcgatatttgtaaatagcgttactttttgagtagtaaccttgtaaccgttttttgcagcgtaacccgtaaagcggttaactgtgtagctatatgtttttggtgtattgtctggcatatggtacaggaaaggtttgcctaaattttctggttgcgtcaacagtcggcaagtatcagggatatttaaaaaattcatcattttgaatctcctagtttaaggactttatttagtggggaaggttaatacctccccgttgatgtgtaatttaatctatctaaaaataagtgtcaacaattttttagagttctgcataaattgcatcgcagatattacgaaaatcgtgatttgacaaattacgctgcaaccaagtggcgaaaaagtcagcgattgcaaagttagcatcatactcgacagataaccacactttaacagcaaatcggaacattttcaaatctaactctttattcattaccttagatttgcctaacatgtctgacatttcatattgcgcgccgtctagcgtgatacctgatgcaacgtcagccaatgaactttttacattttcaacgtctaagataacactttgaatcttcccgataatgttgtgcaatgtttcatcaggcgctgatgatgtcagaacattaatcctgttgacggtcaaacgtgcttttaaaattgataattcacggttattaatagccatttttaagttctccaatttaggattgagtagtaaagctctttatctggggcttactttatcgaactataagccccttgtaaagtactttacattaaatgcagttttttgccttttactataatgtaactcgatgcgctattgctttgcacatcttgataaattctgcgcaaacgtccttcgtagtatactttccaactggttggaatctttttcccgtatcctgtagcagttttttggagtcctgcaacttgccaatccaaaatgtgctgttcagcttgtaaatcctcgaagtgcattacaccgttcacatagtgacgtagtgtgacatttaaagtacgaccgttgttagtagtatacattttaaaagctcctagtttaaggattttattttgacagttcactaattaagacatcaaagattatatcattatctgctacagttgcaacaccagtgatataatcaatttctaagataacaacaactttcgtacataaaccgtcatctatatcattccagacaactttatcacctgcacaaaacattttttgaatctccagtttaggactttatttagtggggaaggtcattccctccccgttgatgtgtaatttaatctatctaaaaaccagtgtcaacagttttgttttcgtttgctcaactcttgaacaattacagcgtagtagtcttttaagtcactgacatttaaagcatttatcagagaattataatcatcgactaactgaaaatcatctttgaagatgtcacttatctgataggttttcgctttatatctgaagctcatatctagccgctttatttgctcatcactgagacgccctagaaagctctctacaaggttattgctaacctgctgtgtgattgccttatttgctagattataggctttatatgccttatatagcgcgaaaatggcaatgattgcagtgataccagtcattaacatggtgtttcctctttacgtgatactttgccacttacagttatctcataggattctaataaatcgcttttagactttactggacttttaaaagtgtcatcatagatattgacttgcaatacaggttttttatactccctgcaactgttccataagcaaatcactacacgacgaccttttattttgccgtgtaaccattccataacatcatggctattccttgatgtttcaatgaaatcaattttgttgagtaatttgctcagattttcacgcacttgatttgataatttcattttagaatctcctagtttaagggctttatttgataaatctctctatctggggcttactttatcgaactataagccccttgtaaagtactttatttgttaattttgaagatagttacgtaaccatcactaccgattatttgaggcgctccagctttttctacttgatagcccatattttcaagatgataaacagcatcatcataaccatatgcgccaccctgataacgtctcttatcacattttatcaggtttttaccacgtccagcaagtgcattcagtacagctttttcacttgatagcgtaccatctaacatggttgccgtgtaatggttgattctattcccgtttacatcatacttaaaattgaatgcgtgaacaacgataatgttgcctttaaaattttcatcaagtgacttctccagtgcgttgcgaaagttgtctttgttgatgaatttaggtgcacgatatgccatttttgaatctccagtttaggactttatttagtggggaaggtcattccctccccgttgatgcaaatactagataaccacgttcaaagagtcaagaaaaaattttaaaaatatttttggcaaaactgtaatgaaacgggcgcacacgactaccacaaaacagaacacaaatcaagtacctaatggtcgagtcgaacctattcatattttgattggacgtcagcttcgtaaagaaactatgcacgatgctattaaaaatttcctggatgttaatccagaggattaatcaaccaagcctgatctcagcagatcaggcttttatttttcttgctttttgctaaaaattttggtataattcttagataaattgatgataaggctatcttcgtatgtgttctagccctcttaataaatctatatgcactacaataaaaagatttttcttgctcaacctggtttatctcttcactatttgt

SEQ ID NO: 3 - protein, 57 aa, Unknown
  Found in bacteriophage isolated from environment
TNSEEINQVEQEKSFYCSAYRFIKRARTHTKIALSSIYLRIIPKFLAKSKKNKSLIC

SEQ ID NO: 4 - protein, 56 aa, Unknown
  found in bacteriophage isolated from environment
TRSFRLLNSLLISKMSVDLFLWSTISHNCPLIFPRRTTIQKHFLQLSYLFGTNCTL

SEQ ID NO: 5 - protein, 77 aa, Unknown
  found in bacteriophage isolated from environment
NIPDTCRLLTQPENLGKPFLYHMPDNTPKTYSYTVNRFTGYAAKNGYKVTTQKVTLFTNIEELPTIAVKLIFTKKTQ

SEQ ID NO: 6 - protein, 180 aa, Unknown
  found in bacteriophage isolated from environment
MKCFHGTTQENFLNLINNGDKPSGAWNCSDMDGNFYVYPENKIYGDDEEEIASEGIQQALGNATITAAFQMKTQNIVILELDIPEDELNDDYSCDNMSSVASFTEYFDVNWIKKVYTTEFNAMYSPFCLPSLDNPNLNYLDESLELLAKSVQQSDSIQVFCDIMDTLTGNIAEEDLKSFF

SEQ ID NO: 7 - protein, 113 aa, Unknown
  found in bacteriophage isolated from environment
MAYVTVITDKAGASWSTQVSDKMSPMQCLKYFEQWNKGEDTSPFQVMQIIHVDNEGNKTTLNSEYYASRFETRSKTMKLLRESGYAHIAALIWDDLLKSQRISYVKPEKIFIS

SEQ ID NO: 8 - protein, 87 aa, Unknown
  found in bacteriophage isolated from environment
MENVELTTLLQIATDKEDVALFHRIESDLFNGEIIKESYEVVGFKDGKAFGFFDLDKGNAVTKYDVLSCEILTKYNEKRLATYANMQ

SEQ ID NO: 9 - protein, 100 aa, Unknown
  found in bacteriophage isolated from environment
PSPLNKVLKLLPKLENLKMATIRATVNEFGFSSLDVKFDIAGTDNAFEMVKGLISQTIKDYNPSDEFWEETKQEVLKDGYTYHYWGEKIVSLYYNKQDYN

SEQ ID NO: 10 - protein, 87 aa, Unknown
  found in bacteriophage isolated from environment
LENLKMATIRATVNEFGFSSLDVKFDIAGTDNAFEMVKGLISQTIKDYNPSDEFWEETKQEVLKDGYTYHYWGEKIVSLYYNKQDYN

SEQ ID NO: 11 - protein, 87 aa, Unknown
  found in bacteriophage isolated from environment
FHCSDASSNVLETKGRQINFKVNCASIVGKFHHFTKDTLGDFIIIRRILEPLFCFLFNELITISIVIPSFFNDGQVVVFLIIVLRRL

SEQ ID NO: 12 - protein, 106 aa, Unknown
  found in bacteriophage isolated from environment
DILFMTCDFYTDTFTRSLRVDIFHNRHTLSTKRYHSDHFTVLLFKVHFNFLQSVFIERIRPFYKDSFKWLFTIFNFSVMPYFKDNLFEPVKIFFGECFSNITPVKY

SEQ ID NO: 13 - protein, 102 aa, Unknown
  found in bacteriophage isolated from environment
MTCDFYTDTFTRSLRVDIFHNRHTLSTKRYHSDHFTVLLFKVHFNFLQSVFIERIRPFYKDSFKWLFTIFNFSVMPYFKDNLFEPVKIFFGECFSNITPVKY

SEQ ID NO: 14 - protein, 154 aa, Unknown
  found in bacteriophage isolated from environment
RLVQYEPVAFKNSNNKILDDESVLFTIGCLHRYRSDYEFIKFLYKGDHYVQQVEGEAVIEGTIFNKLKYVNGVKPVTKMEPEKLAVGIKADGLLHSSEFAVHKMYAESCEWIFCHVLVKAAHRSCNLKDCIETYMEDKTSNDLMARQVIEKVKM

SEQ ID NO: 15 - protein, 151 aa, Unknown
  found in bacteriophage isolated from environment
ANSKASDRKSEYARRIVRNLMELTLKGSELHPMVLKLAKEVDHVVDIIGEEYAEVLIDKVNQQNDKQPIALRMNFENAQPHDSGRWYGDKNFSIYDGHYFHVGNAKFKELVLCQIPYETNWERVGIVKVTAIYEQTKADIIDLEQGAIIQM

SEQ ID NO: 16 - protein, 74 aa, Unknown
  found in bacteriophage isolated from environment
ESKETKKDSKKSTKKEAKQEPEKESTAKIVMDLLESLDSKLLTVPQENYFNKDLYVKFEEISPFGFIPVPTKVM

SEQ ID NO: 17 - protein, 193 aa, Unknown
  found in bacteriophage isolated from environment
SYKKPLLASRKNSEESLLAIFEDGKGKWPSFTPIHERIADIVTKLANNKLWWDSVASAVIYYAGDDTFIPESICPSIMTTIISGNNMYYTRKEVKDLLKHLDAFLSVVEKHDIDKELNARYKLLAADVEKDNFKFYQVEENVCVSINNTNITLKCLKVPSNINTQFLAVCGEVNGFHKVLTVEPQIHEHEESM

SEQ ID NO: 18 - protein, 69 aa, Unknown
  found in bacteriophage isolated from environment
LSISYIKKSLLRCKSSLFVKDSLISEKFLYTMTWSLLIVKLSDEEIVDFVITYHKVKAGIFKRFVVGIV

SEQ ID NO: 19 - protein, 97 aa, Unknown
  found in bacteriophage isolated from environment
EDNFERVFFYNVKNNGVMLDFCTNEFPKNYADYSIYPDAVQKTSLKKSSTNLLGGYLKKIAEEVVSWIIEDTIEEYLDLERELSFDCIPFVVQVKSM

SEQ ID NO: 20 - protein, 113 aa, Unknown
  found in bacteriophage isolated from environment
LHYKAEQEHTALQRELVSKYYGALQEKTLPHESIIDLGSEGLPNFQGHENVNYTEFKFLGNNVSEIPSLEVLEVTFRTYVKDGRSVVNNGVIRVLSGNRTYKFTGFDSAWLKM

SEQ ID NO: 21 - protein, 120 aa, Unknown
  found in bacteriophage isolated from environment
LLNLFLRVALCLPCWVNCNCCCIGCYRATCTSNYTGVTSCISSGCTFFGVSCSSCIEDVFLTFLPLVTCRTCCHNIQRQGLTFSDFLIGWLLSDYWKCIYSNCGCVRCDRATCTCYNASV

SEQ ID NO: 22 - protein, 154 aa, Unknown
  found in bacteriophage isolated from environment
KPYVSDLYIDAEMRRRNKLGNSTVKKGKVTVKNWWQMAEACGVKNGDNHRKIFTSDQMATIGINYTESVLADFENQSTPVKIAKNVAAVFKEVDALLYQEAQEATIKMGMKVPKGAIRITGYGITPIGASDEYAELKLGEFFKIAELGKRSLQM

SEQ ID NO: 23 - protein, 160 aa, Unknown
  found in bacteriophage isolated from environment
KPYVSDLYIDAEMRRRNKLGNSTVKKGKVTVKNWWQMAEACGVKNGDNHRKIFTSDQMATIGINYTESVLADFENQSTPVKIAKNVAAVFKEVDALLYQEAQEATIKMGMKVPKGAIRITGYGITPIGASDEYAELKLGEFFKIAELGKRSLQMIGGRSP

SEQ ID NO: 24 - protein, 55 aa, Unknown
  found in bacteriophage isolated from environment
RHVLVTFSNMVLLFKITQNITLSRLKQLILKLGIRAIPLHYFLRKIHELIMGRVL

SEQ ID NO: 25 - protein, 377 aa, Unknown
  found in bacteriophage isolated from environment
AEGAPNVTVTAANSTVSGGAGTVVCTYSGADATTAGEKTYTATTADLIDEEGKKWQYGTAGTAVVNLTLTEGETIEQNTPQQTIVPLANVTVTAANSTVSGGAGTVVCTYSGADTPLAGSKSYTANTASPIDSGDKKWQYGTAGTATVELSINGGENITQSSPQQTIVPLANVTVTAADSTVSTGAEGSVVCTYLGADSPAVSQKTYTASNASPIDEGDKKWQYGTANTALISLTLTDGENVTQGTPQQTIAPPKIADKLKANLMSFPNVRALWECYITDVDKGNIIAYQLDMFFDATRMQAGVSYLLEDSFPVKYDPKYDFKEVEAIDAEPWYSSYTLPLIAEQYTRFDYRKGAKVNAVFGKVFEKFDPYTKAKIM

SEQ ID NO: 26 - protein, 379 aa, Unknown
  found in bacteriophage isolated from environment
AEGAPNVTVTAANSTVSGGAGTVVCTYSGADATTAGEKTYTATTADLIDEEGKKWQYGTAGTAVVNLTLTEGETIEQNTPQQTIVPLANVTVTAANSTVSGGAGTVVCTYSGADTPLAGSKSYTANTASPIDSGDKKWQYGTAGTATVELSINGGENITQSSPQQTIVPLANVTVTAADSTVSTGAEGSVVCTYLGADSPAVSQKTYTASNASPIDEGDKKWQYGTANTALISLTLTDGENVTQGTPQQTIAPPKIADKLKANLMSFPNVRALWECYITDVDKGNIIAYQLDMFFDATRMQAGVSYLLEDSFPVKYDPKYDFKEVEAIDAEPWYSSYTLPLIAEQYTRFDYRKGAKVNAVFGKVFEKFDPYTKAKIMEK

SEQ ID NO: 27 - protein, 123 aa, Unknown
  found in bacteriophage isolated from environment
KKEAELHANLRFYGKHNPSVDGVVIGKVKYGKGIVYQIDMLFRGLGVIHFMPYLLNESDPINIEPKYTRQESTPTIDEEPWKSLRWTLPTPIVDNLNYREGNQVSNIYSQLFCYFDKASKAKM

SEQ ID NO: 28 - protein, 60 aa, Unknown
  found in bacteriophage isolated from environment
NPVVCLYDEQRRYNQFFLYLGIESSSYYILEVRKSEKCHNTNKGKKPKNSKRMLDGKYFL

SEQ ID NO: 29 - protein, 123 aa, Unknown
  found in bacteriophage isolated from environment
MKNRTNKGQFKKGQSGNPSGRPKGSRNKSSLVKAQLTIDNSAEFAAKLFEAIVTRDAAKLAEFGLNTDDVTIKSMIEVGKTIMTHSAGEMKAIAADTKKTPDNGGQSQTDNKPTFSAVATLKK

SEQ ID NO: 30 - protein, 56 aa, Unknown
  found in bacteriophage isolated from environment
VHAGEHLDFQFHHQLGYKMLTQEQLSKCLQQNLGYCHLFRVLGTRCLKAHPHQHQL

SEQ ID NO: 31 - protein, 61 aa, Unknown
  found in bacteriophage isolated from environment
LCSKVTNKLCTFSVVQSLFIHWTLKDYERIHLEILNISSVTTCVKFCNLALVFFSSILHWL

SEQ ID NO: 32 - protein, 62 aa, Unknown
  found in bacteriophage isolated from environment
LLYLFAPYSIGCSTNQYKAFRLPVQSTDTLSLPTAITDPKEGAVKFARSPPSFNSGILTISL

SEQ ID NO: 33 - protein, 57 aa, Unknown
  found in bacteriophage isolated from environment
LRGLFSPSLKGFLRVSFKGAEDQIQQVRMIFSSQFNFEKFKTWSGSTNLQKTVYSKT

SEQ ID NO: 34 - protein, 59 aa, Unknown
  found in bacteriophage isolated from environment
MVMRQTFNLTGDGFESLHFYQYGSVADKVMQGFHKPYEYGFDSHLNLQLWVSINGNANG

SEQ ID NO: 35 - protein, 58 aa, Unknown
  found in bacteriophage isolated from environment
HMWLKFPFIVTKWVSTSPEIGEPHELNISSEYLQSNCMGQLSPSAPELGTLLVLESTL

SEQ ID NO: 36 - protein, 59 aa, Unknown
  found in bacteriophage isolated from environment
FLLLLARSYTNKRESGFLIKLCPKKVSNYKILLSICYTESTCQPKEKVTETGDKKLPFM

SEQ ID NO: 37 - protein, 540 aa, Unknown
  found in bacteriophage isolated from environment
FYLRRRLMDLNAVKQKRVEDVRKVLAGELGLSDEVKEIIKSFGKDPSKFLPTQILTLLRYTPDQVRLIFKLMTDKNYVAPQPGSQEVFLNTNADLVLYGGAAGAGKTAALLMDSLRFIEDPNYNAVYFRRNTTQLQGGLWPAAKKLFGKFGGIPHEQKMTITFPSGATIKFTYLELEKHAEGHQGIEYSAIYFDEGTHFSASQISYLQTRLRSGAEGDSYMKISMNPDRDHFIYDWVEPFLDEEGYPDPEKCGRIRWYVMNDGVMVSDWERDKILEMFPLEIPQTYTFISGTIDDNPILDFLEPKYRGKLENNTPVNVARLRFGNWKARAEGSNYWQRQWCEIVDSLPEDVFDVRAWDLAATLPSEINPNPDWTAGVKMGKSKKDGCYYIIDVVRFRDRPSGVETQINLTAESDGKRTGIFIPQDPGAAGKSYATSLIRKLAEKGYRARAKPTNKDKVTRFAGFSAASEAGLVKVLRGSWNEAYFQELEGFCGDGKTKDDQVDATSDAFNSLNEVKLFKPPSMGAHTDLVRGNPYEGLRR

SEQ ID NO: 38 - protein, 538 aa, Unknown
  found in bacteriophage isolated from environment
LRRRLMDLNAVKQKRVEDVRKVLAGELGLSDEVKEIIKSFGKDPSKFLPTQILTLLRYTPDQVRLIFKLMTDKNYVAPQPGSQEVFLNTNADLVLYGGAAGAGKTAALLMDSLRFIEDPNYNAVYFRRNTTQLQGGLWPAAKKLFGKFGGIPHEQKMTITFPSGATIKFTYLELEKHAEGHQGIEYSAIYFDEGTHFSASQISYLQTRLRSGAEGDSYMKISMNPDRDHFIYDWVEPFLDEEGYPDPEKCGRIRWYVMNDGVMVSDWERDKILEMFPLEIPQTYTFISGTIDDNPILDFLEPKYRGKLENNTPVNVARLRFGNWKARAEGSNYWQRQWCEIVDSLPEDVFDVRAWDLAATLPSEINPNPDWTAGVKMGKSKKDGCYYIIDVVRFRDRPSGVETQINLTAESDGKRTGIFIPQDPGAAGKSYATSLIRKLAEKGYRARAKPTNKDKVTRFAGFSAASEAGLVKVLRGSWNEAYFQELEGFCGDGKTKDDQVDATSDAFNSLNEVKLFKPPSMGAHTDLVRGNPYEGLRR

SEQ ID NO: 39 - protein, 56 aa, Unknown
  found in bacteriophage isolated from environment
RGLVQFARQLDDPYQISLLRSQRLYVSRQKVSKMGVSEDANQHQLHNMCSSKWSGL

SEQ ID NO: 40 - protein, 488 aa, Unknown
  found in bacteriophage isolated from environment
MADITETQESLPPFRMGEVGSLGLKVKNGRIYEEPRQALRFPESIKTFQLMMRDPAVAASVNIIKMFVRKVNWRFVPPKGKEQDPKMLERADFFNSLMDDMEHDWADFINSVMSFCTYGFCVNEKVYKKRQGKKGKYQSKFDDGLIGWAKLPIRNQSTLDKWYFDEDFRKVTGVRQNLRNVSHIAGAINLGERPLTRKLPRAKFMLFKYDDEYGNPEGRSPLLNAYVPWKYKVQIEEYEAVGVSRDLVGMPKIGLPPDYLDENAEPEKKAFVQYCKTVVNDMIANDRAGLIWPRYIDPDTKEDIFEFSLVSRQGAKAYDTGSIIDRYSKQIMMAFMSDVLAMGQSKYGSFSLADSKTSLLAMSVDILLKQIKNVINRDLVAQTYALNMWDDEEHVQITYDDIETPDLEAIGSYIQKTVAVGALEVDKELSNKLREHIGLPPADESQPVSEKLSPNSQSRSGDGYKTAGEGTAKTPSAKDPSTANKANK

SEQ ID NO: 41 - protein, 64 aa, Unknown
  found in bacteriophage isolated from environment
ASLNGSLILVKWNIIRSGATAADTFIILINTLFTLQLNTGGFPFSCSGLISSLASKKLEKISSM

SEQ ID NO: 42 - protein, 66 aa, Unknown
  found in bacteriophage isolated from environment
LTKSYLTNLESILVFLLLMSLNQYLKSFLQIAKAAQEMVIRPLEKVLQRHLQRKTQAQQIRQINNG

SEQ ID NO: 43 - protein, 66 aa, Unknown
  found in bacteriophage isolated from environment
TLSPPIESTKVIQLNSSAGILPFGNIEPFQLSITLIGNSLEHRKPTFNYLQSFLRILTLLMSAAKM

SEQ ID NO: 44 - protein, 450 aa, Unknown
  found in bacteriophage isolated from environment
VKMSSNIFRLADRLFNQPLLATESLAHSAATYVNNRLLGDVQAAVNFDKPKGEARSLLKVKDDIAIIPIMGGLTHRMTFIDAMCTGGLSSYEGLRRGFDEALADESIKTIVLHIDSGGGEASGCFELARHIMASRGQKKIIAYVDEFACSAAYALASSAEEIIASPDADVGSIGVIMVHQELTKAFEKNGVTINVIKAGEFKGMGSPFQALSEESKERLQKRINDTYATFTGFVAESRNLSEEAVKNTEANVYSAQEALELGLINSIMSQDDFLNYLQGSEEAPVSLNVNNSGEEMTEQEKQELEALRLQVAQMKAKEQEAALSDLTNKISASAEAFGFDAKEAATTILGAGLDNPLSVLFMNAMEGANQKLNETIASHASAMEEKESEITKLKETAGAVLEHSNAMEEMGNDGEADLVEEEKEPAKNASEDTAEQRKLALQNALKSLIK

SEQ ID NO: 45 - protein, 448 aa, Unknown
  found in bacteriophage isolated from environment
MSSNIFRLADRLFNQPLLATESLAHSAATYVNNRLLGDVQAAVNFDKPKGEARSLLKVKDDIAIIPIMGGLTHRMTFIDAMCTGGLSSYEGLRRGFDEALADESIKTIVLHIDSGGGEASGCFELARHIMASRGQKKIIAYVDEFACSAAYALASSAEEIIASPDADVGSIGVIMVHQELTKAFEKNGVTINVIKAGEFKGMGSPFQALSEESKERLQKRINDTYATFTGFVAESRNLSEEAVKNTEANVYSAQEALELGLINSIMSQDDFLNYLQGSEEAPVSLNVNNSGEEMTEQEKQELEALRLQVAQMKAKEQEAALSDLTNKISASAEAFGFDAKEAATTILGAGLDNPLSVLFMNAMEGANQKLNETIASHASAMEEKESEITKLKETAGAVLEHSNAMEEMGNDGEADLVEEEKEPAKNASEDTAEQRKLALQNALKSLIK

SEQ ID NO: 46 - protein, 125 aa, Unknown
  found in bacteriophage isolated from environment
MAYQDFTKLGKREPLNDIILWEQVTPTGHSRKEYTPQASTEYRVGEVLKEDGTKVAAGEEAQADSVCIVNFYSDLQLSYHGQLKVVGIYRDAELKDMLTLESGVNAENVKKALAAKGIDFVPTGL

SEQ ID NO: 47 - protein, 150 aa, Unknown
  found in bacteriophage isolated from environment
IFALSCSAAKDSKVLLIEAEASAKPNSAFSAAVVIKPAPRSLGRLTRNIFAISPALWFSLSVIADWAEAISSFSDSIVLSFSVAPATRSCELAISSIPLSPSASKTSSSFSGALFAESSVASCRLSARWFARFDRILYPVCYHCILIKSL

SEQ ID NO: 48 - protein, 374 aa, Unknown
  found in bacteriophage isolated from environment
DILENFTMLTNSEKSRFFLADLTGEVQSIPNTYGYISNLGLFRSAPITQTTFLMDLTDWDVSLLDAVDRDSRKAETSAPERVRQISFPMMYFKEVESITPDEIQGVRQPGTANELTTEAVVRAKKLMKIRTKFDITREFLFMQALKGKVVDARGTLYADLYKQFDVEKKTVYFDLGNPNADIDASIEELRMHMEDEAKTGTVINGEEIHVVVDRVFFSKLVKHPKIRDAYLAQQTPLAWQQITGSLRTGGTDGVQAHMNTFYYGGVKFVQYNGKFKDKRGKVHTLVSIDGAGAEVGVGHAFPNVSMLGEANNIFEVAYGPCPKMGYANTLGQELYVFEYEKDRDEGIDFEAHSYMLPYCTRPQLLVDVRADAKD

SEQ ID NO: 49 - protein, 372 aa, Unknown
  found in bacteriophage isolated from environment
LENFTMLTNSEKSRFFLADLTGEVQSIPNTYGYISNLGLFRSAPITQTTFLMDLTDWDVSLLDAVDRDSRKAETSAPERVRQISFPMMYFKEVESITPDEIQGVRQPGTANELTTEAVVRAKKLMKIRTKFDITREFLFMQALKGKVVDARGTLYADLYKQFDVEKKTVYFDLGNPNADIDASIEELRMHMEDEAKTGTVINGEEIHVVVDRVFFSKLVKHPKIRDAYLAQQTPLAWQQITGSLRTGGTDGVQAHMNTFYYGGVKFVQYNGKFKDKRGKVHTLVSIDGAGAEVGVGHAFPNVSMLGEANNIFEVAYGPCPKMGYANTLGQELYVFEYEKDRDEGIDFEAHSYMLPYCTRPQLLVDVRADAKD

SEQ ID NO: 50 - protein, 149 aa, Unknown
  found in bacteriophage isolated from environment
MCYTGDPANNPLDRVRILCTDTNNDEILIEQSVLEWFYLESGKDEKKAAIKALKYLLFQVAKMGDEKVGGVYLRNSSRFKSLKAVYDDLVKSSVSGLPYAGGINQCDIDMRRQNPCSVKKYTEYGDAARYEGRDYCNRVNGVFIIERDE

SEQ ID NO: 51 - protein, 68 aa, Unknown
  found in bacteriophage isolated from environment
LFMTTLLKALFQDYPMQVVLISVTLICVVRILALSRNTQNMVMLPDTKAEITATVLMAYLLSSEMNNG

SEQ ID NO: 52 - protein, 133 aa, Unknown
  found in bacteriophage isolated from environment
MRLLNRHSFVVKRKVSEDGYYNDDGDWVASQDIVEVNCKGNIQPYIKGSVKNGTQIALPEGIRLTDTRILYTTYKLRTSDDVEWNESDIVMIDGHEYEVFMTMDWSQQLAHTSHYEYIIIRRDKMNAVRNSRT

SEQ ID NO: 53 - protein, 450 aa, Unknown
  found in bacteriophage isolated from environment
MWNPIVNVDITLNTAGTTREGFGLPLFLASTDNFEERVRGYTSLTEVAEDFDENTAAYKAAKQLWSQTPKVTQLYIGRCAMQYTVSIPDEVTESTDYSITVAVGGGMSQPFQYTSQGEDTAENVLQQFKTQIEADQSIKDKVSVNVTGSNGSATMVISKAGDNDFVKVTTTAQTVSIASTTADTASTALAAIEAYSTDWYFIAAEDRTQQFVLAMASEIQARKKIFFTANSDVAALQGTELNSANDVPAQLAKSMYTRTVCLWHHTAAEDYPEMAYIAYGAPYDAGSIAWGNAQLTGVAASLQPANKRPLTSIQKSALDVRHCNFIDLDGGVPVVRRGITSGGEWIDIVRGVDWLESDLKTSLRDLLINQKGGKITYDDTGITRIRQVIETSLQRAVNRNFLSSYTVNVPKASQVALADKKARILKDVTFAGILAGAILDVDLKGTVAYE

SEQ ID NO: 54 - protein, 150 aa, Unknown
  found in bacteriophage isolated from environment
IGMAMYQQYSPKDVVCSWNGIAIEGFAPDSFLRLQRTSPLITPVVGAGGQVALTRNADKTGTIEIELMQTSLSNQMLSAIQAKQDNMELEEDISSNFVIYDPSGSVLATGINAWLQELPQIELGRDQNSKTWIFGCEKLDYTSTIPASSV

SEQ ID NO: 55 - protein, 148 aa, Unknown
  found in bacteriophage isolated from environment
MAMYQQYSPKDVVCSWNGIAIEGFAPDSFLRLQRTSPLITPVVGAGGQVALTRNADKTGTIEIELMQTSLSNQMLSAIQAKQDNMELEEDISSNFVIYDPSGSVLATGINAWLQELPQIELGRDQNSKTWIFGCEKLDYTSTIPASSV

SEQ ID NO: 56 - protein, 106 aa, Unknown
  found in bacteriophage isolated from environment
LLSICAGGKLRKFFRSKHFQKPNFSVNMGNTLQTPLTDAAVEATYEEADEMKFVLAIYGMEGCKETLDQLFAMTFSDLLSLRQFLEIQRSYKEEIAYNELRRAGKM

SEQ ID NO: 57 - protein, 746 aa, Unknown
  found in bacteriophage isolated from environment
RKNVMAQYTVDSFIVELGFSEKVVKGLQRVEKMSMQAAQRIERNINKAFDVKPNKSSQEALNRIVKNAQSASGRINKALNSSLNLDSQGVKSLKKLETQAKKTAKGINKSLRDAMKVDGKITIKTGRGRGGQSIPPVGGGAPRGPRVDVAQRQMERMFNNNFYSGLTRRLETIGGQGNQMAASFRGSLQNIYNRYKGTGKVGEYEMEVKKLIDVTKRWVIAENARLKSVKESAWLQDRANASLRQLVGGFVSAYALLELSQKTIEAGVKRQSAQLASTAIFGADTQQARMFAASFAHQIGQNYTDTMKQYSNFAAGAQPTLGFQGTQEFYKNAAMFARIRGATDEDLKGIMVAFQQMASKGKVQAEELRGQLGDRLAGAVQLFADAIGKTPQELDKMMQDGKLLAQDVLPKVSERMAELVKQAGGMNAVSKQTATSMGQAKAMWDNTLVALFNNSSEGISQLSNSVAMFLQGSMGSTQALGLVIGNLLKGASNLLDFITDFMYRTSALYYYARAWYKDLDNSQQKLIKSAGEFLGTVVTIGGAVAVVSKAVKLLSGLVGGGIFGKILQRLGVSAAGTAAAGEAAAAAGGVTATRMALGTVGSALMLRGSTDPNAAKNYSEVTLPKPFENAVANITNPKRPMFFDENGQLQFAQYTQDVEGNRKLIDNGLSNWEIIMEKLSTSIDNFANKFNQTPMMMTPSGLPMQTKQTLNVTFNLDGKQIATKMVDITDKNQEDILLSSSYPEEE

SEQ ID NO: 58 - protein, 742 aa, Unknown
  found in bacteriophage isolated from environment
MAQYTVDSFIVELGFSEKVVKGLQRVEKMSMQAAQRIERNINKAFDVKPNKSSQEALNRIVKNAQSASGRINKALNSSLNLDSQGVKSLKKLETQAKKTAKGINKSLRDAMKVDGKITIKTGRGRGGQSIPPVGGGAPRGPRVDVAQRQMERMFNNNFYSGLTRRLETIGGQGNQMAASFRGSLQNIYNRYKGTGKVGEYEMEVKKLIDVTKRWVIAENARLKSVKESAWLQDRANASLRQLVGGFVSAYALLELSQKTIEAGVKRQSAQLASTAIFGADTQQARMFAASFAHQIGQNYTDTMKQYSNFAAGAQPTLGFQGTQEFYKNAAMFARIRGATDEDLKGIMVAFQQMASKGKVQAEELRGQLGDRLAGAVQLFADAIGKTPQELDKMMQDGKLLAQDVLPKVSERMAELVKQAGGMNAVSKQTATSMGQAKAMWDNTLVALFNNSSEGISQLSNSVAMFLQGSMGSTQALGLVIGNLLKGASNLLDFITDFMYRTSALYYYARAWYKDLDNSQQKLIKSAGEFLGTVVTIGGAVAVVSKAVKLLSGLVGGGIFGKILQRLGVSAAGTAAAGEAAAAAGGVTATRMALGTVGSALMLRGSTDPNAAKNYSEVTLPKPFENAVANITNPKRPMFFDENGQLQFAQYTQDVEGNRKLIDNGLSNWEIIMEKLSTSIDNFANKFNQTPMMMTPSGLPMQTKQTLNVTFNLDGKQIATKMVDITDKNQEDILLSSSYPEEE

SEQ ID NO: 59 - protein, 65 aa, Unknown
  found in bacteriophage isolated from environment
AFAIHSLVSTASNLLELSPMECNDLETAINKCPEIPLVCAKPRTIPFRNFPALLRSSKMVSKMYL

SEQ ID NO: 60 - protein, 103 aa, Unknown
  found in bacteriophage isolated from environment
QYLSRSLLWCFSILLAPSNRPVTTVIPPATATTDFATLSRLPKTPPPIKPLIKCLSPTLAAPVAAAPSAAAAAPPTVAVLIASPVTPEANISLPEVSGFAALF

SEQ ID NO: 61 - protein, 75 aa, Unknown
  found in bacteriophage isolated from environment
STSPLLFNISLPRDLQSIIMSFSDVDISLKALLNLWVGIIIVGEPKGICVLCVRFTVKLRSPLCIAVLITSIVSL

SEQ ID NO: 62 - protein, 113 aa, Unknown
  found in bacteriophage isolated from environment
MSQYIPVPDTGWSTQTVTLDGTVFVIELKYKERLDRWFLTLSDVDGNVLLSEKKCLADQSITGRYVIPSLAGELFVERMYGTDLQPTRNNFGREKAFELNYYTQEDMRLMENL

SEQ ID NO: 63 - protein, 139 aa, Unknown
  found in bacteriophage isolated from environment
MKTDFALNLGGDYVATLGSDSVYVAHGDLKITGNQIRIIPEDDKATQVAQRLHIRCLLRAGEVFFNTSAGFPYLQLAKFKQKTSIFDNYMKAYLVETRDVSNIYNYSSSMDNAQRKVTVNFDATTTTDILTDITQEVNI

SEQ ID NO: 64 - protein, 489 aa, Unknown
  found in bacteriophage isolated from environment
MAGLTTTGLQTLRYQEIFDNIKSRLLRDISPNLDVSEDSQLGLFLASIARSLADTHEILSEIYDGGTIDKAEGFNLDDITALNAVYRYVAQATRGQVEFTGTTGATIPSTTRLRSTAGNIFYPVSNITLTPSYCVEAILEVNSLRTDANYVIIIDNVIFSYQPKSSDTITVLLTELANAINGGIVAKAEVINDGSALRVYKDEGDIIARTNPMVVTATTFLTFTKITTIHDVVAEEVGAIPALAGTLIEIETTVDGLDSVYNRYGLTTGRNEETDTELRQRYLESLSVTGVGTLDAIVAAVKRVQGVSDASGVENDTEETSSEGLPPKSFKIVVVGGQNDNVAQAIWDTKPAGIRAYGAIFGTAYDLGGLSHNIYFSRPTPKYAFVKVSYSLYNEESLTIPEEDIRDSIIQGINAYGRTLKVGNDVIPNRIYGYIYNVIKGIEINEIKVALSNSQSVPPSDGQYTTARITVDGDQYTVWESSKYTITKE

SEQ ID NO: 65 - protein, 489 aa, Unknown; identical to SEQ ID NO: 64
  found in bacteriophage isolated from environment

SEQ ID NO: 66 - protein, 75 aa, Unknown
  found in bacteriophage isolated from environment
LTMLPISILSILTASDLLWDTGGLSPCYVVALIVTSPSWYVTHSLLLYVMVLSHYHKLFNIINIVLSKIFPSNCL

SEQ ID NO: 67 - protein, 396 aa, Unknown
  found in bacteriophage isolated from environment
ICRSLIMAVGEIQISALPQASLPIDLSDIFHLKQGIEDKRCTLEQLLAPHASLRNNPHGVTKTQIGLDNVINALQLVAANNLSDIVNVAEARANLQIMSSEEVNNLVQQHIKDKSNPHNTTKAQVGLSNVQNWTTSNLYNEDADKYATARAVNNLYKAVQASYPVGTIHLSMNSANPSTYLICGGTWELVSKGRALVGYDSDTRPAGSVFGASTVTLSVNNMPQHSHSVSLSGGGHTHNATVSISAFDYGTKTTNSFDYGTKATNTTGNHTHSVSGNTSTNGTHNHDVYPRENDGTGNAVADSDGGGARRTSRTSSDGAHSHSFNVTSGASGDHSHTVGIGAHSHTVGIGSHTHSGTVTVSSSEHTHSGNTNSVGGGQAVNIEQPSFVLYVWQRTA

SEQ ID NO: 68 - protein, 390 aa, Unknown
  found in bacteriophage isolated from environment
MAVGEIQISALPQASLPIDLSDIFHLKQGIEDKRCTLEQLLAPHASLRNNPHGVTKTQIGLDNVINALQLVAANNLSDIVNVAEARANLQIMSSEEVNNLVQQHIKDKSNPHNTTKAQVGLSNVQNWTTSNLYNEDADKYATARAVNNLYKAVQASYPVGTIHLSMNSANPSTYLICGGTWELVSKGRALVGYDSDTRPAGSVFGASTVTLSVNNMPQHSHSVSLSGGGHTHNATVSISAFDYGTKTTNSFDYGTKATNTTGNHTHSVSGNTSTNGTHNHDVYPRENDGTGNAVADSDGGGARRTSRTSSDGAHSHSFNVTSGASGDHSHTVGIGAHSHTVGIGSHTHSGTVTVSSSEHTHSGNTNSVGGGQAVNIEQPSFVLYVWQRTA

SEQ ID NO: 69 - protein, 690 aa, Unknown
  found in bacteriophage isolated from environment
PLLRGKFIMADYKLSELNSIDTIRSDDLLHVRVKKRPKMLGDEDRRMTYQDFLASFKLERFVQIAGSTMTGDLGIVKLLYGGKAVFDPTGSSEITVGDVLKTFKINANGLKLTIADASRSATVYHTLNKPSPNELGMRTNEENDARYARLAVNNTFRGTQAILSDNEALIIKNITQGAPLYLRGQDADGTNRWYLGNDNRGTNNLVLKNVKSDVSIAILENLVTVNKTVRITGQVQPSDWTNIDSRYIPAATLSNLAKLSDVNTFRSAQIINQNGLLLQLKNTTQDSQLYFAGFNADNVRRWYIGNGEQKPESFVIHNDRTATTIFMVNDFLLNKTVRITGQVQPSDFSNLDARYYTQSAANARYMLASITGSASEADGGDGVAWDAKTGLYKVTNTTGQSSSLVYHMFLGTGSTPSAQLKFSFKNGGMWYRTSRDAYGFEVNWSKVYTEAQKPTPSEIGAYTKAETDQKIATAISDSTDLNKIYPVGIVTWFNSNVDPNTALPGLTWTYLNNGVGRTIRIAAANGSDVATTGGSDSVTLSVGNLPSHTHSFSATTSSFDYGTKGTNSTGGHSHSVSGTTSAAGNHAHHLGLLLVNGGDAQYGYTTVSNSKTRTLDWLDRRDNKFPNTNTTGNHTHTWSGTTSGAGDHSHSVGIGAHSHTVSGNTGSTGSGSAFSVTNQFYKLMAWVRTA

SEQ ID NO: 70 - protein, 682 aa, Unknown
  found in bacteriophage isolated from environment
MADYKLSELNSIDTIRSDDLLHVRVKKRPKMLGDEDRRMTYQDFLASFKLERFVQIAGSTMTGDLGIVKLLYGGKAVFDPTGSSEITVGDVLKTFKINANGLKLTIADASRSATVYHTLNKPSPNELGMRTNEENDARYARLAVNNTFRGTQAILSDNEALIIKNITQGAPLYLRGQDADGTNRWYLGNDNRGTNNLVLKNVKSDVSIAILENLVTVNKTVRITGQVQPSDWTNIDSRYIPAATLSNLAKLSDVNTFRSAQIINQNGLLLQLKNTTQDSQLYFAGFNADNVRRWYIGNGEQKPESFVIHNDRTATTIFMVNDFLLNKTVRITGQVQPSDFSNLDARYYTQSAANARYMLASITGSASEADGGDGVAWDAKTGLYKVTNTTGQSSSLVYHMFLGTGSTPSAQLKFSFKNGGMWYRTSRDAYGFEVNWSKVYTEAQKPTPSEIGAYTKAETDQKIATAISDSTDLNKIYPVGIVTWFNSNVDPNTALPGLTWTYLNNGVGRTIRIAAANGSDVATTGGSDSVTLSVGNLPSHTHSFSATTSSFDYGTKGTNSTGGHSHSVSGTTSAAGNHAHHLGLLLVNGGDAQYGYTTVSNSKTRTLDWLDRRDNKFPNTNTTGNHTHTWSGTTSGAGDHSHSVGIGAHSHTVSGNTGSTGSGSAFSVTNQFYKLMAWVRTA

SEQ ID NO: 71 - protein, 58 aa, Unknown
  found in bacteriophage isolated from environment
MLDTIPNLPQTQDICLQASLVRLLKQMVVMVLHGMQKQVFIRLLTPLDSLLHLYITCF

SEQ ID NO: 72 - protein, 61 aa, Unknown
  found in bacteriophage isolated from environment
MVGCGIEHQEMPMASKLIGLKFTRKHKNQPLQRLVHTLKLKLTRKLQQQLVTLQTLIKSIQ

SEQ ID NO: 73 - protein, 12 aa, Unknown
  found in bacteriophage isolated from environment
PNTALPGLTWTY

SEQ ID NO: 74 - protein, 85 aa, Unknown
  found in bacteriophage isolated from environment
NITTISLITVCSNAVALCSSKVPKISSIVLERVSVCSTVMCVRPRTSCATSTVMGVRYTNTSMAVCNTTVSTACTRTRCKTNSVL

SEQ ID NO: 75 - protein, 63 aa, Unknown
  found in bacteriophage isolated from environment
LYTNLKSRCMELFIVHQFINLLHMVLINLSISKDTYQVPWSVSSHLTKNFQNKTIRRNIKWKP

SEQ ID NO: 76 - protein, 299 aa, Unknown
  found in bacteriophage isolated from environment
KGVSLPAEIKGAHQYNNIGGIFDSATLHKLDSLTNLQVGIEFTPAHFEENNMLEYVQKMHNQYIQTDGFVGILYRPTYGTMKCLVELIFGYSAINFPLGLFTDVSRQYWQLDVEGENTIYIQFLVHCPALAMSNAAIDAANWANVLMYRDTIDGKMKTVLTELQDVVCGNHGEFSRWQVGYIRGGADELWDQESSYNSSLWRKFDDSWITWREGDNEGWTRYRLEGLKNEGNLFWLAEGIVQRTFVQKSTLLPFGTRMDFRFQPPAFASIVDGTRTTRLEGVSLVHNLINKYSSDAQSM

SEQ ID NO: 77 - protein, 173 aa, Unknown
  found in bacteriophage isolated from environment
ISKVALPSSDGFDCTFVNTRLSYSFSKKFSKGKCTVASSVTLWKTVWNTTPAYGKALTRTPAPPITKTSSSIELSRYFKNAPNDSLLALAYITSSLPKSMPKYGTEFVKIRLARSGRGLLPRVLKVSLPMTTKLFLVSLLNSCMSCLCRGHGIPVGVPNSPFSPLAKINLIIL

SEQ ID NO: 78 - protein, 181 aa, Unknown
  found in bacteriophage isolated from environment
FHGKVQPVYIEFTAKGDESPKSQVKTFVRNDYLKEFFNEFPLHVTAEETVKHFVTHFVVGAYPLANVLVGAGGIVFVDEDISSDLYKLFAGFSEKSAKAYMVDDKGFDIGLYPVSNTLILSAREPLPKSGLTKFTENGMVVLNNKTLRKFEQMDQKHRPWPMGTPTGFEGNEGSAFILKIM

SEQ ID NO: 79 - protein, 71 aa, Unknown
  found in bacteriophage isolated from environment
MWTTDRCSIRLIAVFNFHLFNLKNWFKNQYKVYTVIFFIQNTIKVLRYKVEEAHRISVRSNPDSVPYILYS

SEQ ID NO: 80 - protein, 86 aa, Unknown
  found in bacteriophage isolated from environment
KTFGKKIFWNIVSKVVKSRHYIFHPSPQYSGYAWFAIIENCGTCRTSEEAIHGNVIESVEGQLDTCGCHPCRIPTGDSEYCHEIKM

SEQ ID NO: 81 - protein, 109 aa, Unknown
  found in bacteriophage isolated from environment
DALRLNFNVGFKECVELMYELSVNGTLHIINSVRAQSIGCIKAVQEQSLGRSKIERKFRHAVFDRMEDVDMPCIYSEMEAERDAEWDDQPAYPDFVENMKRRGNAHLTL

SEQ ID NO: 82 - protein, 70 aa, Unknown
  found in bacteriophage isolated from environment
EDKKKRKRSTDILSDINTARVKEREDNLYDDLAKEIQSDDQCIVTFVTFGDTDDIIKGYGSRPHLKKLAM

SEQ ID NO: 83 - protein, 124 aa, Unknown
  found in bacteriophage isolated from environment
FYRKTTKAWTPITYTTKVASIFQNHQEFFFGDLLFIEHQTKLYRPVTEDWTVAHNSCCANLNRFSIDKVWILSKFLWVCWAVLDSHWEDYICQHNGSVNHLTKFVEFCTRLDTSLNHLTVHNKT

SEQ ID NO: 84 - protein, 89 aa, Unknown
  found in bacteriophage isolated from environment
LTRPDLTLPCHTVPNRTLPYLTSLKNPLRRLLSDAGEYPLVYAAQYTHQIFYVNNFLYKSPDSYMCYKTEDCEQNTHCKRCVQKLTCNV

SEQ ID NO: 85 - protein, 384 aa, Unknown
  found in bacteriophage isolated from environment
EYDRIALGVPFQLSGKRTMKQFQVTLPKGIFEEPHEALYQKKEHTTKWSVDVFVNPLTEVVFKGHGRKDTKVGTVKFEETHMKKHKILFYSRHCCEYFSDESISRYMAGEFKAEVWKDHAEDFEEWSNVRRSKVPVVKCGDRFDNLPSTEIIDRREPWTKTSDCIDYWYFKLLHRDPNDENKIMSVIDELDWGHAYIEGDFEDVDKHLRLMAMIDPILETPIKYVTNERSYATFVSGISLHRSIRCRVGDLKPQADAPFKLYKAHSVKAADHALQVGVKETNQASDKTYAYGKRDVQKKYKAAVEFLAQQEANRENKKGINKPEATYEEFMMKGNEKGYTTIVKDGVAVCSWVNFSGDKNQKYLITMKLRQTIGLTAPMVHTFS

SEQ ID NO: 86 - protein, 90 aa, Unknown
  found in bacteriophage isolated from environment
LTIFAISIRCCCLRVWASDNNNIPAYSIPIEFVDFAVLHLWREAVRGIFVFIRWVTASQKNYSKCNHCFHTTLNLIVHDNLPLCMFLNSE

SEQ ID NO: 87 - protein, 101 aa, Unknown
  found in bacteriophage isolated from environment
KRKGGIEVDIVQVDGAVDFKHIISEVTYTWGEIRVLEGERPVIPNRQAVTATSYVVDYCYIYHVLVQMNTEDMLLLLTHNVMKAMDILHQQKLTQADSLLL

SEQ ID NO: 88 - protein, 188 aa, Unknown
  found in bacteriophage isolated from environment
SLKAFEGHLKIAAECYAKHAEEPTDFMGLHYNKGQLRIMARFRKSIKQVGKPLGSPSERSRNCSNQSATCVRLNLYRNDWGDGNKHDVQLTKDPLYGDMYLIVLRNAQYGRGDILLQVSKHGDTNTRYNGARKGGFRSNFNANGERFKWTFVGTEPDYDLLEKLRIETIMEVGDIYKWGARVLRAQAL

SEQ ID NO: 89 - protein, 119 aa, Unknown
  found in bacteriophage isolated from environment
MELHKLFISFPTIDSLLTHPERCFFSSHVGFISVYATLLKLEILIIKCPFSFGVNTDIFYNTSEDFCIPHTPMFNLWFTNTNTSTPCSQAMSTFITLWSWSIHPLNTRLDKRILKEITP

SEQ ID NO: 90 - protein, 910 aa, Unknown
  found in bacteriophage isolated from environment
HVELWSKGVKGTGTVLTESKLGLNKSAQGYMWDTIAKVEQTMSEPCEWSNEDHYALLLRAGIAPKRGNLKTLGEECARRCALNIAENQIQAESGMLLYNLLKHPSKCWLWAGAVKVFGGKSIAATKGFVEELAKKTAKYDAELDDLLKKIKPLRIFYTQKTLAGEQTTAAKITKAFKEDGAGFLLAYIGNKAKKRGKSIEHLLNEDQTARCTLIDQEDNLNFYISNLTHADTGSYVVYLDNESDYRLYKDIEPNLYEDFHKCYYRGDEQRIFEIEEKGQTVAKTFDKDGMFNCLLVLQASNMDVSVVNTGEECIWVARMPEGYVAAGSPVNVIGYQTMRGTSTGFVMAGASLRGDPRIQNLWGKEDDKSNEITRRRHMLTYYKAIKQGLEGEITDYSSETLKPSTRILCPELLDTYKKVSWNHEIYQVGEHEVYSLGCREVMEQWKPHKTIMKKNDKFRCVKVPRGDSDKKYNWDDPIWGVSKLYDKVVAVQTMRSVEFEIPTYPADILGTYRSSELEFHNVVHSNYYKVTKVTKVPKWDKCKKGIKANLENCIARADKMAVFGEKYEEGTVSNTPTYCNRKEINFIKTTPKGFPKIEYTQMEGNRVQQRYKTKPYKTIRKLGDAHGFTEVYENWAKAFEEGTVKGKTKITPPLHPEVESALENTLKDLEKVHFEMLEKDAKWGNIAQQSMWFSTEKARMYTEYTDIGCKKLKLYENDLARKTKANIRIDEVVRTFIEANWVGWHEIEPKRVGVRAGWAALGHAGKYGKPTPRDMWQVRSQILSDQFFNSWPKCKPNDFNWLDPAIHNFVWWDYGLGNHICLRKAKLAFEIGERLGGKIYKTYKHGLIPEDTFVWVPPEEDDSKYDAFAMVHVKTFELLLGDTETDGVLHLNKFDVNEMKVGELIMLRMC

SEQ ID NO: 91 - protein, 154 aa, Unknown
  found in bacteriophage isolated from environment
DKRLKLNSYLKEAYMQKMFVHPDIADFVRALQKLEELDIAAQREYAHHHRRMVDIQHEIELSENYEDDLKCSVFEHMKDVATARRKAKDTVALLDSLKKRLQSGTDLCNLASLINDVETSWDRHYYPRSEKTLDFSSSENLKCSRKKLNQLREK

SEQ ID NO: 92 - protein, 270 aa, Unknown
  found in bacteriophage isolated from environment
ERNNMNIDVEKMLEEKGIDEAFLINAVESMAMILRGNNYTEQFMPASYNLPEDKSDNGAFAEQVNMMLCDKTLALASIVVGVDALAEFIFYEMQKNDYIAMTTEEKDLLLLAGNDELIDLLISSTSAVMAALEKRMEEKLSRYEKLDWGVEELNPRIKEIIQQTKESLEEAVENDSVADIEPLQVKVALMIPAISTMMNIVALIQLSQMMGVSIEFIEEMTGGVSMQAINDVLVNIGASIIEEKLRINFGDDFVKQISAMAEKEYAEQLH

SEQ ID NO: 93 - protein, 247 aa, Unknown
  found in bacteriophage isolated from environment
MSNIIAFTGKARSGKDTSCSIVKNILEDEHGYNVAVMAFADNLKLSASKIFDLTWNDLYGETKETPQVFDLSYPELMFKVTEAMEFTFRDERYHMDFKLMSELTGRLIMELKKVAKPTLLTRLGFSKKYKFSSRQIQQIWGTEVVRKVMGDKFWSKDLEKRMVRFYESCSLRNQYGFVLISDLRFDSEAEWLSRFAHQTIEVKRDNVDKISSHVSENGLSTKYERDIIHNNGTLTDLENKLRAIIKV

SEQ ID NO: 94 - protein, 670 aa, Unknown
  found in bacteriophage isolated from environment
RQNYNEEVLMSKGRKLKEAGQFVGHCACPRCGSSDAGSIYHHDDDSYSMTCFSCNKGFPEWDFDKGQIVSTYSTGSDNKNRTFRGMDLDDVKDNLEAMDLKDRKIPAKVLERLGIKVDIDSDGEIDAHFYPTYKRNEDGKLEHVGYRVRHRYPEDHPKEHLRGKLKDFSGGVGDIKGELAMFGSWIAPEGGNRLFIWEGEMECATAIYMTSLAIKDKSRRKNYCHVSVPSGANIKSIKDNYQYITSFDEIYLCFDNDEAGAKATKEAAGILPIEKVRLFQYPEGVKDLNEWWTKFYKEKDTVLEGFKQRIYNAPRYCPAGIKNFADGFEAMKNRGQIPLIPFPESFGDLNRLTYGGYGLGEITTIAAPSSVGKSAYTREMIYSAWKETDYNIGVIPVEDTYEELMEMLCAIHLSKQISEIPYDERDWDELKGAHAELSKGRRIHIVDHQGAIDQDNLLEFVDYLVNSLDCKIIILDPITLALSRSDTDEEEVLSELLRRCKRYQYAQVNVCHVRKSAGGQKANSEGGDISEEDIKGSGAYFQISMNNILLMRNKVDPDPVKKNLTKIKLTKCRRHGKSTGIAGHTWYNPDTGRLIKASGCGVDIDGAAENIRQQFGIGEAEDHYDDSLPHYEDEVFDRETGEVYTEEQRQSSTIPPVLSEDADDCPFETE

SEQ ID NO: 95 - protein, 661 aa, Unknown
  found in bacteriophage isolated from environment
MSKGRKLKEAGQFVGHCACPRCGSSDAGSIYHHDDDSYSMTCFSCNKGFPEWDFDKGQIVSTYSTGSDNKNRTFRGMDLDDVKDNLEAMDLKDRKIPAKVLERLGIKVDIDSDGEIDAHFYPTYKRNEDGKLEHVGYRVRHRYPEDHPKEHLRGKLKDFSGGVGDIKGELAMFGSWIAPEGGNRLFIWEGEMECATAIYMTSLAIKDKSRRKNYCHVSVPSGANIKSIKDNYQYITSFDEIYLCFDNDEAGAKATKEAAGILPIEKVRLFQYPEGVKDLNEWWTKFYKEKDTVLEGFKQRIYNAPRYCPAGIKNFADGFEAMKNRGQIPLIPFPESFGDLNRLTYGGYGLGEITTIAAPSSVGKSAYTREMIYSAWKETDYNIGVIPVEDTYEELMEMLCAIHLSKQISEIPYDERDWDELKGAHAELSKGRRIHIVDHQGAIDQDNLLEFVDYLVNSLDCKIIILDPITLALSRSDTDEEEVLSELLRRCKRYQYAQVNVCHVRKSAGGQKANSEGGDISEEDIKGSGAYFQISMNNILLMRNKVDPDPVKKNLTKIKLTKCRRHGKSTGIAGHTWYNPDTGRLIKASGCGVDIDGAAENIRQQFGIGEAEDHYDDSLPHYEDEVFDRETGEVYTEEQRQSSTIPPVLSEDADDCPFETE

SEQ ID NO: 96 - protein, 95 aa, Unknown
  found in bacteriophage isolated from environment
MQMTARLKLSDSYKREGFIPFFEEKSLNEFQEECLTKVDRFYRMLYMKCDGGVEVEEKFHHSMITVTITIPRHNIGWLFIIKENVFEYQVYRRIS

SEQ ID NO: 97 - protein, 49 aa, Unknown
  found in bacteriophage isolated from environment
MKHSKAFEQVFGDSLKATAGKPAKYYEEKRVRTGKTARKAASKDKHNFQ

SEQ ID NO: 98 - protein, 285 aa, Unknown
  found in bacteriophage isolated from environment
MSKVVKMKAPVEKYNGTERQTLRYLLKDVWFYYLNTSPRPGKGKSIDKKFPGKDCNYSVSILAEDGNKLFKEFTKSKKNPEGWDKVTTEAVDADDFEEKFGCKPPFEADTYYILKVSRAAAYKDGAVWTAKQSFPVMLIEEVNGKRVAVKQPMKKIKAQASDKHEDDKNYDVIHPDIAVGNGSFGSVILSTHFYTFENNVLTKPIQEQFIIDTLVPYTGGNGANGEPELDEDELAMLGLDGVEDNGEITEEDATDHKPSNNSDDGDDEDLPDPDDEEDEDFDTED

SEQ ID NO: 99 - protein, 359 aa, Unknown
  found in bacteriophage isolated from environment
LYLVWGFFIWRVTMEKYTLTKLPDSVTHVFIDSDSIAYKGACVVEKAKYKYVNKLTAEESEPFDNAKDAARWLADQRILVEELGLTFDEDEWERQTWKEAKSEKEAIMATQQVLQEWLKVVGKERTWVGYLTEKGVHKHKDIKGLEHQYQGNRKDAVTPTHLVACREYLLSRPEFKLLKGGFEADSIVIAKAEKMGKKAALMSIDKDLRQAEGTYCIDMTYEKSPLIFIADNNVGDIWDCPIKSKPKAAKTVGVGFKFLCYQAVAGDNADNYFGLKGAGKVAVMNALDGKETYKECLDAIYELYAKKESYTYVSWDGQTVTKTPLELMEQHFWVAYQERNPKDNFSFKKYNWEPCMNED

SEQ ID NO: 100 - protein, 165 aa, Unknown
  found in bacteriophage isolated from environment
MKIKGFGNCPTYGHWVSLCGEVDPTKHFGFVYLVYCKKTGQYYIGKKQLNSVTKRKVAGKTRKKVVTKESDWMTYETSSEYIKKDIESFGKEFFDFYIIQTYHTKGGLVYGEANLQHKFDVMTKRIDSKLRLFYNANIAAIKFITKETYEDAEKRIHKVMKANCA

SEQ ID NO: 101 - protein, 744 aa, Unknown
  found in bacteriophage isolated from environment
MIKTIKKSNGTVVSFDPERLNKWASWADKRGIIWSEVTMEAMKRVYGGCTTKEMHQAMIDVCVDKQTQEYSDMAGRLLLGIIYKEAFGGFTKVPTLVTFVKNMERAGLWEKMDYSQEELEYLQGYIVHSKDISYGYAVLKQFRDKYGIRDIKTGRLFESPQFMFMGMAMKAFEKQPKHRRLQDVIKLYTYLSDLKINAPTPYLNGLRATKSGYASCCLIKANDTAESLGIAAKVAYDMTTKQAGIGMLMETRTIGDGIRQNTIEHMGKLPYYKLVRSSVEANKQKSRGGSANNFYTALDPQIEDLLRLKHPTTVPSKRINEMDYSFGTNDYFWQCVQYDTDWLLFSYKDAPKLYDMFYTASADEFAMAVGHAVHSGVKHRRVKAREIAKLFIQQRYATGRVYPFFTNNANTHTPFKEPLKMSNLCMEIVLPVYGFEKETDLYRDDAVKEDGEVALCFLASLVAGRISEDEYADVAYYALAMVDSVIDLMDYPYPSMRNHVQKRRSVGIGLTNVAHYLAKNYVNYSSRAGKTKLHELAEMHSYYLHEASLRLAKERGVPEYMKFTKYPEGWVPPKTANKKIDEKHDAKLRYDWDDLAQRIKENGGIRNSVLEAYMPNESSSLATNTTNGLYPIRDFILTKKSATGNVLFIVPDYEELKYVYEIAWGIDTFDLIDCYAIVQKFTGQAISSDFYVDYAKAKKVSLAQALKYMIYANSVGMKTMYYLNSRIGVGKSALQDAYCEGCGV

SEQ ID NO: 102 - protein, 377 aa, Unknown
  found in bacteriophage isolated from environment
LGSFRQKTSLRNDKERRNHAMINQHPIFLGGERKTFDSLNKHYPKIFELYKQQKAQDWSEDEFPFEQSRLDFESVPASMSGVMLEILKWQWEADTQVAKSLAFAFAPFISDDIYATAIMKQSEIENLHALTYSEIVRQCIKNPETILDEINQNVAVQDRLKTVNRVLEELLDEGINYRLSYVRDSLLDKDPLHFHKVILKGLFAVTALEGISFMASFACTFALDAQDKFQGIAQAVQKIMLDEILHTKIDIEVLKETLRDDEWQKAFQQILPEIKVILDEVVESEEKWSYYIFSEGRAVVGLNTKLLHEWVYYNAAPLYDMFGIPRDFVAPKEPPLKYMIKKMEIDKEQNANQEQQNGAYLLNTVVDDLNSGFLEVP

SEQ ID NO: 103 - protein, 80 aa, Unknown
  found in bacteriophage isolated from environment
MTYVIYSKTGCPQCETAKNFAKARGIDHVVRMLGQDYELSDLMEIAQMPVRQMPFIMKTDGQNLKPVGTLQNFMAEVNNA

SEQ ID NO: 104 - protein, 730 aa, Unknown
  found in bacteriophage isolated from environment
NERVSSETFLACKGAIMNKVQIIKKNGSLEEPDIKKVLAAVTKSANRVGYKELPPDVTQALESAFMRILVKSTKQNNLLISVNDIHSIVEGALAEVNHEIYESYSTYRNYRKEVAQNWDELYQKTKDTLFLGDRENANFDSSLISTKGSIIRGYLTKEIFKQYHLTPEELEAIEKGFIYIHDLRDLIFGGINCCLFDIGKVLKGGFEMSGIEYCEPKSVLSALQVIGDVVLSATAQQFGGFTLAEIDKVLVPYAKKSLRYHAEKAASYGIPKEHYHNYVMEQLQIELTQGFQSLEMKLNTVPCSRGDFAFTTLTFGLLDSDMSNEDNRLQYMIASTILDVRMNGQGKSKKPVVFPKLVYIYDQKRHNENICQGHLYSKAIECCSKAMYPDFLSVSGHGAVAEAFERSGKVISPMGCRAFLSPYLNEDGEEFYVGRANIGAVSLNLPMIYQYSKENGLDFWKELDKYLEMIRSFHKKRYEMIANMPASSNPLAFTQGGLYKGTKKPTDRVGWDIVKSFTASFGVTALDELSVLAEGKRLHEVGSCSFAYDVLEYINMKTEEFKNEDGFLYAVYGTPAESLCGTQLKQFREMFGVSKGVSDKEYFTNSFHMNVAADITPFEKQDLEEPFFHICRGGRIQYVRVANPENLSALKSCITRGMLKGFYQGLNFDLAICEHCGNRPKADVEECEVCHSHDISVINRVCGYLGWTKIKGESRMNDAKIAEIRDRISM

SEQ ID NO: 105 - protein, 714 aa, Unknown
  found in bacteriophage isolated from environment
MNKVQIIKKNGSLEEPDIKKVLAAVTKSANRVGYKELPPDVTQALESAFMRILVKSTKQNNLLISVNDIHSIVEGALAEVNHEIYESYSTYRNYRKEVAQNWDELYQKTKDTLFLGDRENANFDSSLISTKGSIIRGYLTKEIFKQYHLTPEELEAIEKGFIYIHDLRDLIFGGINCCLFDIGKVLKGGFEMSGIEYCEPKSVLSALQVIGDVVLSATAQQFGGFTLAEIDKVLVPYAKKSLRYHAEKAASYGIPKEHYHNYVMEQLQIELTQGFQSLEMKLNTVPCSRGDFAFTTLTFGLLDSDMSNEDNRLQYMIASTILDVRMNGQGKSKKPVVFPKLVYIYDQKRHNENICQGHLYSKAIECCSKAMYPDFLSVSGHGAVAEAFERSGKVISPMGCRAFLSPYLNEDGEEFYVGRANIGAVSLNLPMIYQYSKENGLDFWKELDKYLEMIRSFHKKRYEMIANMPASSNPLAFTQGGLYKGTKKPTDRVGWDIVKSFTASFGVTALDELSVLAEGKRLHEVGSCSFAYDVLEYINMKTEEFKNEDGFLYAVYGTPAESLCGTQLKQFREMFGVSKGVSDKEYFTNSFHMNVAADITPFEKQDLEEPFFHICRGGRIQYVRVANPENLSALKSCITRGMLKGFYQGLNFDLAICEHCGNRPKADVEECEVCHSHDISVINRVCGYLGWTKIKGESRMNDAKIAEIRDRISM

SEQ ID NO: 106 - protein, 43 aa, Unknown
  found in bacteriophage isolated from environment
MVWGFSDPQWSFSYHGQMDLWSCFKPFLGLAIAFSALPTKVKL

SEQ ID NO: 107 - protein, 131 aa, Unknown
  found in bacteriophage isolated from environment
MIKLSQKQLEWIKDYASECGSCEKNHVRYSTFHTVFTLYVGDNVLSDSVVDGVPLPNELLDKLAVVTGTWSEEDGHDLSNDVVFYSLENIMNPEYIMLMTCAQDCVPLQNFIKEHCEEFITKQVPCQVVFE

SEQ ID NO: 108 - protein, 161 aa, Unknown
  found in bacteriophage isolated from environment
MNYMEIRPFDTANGEGVRVSLFVAGCKHHCEGCFNRESWKFNAGKEFTYANLYGIIKLMDDKAISGLSILGGEPLDDRNIQEVTNICKRIKTVYPEKSIWLWTGFQLHEKIHLDVMKYVDVVIDGKYDSSKPTVKPYRGSDNQNLWRKEYGWQGDCQWRAE

SEQ ID NO: 109 - protein, 38 aa, Unknown
  found in bacteriophage isolated from environment
MCYRTNLTVGVTEEELKAFCEKIERDTKWESGTVLNQK

SEQ ID NO: 110 - protein, 106 aa, Unknown
  found in bacteriophage isolated from environment
MSKVYNTRKLQIFVLCQFMAKEYNCYYCGTGFISDNDGSYLPFKEAVKLFNEEVSSKKDIEKVKLTYSKKDKKIICLDNFVKVTGEAKEFMDESEISFTSILKVAQ

SEQ ID NO: 111 - protein, 92 aa, Unknown
  found in bacteriophage isolated from environment
MARTDWEVKELVSDAGNGCEVYMIRGHVPDPLAIEMIDHHTSGFYKELGEPSVERTWTKPVPDSTGNCSVIYHIVNPQKCKTAMAVTFVTFD

SEQ ID NO: 112 - protein, 298 aa, Unknown
  found in bacteriophage isolated from environment
LREKIMKTIIEAVVTAQEKFFHTEKFNIIQFPSGEIGGNFSEDFVKFTERNAGKIDNVILTVQGYDKDTLFALALAKDAVDSLVPYKSAMKTVVFGFLPNARYDRHMFKGDSAALKAFANLVNAMGFDAVCALDPHSNVAENLFKCFQSMKQKDVAAHFASDPRIDFLVAPDAGAAKKTEETAKEVDKPYITMSKVRNLKTGEITGMRILDTVDLTDKTVMILDDICDGGRTFIEAAKHLREAGAKRVELYVTHGIFSKGVENLLDNGVDHIYTTNSLGEAKDRGLTHYTQVTVVTID

SEQ ID NO: 113 - protein, 128 aa, Unknown
  found in bacteriophage isolated from environment
PQSMRLKQNLSFIIFVIISATTVACSFNKWVSFNLIMWNGDPSIPPFKESSKTLNVSRLAPLISLTINVTCPYSLSVSNANAKALSATSLRTGYLDAIFVTTKPNKGFARYSRWINLPSEAASFAKAL

SEQ ID NO: 114 - protein, 556 aa, Unknown
  found in bacteriophage isolated from environment
LNHFMQYQQVTQMLTNLVTFTSTLVQQNTCSIHHVVISGSTAPQLMVSLLVFNVLLKITTTGTPPSSNVTKKKQLTKSKLTVFWVKMLLVENIGKHSTTVIYQLKSTLKKAQLFLCVYQLSSRTLFQVSIGRDIWKMLSLLRFGRLVPLQLLHCTTNVSVRSGLTLLVTTTYICLISAMTLLCVVCPALLMTHLTLVTQALKGLIASLRYIQLNASMDSPIQSLILVALYQPLNTLCVQTLLGKVVMSLKKKDASKVSYKPSVASQKLTQLVLQVLFQTLIISGELYQYYQNSVKRLWNVMANWFVLTLETLYILSQDIKQFTSVLKRLITNTASRMPVTQCAPSTSLKILTMLWLGGYVQKAMKWWLTEKTLKPIQCCKMLTSTALQTLHVLRRLMELLRHCTTSLGELLTLKGLRYWMSTSALSMVTLSRWNVQTKSSVCMKWVLQALTCLVALTLTSTLVIPLHLLSKQLLQALVVKRLCLQKTLKQIVVLRNLLLVVHPCGMAKSKLMAMGSRALQMYLTTQLVLYVSLVTLSSSATQLLVIFEVILTSSFK

SEQ ID NO: 115 - protein, 169 aa, Unknown
  found in bacteriophage isolated from environment
TCFAIFHHNVSFVKFYGICHQQFISVDVARCVNYCTRYRLNISSNLCQVVDKPSSNVRFPKLYQILVDAKDITVRDRQFTCVFDQLTQIFHTKCARVYHKTYARVSVLVHSTIGKCKSDFCSKCANTTFLNHKCFVRFCITTNLFRSKTTYCGHPIAFLQFSYIAIPEL

SEQ ID NO: 116 - protein, 792 aa, Unknown
  found in bacteriophage isolated from environment
EESIMRMVNDHAEVIKSSTSLETSQAQITMTPEMFSLLSSGVYTFKERAVIRELSCNAVDAQKEAGRENIPFHVHLPTRFEPYFEVRDFGTGLTHDKVMSLYLNYGASTKNDSNDYIGAMGIGSKSPFAIAQSFTVSSYVDGVVNKYSVYLENGIPQVTKLTTNPTKEPNGLAVRVAVADHRISKFFEEAGNVYSYFAVKPESNIVYDDVLADMNVIAREEGVYDAMIHKQSWRSSGNRTEFNVVMGNIAYPVNMEALLGDDFFKVLPEFFRRSVDLVNIYMPIGSVAIAASREALQMNDTTKNVIIEATKKITEAITKDVIKKVNSQPTLMDAAQAYAELRLNSREMFNAVCPKLEWGGVKLDSLEEELLNIRRGIIYAEDGSVIYERDGKGNIKVDSKGNNIPKVDYLYNPVAYVKFNSLESKIRATALSYTQEASMFNIFGAMRKSQIEQFLFVINDRRNKNGTEKTVGRNQILRGACRDYASESNLFHRYNGIVFVFSTEKELDDLINLHKLDKSLLKIVKMSDKEHHYQRKEAVRGVVKLWKAVPAETKASYKEVSEDLDTIEEPQLYIKAVGDTVDGRCFCSSPEDVAKSVANVTGKTVYVFRKANWKKIPEDWIEVDEKLLNDSLTDVHWINHNRYMTRIYMDGVLDLTSSWIIARNFTFNNRKISRGYCYSRDTIKTIFLEGNEDAVEAMFGKIQYVAAPFAYTYTISVLQDLKSCLDNDTKLYKKIKKAGDRMVIKLTNYLSKRKQENFLLSHLDWNKVSPIEVSKFLGFNVKCVPEGTTIYD

SEQ ID NO: 117 - protein, 788 aa, Unknown
  found in bacteriophage isolated from environment
MRMVNDHAEVIKSSTSLETSQAQITMTPEMFSLLSSGVYTFKERAVIRELSCNAVDAQKEAGRENIPFHVHLPTRFEPYFEVRDFGTGLTHDKVMSLYLNYGASTKNDSNDYIGAMGIGSKSPFAIAQSFTVSSYVDGVVNKYSVYLENGIPQVTKLTTNPTKEPNGLAVRVAVADHRISKFFEEAGNVYSYFAVKPESNIVYDDVLADMNVIAREEGVYDAMIHKQSWRSSGNRTEFNVVMGNIAYPVNMEALLGDDFFKVLPEFFRRSVDLVNIYMPIGSVAIAASREALQMNDTTKNVIIEATKKITEAITKDVIKKVNSQPTLMDAAQAYAELRLNSREMFNAVCPKLEWGGVKLDSLEEELLNIRRGIIYAEDGSVIYERDGKGNIKVDSKGNNIPKVDYLYNPVAYVKFNSLESKIRATALSYTQEASMFNIFGAMRKSQIEQFLFVINDRRNKNGTEKTVGRNQILRGACRDYASESNLFHRYNGIVFVFSTEKELDDLINLHKLDKSLLKIVKMSDKEHHYQRKEAVRGVVKLWKAVPAETKASYKEVSEDLDTIEEPQLYIKAVGDTVDGRCFCSSPEDVAKSVANVTGKTVYVFRKANWKKIPEDWIEVDEKLLNDSLTDVHWINHNRYMTRIYMDGVLDLTSSWIIARNFTFNNRKISRGYCYSRDTIKTIFLEGNEDAVEAMFGKIQYVAAPFAYTYTISVLQDLKSCLDNDTKLYKKIKKAGDRMVIKLTNYLSKRKQENFLLSHLDWNKVSPIEVSKFLGFNVKCVPEGTTIYD

SEQ ID NO: 118 - protein, 788 aa, Unknown; identical to SEQ ID NO: 117
  found in bacteriophage isolated from environment

SEQ ID NO: 119 - protein, 376 aa, Unknown
  found in bacteriophage isolated from environment
LTRESNKMTTKTKAQIDAEIYKLVKEGKLTKTAIAQKFNTSTRSVGRAVERHEATLKGKKKATSTPATKTLKQVAKTFKKKAGKPVEKVVKDTVQKAPVNKLHEAMQKDDKIEYMITGDSVIMTYGSESEIVESTHPNYQEIVVHVVKGEFKKAFELMNIRKSIENFTQGAITIKGDKLFYGAVEMRSTLVDRILHMMKTGDKGFERLVMFFEKLMENPSKDSVEQLWGFVSHLDVEIDEEGYIIGWKKVSTREGKLVDSHTYKVPNDLGNIVEMPRWMVDNNRNVTCSQGLHVGAWDYVRCFSGDTILKVRVHPRDVVSVPTDYNDMKMRSSRYEVAAIVDNQRKVLEAWDGKTEALHVIVGTAGELISQRKREI

SEQ ID NO: 120 - protein, 369 aa, Unknown
  found in bacteriophage isolated from environment
MTTKTKAQIDAEIYKLVKEGKLTKTAIAQKFNTSTRSVGRAVERHEATLKGKKKATSTPATKTLKQVAKTFKKKAGKPVEKVVKDTVQKAPVNKLHEAMQKDDKIEYMITGDSVIMTYGSESEIVESTHPNYQEIVVHVVKGEFKKAFELMNIRKSIENFTQGAITIKGDKLFYGAVEMRSTLVDRILHMMKTGDKGFERLVMFFEKLMENPSKDSVEQLWGFVSHLDVEIDEEGYIIGWKKVSTREGKLVDSHTYKVPNDLGNIVEMPRWMVDNNRNVTCSQGLHVGAWDYVRCFSGDTILKVRVHPRDVVSVPTDYNDMKMRSSRYEVAAIVDNQRKVLEAWDGKTEALHVIVGTAGELISQRKREI

SEQ ID NO: 121 - protein, 113 aa, Unknown
  found in bacteriophage isolated from environment
EVTSNLHTRSQNVTNKVHHFSAIFTKFSKYHEEFFQHFVWRFIRYFLKPSKYRVKINFNIFFAVDNTPLFDRSTFALQNVRVGVFHRIIQTVNYFHWTPHNIIVTVYSARLTK

SEQ ID NO: 122 - protein, 65 aa, Unknown
  found in bacteriophage isolated from environment
VNSTFKNSQVSESDKYTQEESRYIIKGNTGSDVAAALEFYRDGFYQCTIKANNLIDMILLGNKQQ

SEQ ID NO: 123 - protein, 108 aa, Unknown
  found in bacteriophage isolated from environment
MQINGRDFVAVYYEKGKEVGVAQVSYGNGKWLYGTIAVVGTRSDTKTFKDCVDLLEESIQNHWCLIWMTDNEVIERFKKIDINIDSIEHVDLYELTEKVNYESNSSQR

SEQ ID NO: 124 - protein, 261 aa, Unknown
  found in bacteriophage isolated from environment
MGIVKIIKGDIFDAFDKGKFDIIGHGCNCMNFMGAGIAATISKRYPKVYDTDTEVYLFAGGIRHKPCENLLGNFSVARLKQGRIANLYTQLHTGKDARYSALESSLKQLNRYCEVNQLKKVGLPMIGAGIGGLDPQAVTVIINQVMKSVDVYLYVYEAKMYNELHSGWKNYCEPEYFAGVVVFTDNEVTLFRRRKGKIHQSNPPVEKMSLSNALVTHLSKSNHRIAVTFGSDADTYIYARTDEDIETVFSSPEVTFLDAKN

SEQ ID NO: 125 - DNA, 876 bp, Salmonella enterica
atgtcgcgactggcgcaggatatgaaaaaactggcgcaccgcgcaggtggcagccacaaaaccgtccacgaccgcgaacagatggcgcagcgttttgcccgtcatttgctggcgcagaatattcaggtcaccagcacctgccagctcaaagccagacacatcgcgggatatattcatgaatggctggcgcagggtatttctctgcgaacgctacagaatgagatggcaatggtgcgctctattctggctgaagccggacgcacacaactcagccagtcagaattaatcagcaaccagtcgctcggtatcagtggtgccagctgtgacgggacgcaccgggccattccggatgcactttaccatcaggtactggaccgggttcatcatatcgacgcaggcctcgcagccagcctgcaactggcgcgggtgatggggctgcgcggtcaggaagcagtacagtgctgccagtcgctgaaaacctgggataaacagctggaaaaaggggctgaacgtctgccggtgatcttcggcaccaaaggcgggcgaccacgcatgacacaggtgacggaccgggaagcggttcgtcaggcagtgaaagaggcgcttgggattgccggggagcgtaacgggcatctcattgacaagccggatctgaaatctgcaatggactactggcacaaccatctccgcgataccgggctgacgggggaatactctccccactccctgcgctatgcctgggcgcaggatgcgatccgctattacgaagagcagggtctgagccataaagaggcgctggcggtaacttcaacggatctgggccatggtgatgggcggggaatatatattgagcaggtatacgggtatactgattga

SEQ ID NO: 126 - DNA, 876 bp, Klebsiella pneumoniae
atgtcacgtctggcgcaggatatgaaaaaactggcgcaccgtgcgggtggtagccataaaacggtccatgatcgcgaacagatggcgcagcgtttcacacatcatttgctggcgcagaatatccagataacccgaaccgaccatattaaggccaggcacatcacaggctatatcaatgaaagactggcgcagggcatttccccgcgtaccttacagaatgagatggcgatggtgcgctctattctgactgaagccgggcgtacacaactcagccagtcagaattaatcagcaaccaatcgctcggtatcaacggtgcctgccgcgatggaacacatcgagcaattccggatgcgttttaccagcaggtgctggagcggatacagcaaaccgacgctggtctcgcggccagcctgcaactggcaagggtgatgggcctgcgtggtcaggaggcggtacagtgctgccagtcactgaaaacctgggataaacagctggaaaaaggggctgaacgtctgcccgtgattttcggcaccaagggcggacgaccacgcatgacgcaggtgacggaccgggaagcggttcgtcaggcggtgaaagaggcactgaatattgccggggagcgtaacggacatctgattgacaagccggatctgaaatccgcgatggattactggcacaaccacctccgcgatgctggactgacgggcgaatactcgccccactccctgcgctatgcctgggcacaggatgcgatccggtattacgaggagcaggggctgagtcacaaagaggcgctggcggtaacctcaacggatctggggcacggtgacgggcgcgggcggtatatcgagcagatttacgggcagactggctga

SEQ ID NO: 127 - DNA, 366 bp, Bacillus cereus
atggaggaaagagatatgaataaatcaacagttcaaatagctttaagtattacaaaattttcagcacagaagggatggaatgatgaagaattttgggaagcaatcgaattacttcgattaaataaggaagatacaagacaaacaatgatagaaaaattagatgggattccagttacttacgataatacaaatgattatccgattatgttgaaagtagatcatgtggcagaagtattagggatttcgtcaagaaaagcatatgacattatggatcaaaaaggattcccattagtaaaaatcgggcgtaggaaagtggtaccaagggatgcattctttaattggctcgaaaaggaggtgtcagcatag

SEQ ID NO: 128 - DNA, 759 bp, Enterobacter cloacae
atgccgattattcagtctgttgaacgtgcgttgcagatcctcgacctgttcaacgagcaggccaccgagcttaagatcaccgacatcagcaaactgatggggctaagcaagagtaccctccactcgttgctgaaaaccctgcagcttcatggctatatcgatcagaacccggagaacggcaagtatcgcctcggcatgaagctggtcgagcgcggtcatttcgttgtgggctccatcgatattcggcagaaggcgaaaggctggctgacggagctgtcacagcggaccgggcagacgacccatctggggatcctggacgggcgtgaaggggtttatatcgagaagattgaaggcaagctggccgccatcgcctattcgcgcattggccgccgtctgccggttcacgccaccgccatcggcaaggtgttgattgcctggctgggcgaaaccgagctgaacgccctgcttgagggttatcaatacactacctacaccccttccaccctcgcctctcgcgaagccttaatggccgccctggcgcagacccgcgagcagggctatgccctggatagcgaagagaacgagcagggcgtgcgctgcgtggcggtgccggtgtggaaccacgagtcccgcgtcattgccgccctgagcctgtcgacgctgacctcccgcgtggacgacgaagagctggctaatttccgcgagcagcttcagcaggccgggctccagctctcacgcgcgctgggctacccggcctga

SEQ ID NO: 129 - DNA, 246 bp, Vibrio parahaemolyticus
atgcctgtgtgtgctctacctaacgcggacggttttctcgctgtcgttcctgacattgaagcggcttcatgccgtggttatgtcatggtgacggctcaagaatatgacacgttaatgagctacacacagctgactccaggagagatatctcaagcgttcggcttgggtttcaccttggtgttcgttggcggatatctctcaacttacgccatcaagatggcaatacgtttaataaaactactttga

SEQ ID NO: 130 - DNA, 348 bp, Bacillus thuringiensis
atgaaaaattctacagttcaaatcgcgctggcaattacaaagttttcagctcaaaagggatggaaggatgaagagttttgggaggctattgagttacttcaattcaataaagaagatgaaaggcaaacaactgtagaaaagctagatagcattccagttactaatgatagtacagattatccaattatgttaaacgtaacacacttagaagaaatattaggagtagcgaaaaggacagcttacgaaattatggatcaaaaaggattcccgttagtgaaaatcgggcgtaagaaggcggttccaagggatgcattctttaattggttagaaaagggggtgtcagcatga

SEQ ID NO: 131 - DNA, 741 bp, Enterococcus faecium
atggtaaaaaaaataaaagccgaagatctatcagttgaaaaaggaacttacatgatttacgcaaatcctggtatgggaaaaacttattctttaggatttcttccaggtaaatcattggtattagatgttgacggatcatcatcgacattggcaaagcatcctaacaaagaaaatattgaagtgtgggaattagattcttctgatatttggcaagaatggcttgatactatttcagacttagtcgcaaataaatcttcctacgaaaagcaattcgacaatatttgtgtagataacatttctgaattgttcaaagcgcaattggaagatttaggcaagaaaggaaaaaatagtggtgttccttctcaagccgactatcaaagaacagattttatgaatttgcgaggtcttagagcattgaataatttagattgccgaatagtcctgactgcatgggaaacaacagacacatatacagaacctaatggacaattttttacaagatcaatgccagatataagatcaaaaattttaaataattttttaggcttatgtgatgtagttggcagacttgtaataaaaaaagatgacgatggtaatgaaacaagaggcttaatactaaagcctactagtaacgtttatgcaaaaaacagattagatgaacgtagtgggtgtttggtggaacaattggttgttagagcaggcggtgaaccgaatgtatcaactccgaccgtatcagattaa

SEQ ID NO: 132 - DNA, 261 bp, Escherichia coli O157
atgcgtaatatttctgtgtctgcgccagctcctgctgcgccattatttcctgtatcgaaccatcatgagcgatttttacgcctgcctgaggtgatgcacttatgcgggttgtcccgttcgaccgtttacgacctgatcagccgcgatgcgtttccgaagcagatcccgcttggcggtaaaaatgtcgcctgggcacagtctgaggtcagcgcatggatggcggaccgcatcagcgcccgtgggcggggatgtgatgcatga

SEQ ID NO: 133 - DNA, 894 bp, Escherichia coli str. K-12 substr. MC4100
atgaagctcttagcaaaagcacctctaaatctgttacgcgcctttgaagcggctggtcgtaccggagcttttgcgttggccgcctctgagttggaactgtcacccagtgcgatcagccatgccatccgcaaactggaaaacttgcttgatgtacgcctttttcagcgcagtacgcgagaaattacgctgacgaaagaaggtgaaatactgcttgagcacatacagcggggatttaacgaattacagcaggggttggcattagtgacggctgatgaatccaggcctttgcgccttcatactgcaccgagttttgcccatcaatggcttttaccacgtcttggtaagttcatacgtgagaacccaagcatcgatctgcgactttcagccagcacagaatatgcacgttttgaacaggatgattttgatctcgatatagtctacggtgaaccccgcccatcaccctatgagaagatcccgcttgctgttgaagaacttacgccactgtgttctccccaactggctgagcggctaaagaaaccagaggatctctatgcgttgacattgattcagtgcgatgtgcagttgtaccagtggaaaggatggtttgaggcgaataagatgacgccacccaataattatggccttcgatttgaccgtagctttatggcgattgctgccgcagttgacggcttgggagttgtgcttgaatcaaaactactggctgaacgtgaaatcgctagcggtaaactggtatgcccacttgtaaacagtaccagcgaaatacattacatcggtcattatttggttttcccccagcaccaacacatgcattctgcgcttgatgtgttcaaaacctggctactgaatgaactcaatctgggtaaaattcgttga

SEQ ID NO: 134 - DNA, 477 bp, Burkholderia pseudomallei
atgccgaagctccatgaagcggtcgccaacgggctgtccgaacgacttagaacgattcgaaaacgtgccggcctctcgcaagacgaactcgctgcgagggcgtcgctggctcgcacgaacctcgcgagcgtcgaacaaggacggcgggcaaacctccgactctcgaccctttcccgtctagctgatgtactgggcgtggatgtgctcgacttcttctgtgaccggcctgtcggcgaacaggaaccagccggggaagacgccccggctcgggtaatagcgaatgtgaaacggctgcgcagtgaggcagggctcagtcaagaagcactctcggtcaaagcccatcgatttcggacgtacgtcggccggttggagaacgaagcggccagcccaatggtggtcgatttgcaggacctggctgacgcccttggcatctcgattccggcactcctcgagcctagccagcacaaagccaaataa

SEQ ID NO: 135 - DNA, 897 bp, Yersinia pestis
atgttcgtaatgagtcggttaagtcgtgagatgaaaatgctggcaaaacaagctggcggtagccataaaaccgttcatgatcgtattcggattatggacaggtttagccgccatctgctggcccttaatattcaggtgcgtgatgttaaacacctaaaagcaaagcatattgaaagttatattactgaccgtttttcacaggggatcgcgatccgttctcttcataacgaaatggcggcattgcgcaccgttttcagattcgctggtcgggataaaatcgtcttatcagaacgattaaccaacaaagcattggggttaggtggagccagccgcgccggtagcaaattcgctatccctgaggaggtgtatcaagccgctctgcgcaccgcccaacagcaagacaaaggactcgcctgcgcccttcagcttgcccgcctgctggggctgcgttcacaagaggcggtgcaatgcgccaactcgttgaaaacatggcaaaaggagcttgagcagaaccggtctaaactaaccgtggtgtttggcaccaagggaggccgctcacgcgaaacccgcattctggatcctgaagcagtcaaacgggcagtcaaagaagcgcaacagatcgccgaaacacgcggcggcaaacttattgataaacctgacctgaaaaccgcgatgaatttctggcgctcccataccacccgcttagggctgaccggacattactccccccacagcctgcgctatgcctgggcgcaagacgctattaattattacctcgccgcaggatttagccgcattgaagccagagcgttaacgtcaatggatctgggtcatggtgatggccgtggccgctatgttgaacgcgtttatacccgcaaggaggcgaactatgtctga

SEQ ID NO: 136 - DNA, 321 bp, Yersinia pestis
atgattatggccaaagcccgtttacatgatgacgctatggttcagcttcttatggaagaccctgaatttgcgcaagtatatctgcatcaggctcttctagatattgatgaggaaggagggcaagaagcctttctgatggccctacgtcatgtagttgaggcccgaggagggatggcaagtgtggctaaaaaagcgggtgtatcgagagaaacactgtaccggacgttatcaccttcaggtaatccaacactaaaaacactgcttagtgtggtatctgcaacgggatttcagttttcacatcttgcatcgattaccgcatga

SEQ ID NO: 137 - DNA, 417 bp, Erwinia carotovora
atgtcccttggcgaaagattaagagaagagcgtgaacgactaggattcagccaagctgattttgctgagttagttggtgcatcaaggaaatcgcaaataaggtgggaaaaagctgaatcagctcctagcgctgattcaataagtctgtgggccaatgttggcttagacgtgttgtatctgatcactgggcaagctcagaaatcgtcatcaaagacacaaaaaagtagtgaaatagatgaaattctactgggtaagattgttcataaactggacttattagcgaaacatgccaatcgcagatggacacctagcgaactcgttttacagtcatcgaaaatatataactttttaatcaacgagtcggtagttgatgacgataggatcgataacgtgcttaaactggttgttaataattaa

SEQ ID NO: 138 - DNA, 492 bp, Unknown
  found in bacteriophage isolated from environment
atgcgattcagtgacaacggtctaagatttacggcagcatgggaggctttcagcccggtgccgtatttcgcgaccaagaaagagcaggcccgcggcctgtacacttggggttatggtcataccggcactaatccgcctcgaagcattacccgcgcggaagcgctggagctactcaagcgcgatgtggcgtatgccgagaactgggtgaacaaatacgcacatccgagcattaatcaggcgcaattcgacgcgctggtggacctggtaatcaatgctggtccaggtccgatcgtaccggaccatgtcgcgaacgatttcgatgatgcggtgcgactgggtgactgggcgaaggtccgggctatactgccgcagttccgcaagcaaggcgggtcggtgctcttaggtctggtacgtcgcgcaatcggcagacaggcgctattcgatggtaagcagtgggacgttgccgagaaaatcgggcgtaacgcagcataa

SEQ ID NO: 139 - DNA, 414 bp, Unknown
  found in bacteriophage isolated from environment
atgagttttaaatttggtaaaaatagcgaaaaacaattagctaccgttaagccagagctacaaaaagtagctcgtagagctttagaattatctccatatgattttacgatcgtacagggtattcgtacagtagcacaaagtgcccagaatattgctaatggtacttcatttttaaaggatcctagtaaaagtaagcatattactggggatgctcttgattttgctccatacattaatggcaaaattgattggaatgacttagaagcattttgggcagttaaaaaggcttttgaacaagctggtaaagaactaggcattaagcttcgttttggtgctgattggaatgcttcgggagactatcacgatgaaataaaacgtggtacctatgatggcggtcatgtcgaactagtttaa

SEQ ID NO: 140 - DNA, 489 bp, Unknown
  found in bacteriophage isolated from environment
atgaatatatttgaaatgcttcgtaatgacgaaggtcttagactgactttatacaaagacactgaaggcttttggacgattggcataggccatttagtaacaaagaacccgtctttagccgtagctaaagctgaacttgacagaatgatcggtcgtaaatgcaacggtacaattacccttgatgaggctgaaaagctatttaatgaagacgttgataaagccgtccgcggaatcttgggtaatgctaaacttaaaccggtatatgattctttagatgcagttcgtcggtgtgcattggtcaatatggtcttccaaatgggtgtagcaggcgtcgctggttttactaattctcttcgtatgcttaaacagaaacgttgggatgaagcggcagtaaatctagctcaatctaaatggtatcgtcagacacctaatcgcgcgaaacgcgtaatctcaacatttaaaacaggaacttggaaagcgtatatatga

SEQ ID NO: 141 - DNA, 465 bp, Unknown
  found in bacteriophage isolated from environment
atgcaactctcaagaaaaggtttagaagctattaagttctttgaaggtcttaagctagaggcttacgaagattctgctggaatccctacaattggatatgggactatccgtattgctggtaagcctgttaagatgggaatgaaaatcaccgctgaacaagctgaacagtatcttctcgcggatgttgaaaagttcgttgcagcagtgaacaaagctatcaaggttccaacttctcagaatgagttcgatgcacttgtaagtgaaacatacaacatcggtatcacagctatgcaggactcaacatttatcaagcgccacaacgatggtaataaggtaggttgtgcagaagctatgcagtggtggaacaaagttacagtcaaaggtaagaaggtcacttcaaacggcctgaaaaacagacgtaggatggaagctgacatttatcttgacagtgtatatccaaagtaa

SEQ ID NO: 142 - DNA, 486 bp, Unknown
  found in bacteriophage isolated from environment
atgcgattcagtgacaacggtctaagatttacggcagcatgggagcaattcagcccagtgccgtatttcgcgactaagaaagagcaggcccgcggcctgtacacctggggttatggtcataccggcaccaacccgcctcgaagcattacccgagcggaagcgctggagctactgcgcaaggatattgccgtagcagagacgtttgtgcagcgcgcaatgccgatcggggcattcaaccaagcccagtacgatgcgctcgtagacctggtgttcaacgtaggtccgcgtgcaatcggcgcgaatactggaactggtcgcgccgttcgtgctggggatgttcagacgatccgtgttaaactgccacaattccgtatgcaagccggtgtagttgttaagggtctggtgcgtcgcgctaatggacgccttgcgctattcgacggaaagcagtgggacgttgccgagcgtatcggtcgtaattcagcgtaa

SEQ ID NO: 143 - DNA, 498 bp, enterobacter cloacae A1S1
atggacatttttggtatgcttcgtattgacgaaggctgtaaattagaattatacaaagacactgaaggtttttggactattggcataggacaattaattaccaaaaacccttcatacaatgtagcacgtgatgagcttgatcgattaatgggtcgggtttgtaatggtcgtatcactcaacaggaagcagaaaatcttttcaattcttctgtagaaaaagctcgtaaaggtatcttggctaatgctactctaaaacctgtttatgatgtgctagatgaagttcgtcgttgtgcgttaattaacatggtgttccagatgggtgttgccggtacagcaggtttccctaaaggtatgcgtctgttaaaagcgaaacagtgggacaaagccgcaatcgagttagctgattctcgctggtacaaatctcaaacccctaatcgcgccaaacgcgtgattgcaacttttaaaactggtacttggaaagcatatgaaaacctataa

SEQ ID NO: 144 - DNA, 885 bp, Staphylococcus aureus
acacgtttaacaacacaagaattattatatctaaatgaactaaaattagcaataccttgtaaataaaaaatgtttatatttttcactattatagagctatttatctaaaaaggttcaataagacttaaatacgaattcaggcaacttaattgtgttaaatacagttttgaatgcctaactgtatttcttttctctttaaaatacagttaagtacattataagatgttgtgcggataaacaaactaattgcatcaaatttattttaaaataacaacaacaaaacgttaagagaataacatttcggtgatttaaaagctacgcacgtttttgttatcttcaaatttaaattttaaggagtgttttcaatggaaggtttatttaacgcaattaaagataccgtaactgcagcaattaataatgatggcgcaaaattaggcacaagcattgtgagcatcgttgaaaatggcgtaggtttattaggtaaattattcggattctaatttcaatatgttatgtaagtaatcagtattatttcaaaggtgagggagagatttaaatgactggactagcagaagcaatcgcaaatactgtgcaagctgcacaacaacatgatagtgtgaaattaggcacaagtatcgtagacatcgttgctaacggtgtgggtttactaggtaaattatttggattctaatataataactaatattctttaaaataaactgggtgagcatactttaatgttatgcactcagtttattttatttgcagaaatttgagcctctgttaagatttagatacatagacaatataggagatggggaaattgggatataaaaatattttgatagactttgatgatacaattgttgatttttatgatgcaga

SEQ ID NO: 145 - DNA, 1395 bp, Unknown
  found in bacteriophage isolated from environment
atgaccatcgacgttgccatcgcctttgccgacgctaaccactccacctacctcgaagacctgaaaggcctcgcgcgcataccgagcgtcagcttcccgggcttcgacgctgccgaagtggagcggtccgcggaacacgtcgcggacctgtgccggaatcgtggcctcgagaacgtcgaagtccttcgcgtccccggcgcccacccctacgtctacggcgactggctacatgcgccaggcaagccgacgctgcttctctacgcgcaccacgatgtgcagccgccgggccgcgaggagctgtggctcacgccgcccttcgagccgacggagcgcgatgggcgcctgtacggccgcggctgcgctgatgacaaagccggcgctatcacgcacatcgctgcgattgcagcgtggctgggcgcgaccggctcgctgccgctgaacgtgaagctgatcatcgaaggggaggaagagatcggctctgagcaccttgagtcgtttctggaaacccatgcttcgaagctgatggcggacgccatcgtgctcaccgacactgccaacttcgatgccgacacgccgtcgatcacagtggccctccgcggccttgtcgcagtcgacgtcacggtgcggtccatggaccatccattgcactccggcctgtggggcgggccgataccggacccggtgcaggccctcgcccgcatgattgccgcctgcactgacgcgacggggcggatgaccatccccggcatatacgacgacgtcgcggagcccggcgctgccgccgaggcaagcctcaaggtgctggagtaccctgaagcgctcttccgcgagcacaccggcctcaaccctgagtcgcagctactggtggcgtctacagacgtcctgcgctcgaactggtaccagccgtcgctctccgtgaacgccatccaggcttcgtcgcgcagggacgtcgcgaacatcatcaacgacagtgcctgggcgcacgtcggcatccgcatcgtgcccaacatggacgcgcgcaagaccctcgaggcgctgaaagcgcacctcgcagctaatgcgccgtggggcgtgcgcgtcgaattctccaacgaagccgccagccccgcctggaccacggataccgacgcaccggccttcgcagccgcccgccgcgccctcgaacgcggctacgccaagcccgccatcgacatgggctgcggcggctccataccctttgtcggccccttcgccgctgccctcaagggcgccccggccctgcttatcggcgtcgaagacccgctgtcaaacgcccacagcgagaacgagagcctgcatctcggcatgttccgcaaagcaattgcgtcggcggtagcgctgtacggagaaatcgcggcgctagacagcgtgaagtag

SEQ ID NO: 146 - DNA, 1029 bp, Unknown
  found in bacteriophage isolated from environment
atggccgaccgcagcgacgtggcaccccgacctccgcgggcccggaccggcgacgccatccgcgtgcccgcggccgcgctcgccagcgcggtcgccgcgctgggccgccgggcggtcggccggaagcggcggcccgcctggagcgcgcggctggaagccaccatcgccgcctaccgcggatcgtggtcgaccatgcccgcgctcgggatcgtgcgctggcgcaacgtcggcgaagcgctgagccgcctccgcacggacggcctccgcccgcgcttcacccgcttcgagagcggccagtacggcatccagggcacctggctcgagccggccggcgccagtggggccgtgctgctctacttccacggcgggggcttctgcttcggctcgatccggacccacggtccgctcatcggcgcgctggcccgggcggcgcgggcgcgcacgttcgccgccgagtatcgcctcgcgcccgagcacacggcgcccgccgcccacgacgacgcgctgtcggcctaccggcacctgctcggtgaaggcatcgcgccgcggcagatcgtgctcgccggcgattcgtccgggggcacgctggtcctcagcacgctgctggcgctgcgcgacgccggggaggcgatgccagcggccggcatcgccatctcgccctgggtggacctgtcgtgctcgggcgcgagcttcaaggccaacgcccccttcgacttcgtcggcgagccgcactgtcggctggcggcggtcagctatctgggcgccgtcgatccgcggcggcccgacatctcgccgctgttcgccgacctgcgcggcctgccgccgctgctgatccaggccggcggcgccgaggtcctggtcgaccagatccgggcgttcgcgggtcgcgcccgcgacgccggcgtcgacgtcacgctctcggtctacgacgacatggtgcacgtgtggcacctgatgcgcgacgtcacgcgggacggccagcgggccatcgacgaggccgccgcgttcgcgcgcgcccacgcctcgtga

SEQ ID NO: 147 - DNA, 1086 bp, Unknown
  found in bacteriophage isolated from environment
ttgctgttccgcctcctcgccgtcgccgacggacacaccccggccttgaccccgaaactcgtggaccccagggccgggccggtgaccttcaagctcgccgcgcatgacctcgacaagcgtgaccccgccctggtgctcaagggccgcgtcctcgacgaggacaggaagcccgtggccggcgccgtggtcgagccctgggggttccagaaagggcagcggggtcagggcggcgggctgaaggggttcgaccatctcgccgtcaccaacaacaagggggagttccggctgggcgtccccgagcccgggttagaggtatgggtccaggtcggggcgcggttcatggccgcccagaaagcatggctccttgccggccccaaggcgcatgacttgaccctcgtcaccggcgcctccgtcaccggccgcctgctgaaggacggcaagccgctcgccggcgtcgcggtcgggctggcgcagaagagccgcgaccccttcaccttcgtcggcgacttccaggcggccaccgatgagatgggtgtctttcgcatcccgcacgtcctgccgagagtcgagtacttcctctacggcaagatggacagcctgcgggcacacggggccatcgcggtgaaggaggtgcggaccggcgccagcggggcggaggtggatgtgggcgacctggccgtggcccctgggcacaagctgacgggccaggtggtcctggcggacgacaagccggtgcccgaaggggcgagggtgctcgtgtcgcgcgaggaggcgtgggacacgcagcaggcggtcgtcgacaaggatggacatttcgcgttggaggggctgccgccggagcggtattccctctcggtgcgggtgcccggctaccgcctctcgccgaagaacggcagctacgacctgctgggcggtttccgcctgctgggtgtggtccgctcggacatcaagtcgctgcgggtcctgctggagaagggggaggggccggaccgacttcccacatttgaccagaagcggcacaaggagtaccaacgcctccgggacgcgcccttgcgcggggccccggaagagggccgcgccaagtga

SEQ ID NO: 148 - DNA, 483 bp, Unknown
  found in bacteriophage isolated from environment
atgaaaaaattaatggcaaccctgattctggcaggcttggcaacttccgtatctgcagccggtatccatattgaagacggttgggcacgtgccactgtcgaaggtatgaaaatgggtggcgcattcatgaaaatccaaaacgatgaagccaaacaagacttcttggtaggtggcagcagccctgttgcagaacgcgttgaagtacacacccatattaacgacaacggtgttatgcgtatgcgcgaagttaaaggcggcgtgcctttggctgcaaaaggcgtaaccgagctgaaaccaggcagctaccacgttatgttcatgggtttgaaaaaacaattgaaagaaggcgaaaaagtgcctgtaaccctgaaattcaaaaacgcgaaacctcaaactgtcgagctggaagtgaaaaccgcaccacaatccggtatggatcacggtcacgaccacggtgcacacggtggcgcacatcagcactaa

SEQ ID NO: 149 - DNA, 828 bp, Bombyx mori
acattctgtctcgagcagcgaaacctgtcacaattcaaaatgtattccaaggtgttgttatccgctgcactccttgtatgcgtgaacgctcaagtttctatgcctcctggttacgcagagaagtatccgatcaccagccaattttcaaggtcagtccgacaccctcgcgatattcacgactttgtcacttgggacagggaaatggggggagggaaggtcttcgggactttgggagagagcgaccaaggactttttggtaaaggtggttacaacagggagttcttcaatgatgaccgcggcaaactgaccggacaggcttacggcaccagagtattaggacctggaggcgacagtaccagttacggtggtcgtctagactgggccaatgagaacgccaaggctgctattgacttgaacaggcaaattggtggcagcgctgggatcgaagcatcagcttccggcgtgtgggatcttggtaagaatactcacttgtcagctggcggagtggtctctaaggagttcggtcacagaaggcctgatgtcggtttacaggcccagattactcacgagtggtaattgccaacagcattatcaaagcagctatataagtgccgcttagacaagcattcttgcctcgtaagcctagaagtatgaataaaattttgtctaaaaacattgttataaaaatcgattgatttgtaactatttatctgcgttctgtaatttcctctttaaatcaccaacattcatctaacattttttaaatatgaataaaaataaatttcttaatataaaaaaaaaaaaaaaaaaaaaaaaaaaaaaaaaaaa

SEQ ID NO: 150 - DNA, 805 bp, Bombyx mori
acattcggtcttgaggagcgaaacttgatacaactcaaaatgaatacaaatctgttttatatcttcgctacaactctggtgtgtgttaacgcagaagtttacggaccttctgattacgcggaagattactcgatcagcgggcaatcctcaaggcgacaccctcgtgacgtcacttgggacaaacaaatggggggaggcaaggtctttggcactttgggacaaaacgatgatggactctttggaaaagctggttacaacagagagatctttaatgacgaccgcggcaaactaaccggtcaggcttacggcaccagagttttaggacctggaggcgacagcaccaactacggcggacgcctagactgggcgaacaagaatgcacaagccactattgacctaaatagacaaatcggtggcagatctgggatgacagcatcaggctccggtgtgtgggatctggataagaacacccacttttctgccggtggtatggtctcgaaggagttcggtcacaaaagaccagacgtcggtcttcaagcagagatccgccatgattggtgatcacctcaacacttaaacaataatcttttgccaaataaattagttttttttaattattcaatataggttattattgattgttttatctaaaatcgaaatttacgaagtatgtctaactgtaagtttgtttcataatgtaattagacaaataggatctcgtgctttgttatgacggttagaagtacaatttaataaaaaaattacgtttcctaaaaaaaaaaaaaaaaaaaaaaaaaaaaaaaaa

SEQ ID NO: 151 - DNA, 879 bp, Bombyx mori
acattctgaattaactagccaaaccacaaacaactcaaaatgaattccaaattgctgtttttcatcgctacagtcctggtgtgtgtaaacgcagaagtatacaggtctcctgattacgaagaagaataccccatcagaggcctattttcaaagcgacacccccgtgacgtcacgtgggacacgaaaatgggaggagggaaggtcttcggcactttgggacagaacgacgatggactcttcggtaaagccggttacaacagagagattttcaatgatgaccgcggtcaactaaccggccaggcttacggcaccagggtcttaggacccggaggtgacagcactaactacggtggacgtctggactgggccaacaagaacgcacaagccgccattgacataaacagacaaatcggaggcagatctggaatgacagcctcgggctccggtgtgtgggatcttgataagaacacccacatctcagccggcggtatggtctcgaaggaatttggtcacagaagaccagacgtcggtcttcaagcagagatccggcatgagtggtgattgtcaacacaatcccatcattcatagaattatgtttgtctttcgccaaaaaaagctactattttttttctgtgtatattttgtaattactagtgtaaaatgttatttttgattgatttaagaaaatgtcaatattattaacgaaatatgtcttacatattatgtactgttaacttgttttcttaatgtaattatgcaaacagtattttgctttatctcatactttgtgataaattaattaaaccatctagataataaataataataataaaaaattaatagtttaaaaaaaaaaaaaaaaaaaaaaaaaaaaaaaaa

SEQ ID NO: 152 - DNA, 840 bp, Bombyx mori
acattctgtctcaagcagcgaaacctgacacaagtcaaaatgaattccaaactattatatttcttcgccacggtcctggtgtgtgtcaacgcagaagtttattgggaggacgaagaaggatatccagttagcggtcagttttcaaagcgacacccgcgtgacgtcacctgggacaaacaagtggggggagggaaggtcttcggcacgctgggccaaaatgatgatgggctttttggtaaagccggttacaacagagagatcttcaatgatgaccgcggcaaattgaccgggcaggcctacggcaccagggtcctgggacccgcaggggacagtacaaactacggtggacgtctagactgggccaataagaatgcggaagctgctattgatataaatagacagatcggaggcagatctggaatgacagcaacaggctcaggagtgtgggatcttgacaagaacacccgcctctcagccggcggtatggtctcgaaggaattcggtcacagaagaccagacgtcggcgtccaggcagagttccgtcatgattggtgatcaagaagatcccatcaagacatcatagatctaaacaataatcttttgccaaagaaatcgatactttcactctgcatttttttaaattactaagaactgaatcaaattttagaattattataattaaagttatatattttagtgataaattatttggataatgtaagtaaatagatatagttacagtattctactttacttgtacttttaataaaacaaattgaattcaatggaaaaaaaaaaaaaaaaaaaaaaaaaaaaaaaaaaaaaaaaaaaaaaaaaaaa

SEQ ID NO: 153 - DNA, 30 bp, Artificial Sequence
  Multiple cloning site
gccggcagtggatccccggggaagatattc

SEQ ID NO: 154 - DNA, 15 bp, Artificial Sequence
  Primer
gctacactgctgaga

SEQ ID NO: 155 - DNA, 15 bp, Artificial Sequence
  Primer
tctcagcagtgtagc

SEQ ID NO: 156 - DNA, 13 bp, Artificial Sequence
  Primer
catcagatgctgg

SEQ ID NO: 157 - DNA, 13 bp, Artificial Sequence
  Primer
ccagcatctgatg

SEQ ID NO: 158 - DNA, 35 bp, Artificial Sequence
  Primer
taatactctacagacaccactaactgatgctgctg

SEQ ID NO: 159 - DNA, 54 bp, Artificial Sequence
  Primer
ctcgtttcaacatcttttattttgtacatacaagggattaagcagttcttaccc

SEQ ID NO: 160 - DNA, 54 bp, Artificial Sequence; identical to SEQ ID NO: 159
  Primer

SEQ ID NO: 161 - DNA, 29 bp, Artificial Sequence
  Primer
atgtacaaaataaaagatgttgaaacgag

SEQ ID NO: 162 - DNA, 32 bp, Artifical Sequence
  Primer
caccccttaattaaataaagtgtattagggtc

SEQ ID NO: 163 - DNA, 53 bp, Artificial Sequence
  Primer
cactttatttaattaaggggtgatgactgattgttaagatggtgttaatattc

SEQ ID NO: 164 - DNA, 29 bp, Artificial Sequence
  Primer
atgtacaaaataaaagatgttgaaacgag

SEQ ID NO: 165 - DNA, 33 bp, Artificial Sequence
  Primer
tcaccccttaattaagtaaagtgtattagggtc

SEQ ID NO: 166 - DNA, 55 bp, Artificial Sequence
  Primer
agaccctaatacactttacttaattaaggggtgatgactgattgttaagatggtg

SEQ ID NO: 167 - DNA, 33 bp, Artificial Sequence
  Primer
gtggtaaggtaaggtatggaaggatggcagtag

SEQ ID NO: 168 - DNA, 1173 bp, Staphylococcus phage SP5
atgtacaaaataaaagatgttgaaacgagaataaaaaatgatggtgttgacttaggtgacattggctgtcgattttacactgaagatgaaaatacagcatctataagaataggtatcaatgacaaacaaggtcgtatcgatctaaaagcacatggcttaacacctagattacatttgtttatggaagatggctctatattcaaaaatgagccccttattatcgacgatgttgtaaaaggattcattacctacaagatacctaaaaaggttatcaaacacgctggttatgttcgctgtaagctgtttttagagaaagaagaagaaaaaatacatgtcgcgaacttttctttcaatatcgttgatagtggtattgaatctgctgtagcaaaagaaatcgatgttaaattggtagatgatgctattacgagaatcttaaaagataacgcgacagatttattgaacaaagactttaaagagaaaatagataaagatgtcatttcttacatcgaaaagaatgaaagtagatttaaaggtgcgaaaggtgataaaggcgaaccgggacaacctggagcaaaaggtgaagtaggtaaaaaaggagaacaaggcgcacccggtaaaaacggtactgtagtatcaatcaatcctgacactaaaatgtggcaaattgatggtaaagatacagatatcaaagcagaacctgagttattggacaaaatcaatatcgcaaatgttgaagggttagaagataaattgcaagaagttgaaaaaatcaaagatacaactctcaacgactctaaaacgtatacggatacaaaaattgctgaactagttgatagcgcgcctgaatctatgaacacattaagagaattagcagaagcaatacaaaacaactctatttcagaaagcgtattgcaacagattggctcaaaagttagtacagaagattttgagagattcaaacaaacactaaatgatttatatgctccaaaaaatcataatcatgacgagcggtatgttttgtcatctcaagcttttactaaacaacaagcggataatttatatcaactaaaaagcgcatctcaaccgacggttaaaatttggacaggaacagaaaatgaatataactatatatatcaaaaagaccctaatacactttatttaattaaggggtga

SEQ ID NO: 169 - DNA, 1173 bp, Staphylococcus phage SP6
atgtacaaaataaaagatgttgaaacgagaataaaaaatgatggtgttgacttaggtgacattggctgtcgattttacactgaagatgaaaatacagcatctataagaataggtatcaatgacaaacaaggtcgtatcgatctaaaagcacatggcttaacacctagattacatttgtttatggaagatggctctatattcaaaaatgagccccttattatcgacgatgttgtaaaaggattcattacctacaagatacctaaaaaggttatcaaacacgctggttatgttcgctgtaagctgtttttagagaaagaagaagaaaaaatacatgtcgcgaacttttctttcaatatcgttgatagtggtattgaatctgctgtagcaaaagaaatcgatgttaaattggtagatgatgctattacgagaatcttaaaagataacgcgacagatttattgaacaaagactttaaagagaaaatagataaagatgtcatttcttacatcgaaaagaatgaaagtagatttaaaggtgcgaaaggtgataaaggcgaaccgggacaacctggtgcaaaaggtgaagcaggtaaaaaaggagaacaaggcgcacccggtaaaaacggtactgtagtatcaatcaatcctgacactaaaatgtggcaaattgacggtaaagatacagatatcaaagcagaacctgagttattggataaaatcaatatcgcaaatgttgaagggttagaagataaattgcaagaagttaaaaaaatccaagatacaactctcaacgactctaaaacgtatacggattcaaaaattgctgaactagttgatagcgcgcctgaatctatgaacacattaagagaattagcagaagcaatacaaaacaactctatttcagaaagtgtattgcaacagattggctcaaaagttagtgcagaagattttgaggaattcaaacaaacactaaatgatttatacgctccaaaaaatcataatcatgacgagcggtatgttttgtcatctcaagcttttactaaacaacaagcggataatttatatcaactaaaaagcgcatctcaaccgacggttaaaatttggacaggaacagaaaatgaatataactatatatatcaaaaagaccctaatacactttacttaattaaggggtga

SEQ ID NO: 170 - DNA, 34 bp, Artificial Sequence
  Primer
aagactcggaagaaggtagtcactaaggaaagtg

SEQ ID NO: 171 - DNA, 56 bp, Artificial Sequence
  Primer
ccgtaaatcttagaccgttgtcactgaatcgcatgtcaagttttacatagaaatcc

SEQ ID NO: 172 - DNA, 38 bp, Artificial Sequence
  Primer
atgcgattcagtgacaacggtctaagatttacggcagc

SEQ ID NO: 173 - DNA, 37 bp, Artificial Sequence
  Primer
ttatgctgcgttacgcccgattttctcggcaacgtcc

SEQ ID NO: 174 - DNA, 55 bp, Artificial Sequence
  Primer
ttgccgagaaaatcgggcgtaacgcagcataaaaggtgatgtgggtcttgatagg

SEQ ID NO: 175 - DNA, 35 bp, Artificial Sequence
  Primer
gcaacactgtatcggctacttcaaagtcttctctg

SEQ ID NO: 176 - DNA, 492 bp, Unknown; identical to SEQ ID NO: 138
  found in bacteriophage isolated from environment

SEQ ID NO: 177 - DNA, 53 bp, Artificial Sequence
  Primer
catatcctgcgccagtcgcgacatacaagggattaagcagttcttacccaagc

SEQ ID NO: 178 - DNA, 34 bp, Artificial Sequence
  Primer
atgtcgcgactggcgcaggatatgaaaaaactgg

SEQ ID NO: 179 - DNA, 27 bp, Artificial Sequence
  Primer
tcaatcagtatacccgtatacctgctc

SEQ ID NO: 180 - DNA, 50 bp, Artificial Sequence
  Primer
ttgagcaggtatacgggtatactgattgatgactgattgttaagatggtg

SEQ ID NO: 181 - DNA, 51 bp, Artificial Sequence
  Primer
catatcctgcgccagtcgcgacatgtcaagttttacatagaaatcctgtca

SEQ ID NO: 182 - DNA, 34 bp, Artificial Sequence; identical to SEQ ID NO: 178
  Primer

SEQ ID NO: 183 - DNA, 27 bp, Artificial Sequence
  Primer
tcaatcagtatacccgtatacctgctc

SEQ ID NO: 184 - DNA, 52 bp, Artificial Sequence
  Primer
ttgagcaggtatacgggtatactgattgaaaggtgatgtgggtcttgatagg

SEQ ID NO: 185 - DNA, 34 bp, Artificial Sequence
  Primer
acaaatagtgaagagataaaccaggttgagcaag

SEQ ID NO: 186 - DNA, 45 bp, Artificial Sequence
  Primer
ttgacgttgaatctggagtcgataggtgcgacaggttaccaatgg

SEQ ID NO: 187 - DNA, 41 bp, Artificial Sequence
  Primer
gtcgcacctatcgactccagattcaacgtcaaggtctcacc

SEQ ID NO: 188 - DNA, 35 bp, Artificial Sequence
  Primer
ttccaagacggattcgaaccgtcactagtacaagg

SEQ ID NO: 189 - DNA, 47 bp, Artificial Sequence
  Primer
ttaatgatgttatctcgataacgtcgacatggagactcagtaaatgg

SEQ ID NO: 190 - DNA, 44 bp, Artificial Sequence
  Primer
tctccatgtcgacgttatcgagataacatcattaaggttgtacc

SEQ ID NO: 191 - DNA, 49 bp, Artificial Sequence
  Primer
ttgaataaaccgttatcgccttcttaaagcaacctgtattgcgttctgc

SEQ ID NO: 192 - DNA, 50 bp, Artificial Sequence
  Primer
ttgctttaagaaggcgataacggtttattcaacaaaccctcatttcattg

SEQ ID NO: 193 - DNA, 49 bp, Artificial Sequence
  Primer
tctgtgtagttcggccaactgtagtgtgcgaatgatgcagcgaacattc

SEQ ID NO: 194 - DNA, 50 bp, Artificial Sequence
  Primer
ttcgcacactacagttggccgaactacacagataccatgaagcagtactc

(19) United States
(12) Patent Application Publication
(10) Pub. No.: US 2022/0348886 A1
(43) Pub. Date: Nov. 3, 2022

(54) Genetically Engineered Bacteriophage

(71) Applicant: Cytophage Technologies Inc., IIe Des Chenes East (CA)

(72) Inventor: Steven Theriault, Ile Des Chenes East (CA)

(73) Assignee: Cytophage Technologies Inc., IIe Des Chenes East, MB (CA)

(21) Appl. No.: 16/962,881

(22) PCT Filed: Jan. 21, 2019
(86) PCT No.: PCT/CA2019/050074

§ 371 Date: Jul. 17, 2020

Related U.S. Application Data
(60) Provisional application No. 62/619,461, filed on Jan. 19, 2018.

(51) Int. Cl. C12N 7/00; C12N 15/62; C12N 15/52; C12N 15/87; A01N 63/40; A01P 1/00

(52) U.S. Cl. CPC C12N 7/00; C12N 15/62; C12N 15/52; C12N 15/87; A01N 63/40; A01P 1/00; C12N 2795/00021; C12N 2795/00032; C12N 2795/00062; C12N 2800/10

(57) ABSTRACT

There is disclosed a method of engineering bacteriophages comprising: identifying a bacteriophage with only one attachment gene; isolating said bacteriophage; removing said attachment gene from the genome of said bacteriophage; and inserting a non-natural attachment gene into the genome of said bacteriophage wherein said non-natural attachment gene is specific for attaching to a selected bacteria. There is also disclosed a mutant bacteriophage comprising a heterologous nucleic acid sequence encoding a first specific attachment gene, the first specific attachment gene being different than an inactivated attachment gene and being specific for a selected bacteria. In another embodiment, there is disclosed a method of eliminating a microbial contaminant, the method comprising: obtaining one or more lytic enzymes produced by a mutant bacteriophage; applying the one or more lytic enzymes to a bacterial contaminant, without prior infection of the bacterial contaminant with a bacteriophage, to eliminate the bacterial contaminant.

### CROSS-REFERENCE TO RELATED APPLICATIONS

This application is the National Stage entry under 35 U.S.C. § 371 of International Application Number PCT/CA2019/050074 filed on Jan. 21, 2019, published on Jul. 25, 2019 under publication number WO 2019/140534 A1, which claims the benefit of priority of U.S. Provisional Patent Application Ser. No. 62/619,461 filed Jan. 19, 2018.

### SEQUENCE LISTING

The instant application contains a Sequence Listing which has been submitted electronically in ASCII format and is hereby incorporated by reference in its entirety. Said ASCII copy, created on Jan. 21, 2019, corrected Sep. 24, 2020, is named RMDOCS-#6135266-v1-TXT_Copy_of_SEQ_Listing_ -_Corrected_Sept_2020.TXT and is 524,473 bytes in size.

### FIELD OF THE INVENTION

Prevention, diagnostics and treatment of human, animal, and plant bacterial infections.

### BACKGROUND

Bacteria are unicellular, biological entities that are mostly not harmful to humans—less than one percent of the different types make people sick. Many bacterial species are beneficial to humans, such as those that help to digest food, destroy disease-causing cells, and provide needed vitamins.

Infectious bacteria (the harmful one percent) cause illness in humans and animals. They reproduce quickly in the body and produce toxic proteins that cause tissue damage and illness.

Bacteriophages (also referred to as phages) were discovered by Ernest Hankin in 1896, and utilized as antibacterials against cholera. These bacteria-specific viruses can infect and destroy bacterial cells.

Bacteriophages are composed of proteins that encapsulate a DNA or RNA genome. Bacteriophages replicate within bacterium by injecting their viral genetic material (DNA or RNA) into the host cell effectively taking over the cells functions for the production of progeny bacteriophage leading to the rupture of the cell wall and subsequent bacterial cell death.

Bacteriophages continued to be used as antibacterials until the 1930's. However, it was found that bacteria naturally build up resistance to bacteriophages. With the introduction of chemical antibiotics, use of bacteriophages was abandoned.

While antibiotics are the usual treatment, bacterial mutations conferring antibiotic resistance are becoming increasingly common in pathogenic bacteria world-wide. Methicillin-resistant Staphylococcus aureus (MRSA) bacteria, for example, is an increasingly common form of infection, often acquired through transmission in hospitals. MRSA infections are extremely difficult to treat using conventional antibiotics.

Bacteriophages can be very specific to the type of disease-causing bacterial species. Most bacteriophages have structures that enable it to bind to specific molecules on the surface of their target bacteria.

A key advantage of bacteriophages is that they enable the elimination of antibiotic-resistant bacteria without the need for increasingly toxic antibiotics or harmful or irritating chemical-exposure to humans, animals and the environment (see, e.g. U.S. Pat. No. 6,699,701 to Intralytix).

In natural settings, bacteriophages can be isolated from the environment in which the particular bacterium grows following a paired relationship, for example from sewage or feces. Repositories of different types of natural bacteriophages have been created to provide access to bacteriophages to treat difficult infections by specific bacterial species.

One problem with using bacteriophages has been that the patient's own body will often have an immune response against the bacteriophages and eliminate the bacteriophages from blood. U.S. Pat. Nos. 5,660,812, 5,688,501, 5,811,093 and 5,766,892 all show methods of selecting or generating (using mutations) bacteriophages to improve the bacteriophage half-life within the blood of a patient to be treated.

Another problem associated with prior uses of phages to disinfect or treat bacterial contaminants or diseases, are that bacteria can become resistant to bacteriophages. The presence of, for example, a prophage within a bacterium may block the expression of genes from an infectious bacteriophage, thus preventing replication of the infectious bacteriophage and preventing lysis and killing of the bacterium. A prophage may also cause the destruction of incoming phage DNA.

This has previously meant that either the bacteriophage needs to be matched to the bacterium, often requiring complicated genetic analysis of the bacterium, or a number of different phages need to be used in combination. The production of panels of different bacteriophages, such as panels of vir mutants derived from temperate bacteriophage, is disclosed in WO 03/080823.

Currently, only natural bacteriophages exist, and natural bacteriophages that have been mutated and selected for specificity against certain bacteria (see, e.g. U.S. Pat. No. 8,685,697 to Intralytix).

### SUMMARY OF INVENTION

The invention is a template or platform technology for creating customized genetically modified bacteriophages that target and destroy specific bacterial organisms found in humans, animals and agricultural crops, as well as on surfaces in healthcare or food processing facilities.

Specific products can be developed using this template technology such as a disinfectant spray for MRSA, food additive to prevent antibiotic use in animal feed, and treatment of bacterial infections in humans. The invention thus encompasses genetically modified bacteriophages as well as including gene products derived from bacteriophages, used to treat and or remove bacterial infections utilizing bacteriophages.

According to one aspect, there is disclosed a method to manipulate the viral genome to cause functional changes in the life cycle of the virus.

In one embodiment, the invention provides a method of engineering bacteriophages comprising:
identifying a bacteriophage with only one attachment gene
isolating said bacteriophage;
removing said attachment gene from the genome of said bacteriophage; and
inserting a non-natural attachment gene into the genome of said bacteriophage wherein said non-natural attachment gene is specific for attaching to a selected bacteria.

In another embodiment, the invention provides a method of engineering bacteriophages comprising:
isolating a bacteriophage;
removing any attachment gene from a genome of said bacteriophage;
inserting a first unique open reading frame encoding one or more attachment genes and inserting a second unique open reading frame encoding one or more genes useful for overcoming bacterial defenses;
inserting a non-natural attachment gene into said first open reading frame, wherein said non-natural attachment gene is specific for attaching to a selected bacteria. The one or more genes useful for overcoming bacterial defenses are endolysins, bio-file reducers, glycocalyx penetrators, or any combination thereof.

In another embodiment, the invention provides a method of engineering bacteriophages comprising:
isolating a bacteriophage;
removing any attachment gene from a genome of said bacteriophage;
inserting a multiple restriction enzyme cassette in said genome; and
inserting a non-natural attachment gene into said cassette, wherein said non-natural attachment gene is specific for attaching to a selected bacteria.

In another embodiment, the invention provides a method of engineering bacteriophages comprising:
isolating a bacteriophage;
removing all natural attachment genes from the genome of said bacteriophage; and
inserting a non-natural attachment gene into the genome of said bacteriophage;
wherein said non-natural attachment gene is specific for attaching to a selected bacteria.

In another embodiment, the invention provides a method of growing bacteriophages comprising:
preparing a yeast culture comprising yeast and yeast nutrients;
infecting said yeast with bacteriophages;
screening said yeast with colony-PCR for positive transformants.

The bacteriophage may comprise a non-native attachment gene, wherein said non-native attachment gene is specific for attaching to a selected bacteria. The bacteriophage may have no native attachment genes. The bacteriophage may be lytic. The non-native attachment gene is specific for pathogenic/non-pathogenic bacteria. The bacteriophage may be used for cleaning, treating, or preventing a bacterial contaminant.

The invention also teaches bacteriophage for diagnosis of the presence or absence of a specific bacteria.

The invention also teaches a method of producing a mutant bacteriophage, the method comprising inactivating an attachment gene from a selected bacteriophage, the selected bacteriophage being isolated from bacteriophages from the environment; inserting, into the selected bacteriophage, a first heterologous nucleic acid sequence comprising a first open reading frame encoding a first specific attachment gene, the first specific attachment gene being different than the inactivated attachment gene and being specific for a selected bacteria, to produce the mutant bacteriophage. A second heterologous nucleic acid sequence may be inserted in a second open reading frame encoding a gene useful for overcoming bacterial defenses. The gene for overcoming bacterial defenses may be a biofilm degrading gene, a glycocalyx degrading gene, a gene encoding an antibacterial protein, and a gene for an enzyme that disrupts the bacterial wall, to produce the mutant bacteriophage. The step of inactivating may inactivate all attachment genes from the selected bacteriophage.

The invention also teaches a bacteriophage which is a lytic bacteriophage, a bateriohage with a small genome size, or a bacteriophage with structural and functional genes to lyse gram negative and gram-positive bacteria, or any combination thereof.

The invention also teaches an anti-microbial composition for sanitizing or decontaminating a surface.

The invention also teaches a method of eliminating a microbial contaminant, the method comprising: obtaining one or more lytic enzymes produced by the mutant bacteriophage; applying the one or more lytic enzymes to a bacterial contaminant, without prior infection of the bacterial contaminant with a bacteriophage, to eliminate the bacterial contaminant.

### BRIEF DESCRIPTION OF THE DRAWINGS

FIG. 1 shows an overview of a phage engineering platform, according to an embodiment of the present invention.

FIG. 2 shows an overview of a method to generate mutant bacteriophage using a cell free cloning method, according to an embodiment of the present invention.

FIG. 3 shows an overview of a method to generate mutant bacteriophages using yeast strain, according to an embodiment of the present invention.

FIG. 4 is an agarose plate of the titration of pp8 against E.coli DH5 alpha after rescue from the genetic template. Phage was spot plated on a lawn of E. coli. Concentration was determined to be 10⁸ for isolate one and 10⁶ for isolate two phage units per 10 ul.

FIG. 5 shows a schematic representation of the entire genome of the disclosed mutant bacteriophage, according to an embodiment of the present invention.

FIG. 6 shows the nucleotide sequence of the entire genome of PP8 and the proteins encoded therein along with the restriction endonuclease sites according to an embodiment of the present invention.

FIG. 7 is a detailed description of the PP8 molecule and proteins with annotations according to an embodiment of the present invention.

FIG. 8 is a gel electrophoresis photograph of PP8 DNA digestion using enzymes specific to remove inserts. EcoRI for ORF1 and ORF2 and TspRI for ORF 3 and ORF 4, where Lane 1: 1 kb DNA ladder (NEB), Lane 2: space, Lane 3: undigested PP8 DNA, Lane 4: Digested PP8 ORF1 insertion SP5 attachment gene (46090) band size 1.1kb, Lane 5: Digested PP8 ORF2 insertion Endolysis gene (73195) band size 2.1, Lane 6: Digested PP8 ORF3 insertion SP6 attachment gene (19991) band size 1.2kb, Lane 7: Digested PP8 ORF4 insertion endolysis gene (60431) band size 2.1

FIG. 9a—Shows a gel electrophoresis photograph where Lane 1: 1kb DNA ladder (NEB), 2: space, 3: Extracted bacteriophage genome control, 4: Bacteria control (mock—bacteriophage infected), 5 - 7: Purified bacterial colonies with potential integration. Expected band size: 554 bases.

FIG. 9b—is a gel electrophoresis photograph where Lane 1: Extracted bacteriophage genome control, 2: Bacteria control (mock - bacteriophage infected), 3-5: Purified bacterial colonies with potential integration. Expected band size: 613 bases.

FIG. 10 shows an overview of the disclosed method for modifying the binding sites, according to an embodiment of the present invention.

FIG. 11 shows the results of the MRSA phage treatment experiment where bacteriophage PP8 (SR5) insertion lysis of MRSA patient samples 1-6. Bacteriophage at a concentration of 10⁷ was used to develop a kill curve of 6 MRSA positive patient samples. These samples were named patient 1-6.

FIG. 12 shows the titration of PP8/SP5 against Staphylococcus aureus. Phage was spot plated on a lawn of Staphylococcus aureus. Concentration was determined to be 10⁵ phage units per 10 ul.

FIG. 13 shows the titration of PP8/SP6 against Staphylococcus aureus. Phage was spot plated on a lawn of Staphylococcus aureus. Concentration was determined to be 10⁸ phage units per 10 ul.

FIG. 14 shows the results of the new MRSA phage treatment where PP8 (SR5, SR6) insertion kill curve of MRSA patient samples 1-6. Bacteriophage at a concentration of 10⁵ were used to develop a kill curve of 6 MRSA positive patient samples. Patient samples were tested for survivability at a concentration of 10⁶.

FIG. 15 is a photograph showing a PP8 SP5/SP6 bacterial challenge. Bacteriophage PP8 SP5/SP6 was flooded onto the agarose plate. Bacterial strains were tested for lysis. 50) E. coli O9 51) E.coli O1 52) E.coli O28 53) E.coli DH5 alpha 54) Salmonella Enterica 55) Listeria monocytogenes 56) Entercoccus durans 57-61) MRSA patient sample 1-5 respectively.

### DETAILED DESCRIPTION

The methods and techniques of the present disclosure are generally performed according to conventional methods well known in the art and as described in various general and more specific references that are cited and discussed throughout the present specification unless otherwise indicated.

The terms “polypeptide”, “peptide”, and “protein” are typically used interchangeably herein to refer to a polymer of amino acid residues. Amino acids may be referred to herein by either their commonly known three letter symbols or by the one-letter symbols recommended by the IUPAC-IUB Biochemical Nomenclature Commission. Each protein or polypeptide will have a unique function. The invention includes polypeptides and functional fragments thereof, as well as mutants and variants having the same biological function or activity.

In some embodiments, polymeric molecules (e.g., a polypeptide sequence or nucleic acid sequence) are considered to be “homologous” to one another if their sequences are at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% identical.

In some embodiments a fragment of a nucleic acid sequence is a fragment of an open reading frame sequence. In some embodiments, such a fragment encodes a polypeptide fragment (as defined herein) of the protein encoded by the open reading frame nucleotide sequence.

The term “nucleic acid fragment” as used herein refers to a nucleic acid sequence that has a deletion. In some embodiments a fragment of a nucleic acid sequence is a fragment of an open reading frame sequence. In some embodiments, such a fragment encodes a polypeptide fragment (as defined herein) of the protein encoded by the open reading frame nucleotide sequence.

The term “construct” refers to a nucleic acid sequence encoding a protein, operably linked to a promoter and/or other regulatory sequences.

The term “genomic sequence” refers to a sequence having non-contiguous open reading frames, where introns interrupt the protein coding regions.

As used herein, the terms “encoding”, “coding”, or “encoded” when used in the context of a specified nucleic acid mean that the nucleic acid comprises the requisite information to guide translation of the nucleotide sequence into a specified protein. The information by which a protein is encoded is specified by the use of codons. A nucleic acid encoding a protein may comprise non-translated sequences (e.g., introns) within translated regions of the nucleic acid or may lack such intervening non-translated sequences (e.g., as in cDNA).

The term “percent sequence identity” or “identical” in the context of nucleic acid sequences refers to the residues in the two sequences which are the same when aligned for maximum correspondence. For instance, polynucleotide sequences can be compared using the computer program, BLAST (Altschul et al., J. Mol. Biol. 215:403-410 (1990); Gish and States, Nature Genet. 3:266-272 (1993).

The term “substantial homology” or “substantial similarity,” when referring to a nucleic acid or fragment thereof, indicates that, when optimally aligned with appropriate nucleotide insertions or deletions with another nucleic acid (or its complementary strand), there is nucleotide sequence identity in at least about 70%, 80%, 85%, or at least about 90%, or at least about 95%, 96%, 97%, 98% or 99% of the nucleotide bases, as measured by any well-known algorithm of sequence identity, such as BLAST, as discussed above.

As used herein, “heterologous nucleic acid sequence” is any sequence placed at a location in the genome where it does not normally occur. In some embodiments, the heterologous nucleic acid sequence is a natural phage sequence, albeit from a different phage.

A particular nucleic acid sequence also encompasses conservatively modified variants thereof (such as degenerate codon substitutions) and complementary sequences, as well as the sequence explicitly indicated. Thus, a nucleic acid sequence encoding a protein sequence disclosed herein also encompasses modified variants thereof as described herein. Substantially similar nucleic acid fragments of the instant invention may also be characterized by the percent identity of the amino acid sequences that they encode to the amino acid sequences disclosed herein, as determined by algorithms commonly employed by those skilled in this art.

An “orgin bacteriophage” is a phage isolated from a natural or human made environment that has not been modified by genetic engineering. A “mutant bacteriophage” is a bacteriophage that comprises a genome that has been genetically modified by insertion of a heterologous nucleic acid sequence into the genome, or the genome of the phage. In some embodiments the genome of a origin bacteriophage is modified by recombinant DNA technology to introduce a heterologous nucleic acid sequence into the genome at a defined site.

“Operatively linked” or “operably linked” expression control sequences refers to a linkage in which the expression control sequence is contiguous with coding sequences of interest to control expression of the coding sequences of interest, as well as expression control sequences that act in trans or at a distance to control expression of the coding sequence.

A “coding sequence” or “open reading frame” is a sequence of nucleotides that encodes a polypeptide or protein. The termini of the coding sequence are a start codon and a stop codon. The disclosure also includes native, isolated, or recombinant nucleic acid sequences encoding a protein, as well as vectors and/or (host) cells containing the coding sequences for the protein.

Fragments and variants of the disclosed nucleotide sequences and proteins encoded thereby are also encompassed by the present invention. By “fragment” a portion of the nucleotide sequence or a portion of the amino acid sequence and hence protein encoded thereby is intended. Fragments of a nucleotide sequence may encode protein fragments that retain the biological activity of the native protein. Accordingly, the present disclosure relates to any nucleic acid fragment comprising a nucleotide sequence that encodes all or a substantial portion of the amino acid sequences encoded thereby.

The present technology uses synthetic biology to generate bacteriophages that can bind to specific bacterial strains. Since bacteriophages must attach to host bacterial cells to initiate infection of the bacteria, genetic selections or manipulations in the viral DNA or RNA can define binding characteristics, thus expanding the range of host cells beyond the natural paired relationship. According to one embodiment there some characteristics of the disclosed bacteriophages, including the following.

The phages are safe, non-corrosive, and non-toxic. The phages can be engineered so that they do not affect helpful bacteria, animal or human cells. Thus, there is no interference with the food chain, as with antibiotics.

The phages are designed, not discovered in nature. Thus, the technology is adaptable to any bacterial infection. Undesirable genetic components are eliminated. In contrast, the present methods of isolating natural phages for specific bacteria is like finding a “needle in a haystack” for target bacteria.

The phages are engineered to avoid mutation/adaptation of target bacteria resulting in superior kill rates and no resistance. Accordingly, the phages have superior efficacy over known phages. The phages also prevent biofilm formation.

According to one embodiment, the platform is versatile. The disclosed bacteriophages can be used to solve any bacterial problem. The disclosed bacteriophages have application in human health (personalized medicine, disinfectants, and diagnostics) such as for example, in MRSA and VRE, animal health (livestock medicine, diagnostics) such as for example, ear drop for treating dog ear infections of Staphylococcus intermedius, and food safety (produce cleansing, detection of bacterial contamination) such as for example, E. Coli, C. Jejuni, Salmonella, and Listeria.

According to one embodiment, the bacteriophages can not only be used for the treatment of antibiotic-resistant bacterial infections but also for prevention of bacterial-contamination in the environment and in food which may negatively affect human and animal health.

For example, the phages are useful for human health. Methicillin-resistant Staphylococcus aureus (MRSA) bacteria are an increasingly common hospital-acquired infection, often acquired through contact with contaminated surfaces. For facilities with a confirmed MRSA problem, this product can be used to thoroughly clean surfaces and reduce the development of new infections. According to an embodiment, there is provided a multi-strain MRSA-specific disinfectant cleanser that can be used on porous and non-porous surfaces in hospitals including beds, curtains, tables, chairs, diagnostic and monitoring equipment, and medical instruments.

According to an embodiment, the disclosed bacteriophages can be used to reduce or eliminate any bacteria and/or resistant bacteria that are pathogenic to humans and/or animals. In aspects, the advantages of using this disinfectant over the commonly-used disinfectants, such as bleach, are multiple. First, bacteriophage are more effective in destroying bacteria than conventional means. Second, phages can be left on surfaces to destroy new bacterial contamination events, surviving for roughly 24 hours. Third, unlike bleach, bacteriophages do not leave a corrosive residue, and thus do not harm instruments, fabrics, and skin. Fourth, bacteriophages, customized for harmful bacteria, are non-toxic, unlike cleaning solutions.

The phages are also useful in animal health treatments. For example, bacteriophage are tailored to address bacterial infections in chickens, replacing the antibiotic(s) commonly used, resulting antibiotic-free chickens—a commercial benefit in today's marketplace. This treatment also contributes to reducing the growing number of antibiotic-resistant infections that occur as bacteria mutate and evolve to be unaffected by antibiotics.

The phages are also useful in food safety. For example, bacteriophage-cleansing spray can be applied on agricultural crops for the prevention of food-borne illnesses from bacterial contamination during plant cultivation or during harvesting, such as Escherichia coli-contamination of strawberries.

The template technology is utilized to generate bacteriophages with various specific binding domains (thus selecting host range). The technology provides bacteriophages in high concentrations.

In some embodiments, bacteriophage-derived gene products may be useful for “lysis-from-without” whereby bacteria can be eliminated without having to become infected.

According to an embodiment there is provided a method of eliminating a bacterial contaminant without prior infection of the bacterial contaminant with a bacteriophage, the method comprising obtaining one or more lytic enzymes produced by the disclosed bacteriophage; applying the one or more lytic enzymes to a bacterial contaminant to eliminate the bacterial contaminant.

A bacteriophage or phage is defined as a virus that infects bacteria. Bacteriophages have a high specificity to their corresponding host bacteria. To infect bacteria, the bacteriophage attaches to specific receptors on the surface of the bacteria. This attachment determines the host range of each bacteriophage, and normally is restricted to some genera, species, or even subspecies of bacteria. This bacteriophage specificity could provide clinicians, laboratory technicians, technicians in the field, as well as consumers, with the ability to identify (detect or diagnose) specific types of bacteria by exploiting this bacteriophage characteristic.

Bacteriophages experience two types of natural life cycles, or methods of viral reproduction, known as the lytic cycle and the lysogenic cycle. In the lytic cycle, host cells will be broken and suffer death after replication of the virion. In contrast, the lysogenic cycle does not result in immediate lysing of the host cell and consequential host cell death; rather, the bacteriophage genome integrates with the host DNA, or establishes itself as a plasmid, and replicates along with the organism's genome. The endogenous bacteriophage remains dormant until the host is exposed to specific conditions (e.g., stress) at which point the bacteriophage may be activated, initiating the reproductive cycle resulting in the lysis of the host cell.

Endolysins are produced during the last stage of the phage lytic cycle from within their host and most are released into the periplasmic space (Borysowski et al., 2006). From there on, endolysins cleave covalent bonds in the peptidoglycan to release viral progeny (Fischetti, 2008). Within the endolysin subgroup, there are five classes: amidases, endopeptidases, muramidases, glucosaminidases and transglycosylases (Gasset, 2010).

According to an embodiment, there is provided the use of lytic enzymes or enzybiotics from bacterial viruses to combat antimicrobial resistance. An enzybiotic is defined to be a protein that degrades the bacterial cell wall, meaning that it is not subjected to bacteriophage proteins (Borysowski and Gorski, 2010). The term enzybiotics was first conceived in the paper ‘Prevention and elimination of upper respiratory colonization of mice by group A streptococci by using bacteriophage lytic enzyme’ (Nelson et al., 2001). The bacteriophage lytic enzymes are specific. Phage derived lytic enzyme and their destructive activity against certain components of the cell wall found in pathogenic bacterial strains but not the natural microbiota of animals (Gasset. 2010). Two examples include group C streptococcal lysin, effective in lysing group A streptococci but has no effect on normal oral streptococci (Fischetti, 2006). A more relevant example is attained from the use of the outer membrane protein FyuA, commonly expressed in pathogenic Gram-negative Escherichia coli. The fusion of FyuA binding domain to T4 lysozyme results in translocation of the fusion from the outer membrane to the periplasmic space where the lysozyme can destabilize the bacterial cell wall (Lukacik et al., 2013).

According to one embodiment, there is disclosed a method for providing an endolysin protein or plurality of endolysin proteins, which overcome the issues with whole bacteriophages. The one or more endolysins specifically targets and degrades the bacterial cell wall (peptidoglycan) from both within the cell or from outside of the cell resulting in lysis. In aspects, there is provided a method to generate various clones of endolysin genes from numerous bacteriophages and using high-throughput screening, and to evaluate the success of the endolysin clones against one or bacteria, such as for example, Escherichia coli strains, Salmonella typhimurium and Campylobacter jejuni.

Thus, the technology extends the number of bacterial strains that may be treated with bacteriophage or bacteriophage gene products with and without infection.

Bacteriophages multiply themselves by infecting and killing bacteria. During this process, bacterial cell wall components are released along with the bacteriophages. These components may be toxic to humans, animal and bacteria. Thus, large scale preparations of bacteriophages using bacteria require post-manufacturing treatments using harsh organic chemicals to reduce the toxicity to acceptable levels for clinical treatment.

Therefore, according to one embodiment, there is provided a method to grow the disclosed bacteriophages in large-scale use of bacteria by using yeast strains, such as for example, Kluyveromyces lactis and Pichia pastoris. The disclosed methods circumvent the liberation of toxic end products.

### Generating Mutant Bacteriophages

Environmental samples were isolated and fully characterized to determine candidates which meet certain criteria. Preferably, a suitable origin bacteriophage is selected from candidates which includes one or more of the following features:
lytic phages;
genetically different than known phages; and
carries only one attachment gene

According to an embodiment, there is provided a method to genetically modify one or more suitable origin bacteriophages.

In one embodiment, the origin bacteriophage includes one attachment gene. In another embodiment, the origin bacteriophage includes more than one attachment gene.

In one embodiment, the method generates bacteriophage platforms configured to allow for further interchanging of one or more desired proteins, such as for example, attachment proteins.

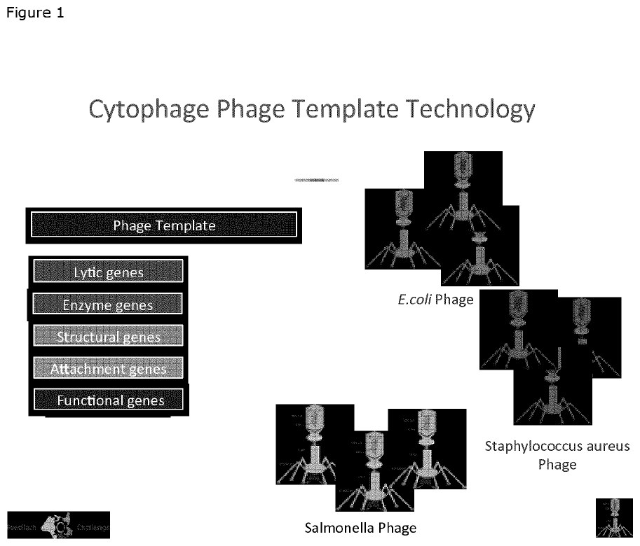

According to one embodiment, the bacteriophage genomes are manipulated to change the virus' life cycle, creating gain of function, loss of function or for virus identification (reporter genes). A summary is shown in FIG. 1.

In one aspect, the origin bacteriophage is a lytic phage. In one aspect, the origin bacteriophage is a lytic phage that carries one or more attachment gene. In another aspect, the origin bacteriophage is a lytic phage that carries only one attachment gene. In one aspect, the origin bacteriophage carries only one attachment gene.

According to one embodiment, there is provided a method to produce a mutant bacteriophage. In one aspect, the method comprises modifying the phage binding sites of an origin bacteriophage so that the mutant bacteriophage can attach to different serotypes. In one embodiment, the mutant phage is then rescued and the new binding domain is determined.

According to one embodiment, the engineered bacteriophage comprises only lytic genes, wherein any and all lysogenic genes have been removed to ensure integration cannot occur.

According to one embodiment, there is provided a method of ‘cell free cloning’ to provide a template (or platform) technology that allows for the modification/insertion/deletion of viral genes. The platform was generated by constructing a mutant bacteriophage (defined as a phage which was generated from known and unknown genetic codes) using isolated environmental samples.

Genetic comparison of unknown phage types from environmental samples were tested against known phage types allowing us to isolate known gene types.

In one embodiment, there is provided a mutant bacteriophage where genes of interest were added and where unwanted genes were deleted. Together with noncoding regions, the mutant bacteriophage is a genetic platform that carries at least two unique open reading frames (ORF).

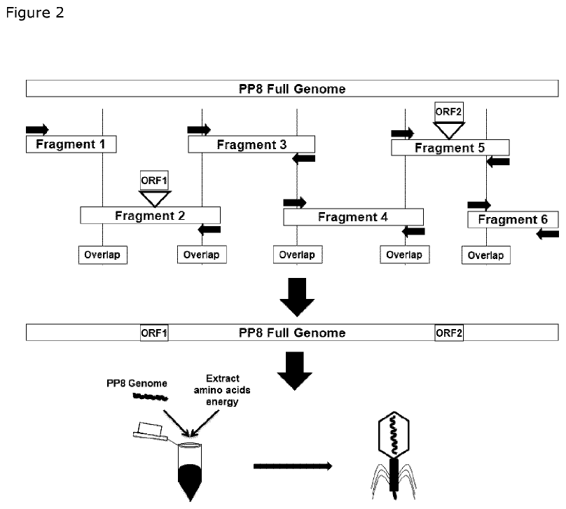

These unique ORFs can be used to add genes of interest. With reference to FIG. 2, the genomic compliment is divided into fragments with overlapping sections to adjacent fragments obtained by PCR amplification. Foreign genes are inserted within respective fragments. Fragments were combined using bacterial cellular extracts exploiting the homologous recombination methodology, where extracts contain the necessary components to link fragments together into one contiguous fragment via homology. Rescue of bacteriophages from the fully assembled genomes is achieved by cell-free translation. This method involves mixing DNA of choice along with toxin free cellular extracts from E. coli along with amino acids and energy, the transcription and translation proteins and enzymes from the extract drives expression from the DNA leading to generation of bacteriophage.

In aspects, the mutant bacteriophage is a genetic platform that carries four unique open reading frames (ORF).

In one embodiment, the first ORF can be used to insert an attachment gene for a bacteria. In one aspect, the attachment gene can be selected from, but not limited to, the following proteins:
DNA-binding phage protein of Enterobacteriaceae (>CP007523.1:3585236-3586111 Salmonella enterica subsp. enterica serovar Typhimurium str. CDC 2011K-0870, complete genome) SEQ ID No: 125
DNA-binding phage protein (>CP002910.1:3892390-3893265 Klebsiella pneumoniae KCTC 2242, complete genome) SEQ ID No: 126
DNA binding protein (>CM000724.1:300852-301217 Bacillus cereus BDRD-ST26 chromosome, whole genome shotgun sequence) SEQ ID No: 127
Phage DNA-binding transcriptional regulator (>CP003678.1:c575894-575136 Enterobacter cloacae subsp. dissolvens SDM, complete genome) SEQ ID No: 128
Phage ssDNA binding protein (>CP009983.1:941901-942146 Vibrio parahaemolyticus strain FORC_008 chromosome 2, complete sequence) SEQ ID No: 129
DNA binding protein (>CM000749.1:288493-288840 Bacillus thuringiensis. T04001 chromosome, whole genome shotgun sequence) SEQ ID No: 130
phage nucleotide-binding protein (>CP006620.1:c2486999-2486259 Enterococcus faecium Aus0085, complete genome) SEQ ID No: 131 DNA-binding protein Bacteriophage P4 (>AE005174.2:318190-318450 Escherichia coli 0157:H7 str. EDL933 genome) SEQ ID No: 132
CP4-6 prophage; putative DNA-binding transcriptional regulator (>HG738867.1:c269405-268512 Escherichia coli str. K-12 substr. MC4100 complete genome) SEQ ID No: 133
DNA-binding protein (Burkholderia pseudomallei K96243 chromosome 1, complete sequence) SEQ ID No: 134
Putative DNA-binding prophage protein (>AL590842.1:c1239408-1238512 Yersinia pestis CO92 complete genome) SEQ ID No: 135
Putative DNA-binding prophage protein (>AL590842.1:1235071-1235391 Yersinia pestis CO92 complete genome) SEQ ID No: 136
Putative phage-related DNA-binding protein (>BX950851.1:4152092-4152508 Erwinia carotovora subsp. atroseptica SCRI1043, complete genome) SEQ ID No: 137

In one embodiment, the second ORF is used to insert a gene encoding a protein useful for overcoming bacterial host defenses.

For example, the second ORF can be for introducing is to add enzymatic functions to combat bacterial defenses. In one aspect, the second ORF can be used to add endolysin genes, and/or biofilm degrading genes.

In an embodiment, the endolysin genes are selected from:
PP1 phage endolysin SEQ ID No: 138 which is similar to Escherichia phage B2: 93% identical and 100% query coverage Accession Number:MG581355; Enterobacteria phage JL1: 92% identical and 100% query coverage Accession Number: JX865427; Shigella phage EP23: 91% identical and 100% query coverage Accession Number: JN984867; Sodalis phage: 91% identical and 100% query coverage Accession Number: GQ502199.
PP2 phage endolysin SEQ ID No: 139 which is similar to Escherichia phage phiLLS: 99% identical and 100% query coverage Accession Number: KY677846; Salmonella phage Stp1: 98% identical and 100% query coverage Accession Number: KY775453; Salmonella phage SPO1: 98% identical and 100% query coverage Accession Number: KY114934; T5 phage-like pork29:97% identical and 100% query coverage Accession Number MF431732.
PP3 phage endolysin SEQ ID No: 140 which is similar to Enterobacteria phage ATK48: 99% identical and 100% query coverage Accession Number: KT184310; Shigella phage SHSML-52-1: 99% identical and 100% query coverage Accession Number KX130865; Escherichia phage APCEc01: 99% identical and 100% query coverage Accession Number: KR422352.1; E. coli 0157 typing phage 6: 98% identical and 100% query coverage Accession Number: KP869104; Shigella phage Shf125875: 98% identical and 100% query coverage Accession Number KM407600; Shigella phage phi25-307: 98% identical and 100% query coverage Accession Number: MG589383; Klebsiella phage vB_Kpn_F48:73% identical and 98% query coverage Accession Number: MG746602;
PP7 phage endolysin SEQ ID No: 141 which is similar to Salmonella phage ST11: 95% identical and 100% query coverage Accession Number: MF370225; Salmonella phage Meda: 95% identical and 100% query coverage Accession Number MH586731; Salmonella phage Si3: 95% identical and 100% query coverage Accession Number: KY626162; Escherichia phage EC6: 95% identical and 100% query coverage Accession Number: JX560968; Bacteriophage Felix 01: 95% identical and 100% query coverage Accession Number AF320576; Enterobacteria phage KhF2: 94% identical and 100% query coverage Accession Number: KT184314; Salmonella virus VSe102: 94% identical and 100% query coverage Accession Number: MG251392; Salmonella phage Mushroom: 94% identical and 100% query coverage Accession Number KP143762; Staphylococcus phage SA1: 94% identical and 100% query coverage Accession Number: GU169904; E. coli 0157 typing phage 15: 94% identical and 100% query coverage Accession Number: KP869113; Citrobacter phage Mijalis: 83% identical and 99% query coverage Accession Number KY654690; Shigella phage Sf14: 82% identical and 99% query coverage Accession Number: MF327003;
PP11 phage endolysin SEQ ID No: 142 which is similar to Enterobacteria phage HK578: 79% identical and 97% query coverage Accession Number: JQ086375; Escherichia phage Sloth: 78% identical and 97% query coverage Accession Number KX534339; Escherichia phage Envy: 78% identical and 97% query coverage Accession Number: KX534335
Enterobacter cloacae A1S1 phage endolysin SEQ ID No: 143

In one embodiment, the biofilm degrading genes and glycocalyx degraders are selected from:

| Protein Name | Accession Number |
|---|---|
| Cathelicidin antimicrobial peptide | NM_004345.5 |
| LL-37 | |
| Histatin 3 (HTN3) | NM_000200.2 |
| Nisin | M24527.1 |
| Dispersin B | NZ_NRDE01000005.1 |
| Endo-1,4-β-glucanase (callulase) | NM_001247953.1 |
| Aureolysin | EF070234.1 |
| NucB | HQ112343.1 |
| Serine protease (SspA) | AF309515.1 |
| LapG protease | KT186446.1 |
| Melittin | NM_001011607.2 |
| Endo-1,4-β-mannosidase (manA) | AM920689.1 |
| α-amylase | A17930.1 |

For example, the second ORF can be for introducing antibacterial proteins used in template to address bacterial lysis. In one aspect, an example protein is a bacterial cell wall degrader used to degrade Staphylococcus aureus (>ENA|JQ066320|JQ066320.1 Staphylococcus aureus strain JP1 Psm betal (psm beta1) and Psm beta2 (psm beta2) genes, complete cds). SEQ ID No: 144

In other aspects, the second ORF can be for introducing enzymes which target the key linking chemistries (amide, ester and glycolytic bonds) found in bacterial cell walls. Examples include:
M20 family peptidase [uncultured bacterium], ACCESSION AHZ45606 (uncultured bacterium, >KF835382.1:c34024-32630 Uncultured bacterium clone SZR5 genomic sequence) SEQ ID No: 145
Lipolytic enzyme (uncultured bacterium ACCESSION AHZ45613 >KF835383.1:7038-8066 Uncultured bacterium clone WZR9 genomic sequence) SEQ ID No: 146
peptidase M56 ([uncultured bacterium] ACCESSION AHZ45657 Uncultured bacterium clone WZR18 genomic sequence (>KF835385.1:c14123-13038 Uncultured bacterium clone WZR18 genomic sequence) SEQ ID No: 147;
Another example is Uncultured bacterium clone HOAb112C long-chain fatty acid CoA-ligase gene ([uncultured bacterium] DBSOURCE accession KF955286.1) SEQ ID No: 148;
Bombyx mori BmGloverinl mRNA for gloverin-like protein 1, complete ACCESSION AB190863 SEQ ID No:;149
Bombyx mori BmGloverin2 mRNA for gloverin-like protein 2, complete ACCESSION AB190864 SEQ ID No: 150;
Bombyx mori BmGloverin3 mRNA for gloverin-like protein 3 ACCESSION AB190865 SEQ ID No: 151; and
Bombyx mori BmGloverin3 mRNA for gloverin-like protein 4 ACCESSION AB190866 SEQ ID No: 152;

According to an embodiment, there is provided a method of producing a mutant bacteriophage, the method comprising inactivating at least one attachment gene from a selected bacteriophage, the selected bacteriophage can be isolated from bacteriophages from the environment. The method further comprises inserting, into the selected bacteriophage, one or more a heterologous nucleic acid sequences comprising one or more attachment genes. The one or more inserted attachment genes being different than the inactivated native attachment gene and is/are choosen because of its specificity for a selected bacteria, to produce the mutant bacteriophage. In some embodiments, the provision of the selected attachment gene(s) expands the range of possible host cells (i.e. bacteria) beyond the natural paired relationship.

According to an embodiment, there is provided a method of producing a mutant bacteriophage, the method comprising inactivating at least one attachment gene from a selected bacteriophage, the selected bacteriophage can be isolated from bacteriophages from the environment. The method further comprises inserting, into the selected bacteriophage, a first heterologous nucleic acid sequence comprising a first open reading frame encoding a first specific attachment gene. The first specific attachment gene is different than the inactivated attachment gene and is choosen because of its specificity for a selected bacteria, to produce the mutant bacteriophage.

In another embodiment, the method further comprises inserting a second heterologous nucleic acid sequence in a second open reading frame encoding a gene useful for overcoming bacterial defenses. In aspects, the gene for overcoming bacterial defenses may be a biofilm degrading gene, a glycocalyx degrading gene, a gene encoding an antibacterial protein, and a gene for an enzyme that disrupts the bacterial wall, to produce the mutant bacteriophage. In one aspect, the first open reading frame further encodes a second specific attachment gene that is different than the first specific attachment gene.

In some embodiments, the method inactivates all the attachment genes from the selected bacteriophage. In aspects, the step of inactivating comprises making an inactivating mutation in at least one native attachment gene. In some aspects, the inactivating mutation is a point mutation.

According to an embodiment, there is provided an anti-microbial composition for sanitizing or decontaminating a surface. In aspects, the anti-microbial composition comprises the disclosed mutant bacteriophage.

According to an embodiment, there is provided a method of decontaminating a surface suspected of containing a bacteria. In aspects, the bacteria is an infectious or a non-infectious bacteria. The method comprising applying the disclosed anti-microbial composition comprising the disclosed mutant bacteriophage to the surface. In aspects, the amount is effective to decontaminate the surface of at least substantially or all of the contaminating bacteria.

In aspects, the surface is a biological surface (animal or plant).

According to an embodiment, there is provided a method to generate specific mutant bacteriophage gene products. In aspects, there is provided a method of eliminating or substantially eliminating a microbial contaminant (an infectious or non-infectious bacteria), the method comprising: obtaining one or more lytic enzymes produced by the disclosed mutant bacteriophage and applying the one or more lytic enzymes to a bacterial contaminant. In some aspects, the elimination is accomplished without prior bacteriophage infection of the microbial contaminant and therefore leads to result of lysis from without.

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

### EXAMPLES

### Example 1—Bacteriophage Isolation

Samples from sewer and waste, environmental soils, and animal feces were collected and purified to be used for the isolation of bacteriophages. Purified samples were then screened for the presence of bacteriophages against specific bacteria. Protocols and methods for isolating bacteriophages from water samples were adapted from Bonilla et al. (2016) and Bourdin et al. (2014); solid and soil sample methods adapted from Sillankorva (2018), Pausz et al. (2009), and Van Twest & Kropinski (2009).

Solid samples were rehydrated using sterile water for a minimum of 1 hour to allow the bacteriophages to disseminate. Samples are then centrifuged to remove solid materials and large particulates and the supernatant is collected. The centrifuged environmental samples and water samples were then further processed and purified using filters (0.2 μM) to remove bacteria and smaller unwanted particulates. Filtered samples can be further concentrated using filter tubes or stored at 4° C. for future use.

Filtered samples were then tested against bacterial strains of interest using an agar overlay plaque assay technique (Kropinski et al. 2009). Liquid agar overlay was inoculated with filtered environmental sample and the bacterial strain of choice and mixed. It was then poured onto an agar culture plate (bacterial strain dependent) and allowed to harden. Plates were then incubated overnight (conditions are bacterial strain dependent) and observed the next day for plaques against the chosen strains. Plaques containing bacteriophages were then picked and further processed by 3-rounds of subsequent plaque assay overlays to purify the selected phage(s).

Using the method outlined above, numerous (hundreds) EV samples were collected and tested for suitability to develop the template. The function and structural genes were characterized for each EV sample, tested for integration (as detailed below). Candidate phages with a low copy number of lysogenic genes, and the structural and functional genes to allow for gram negative and gram-positive lysis was identified. A selected bacteriophage named PP8 was sequenced and gene structure and function were examined as detailed below. PP8 was selected as it had the desired genes. Although it also had lysogenic genes, these were removed using ORF replacement.

### Example 2—Platform Development

Using environmental sample EV31/PP8, after bacteriophage isolation we purified genomic material with PureLink viral DNA/RNA extraction kit. The full-length genome was amplified (EV31/Full/F/pYESIL and EV31/Full/R/pYESIL see sequence EV31) to have 30bp homology with the pYESIL Sapphire vector. PCR amplification was performed using Phusion high-fidelity DNA polymerase (modification use of touchdown technique for primer annealing starting at 69C and dropping by 0.5C each cycle). PCR products were separated on agarose gels and bands were excised, extracted, and assembled. The resulting construct EV31pYES (unmodified) allowed for the genetic modification of EV31 and the determination of function of mutations in a phage rescue based system.

### Example 3—Full-length Genome Assembly

In brief, the following provides for a method for the genetic manipulation of yeast (Kluyveromyces lactis and Pichia pastoris) cells to include T7 DNA (deoxyribonucleic acid)-dependent RNA (ribonucleic acid) polymerase transcription from Escherichia phage T7 followed by expression of bacteriophage in yeast. There is also provided a method for the genetic manipulation of yeast (Kluyveromyces lactis and Pichia pastoris) cells to include transcriptional components from bacteria (Escherichia coli) and RNA (ribonucleic acid) polymerase (P) inside of yeast followed by expression of the bacteriophage in yeast.

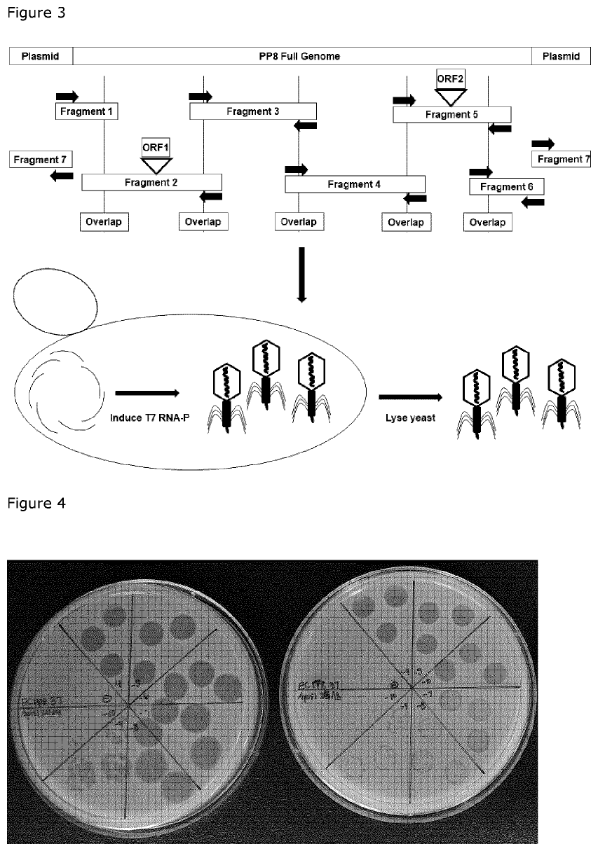

With reference to FIG. 3, the genomic compliment was divided into fragments with overlapping sections to adjacent fragments obtained by PCR amplification. Foreign genes were inserted within respective fragments. Fragments were combined via homologous recombination into full-length genomes and a yeast-based plasmid (as an additional PCR fragment) with a T7 promoter inside of yeast strain Pichia pastoris. The stable plasmid under T7 promoter control drove the rescue of bacteriophages upon induction of the P. pastoris which contains T7 RNA polymerase cells are then lysed using enzymatic and mechanical means to release fully-formed bacteriophage particles.

Homologous recombination of EV31pYes (unmodified) with pYESIL vector was achieved using 100 ng of each PCR product and transformed into chemically-competent yeast cells. pYESIL vector (100 ng) and EV31 (100 ng) were combined. Competent yeast cells were added and mixed gently followed by the addition of 600 μl of polyethylene glycol (PEG) and lithium acetate (LiAc) solution then mixed gently. The mixture was incubated at 30C for 30 minutes, inverting in 10 minutes intervals. Immediately after incubation, 35.5 μl of dimethyl sulfoxide (DMSO) was added, mixed by inversion and subjected to heat-shock for 20 min at 42C (with occasional inversion). Tubes were then centrifuged at 200-400 xg for 5 minutes, supernatant was discarded and the cell pellet was resuspended in 1 ml sterile 0.9% sodium chloride (NaCl). Visualization of transformation was achieved by spread-plating 100 μl onto selective agar plates (media without tryptophan) and a 3-day incubation period at 30C. Colony-PCR screening can determine the presence of positive transformants. Homologous recombination was achieved by standard cloning techniques to make S. cerevisae strain 5150, chemically-competent. Briefly, using the Gietz and Schiestl 2007 protocol, a spread plate of a single yeast colony from stock was created and incubated overnight at 30C. The next day, 50 μl equivalent of cells was scraped and washed in a tube with 1 ml of sterile nuclease-free water followed by a 13,000xg spin for 0.5 minutes. The following was added to the cell pellet in order: 240 μl of PEG-3350 (50% w/v), 36 μl LiAc (1M), 50 μl single—stranded carrier DNA (2 mg/m1 of pig sperm) and 34 μl plasmid-nuclease free water mixture (<lug plasmid). It was gently vortexed to mix, incubated at 42C for 20-180 minutes (timing is dependent on strain). For EV31, 45 minutes was used. After transformation, it was spun for 13,000xg for 0.5 minutes, then the supernatant was removed and the pellet was resuspended in 1 ml sterile nuclease-free water. The mixture was spread onto selective media plates, yeast synthetic drop out media without uracil and incubated at 30C for 3-4 days. Verification of clone was carried out using Colony-PCR screening.

FIG. 4 shows the titration of PP8 after rescue from the genetic template.

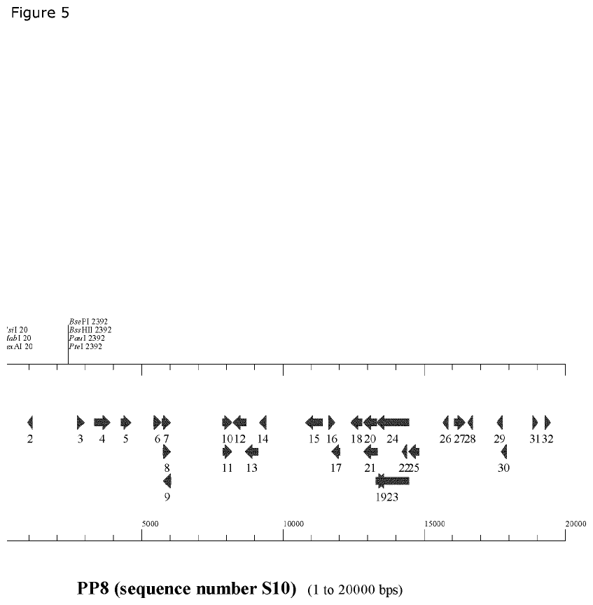

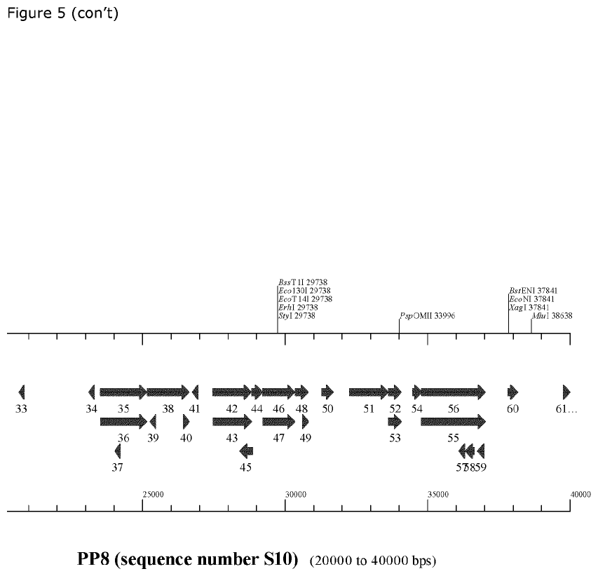

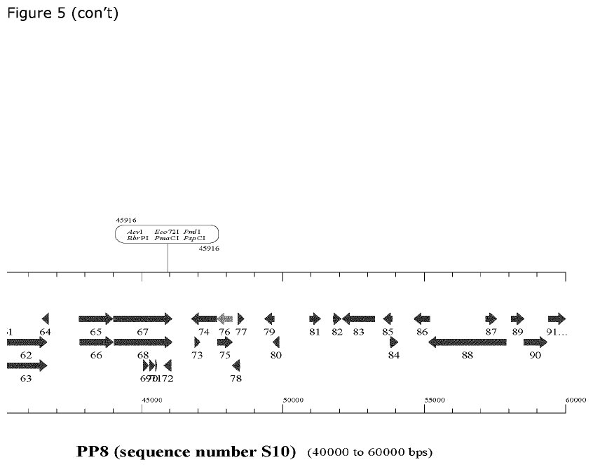

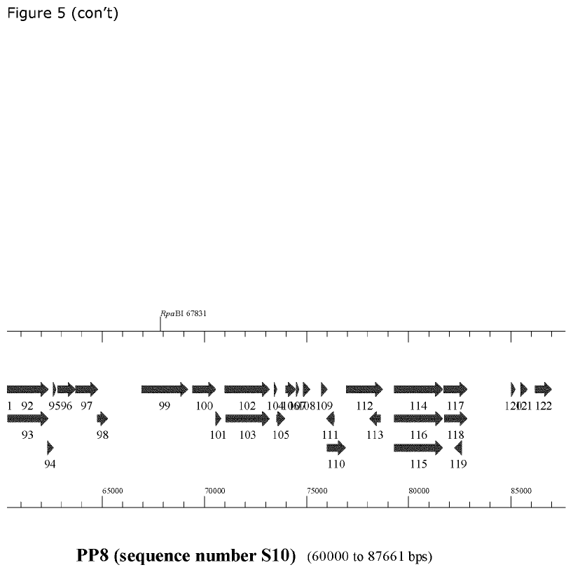

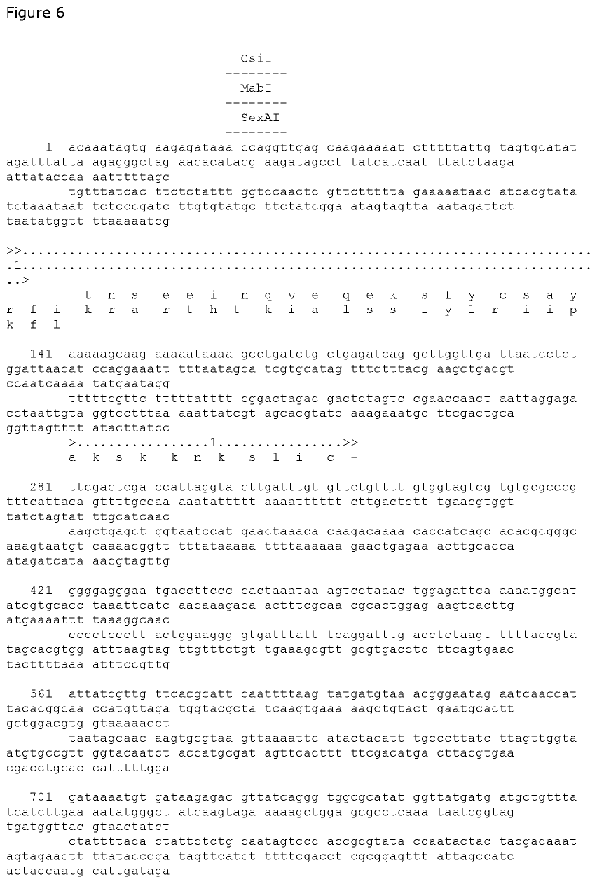

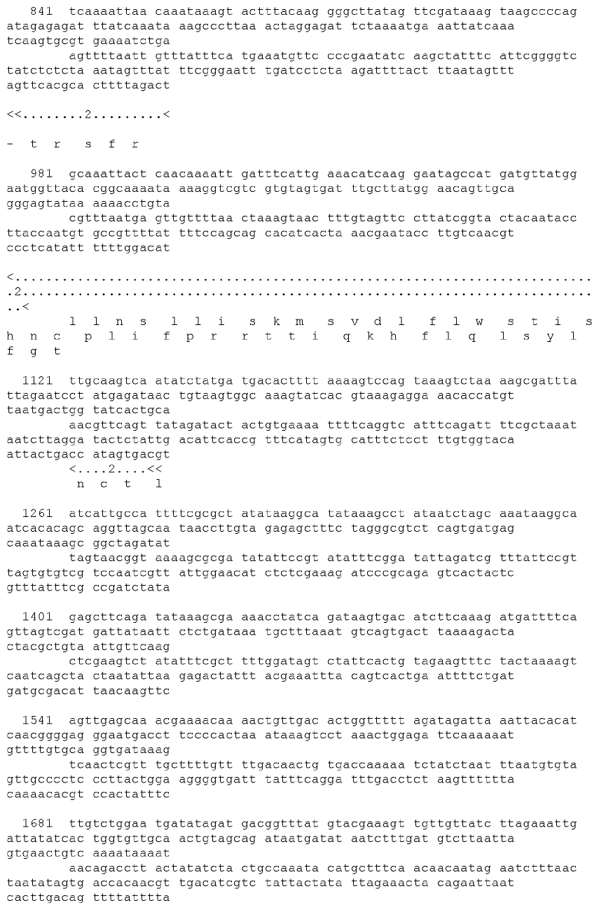

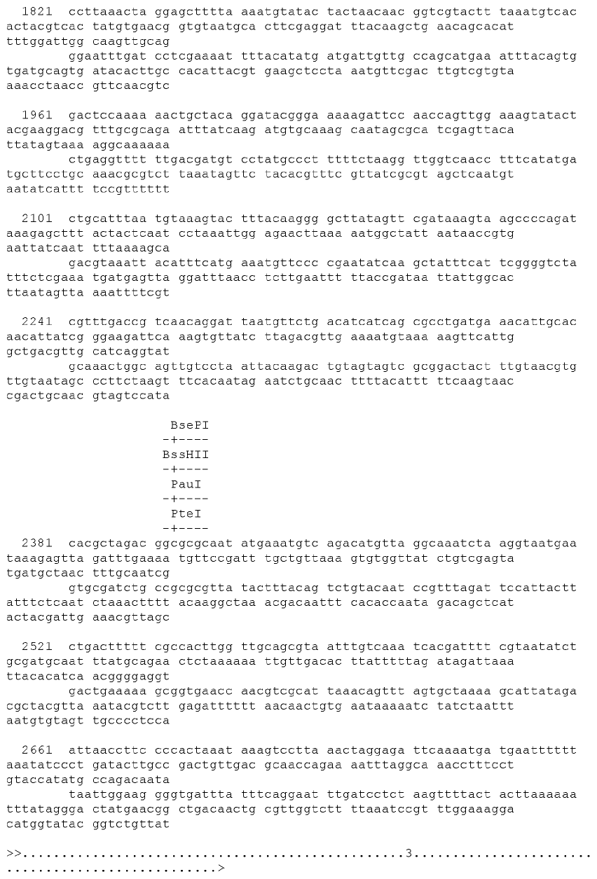

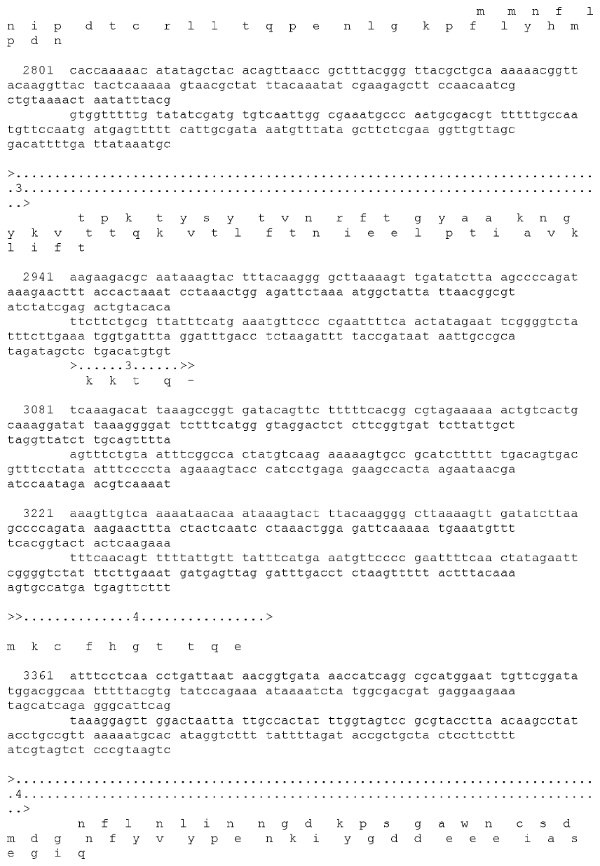

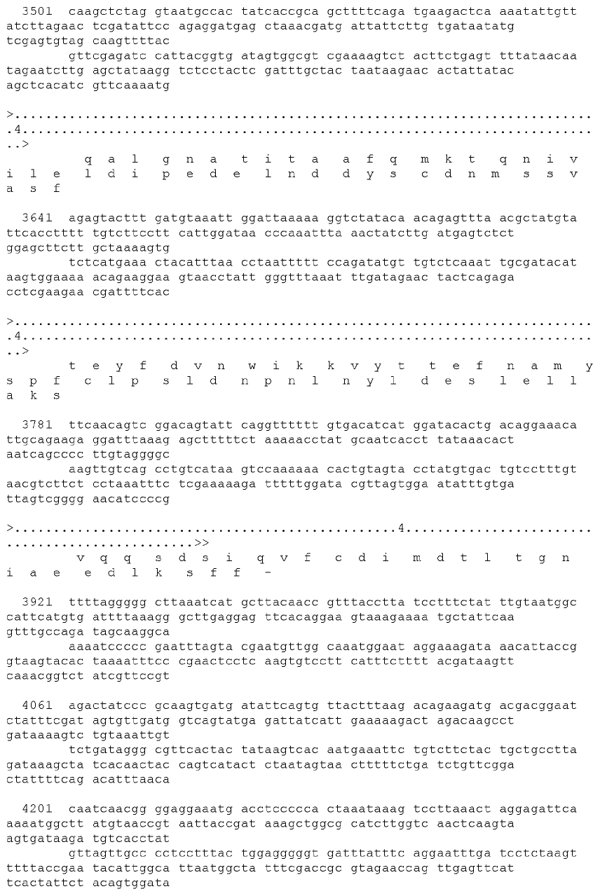

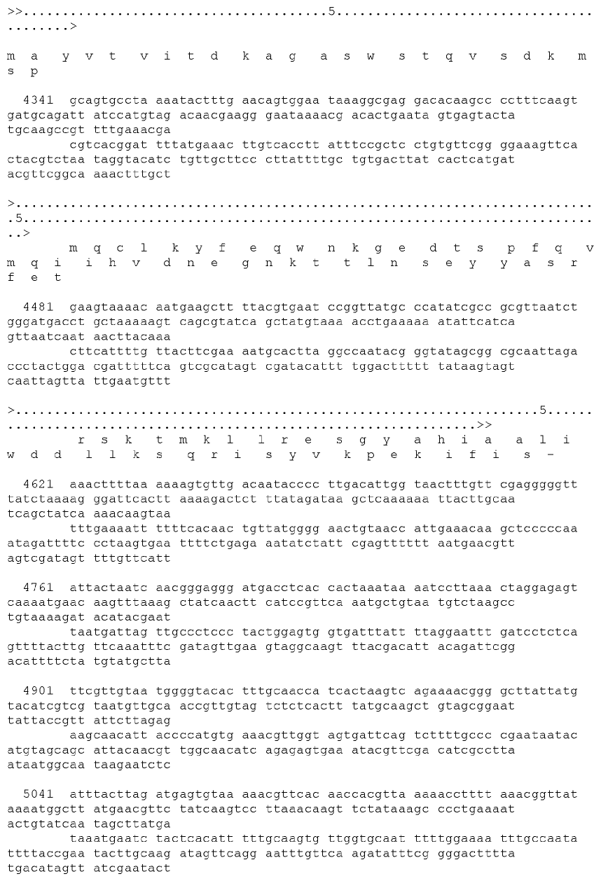

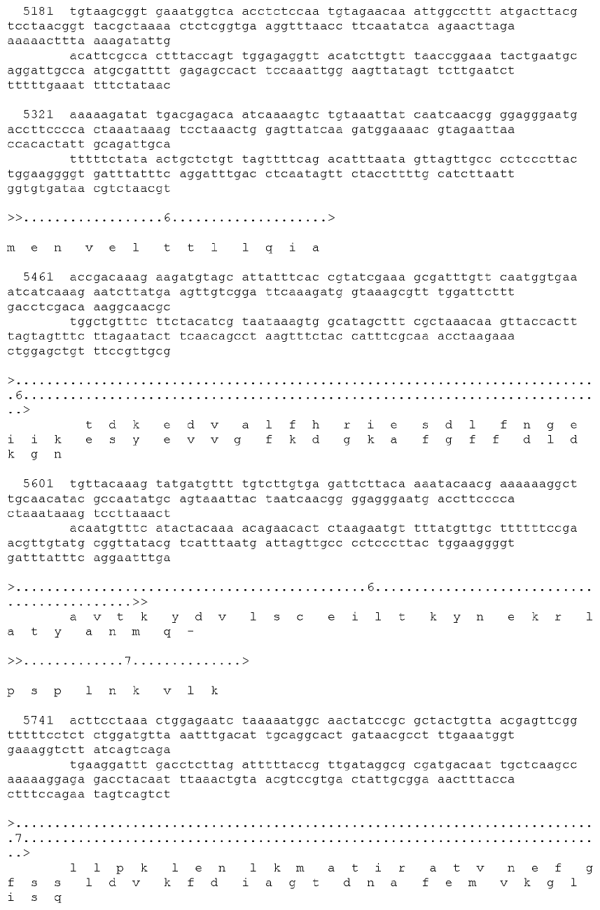

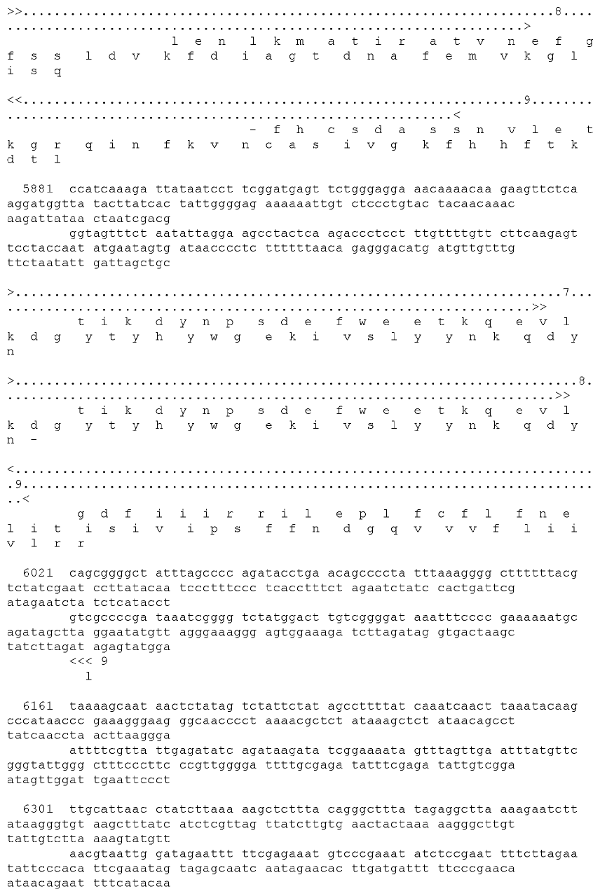

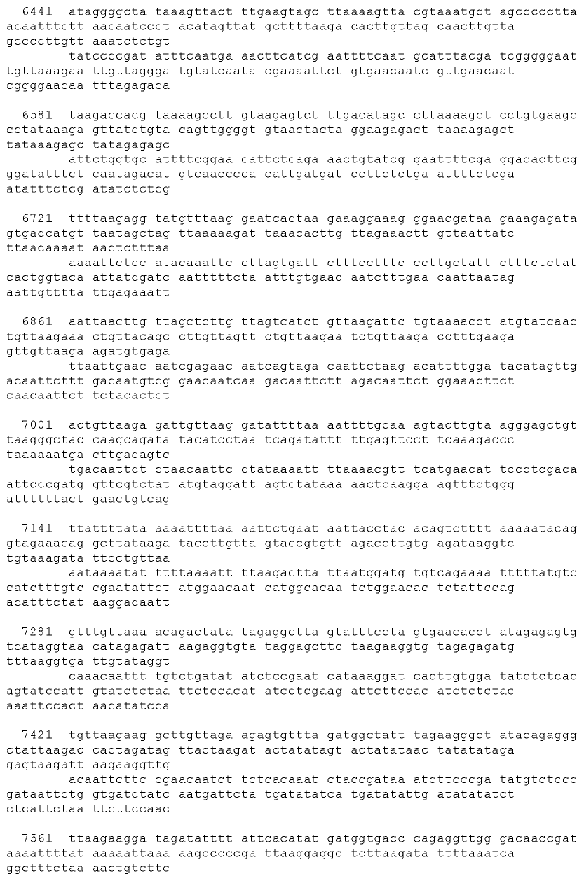

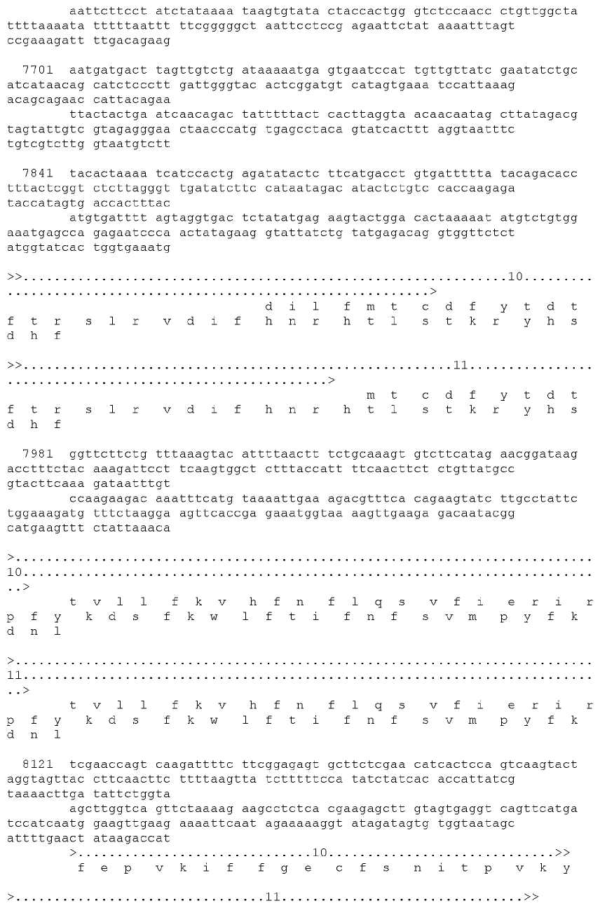

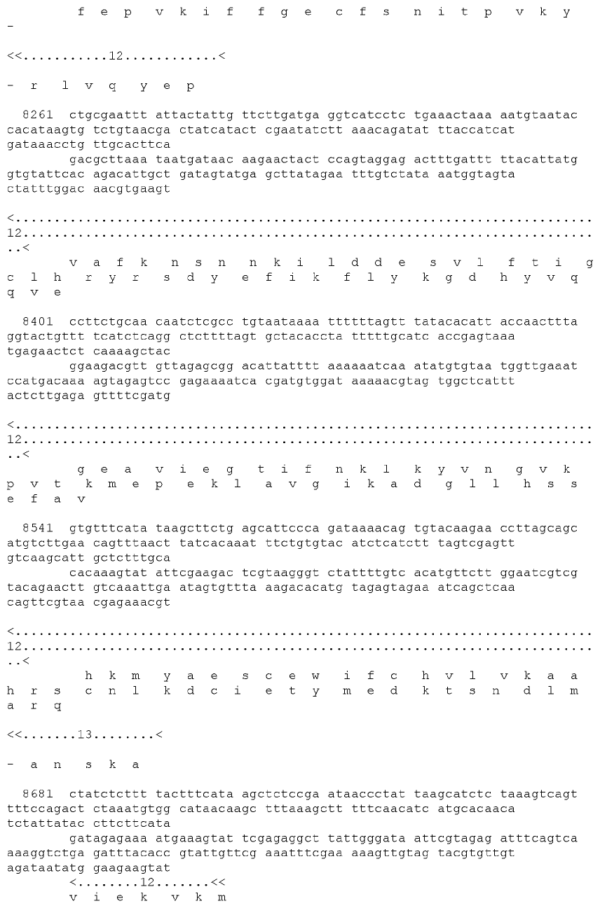

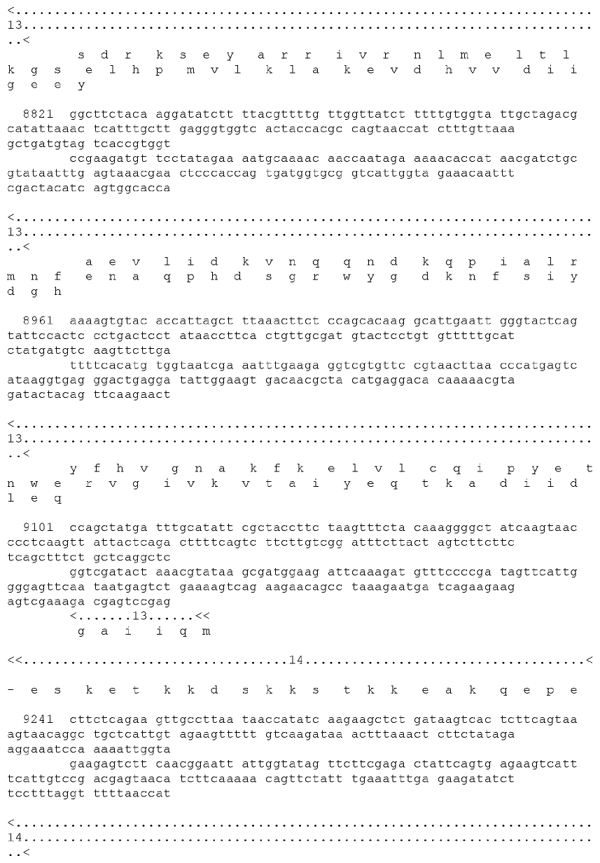

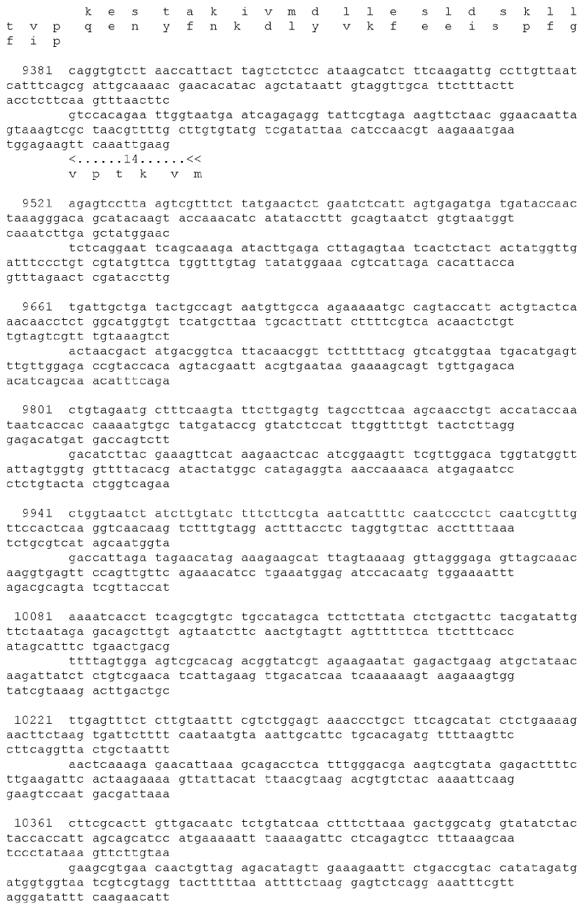

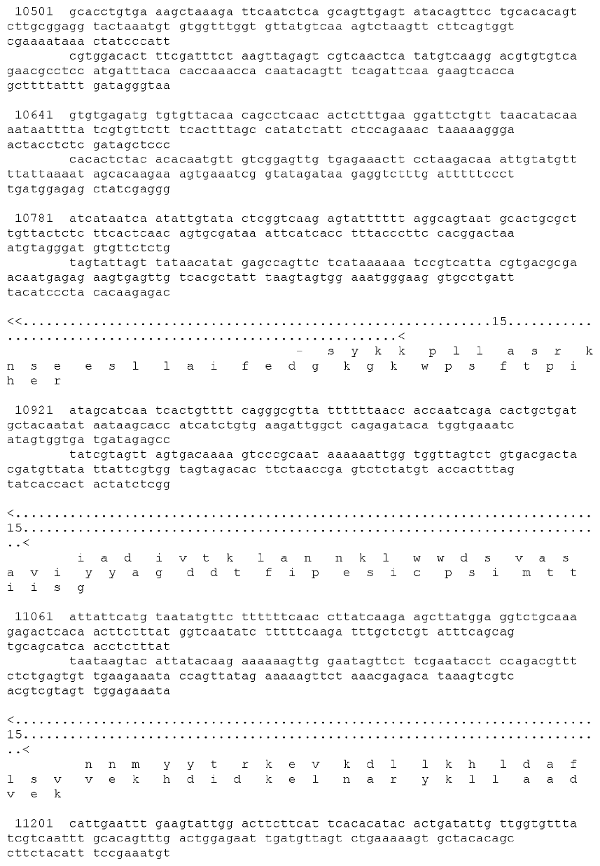

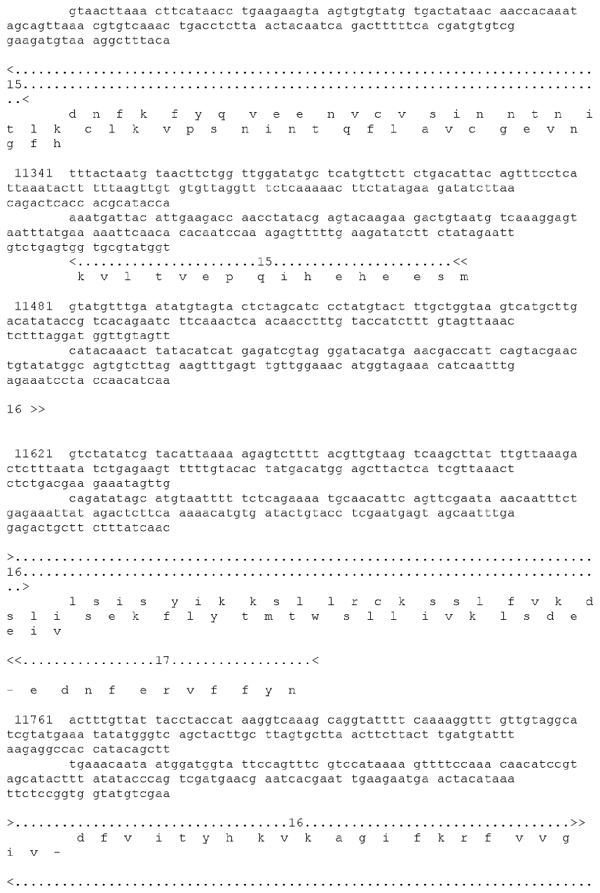

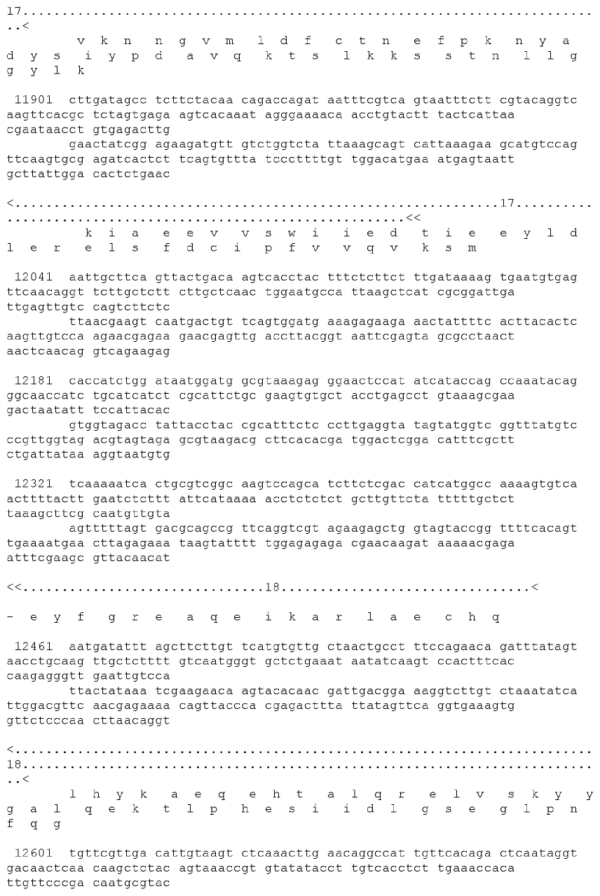

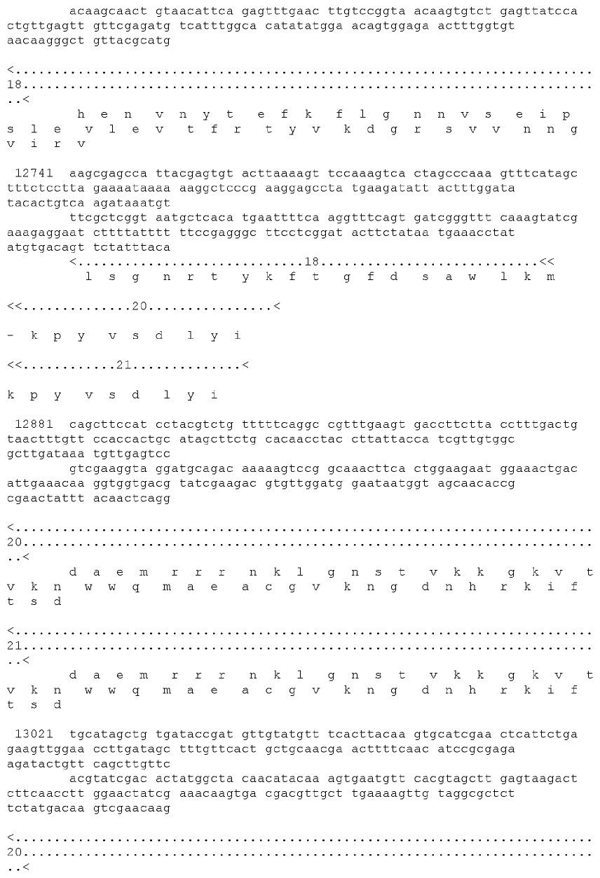

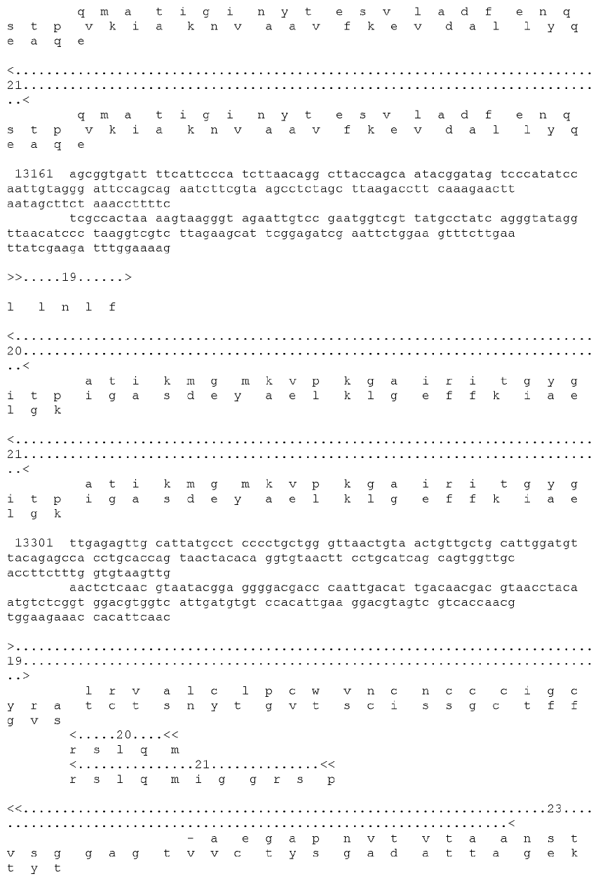

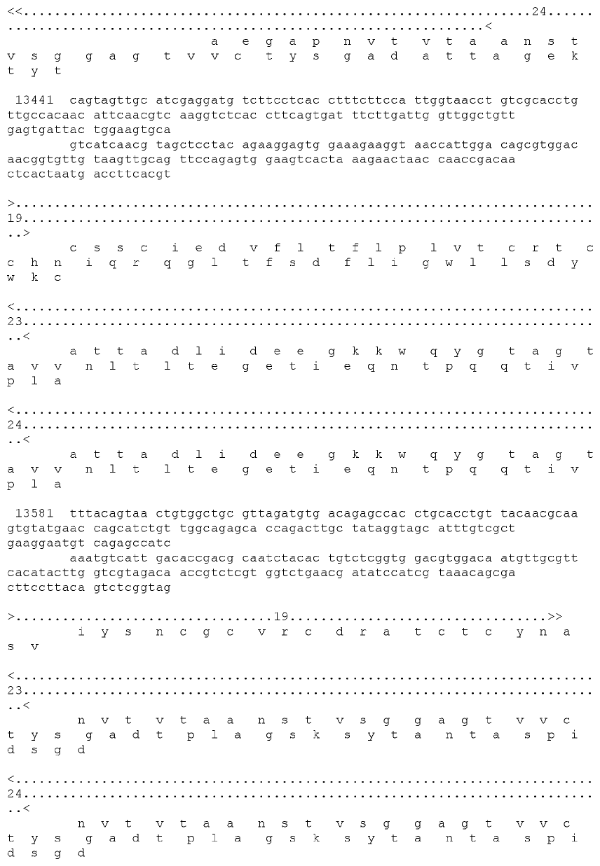

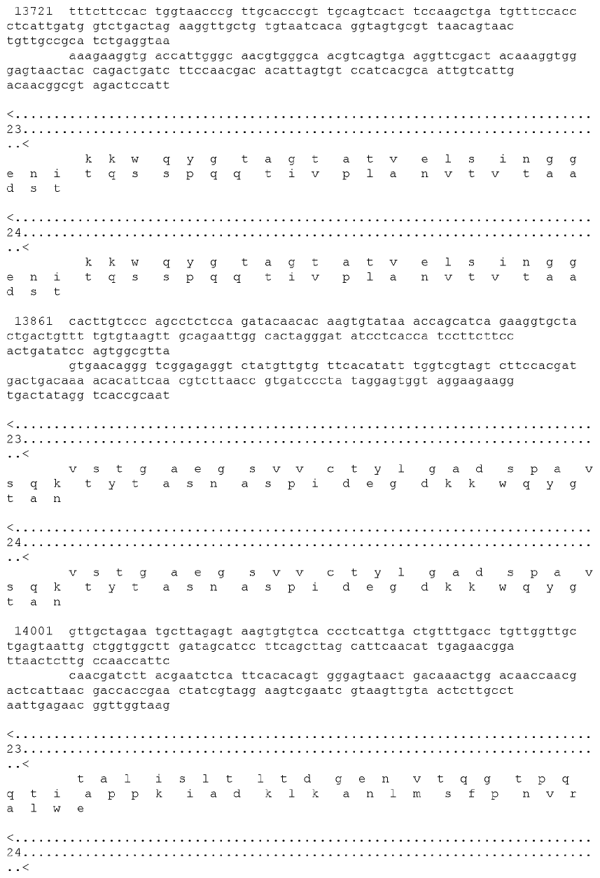

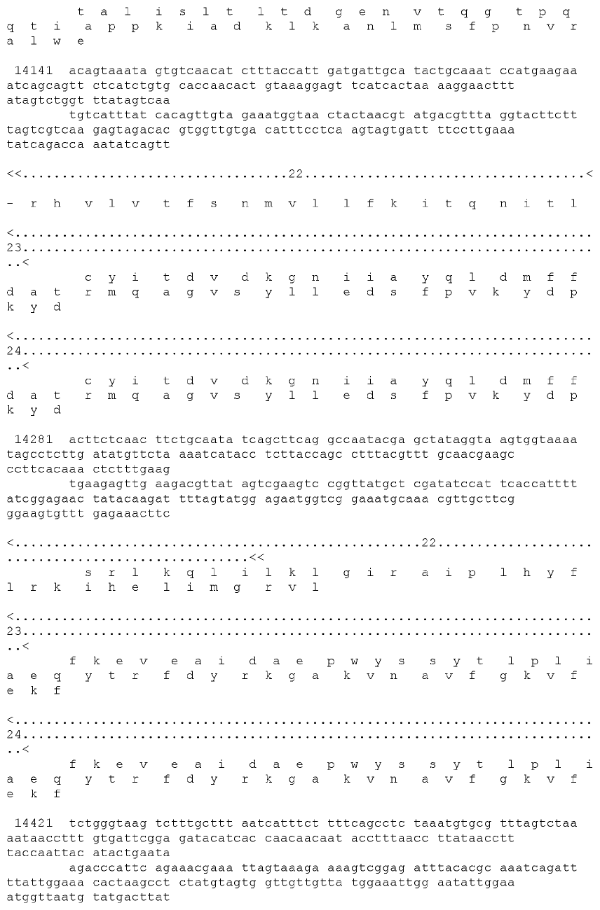

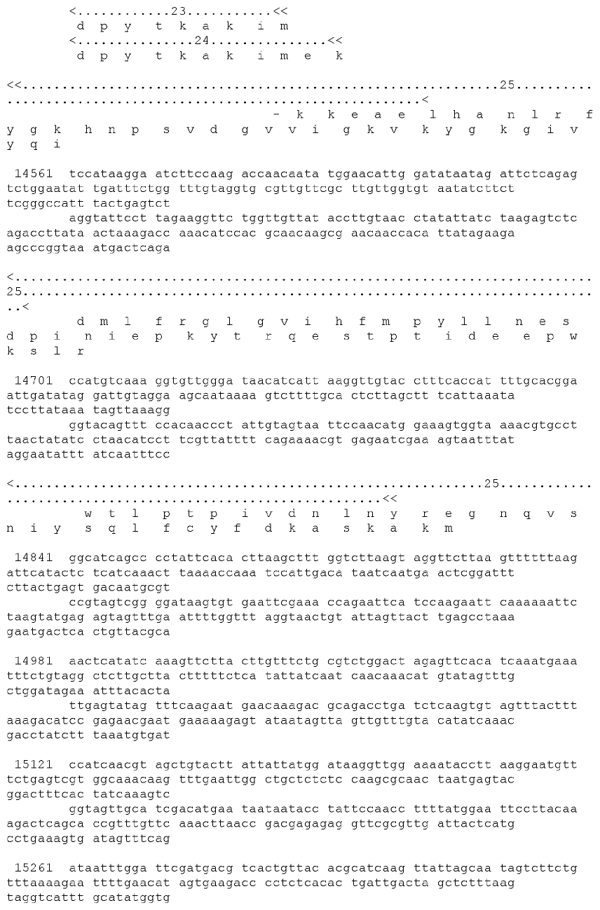

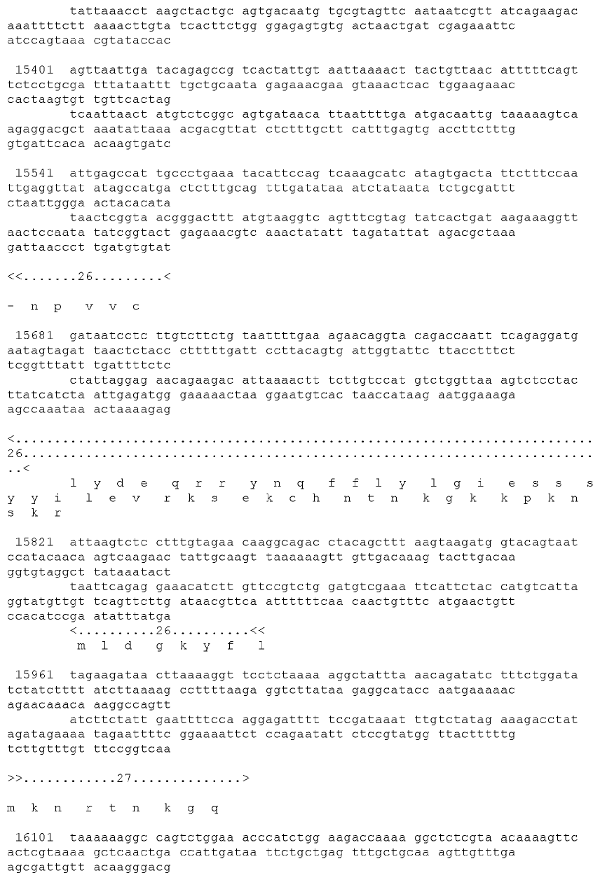

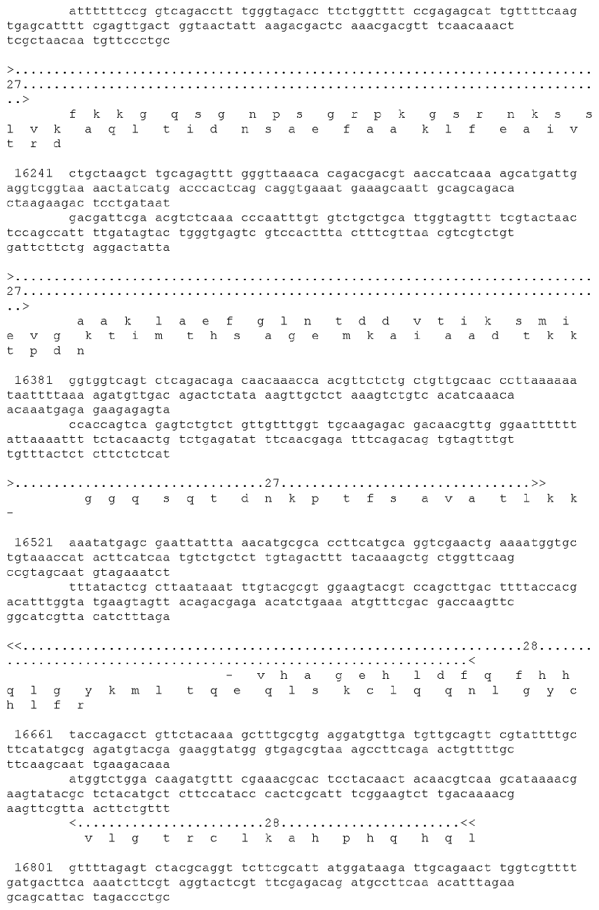

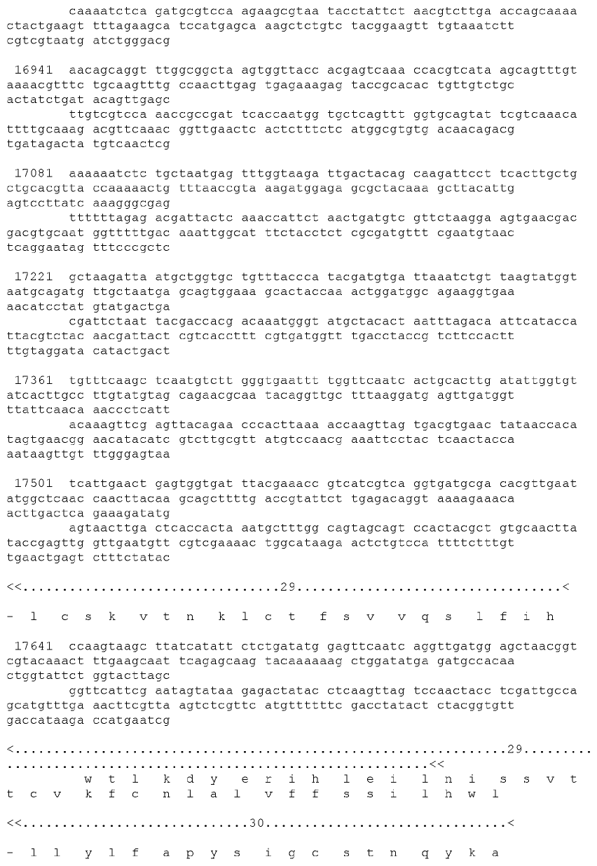

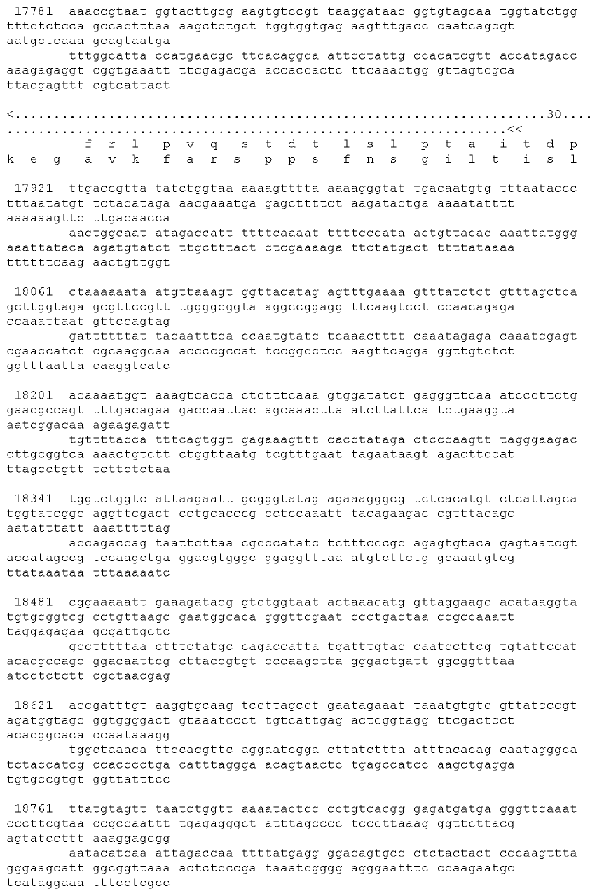

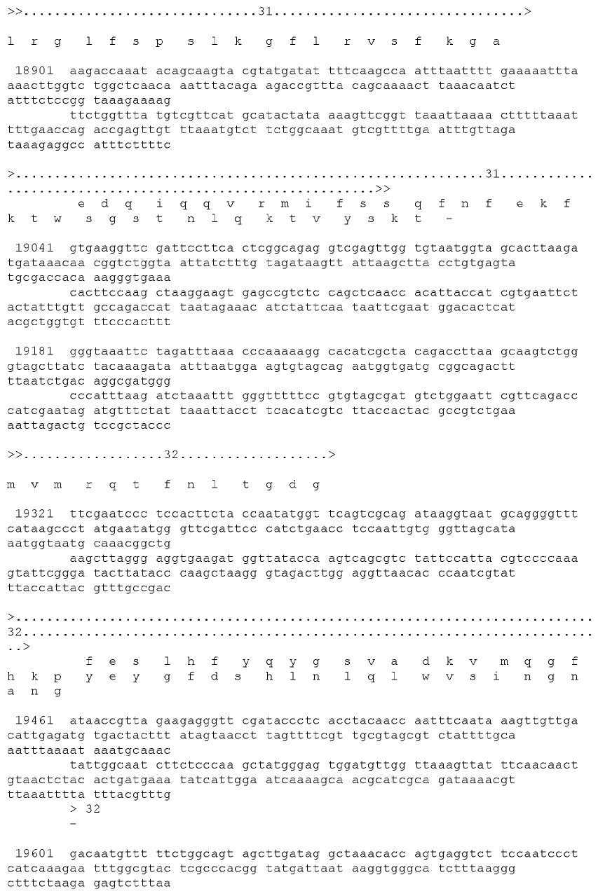

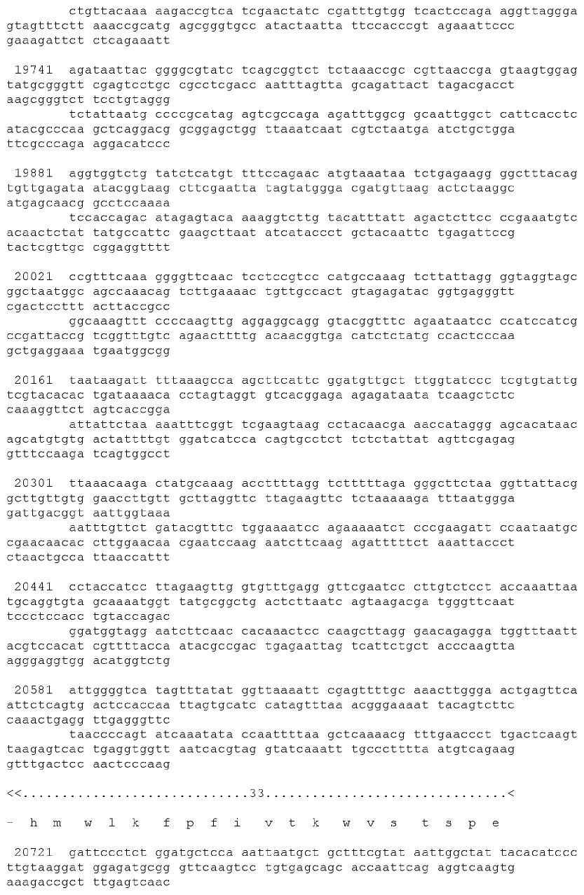

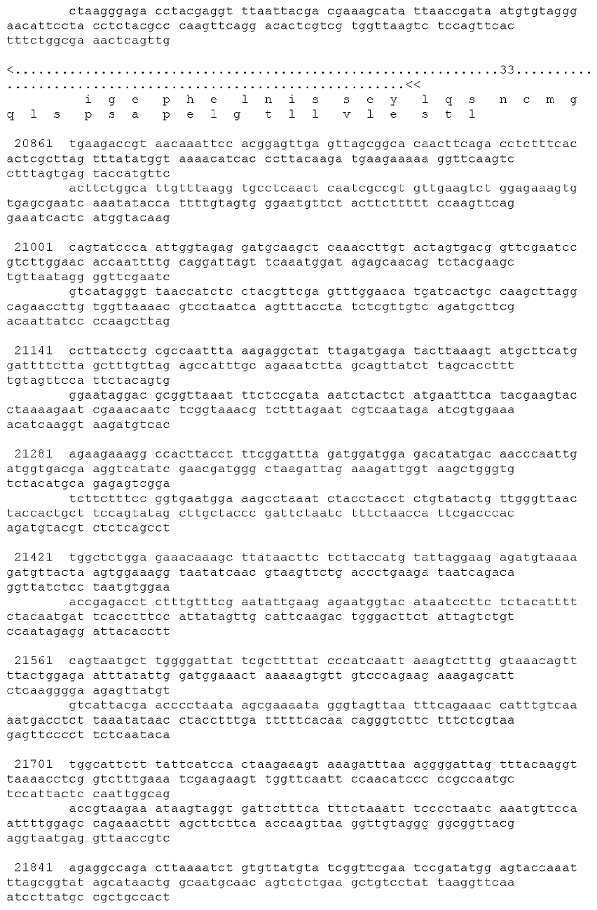

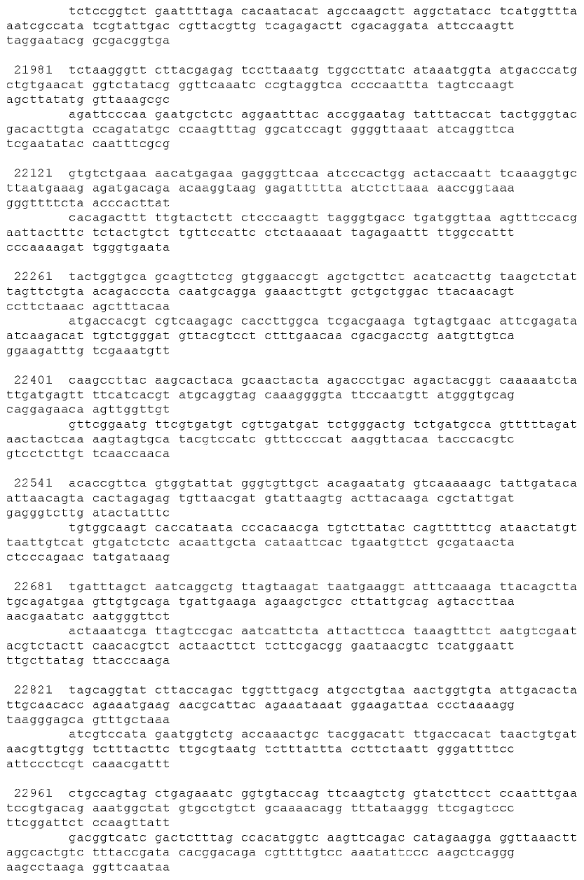

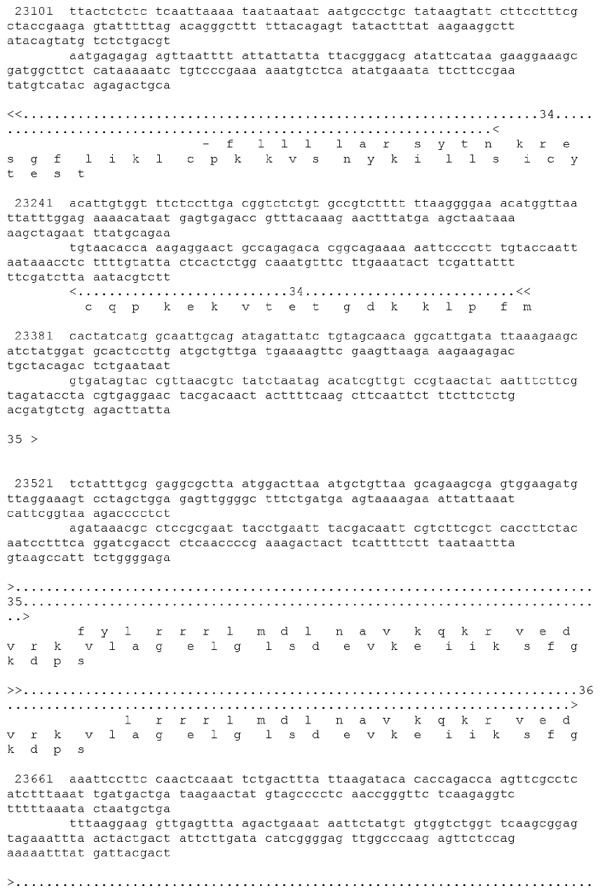

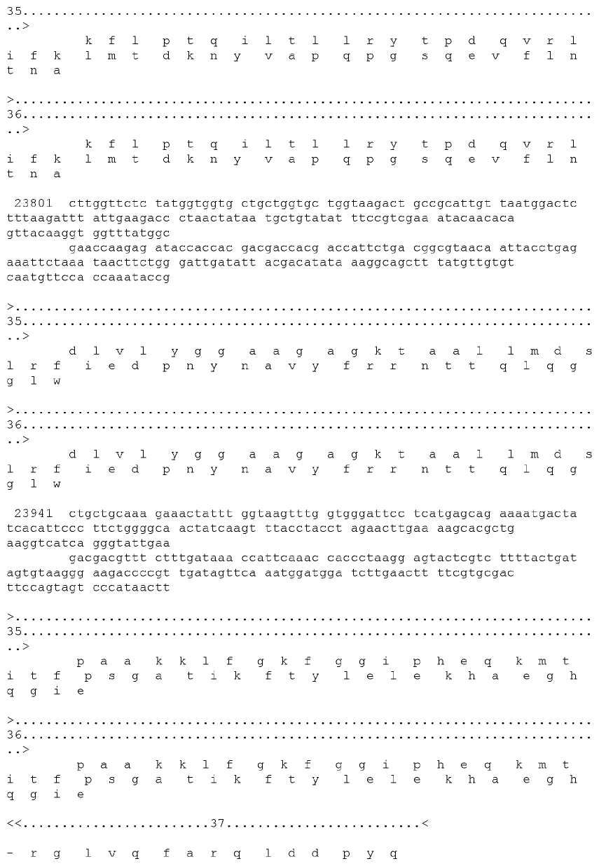

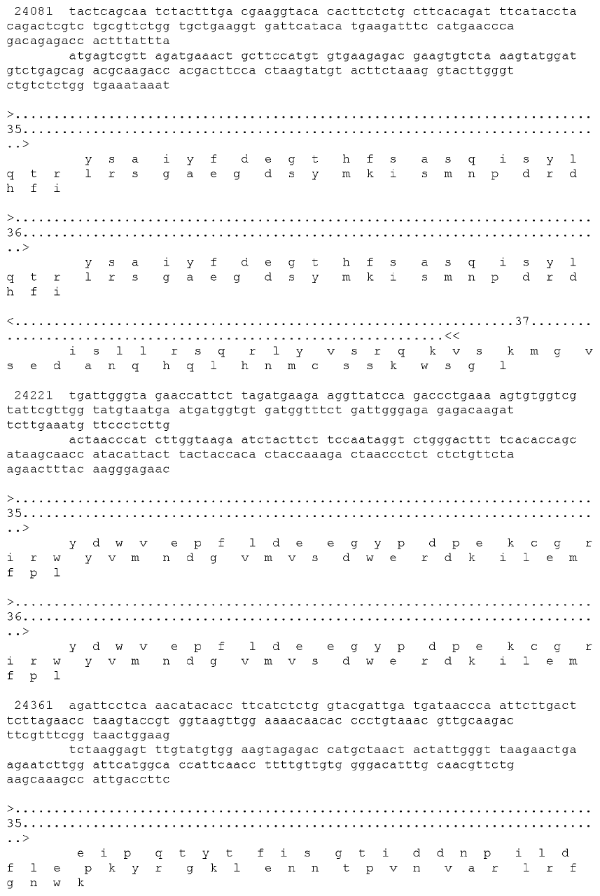

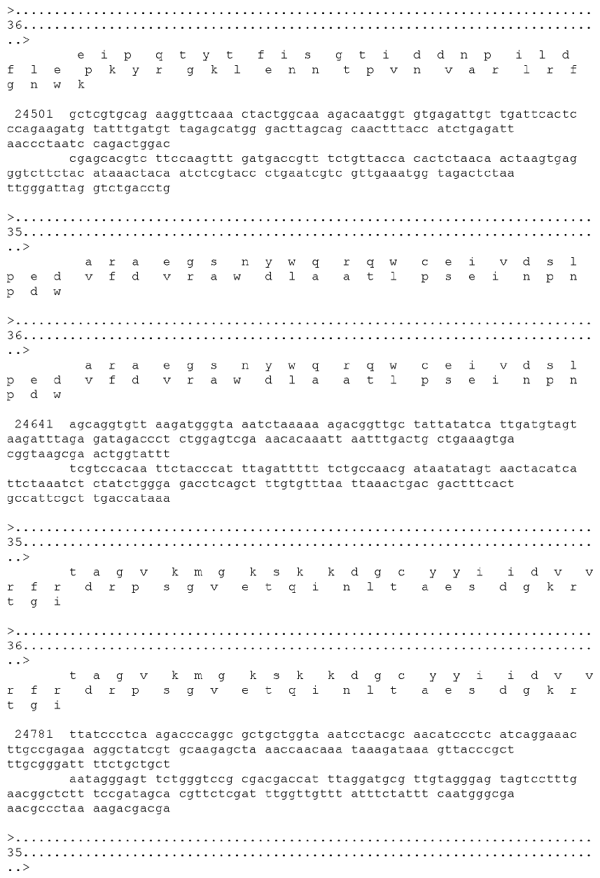

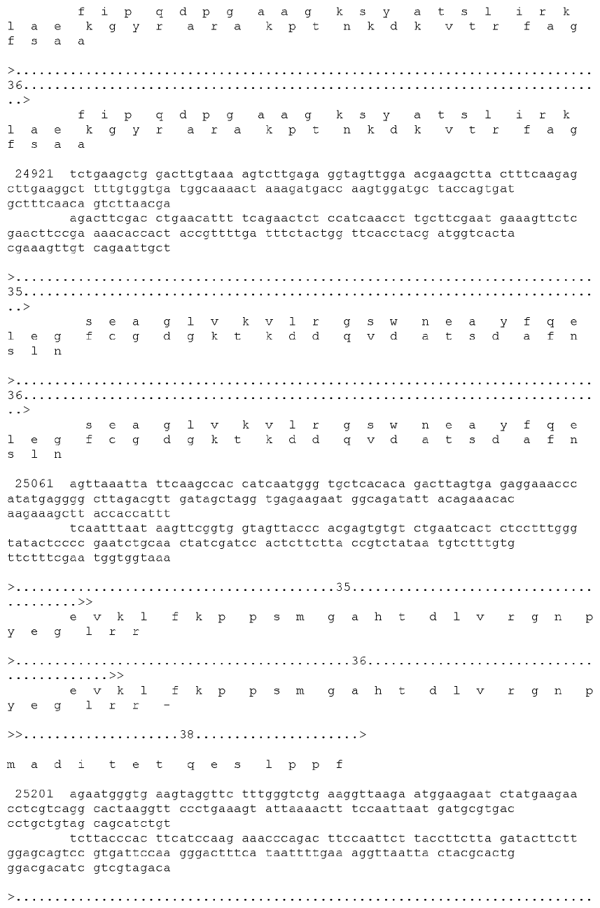

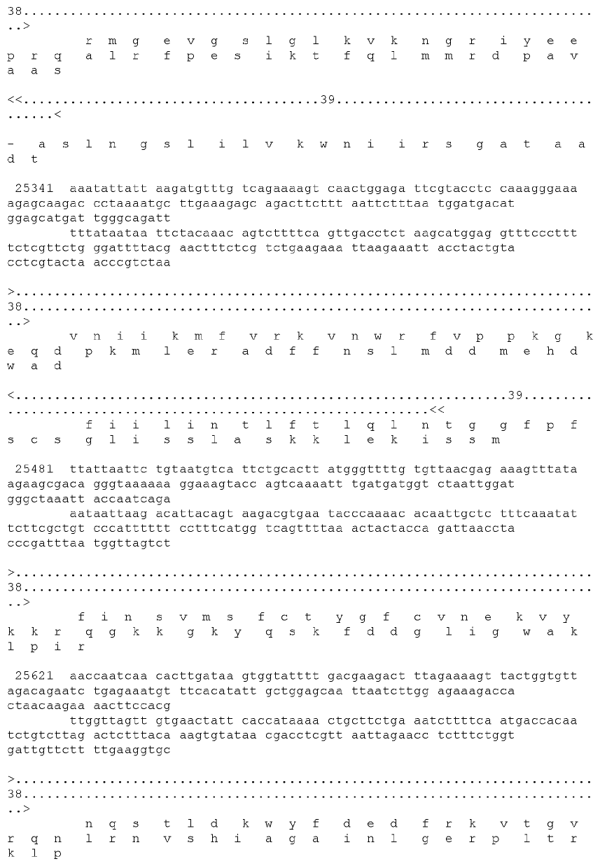

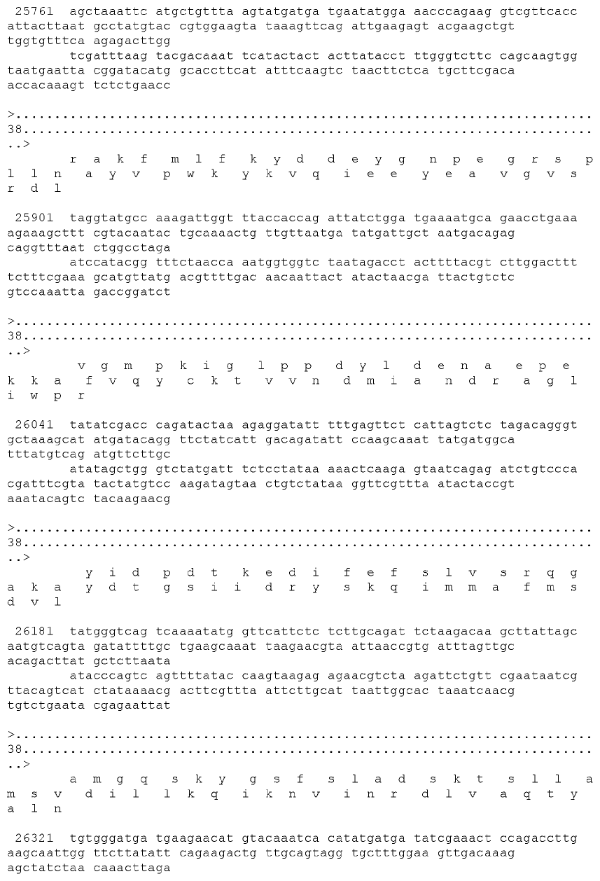

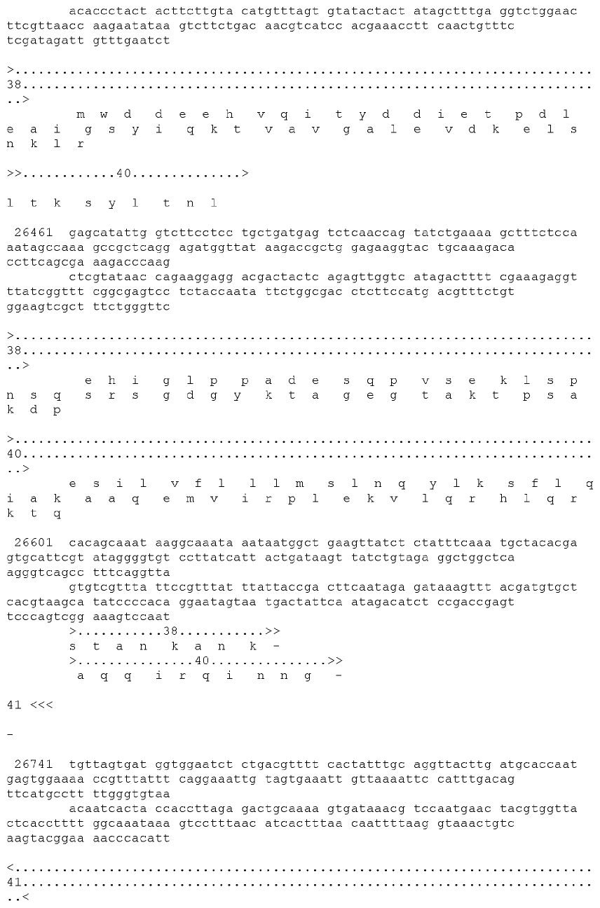

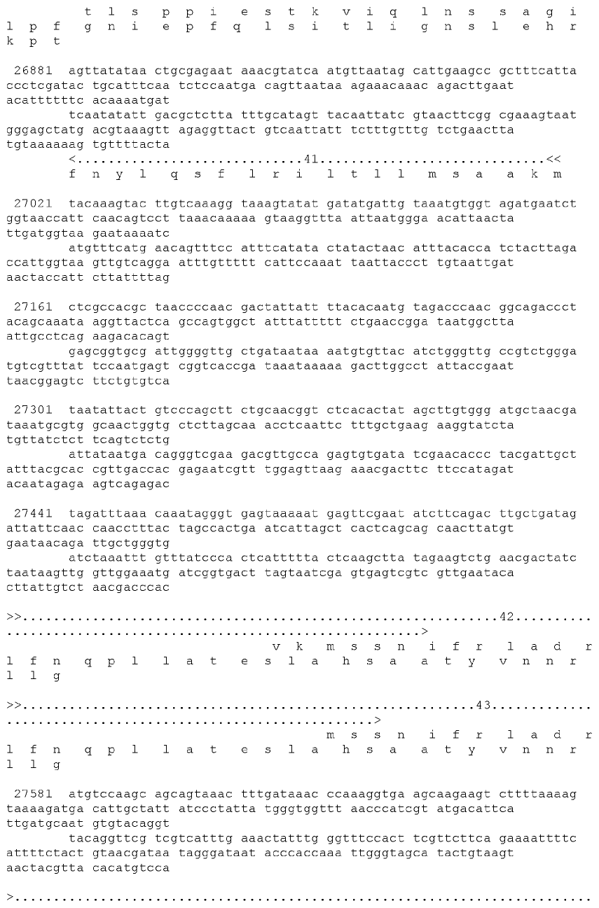

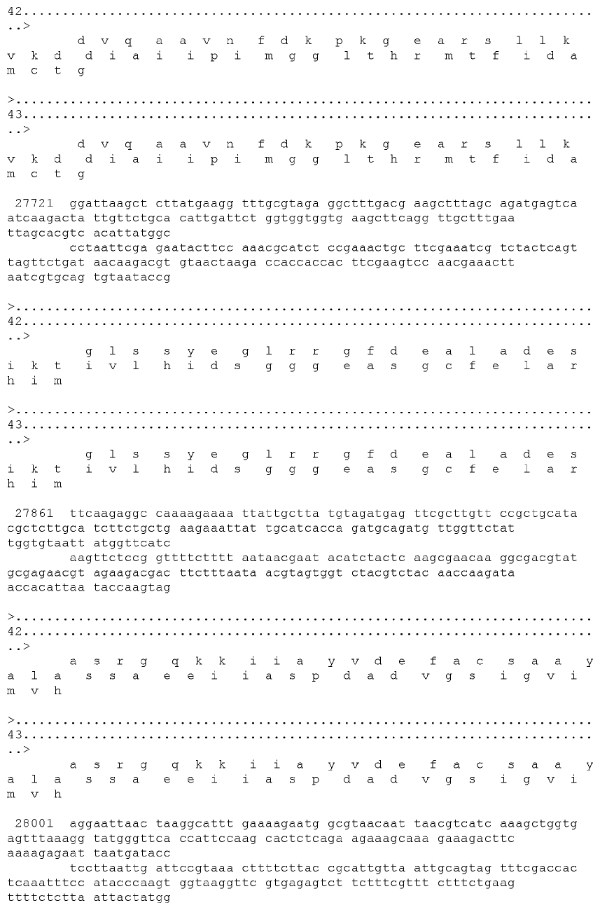

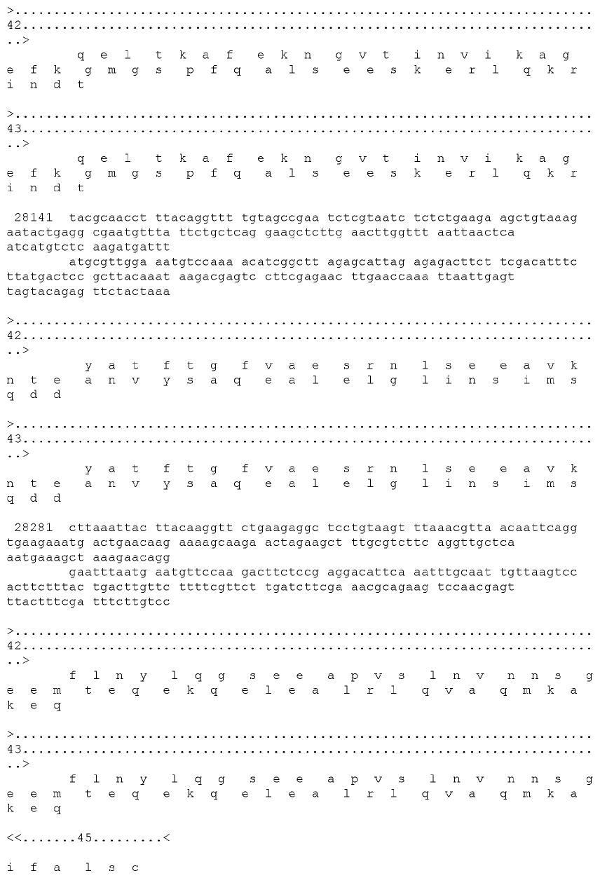

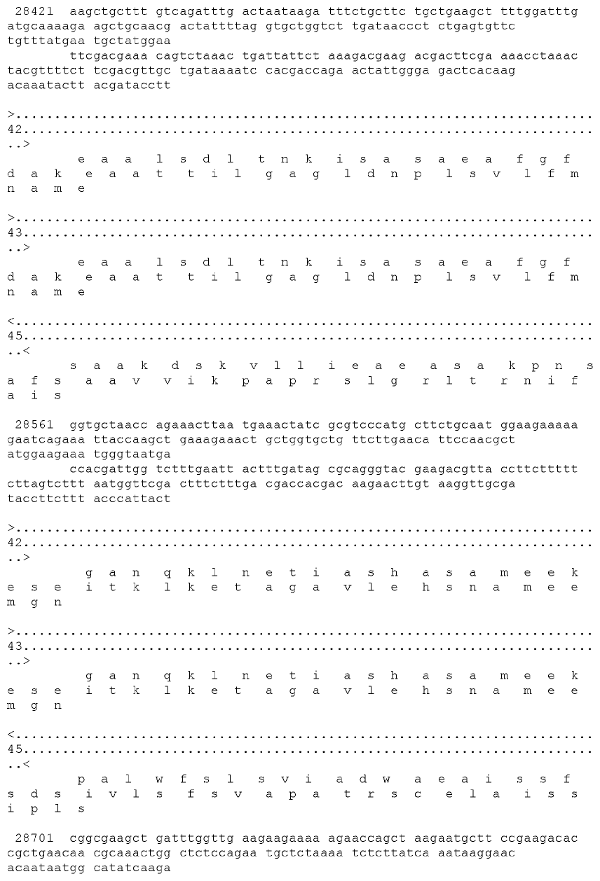

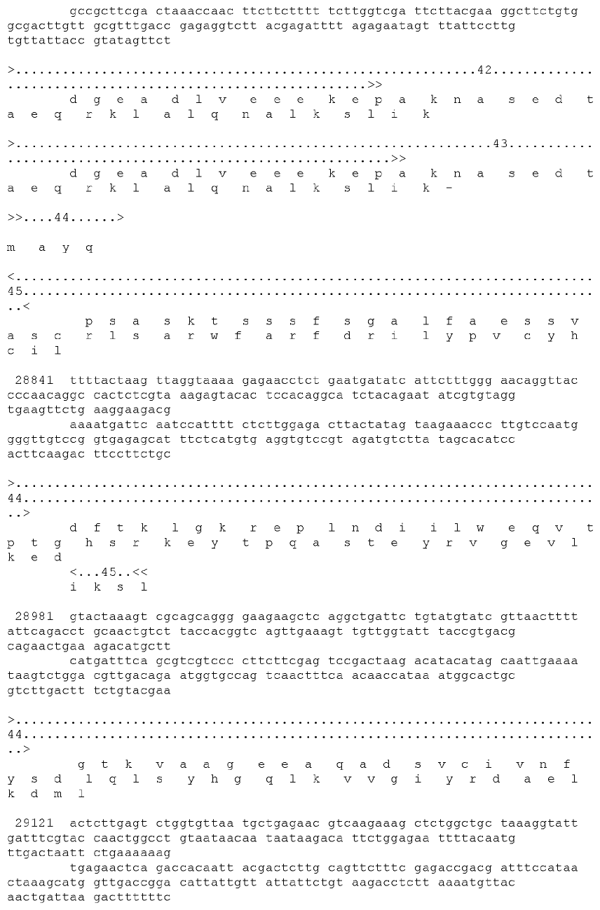

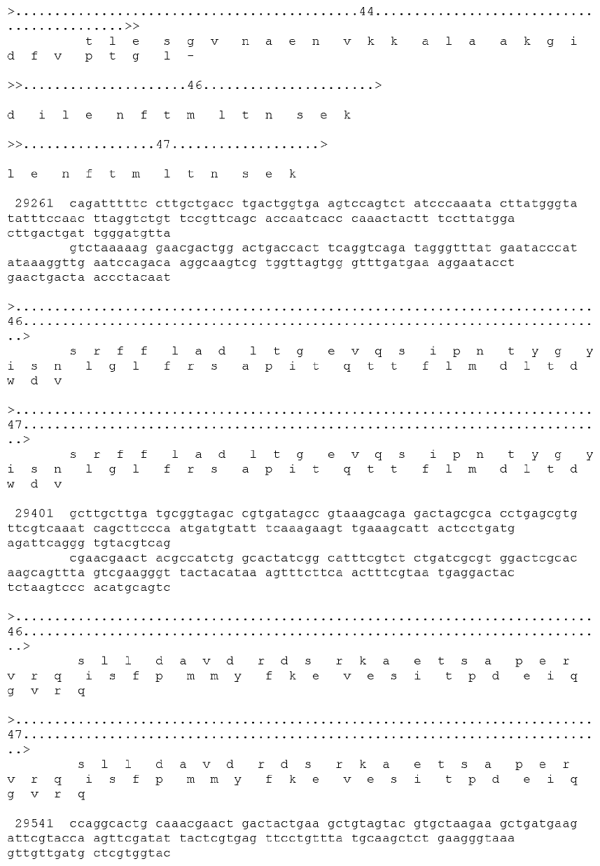

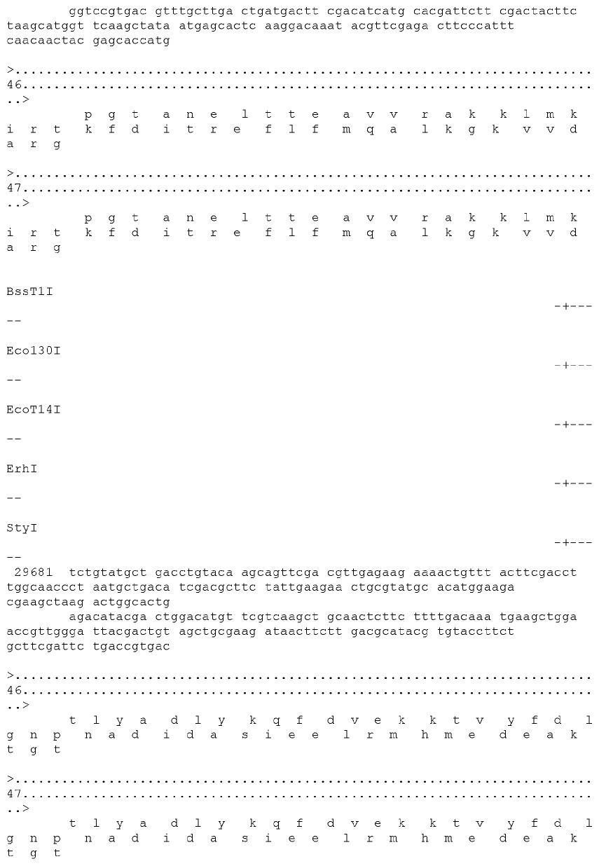

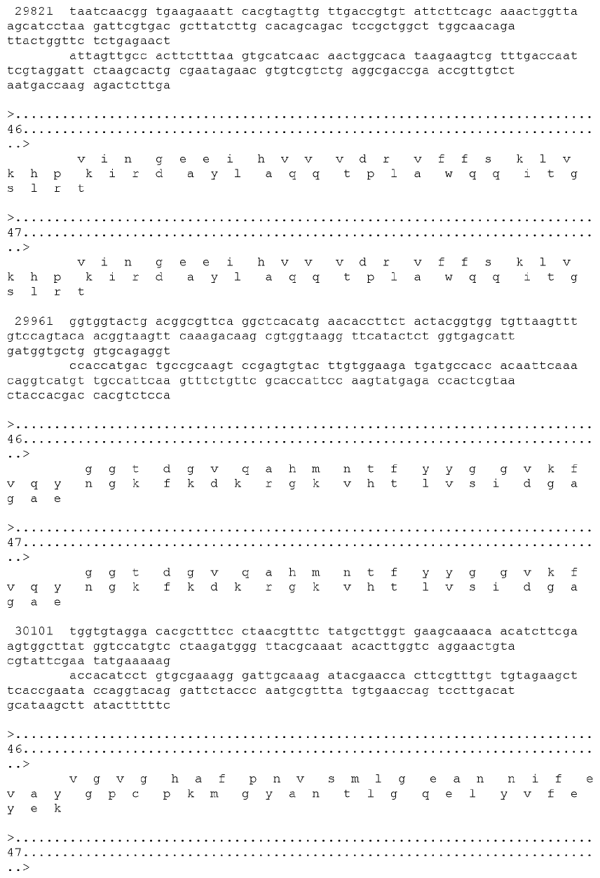

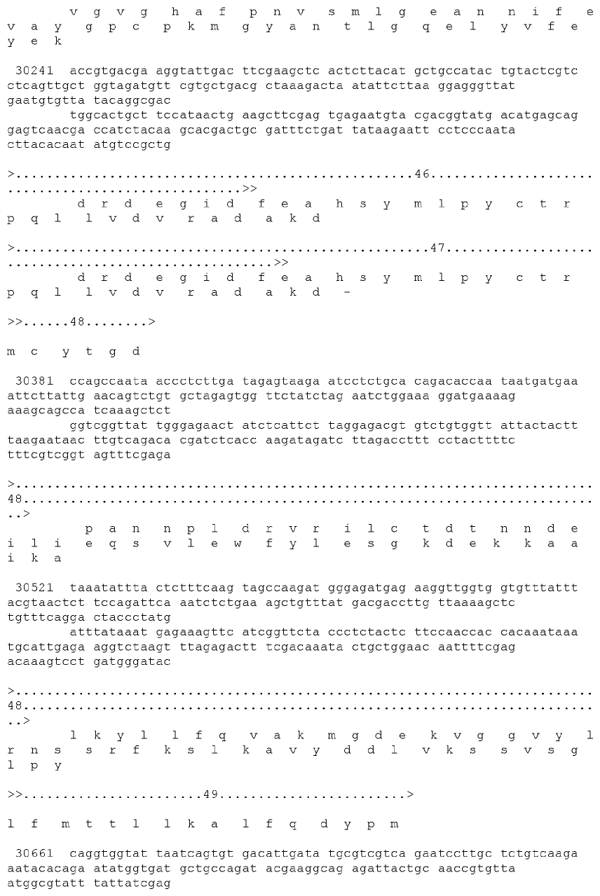

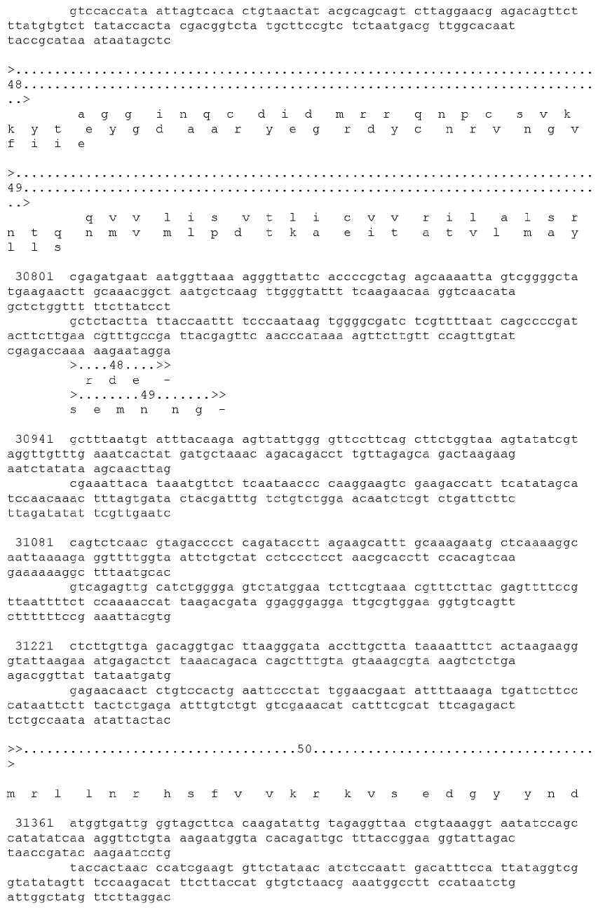

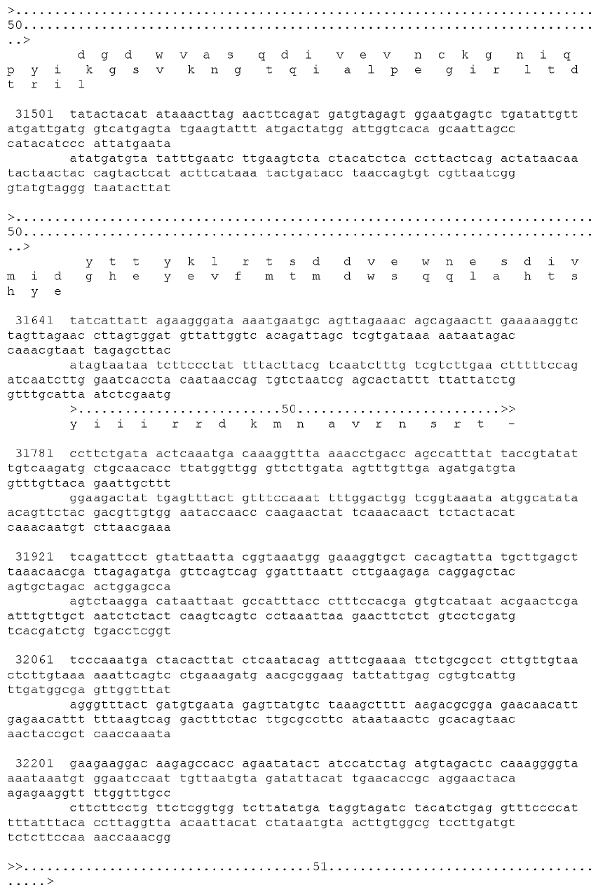

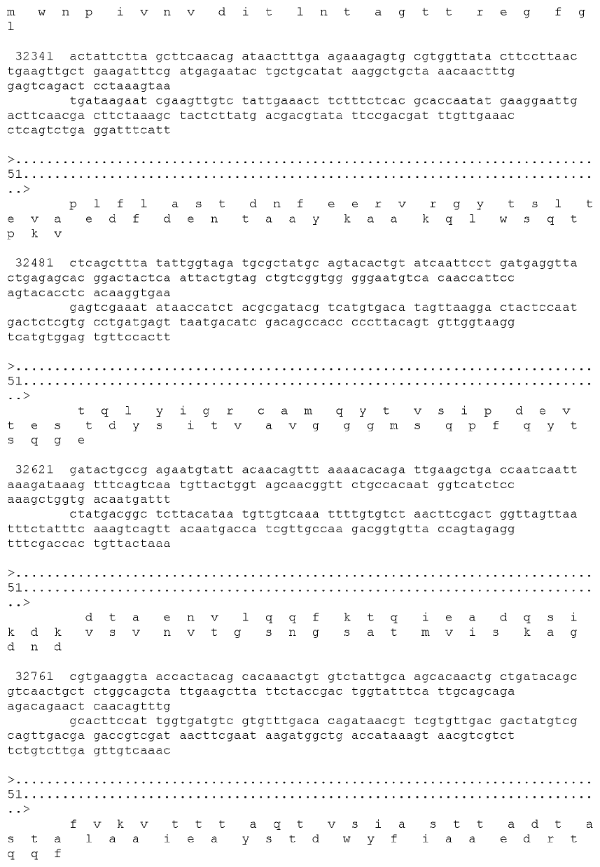

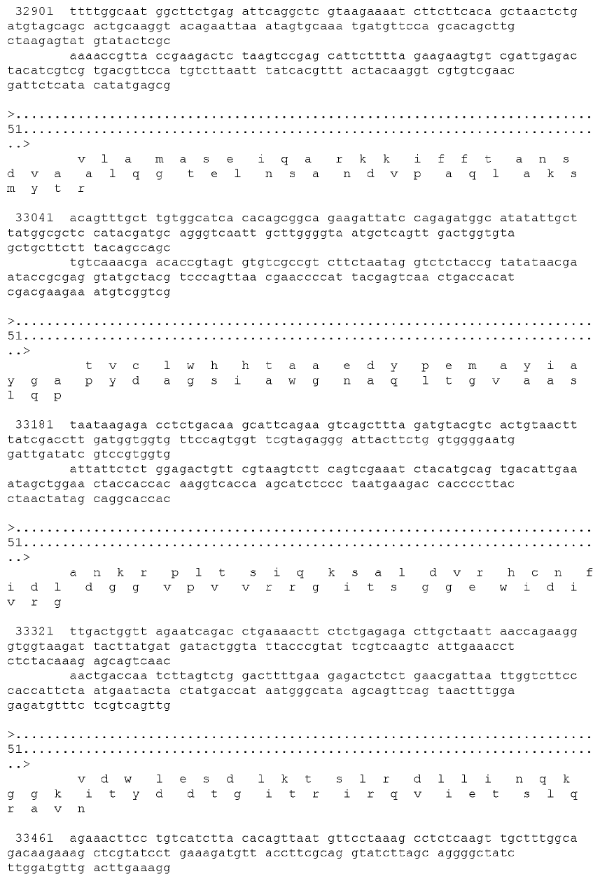

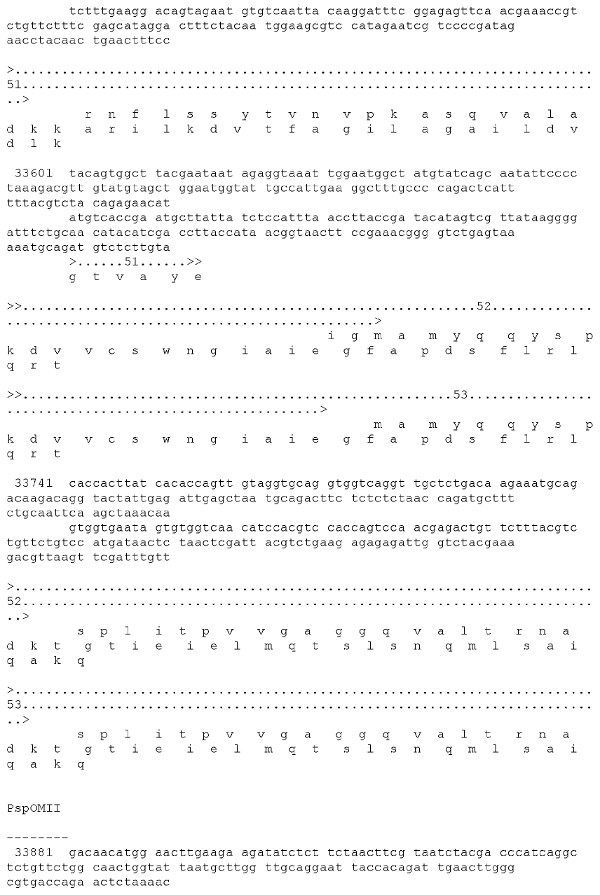

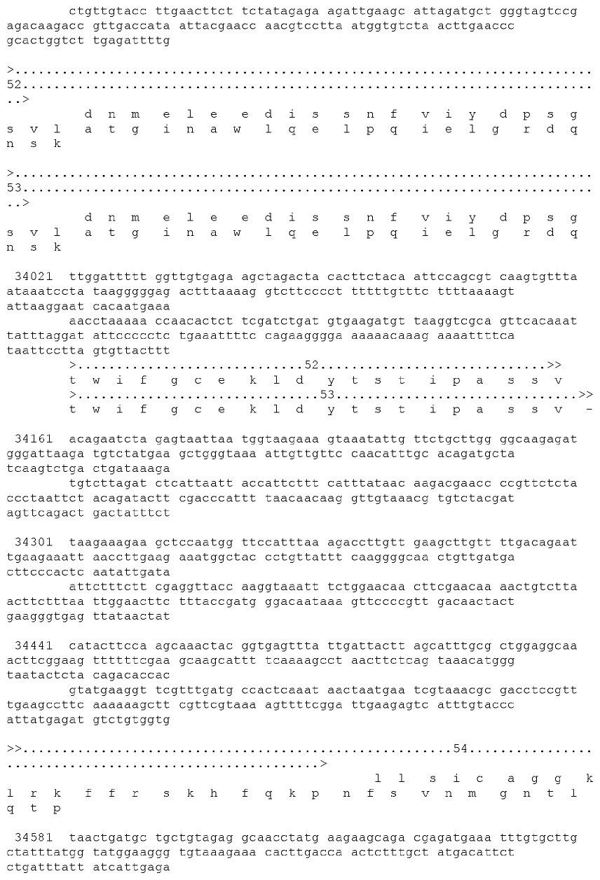

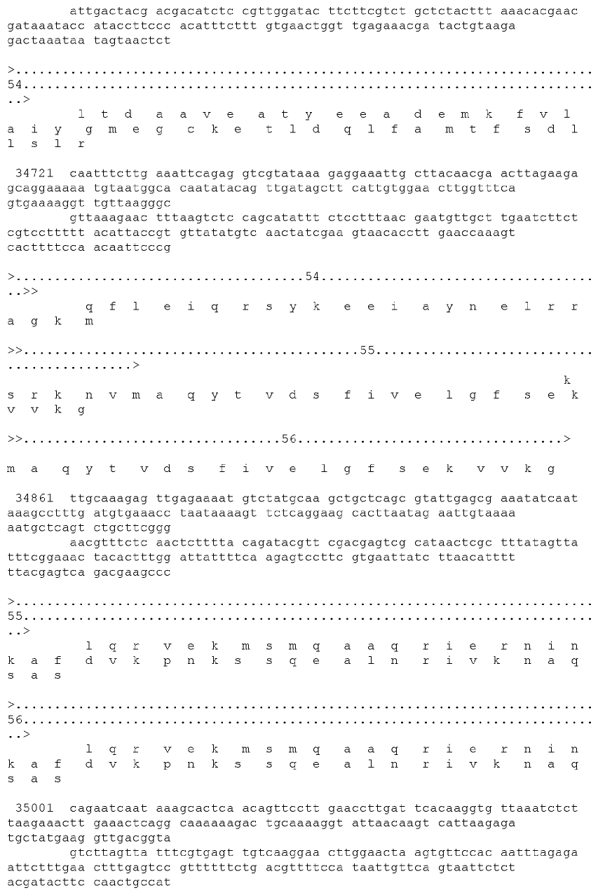

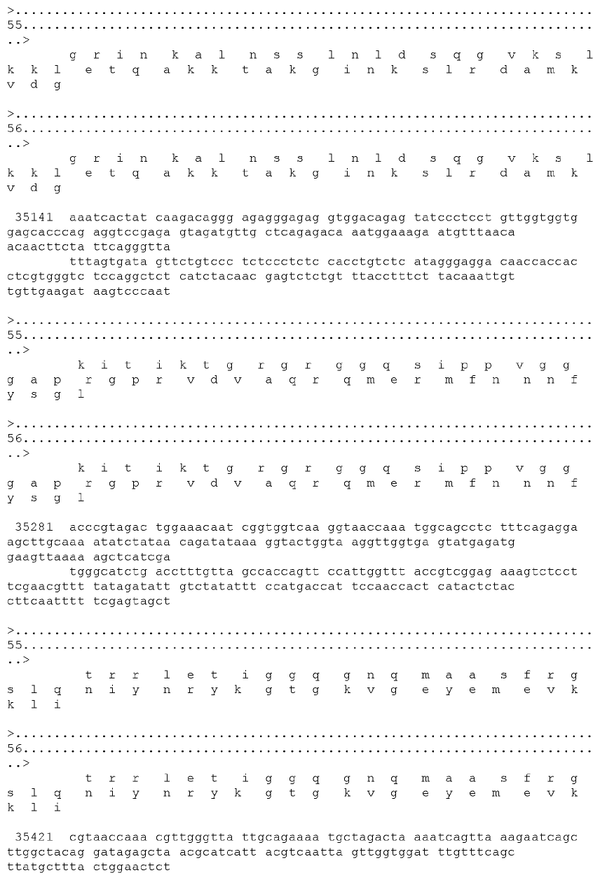

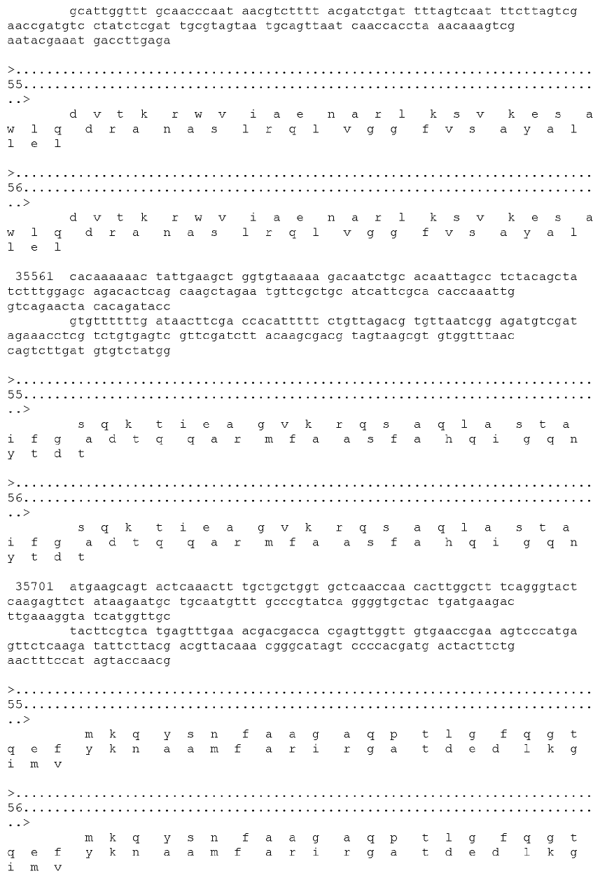

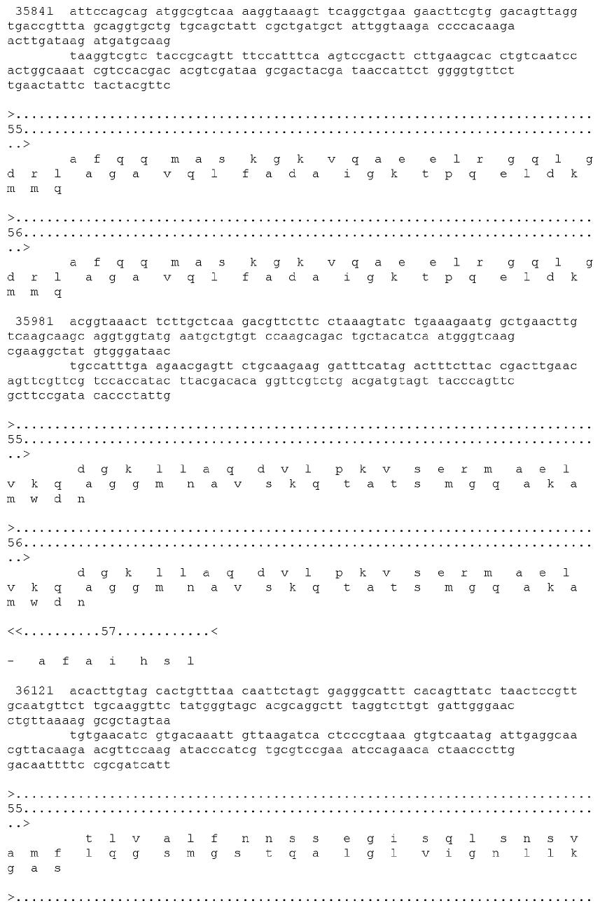

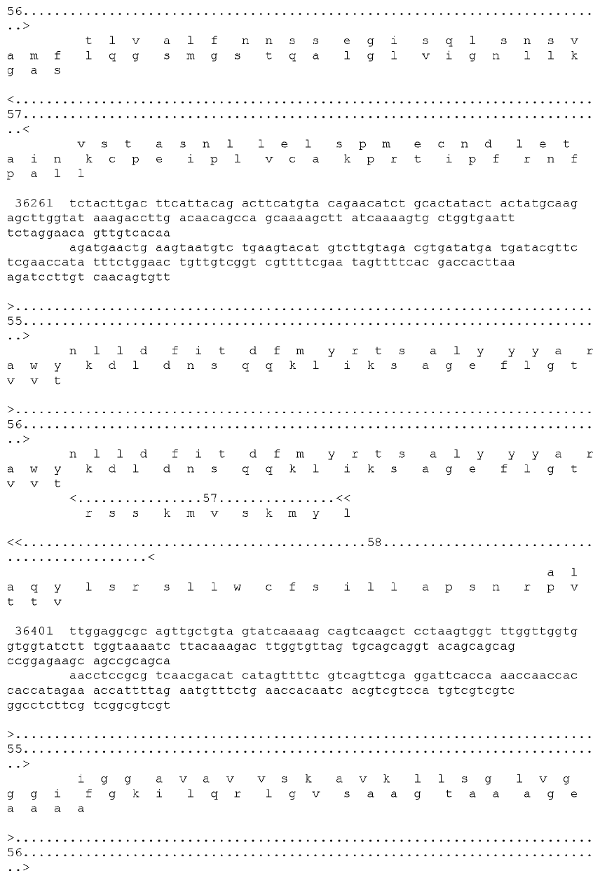

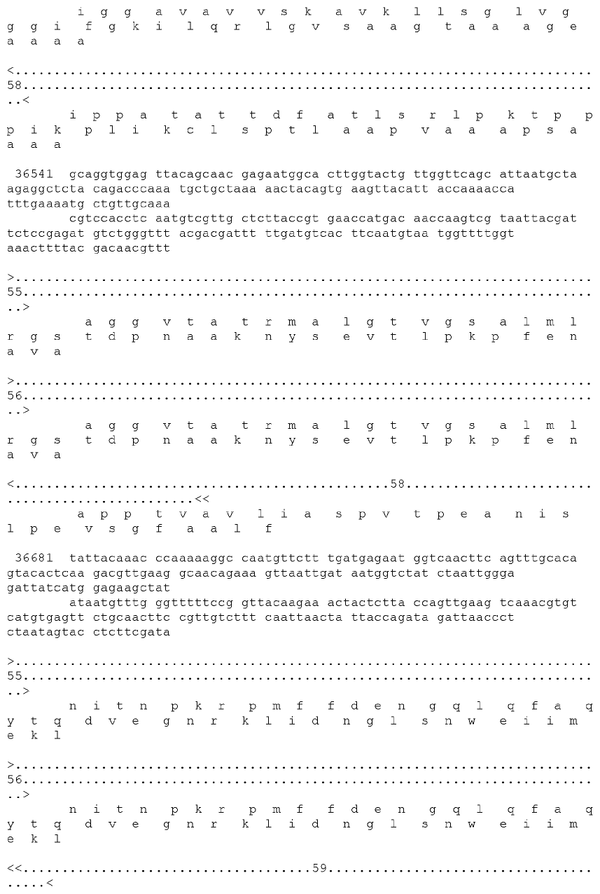

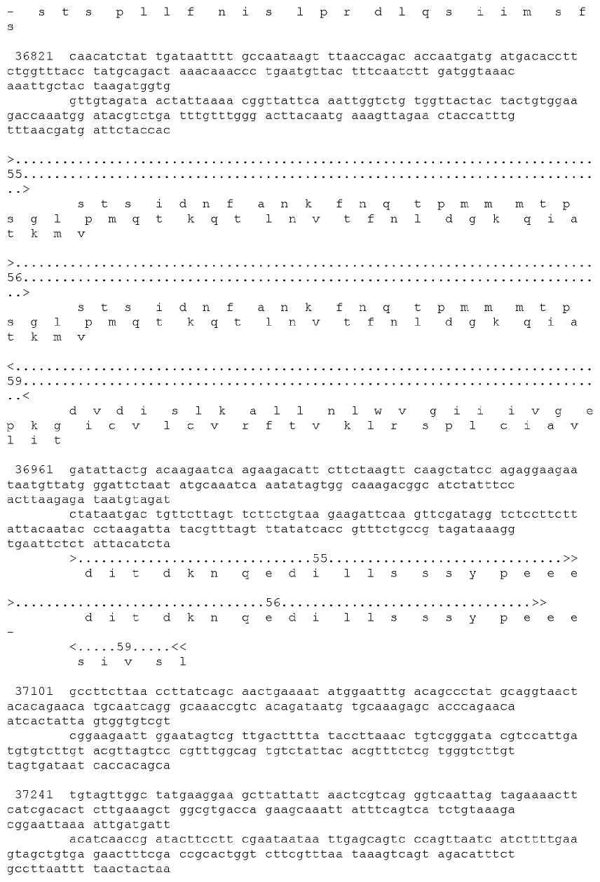

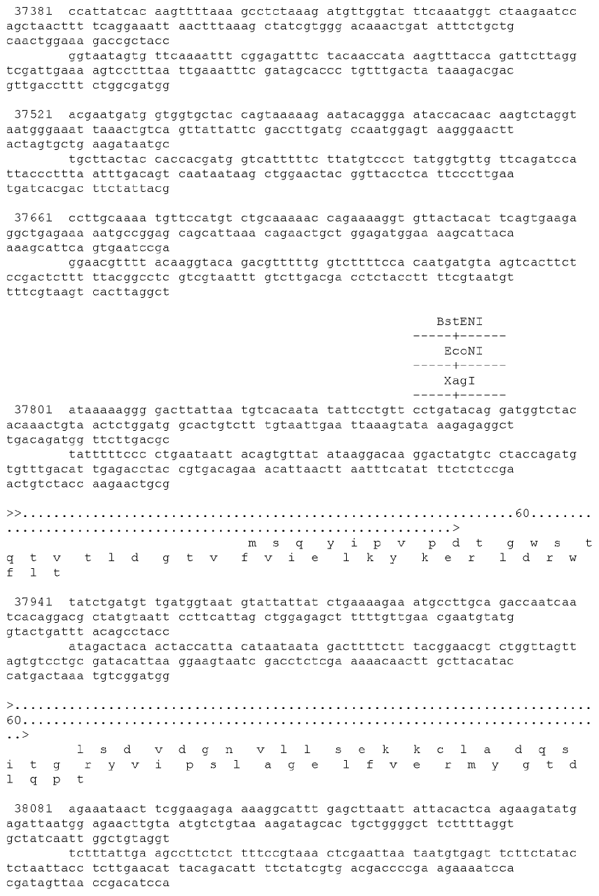

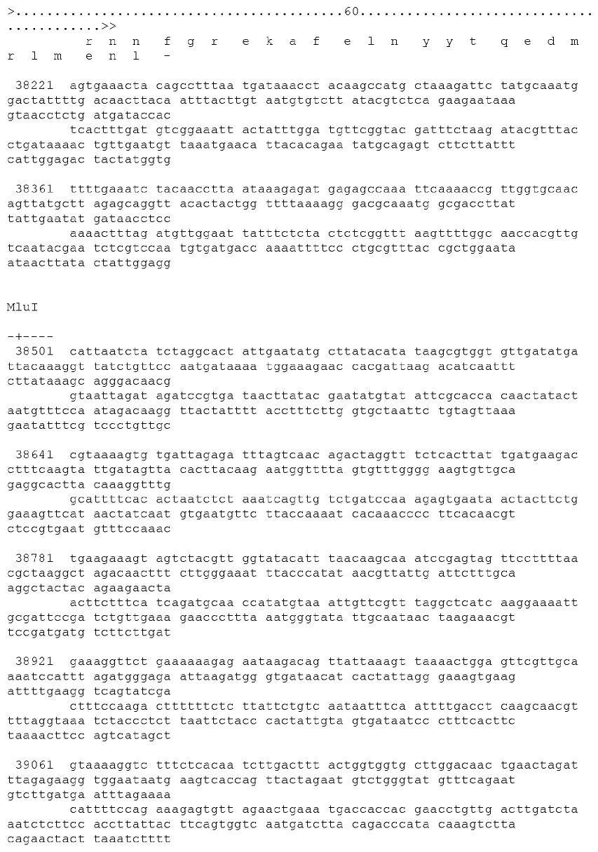

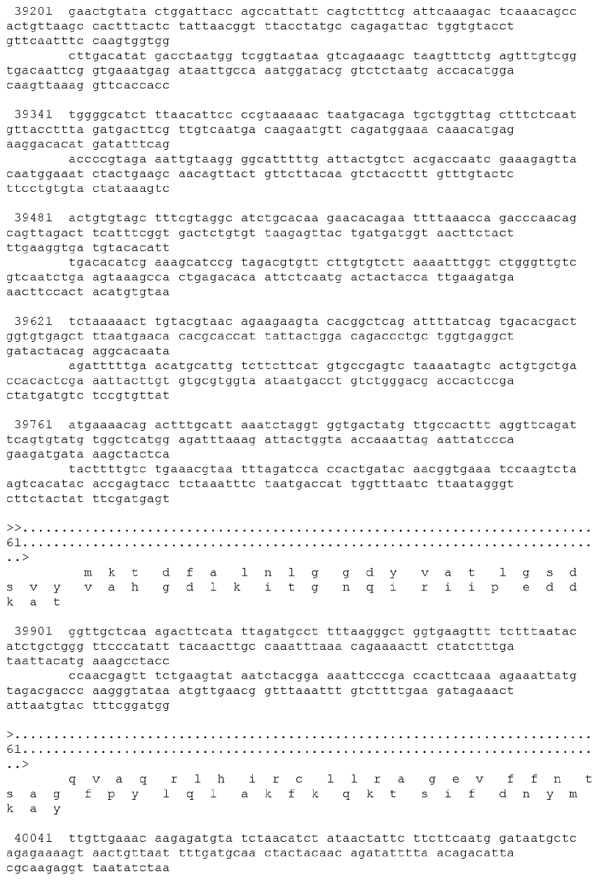

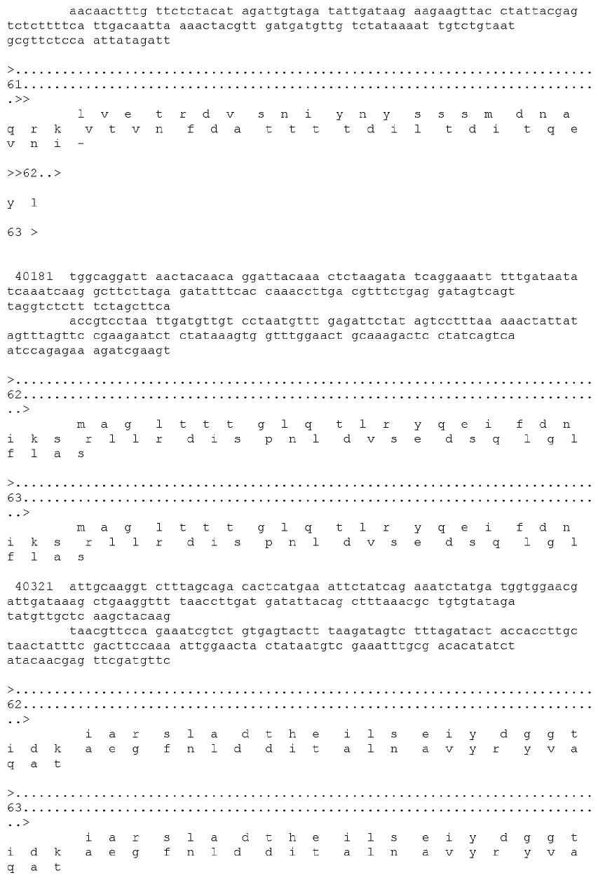

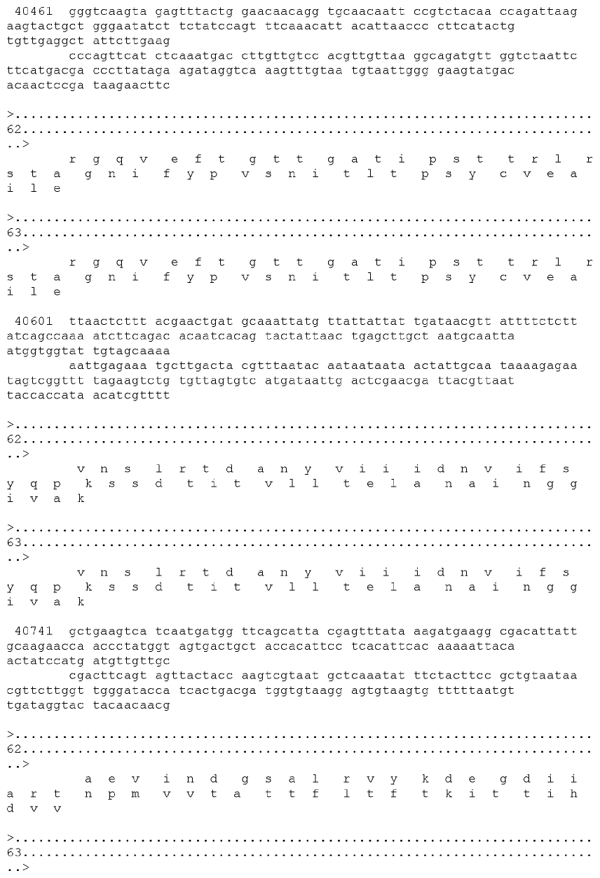

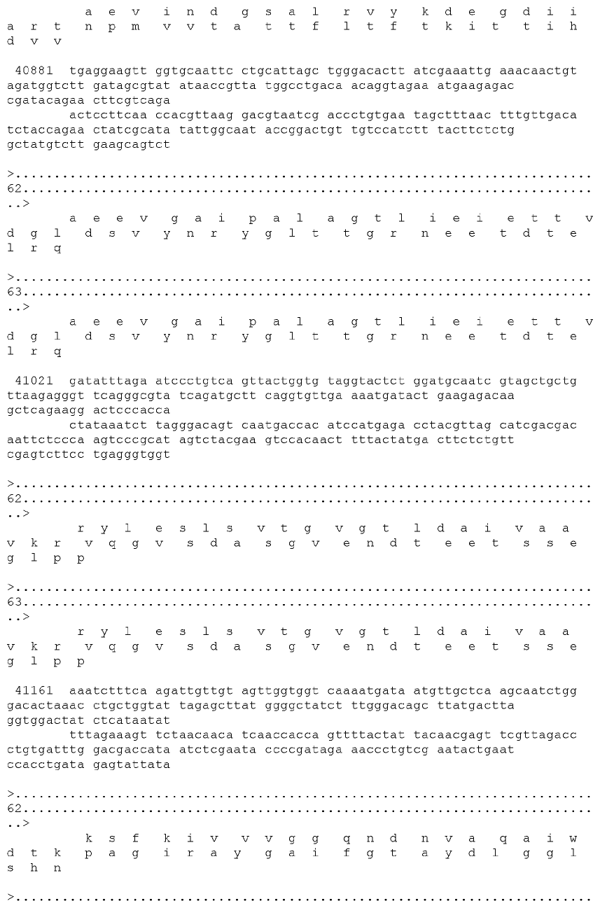

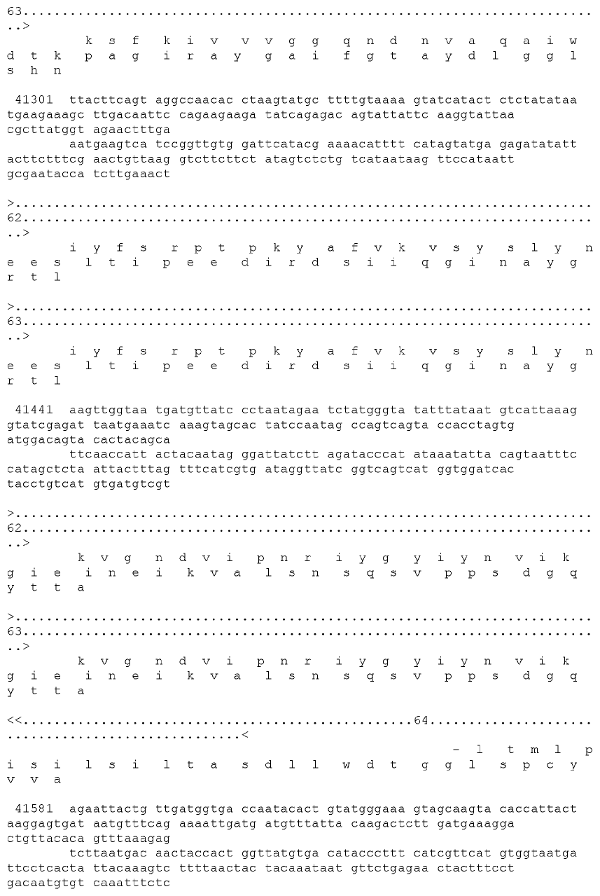

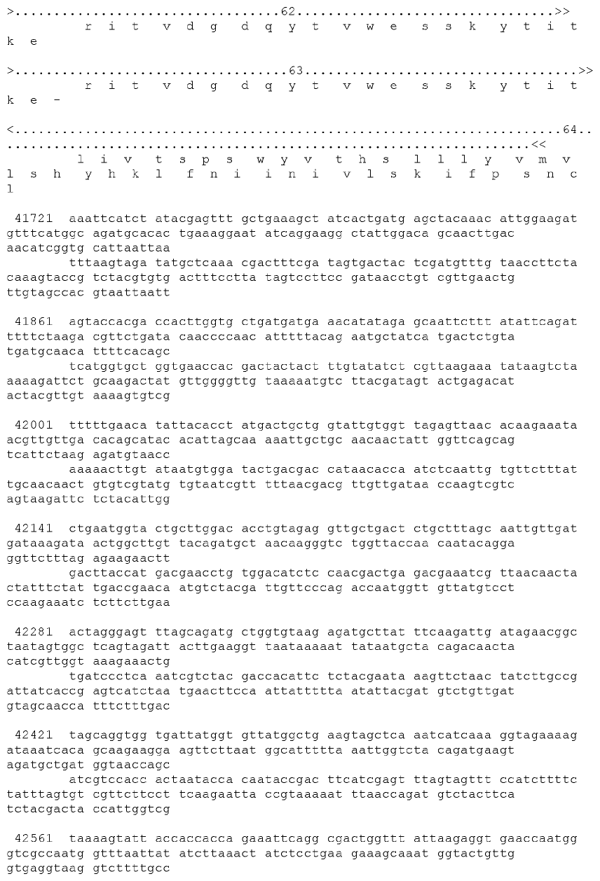

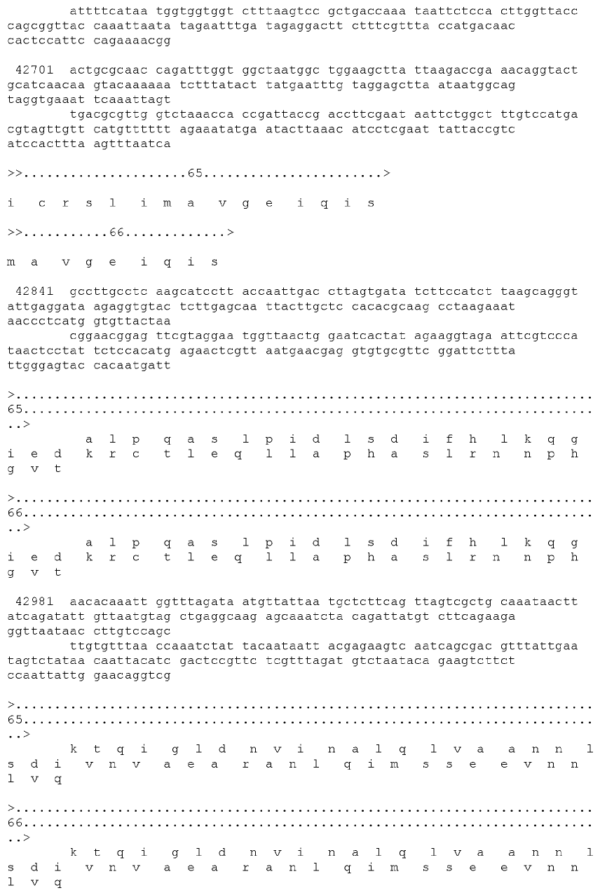

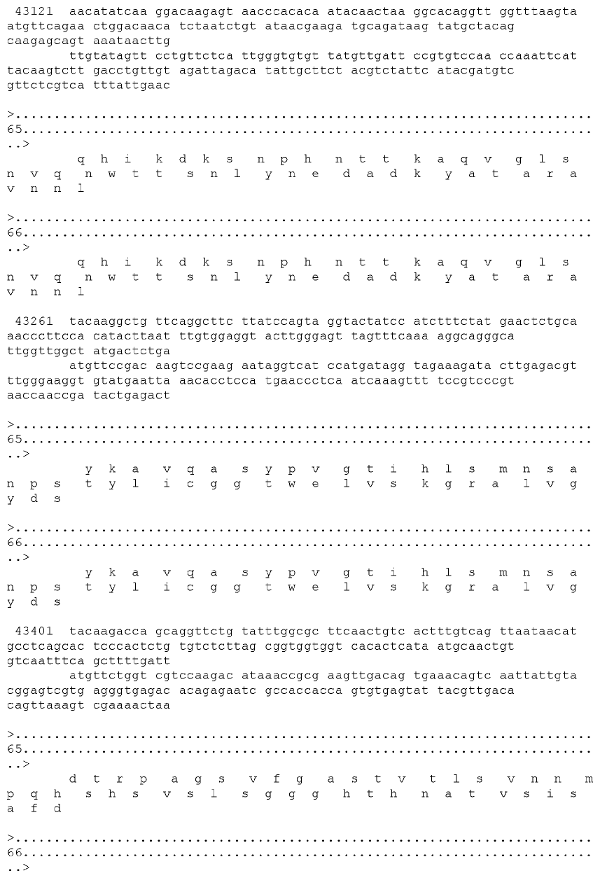

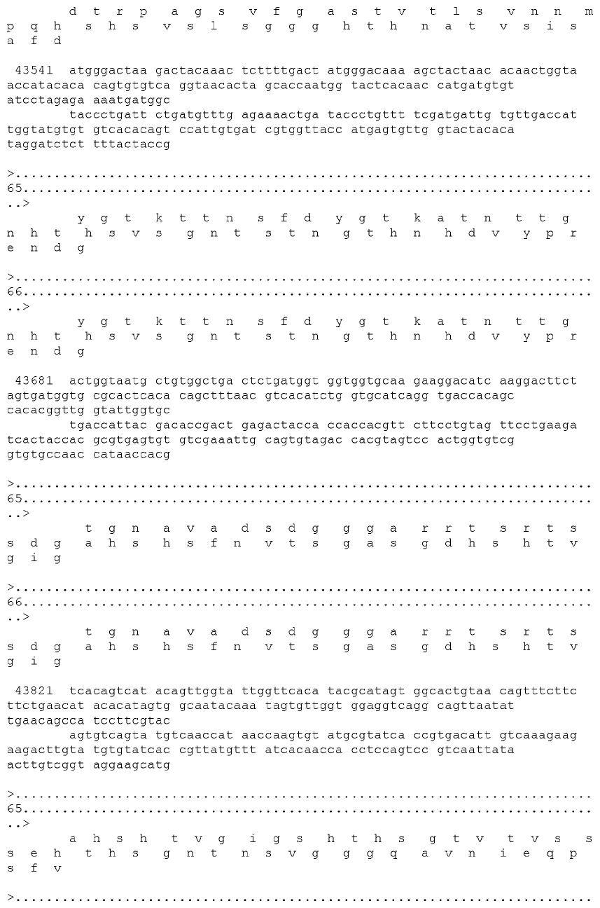

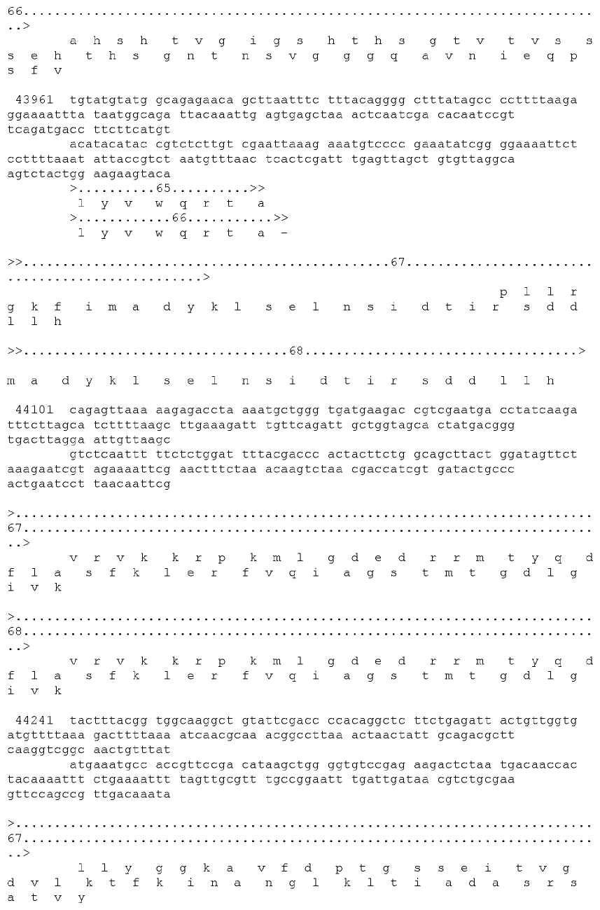

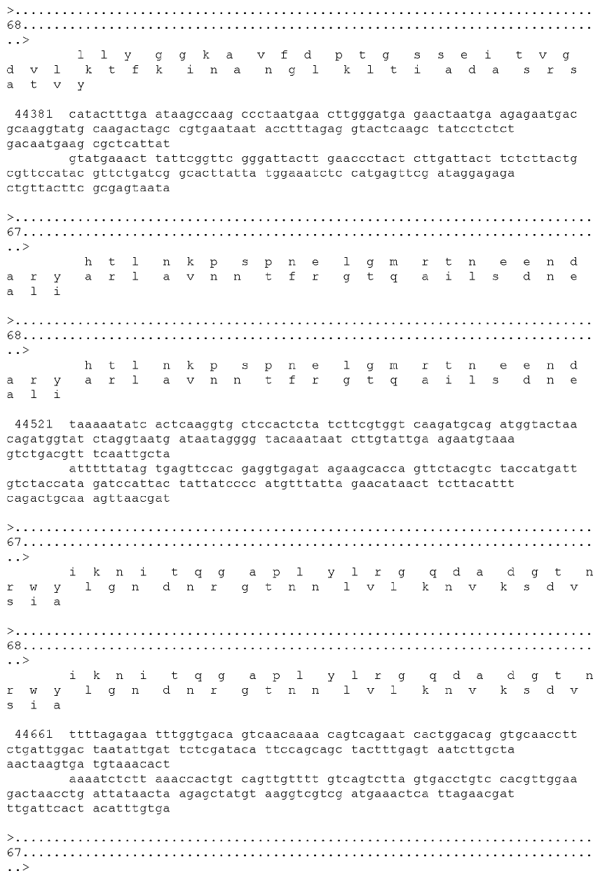

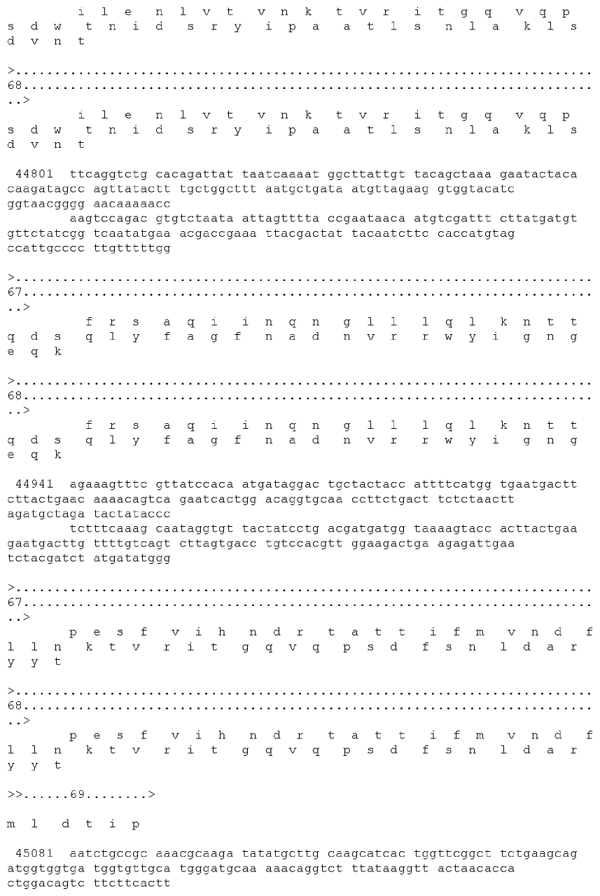

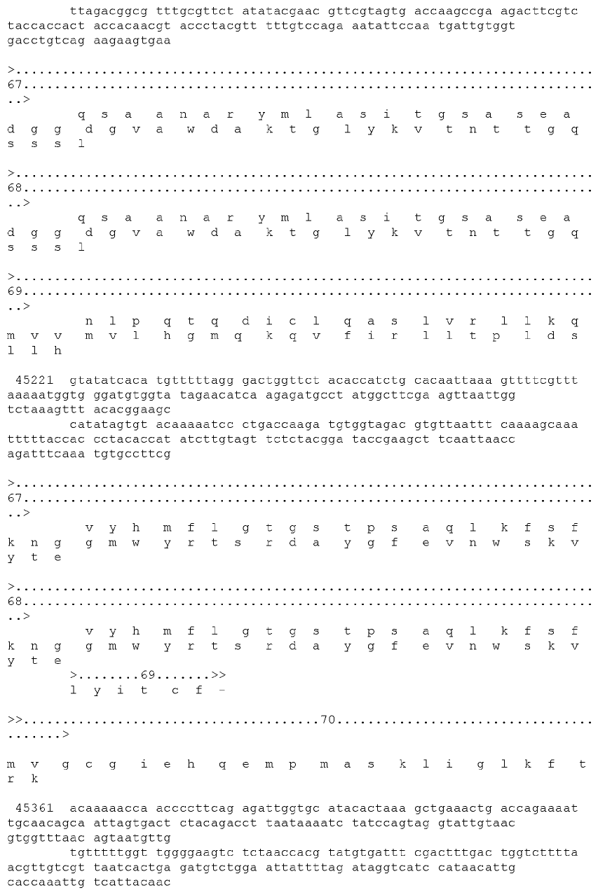

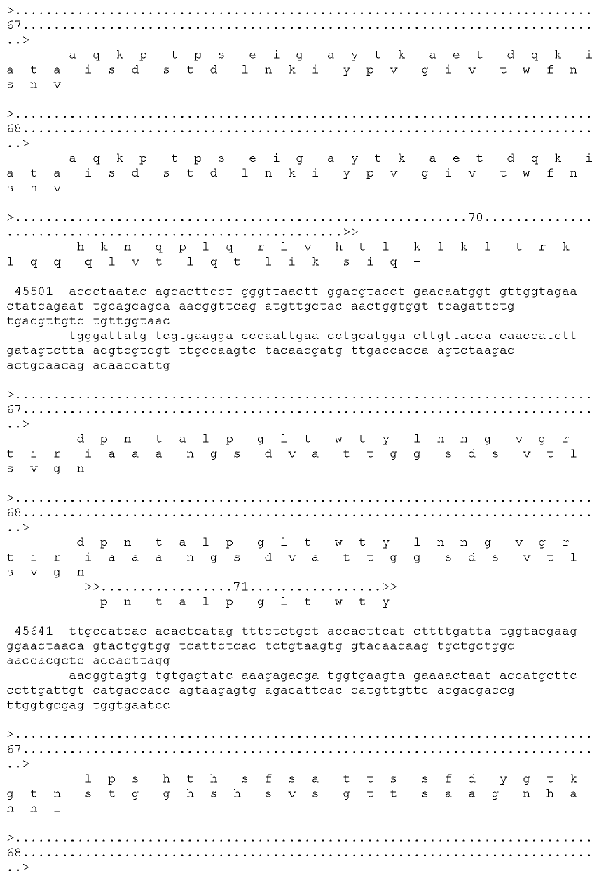

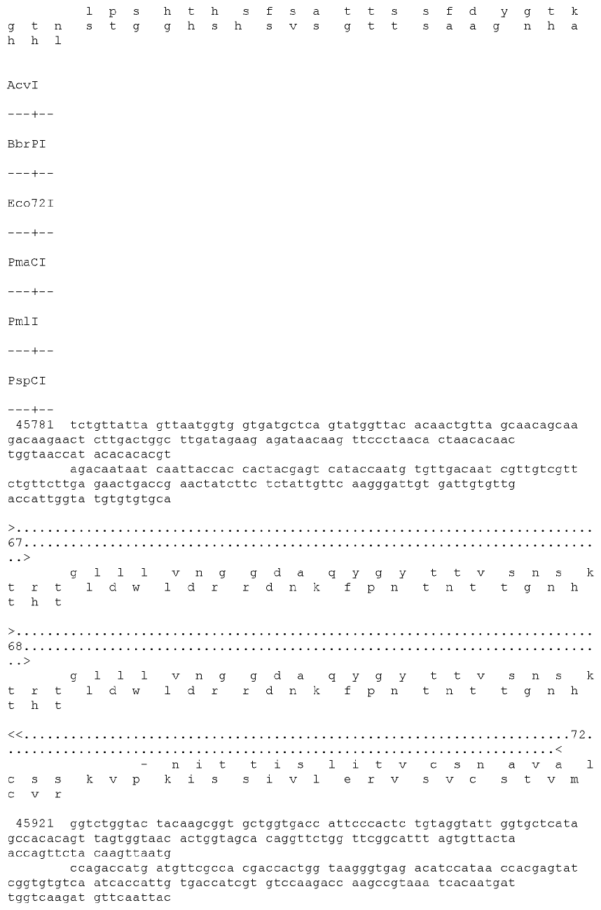

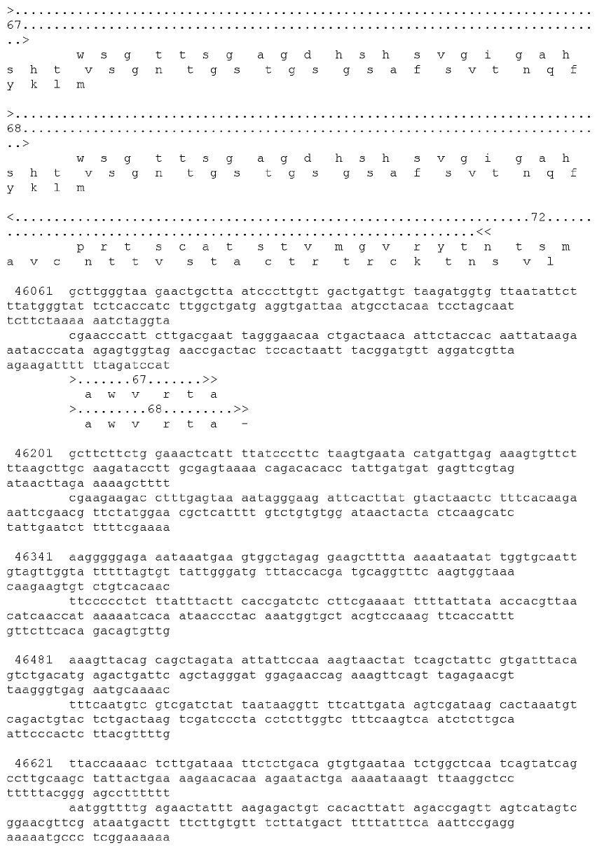

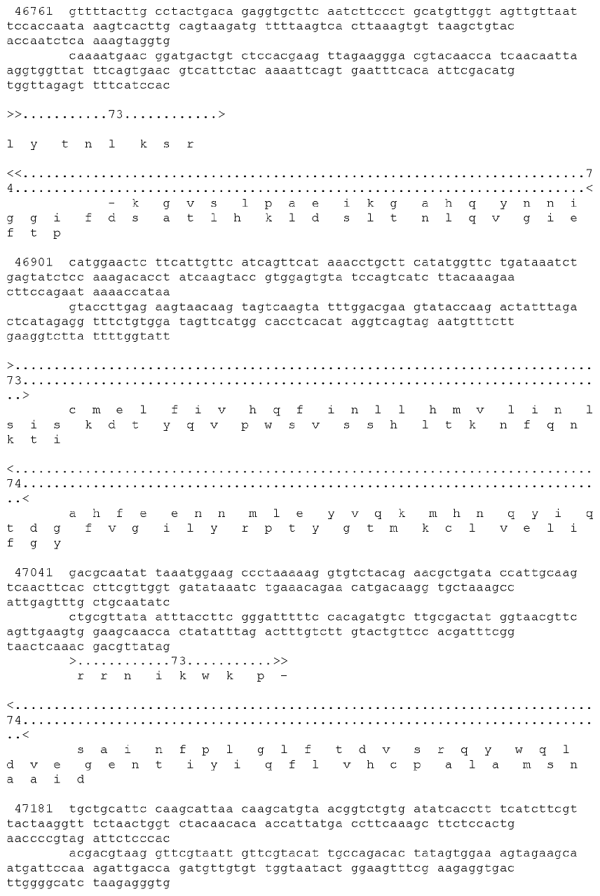

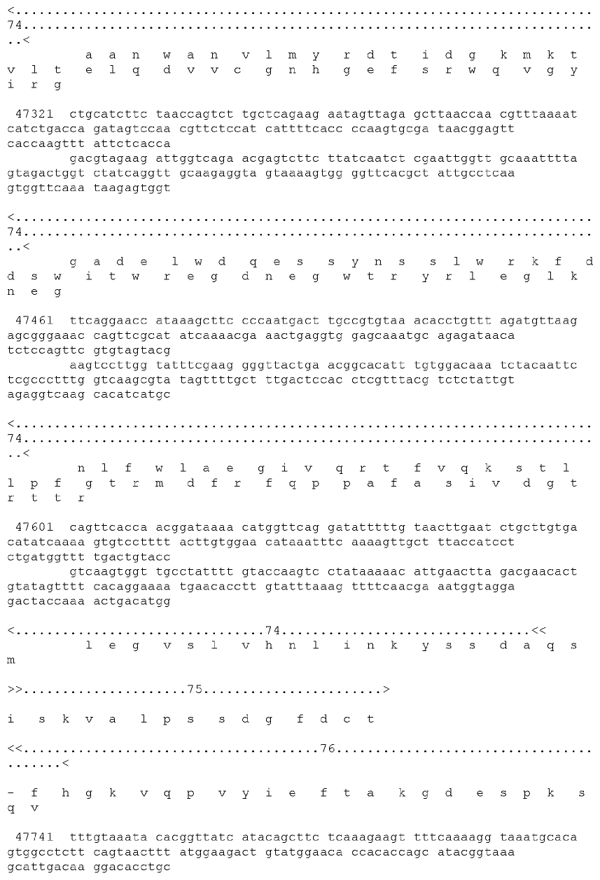

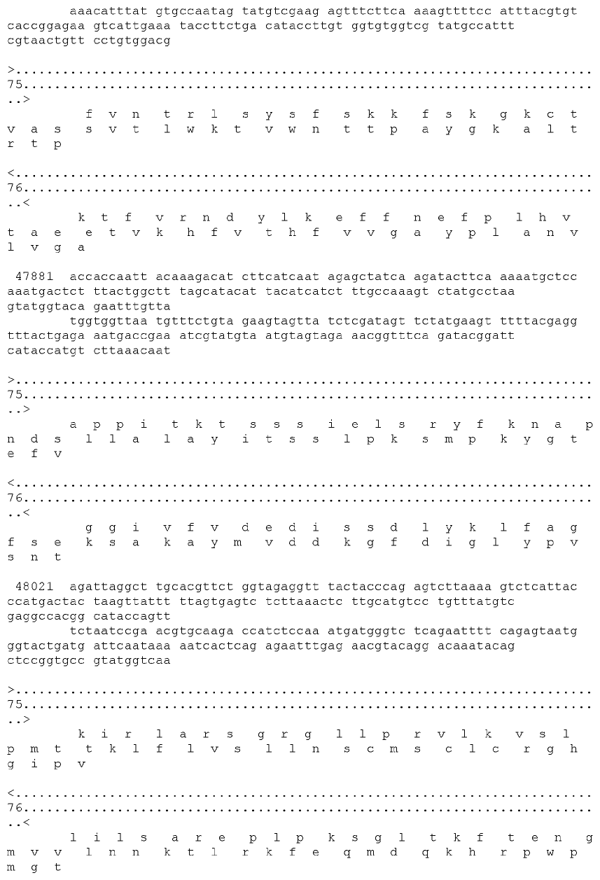

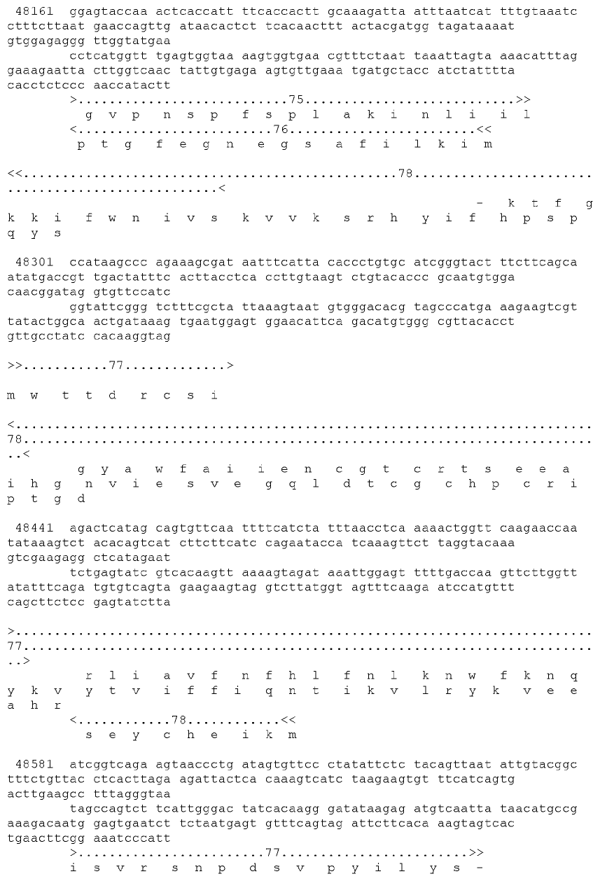

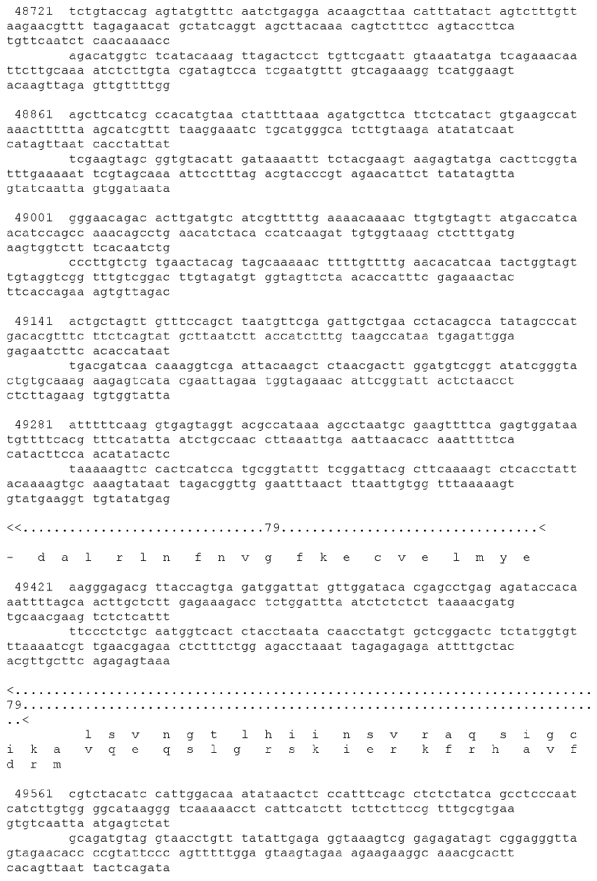

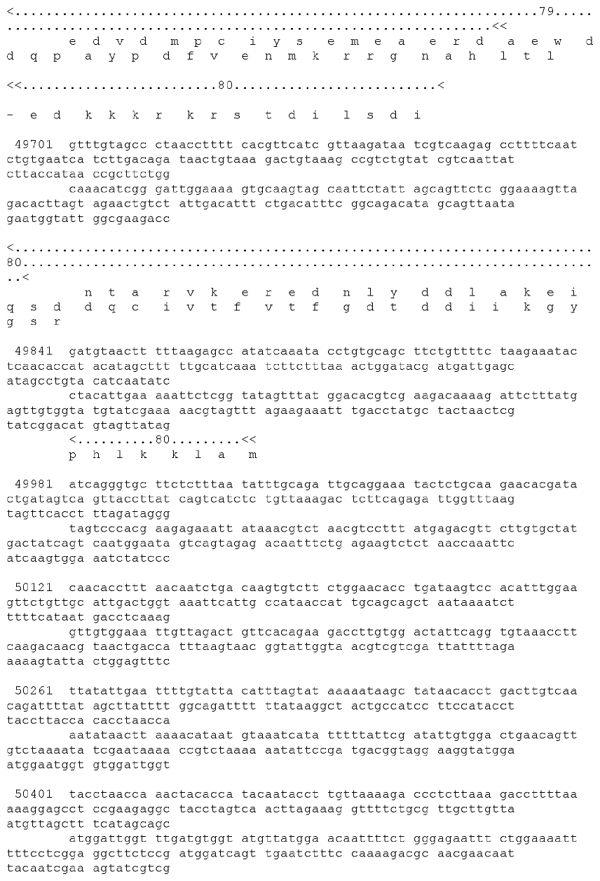

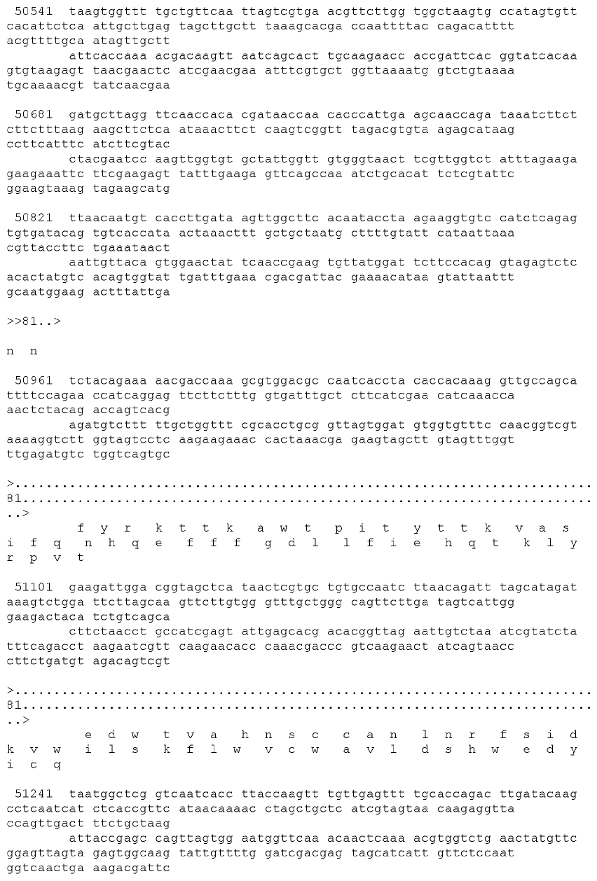

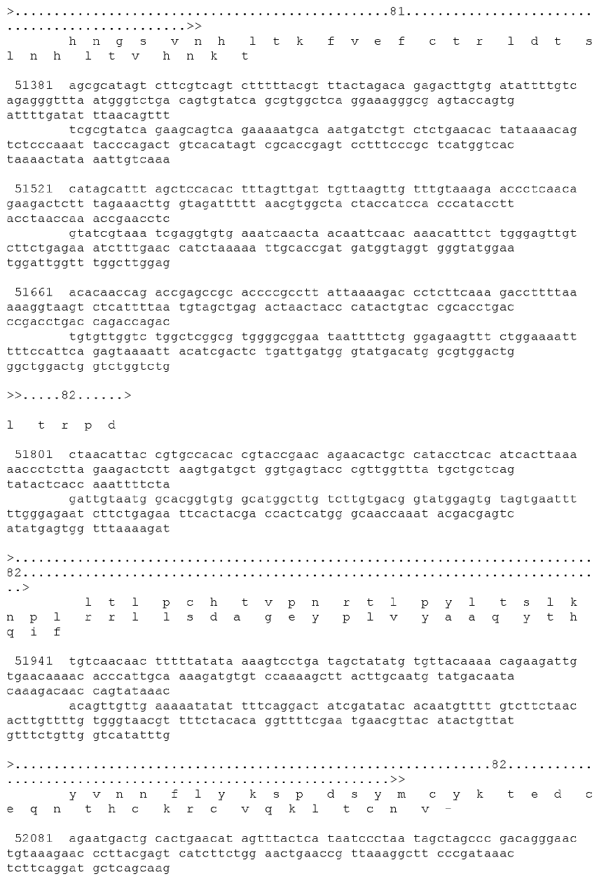

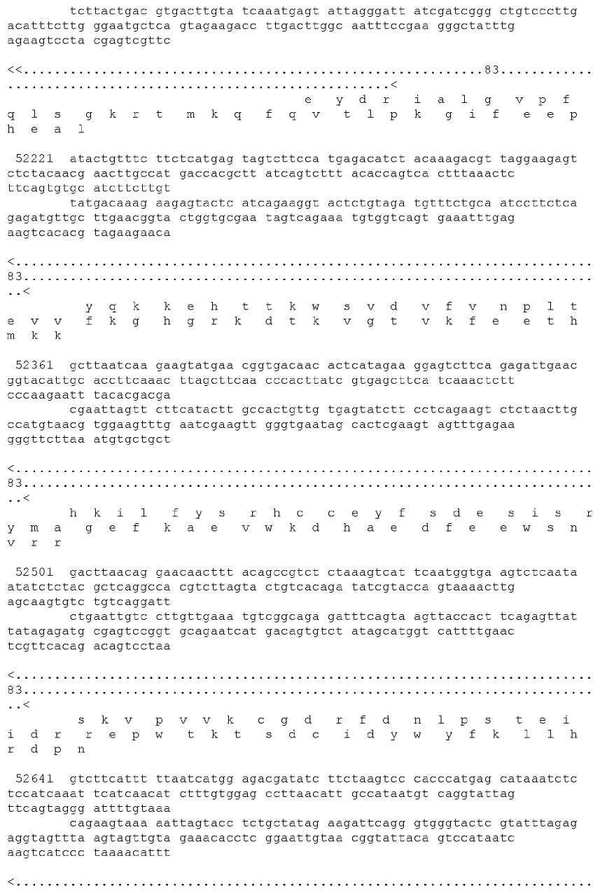

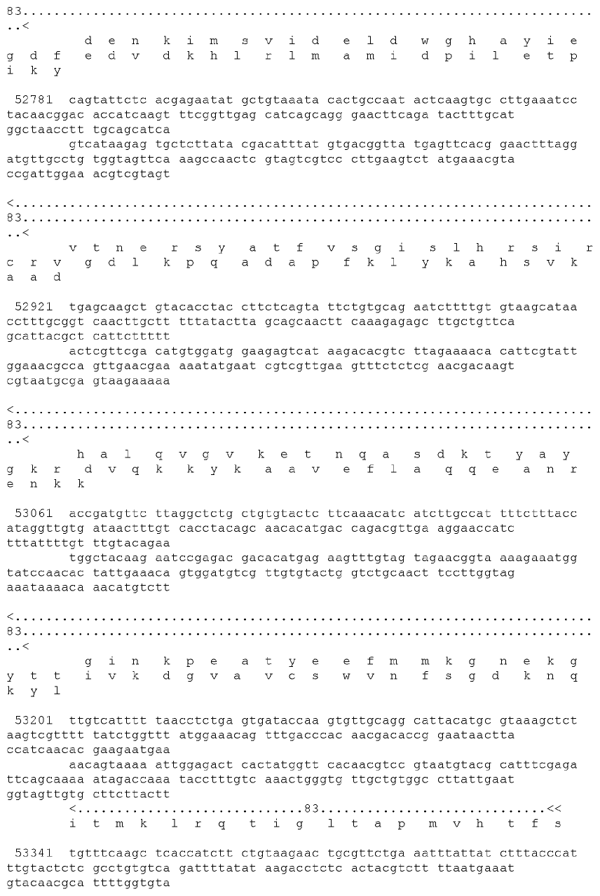

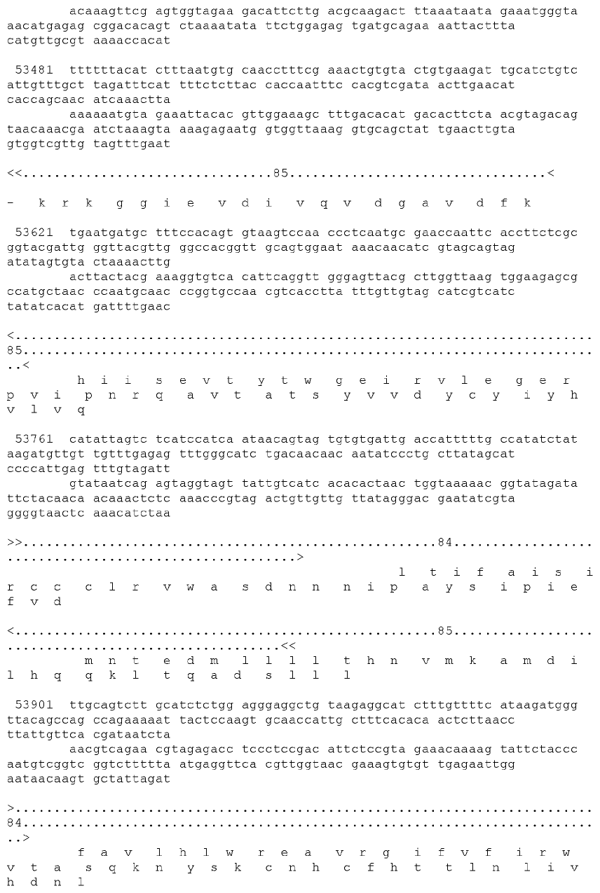

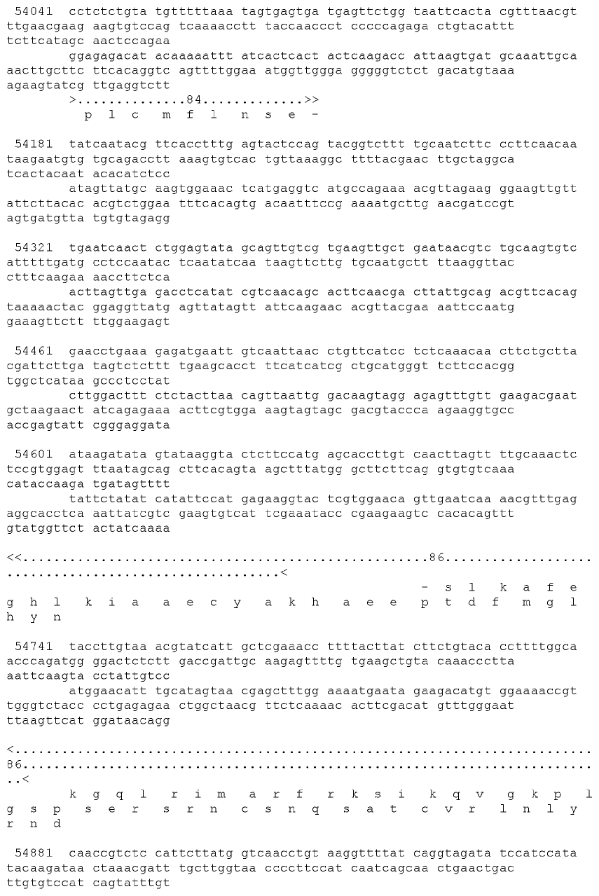

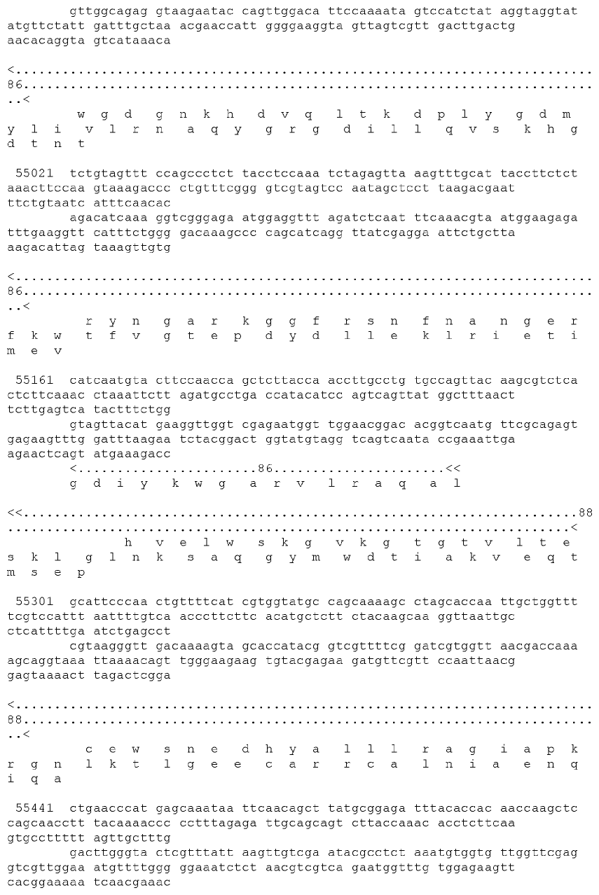

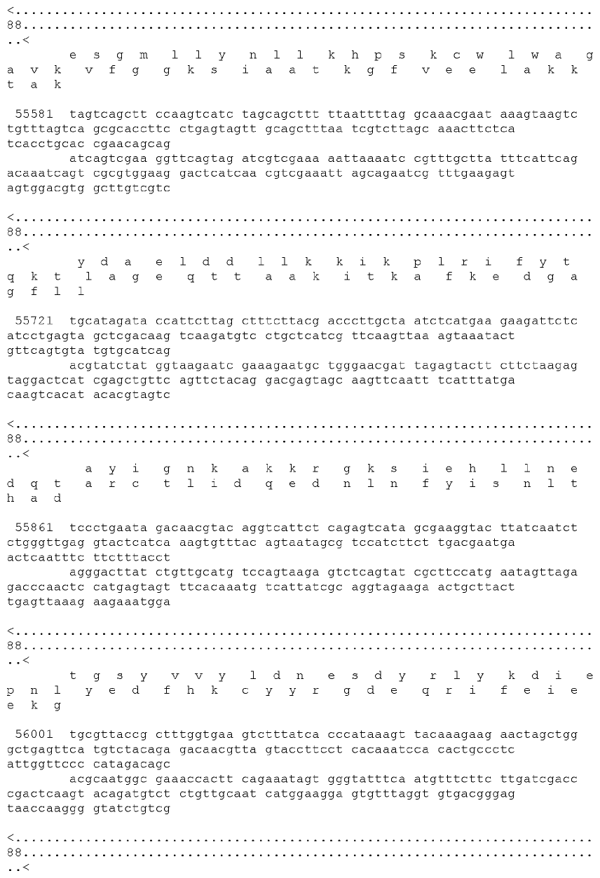

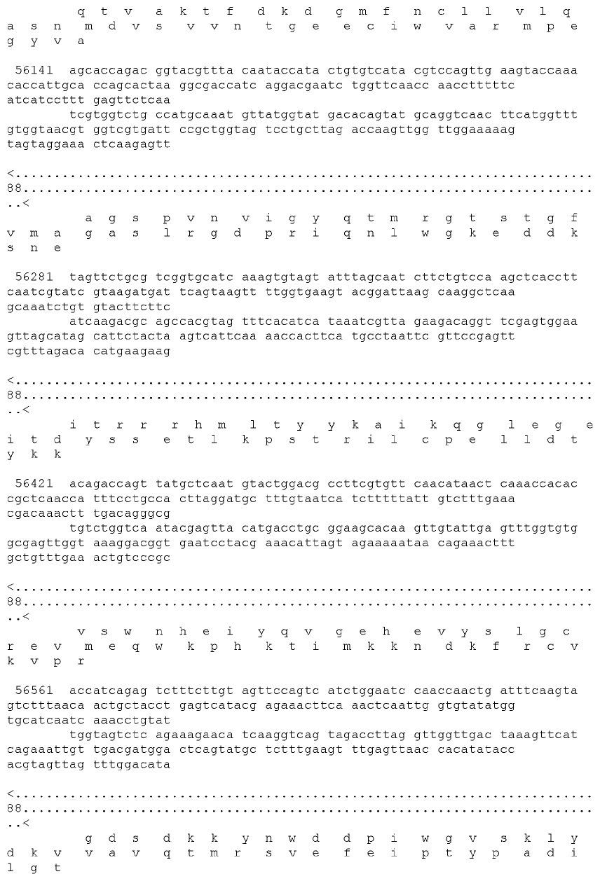

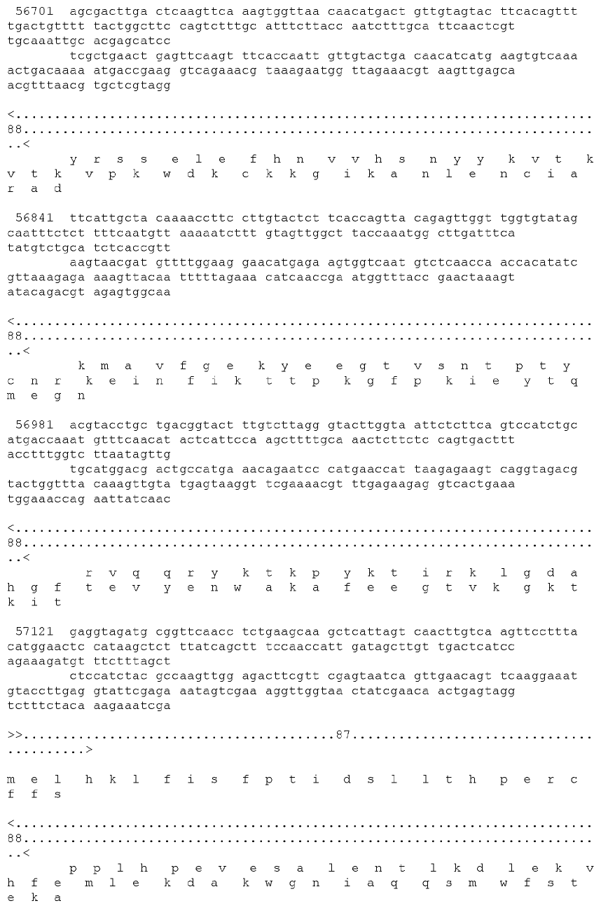

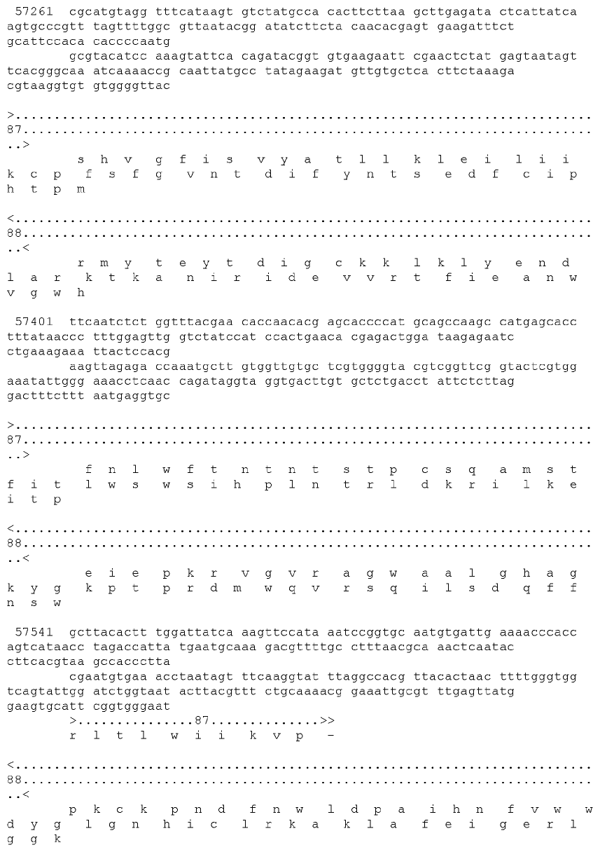

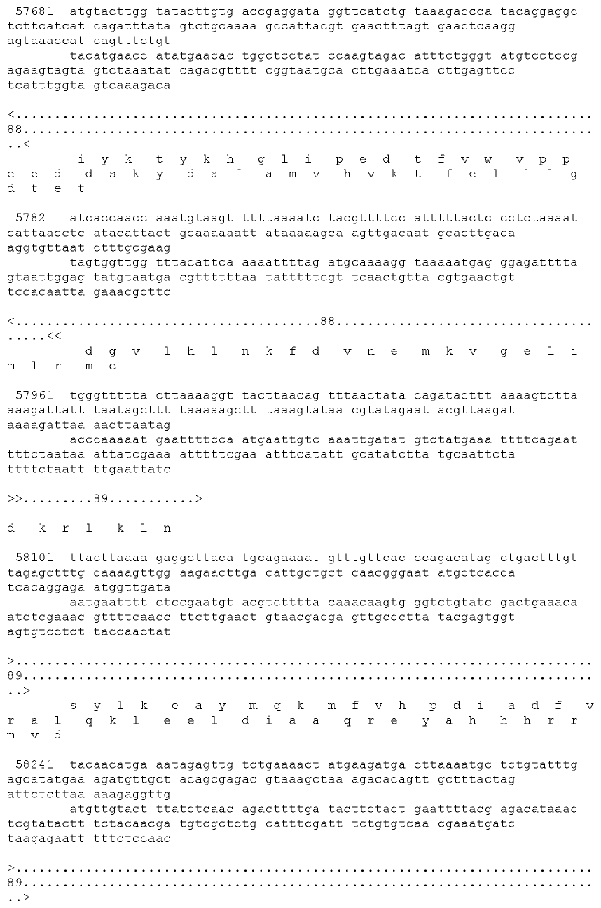

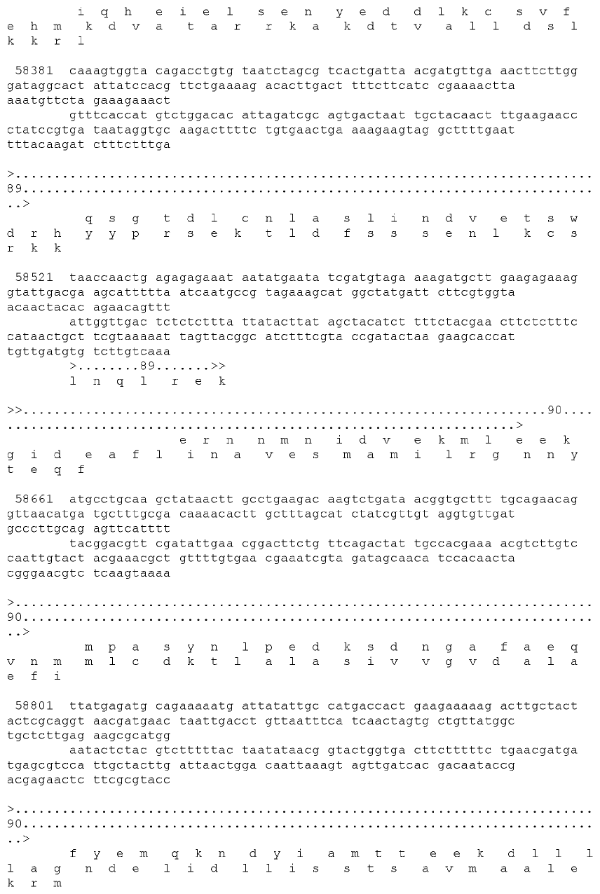

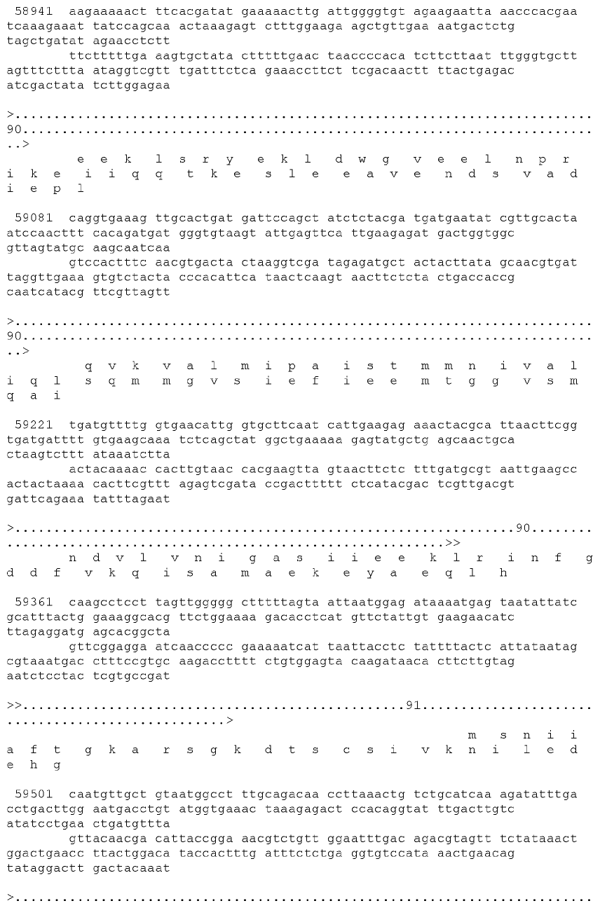

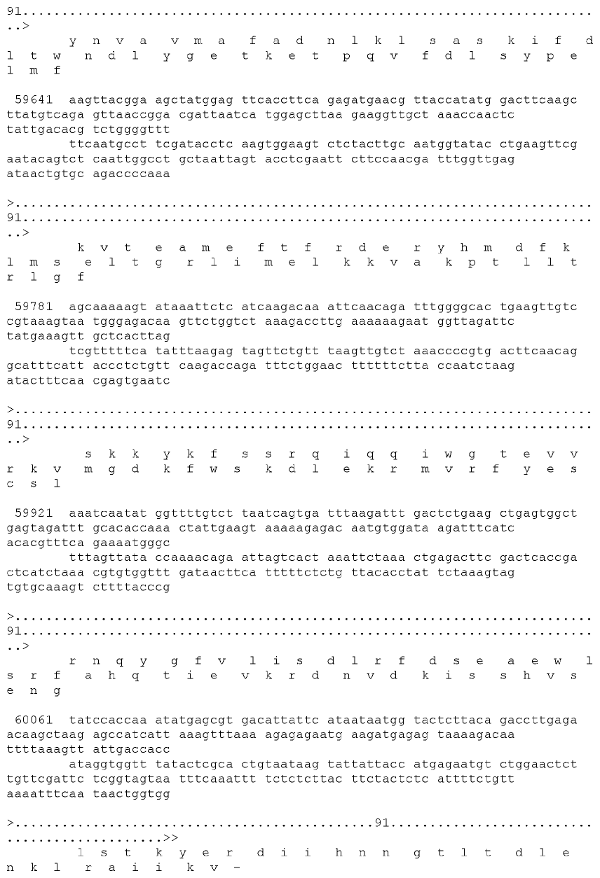

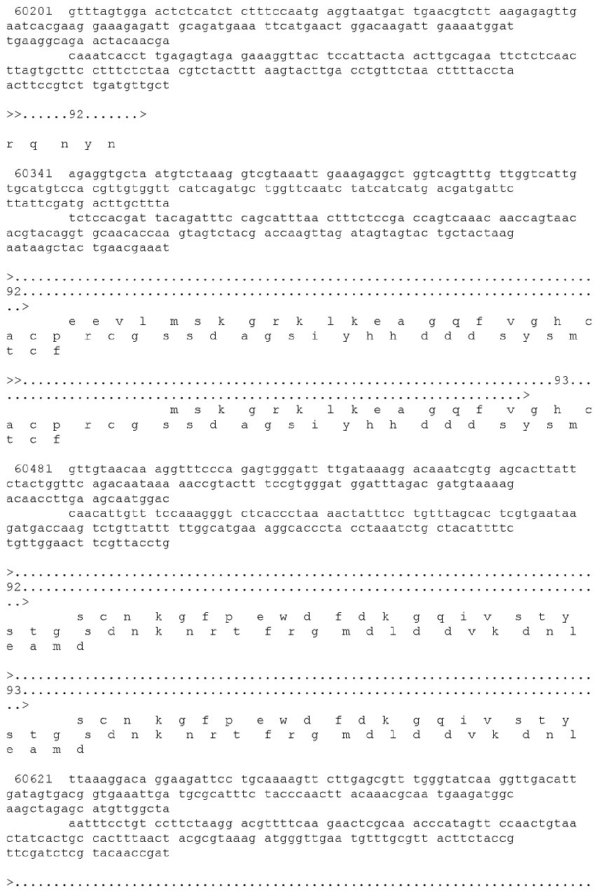

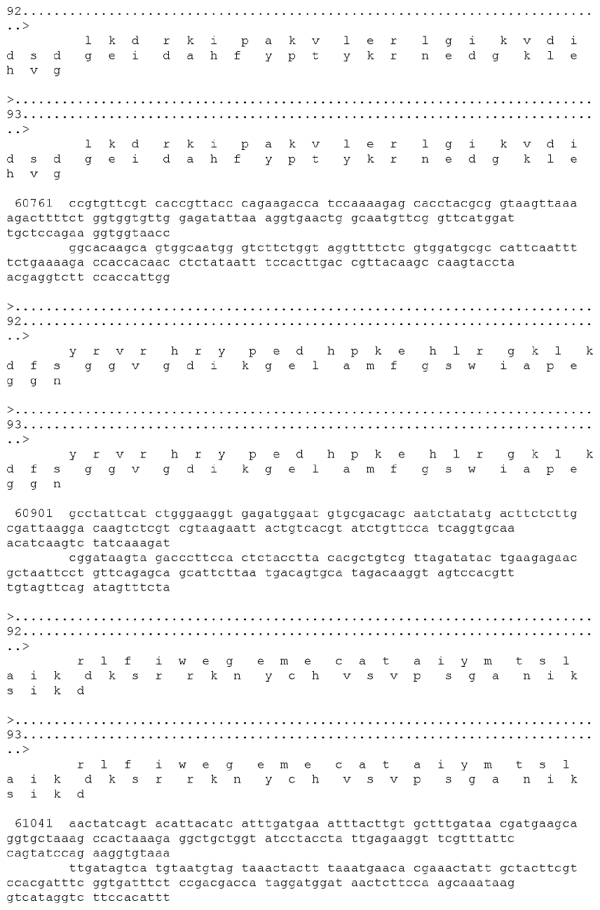

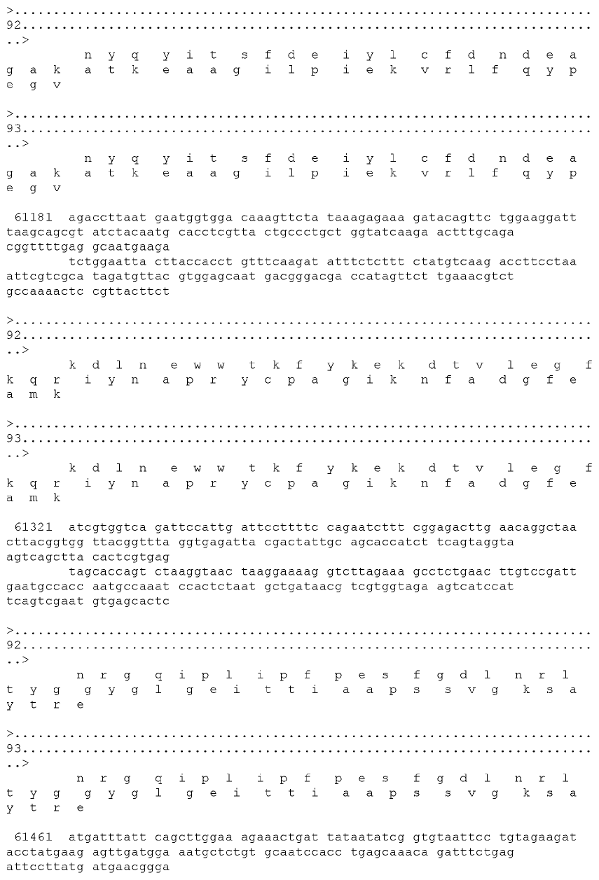

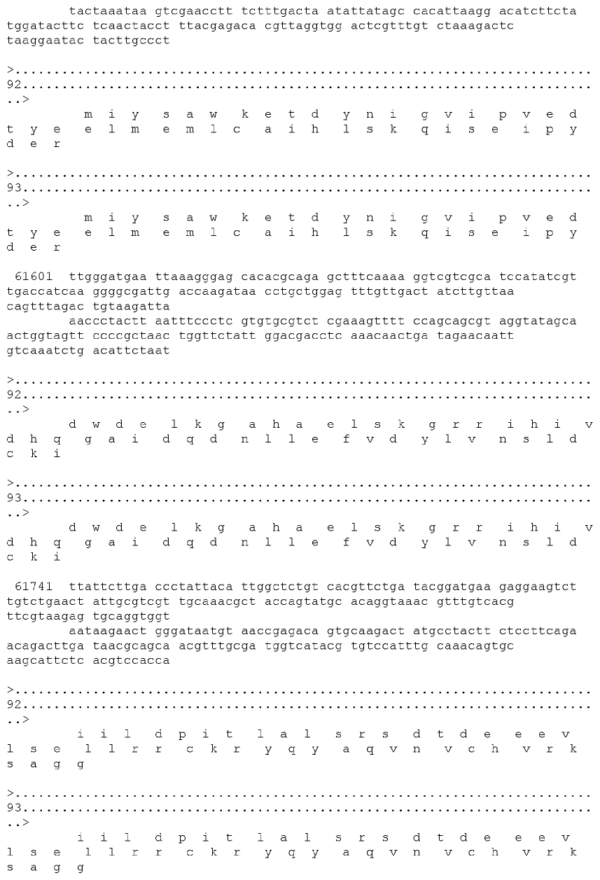

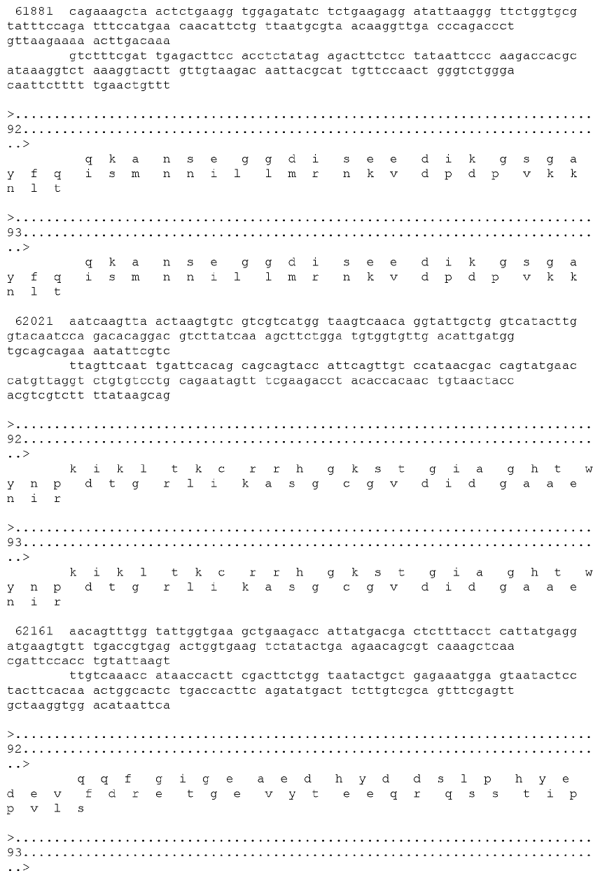

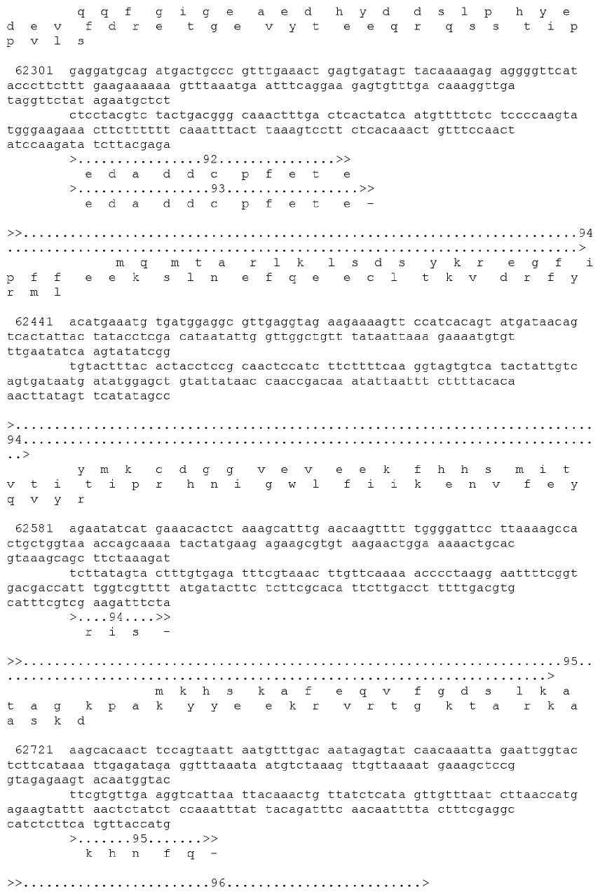

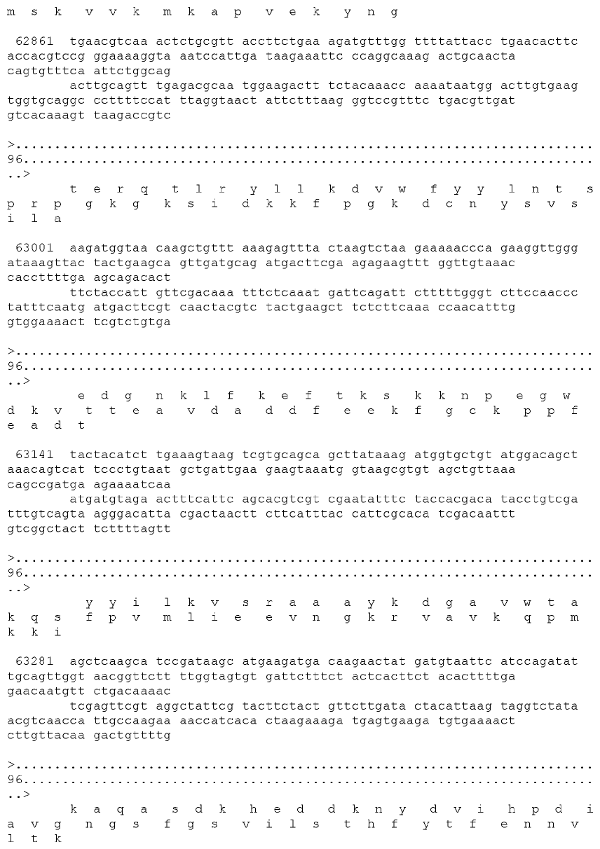

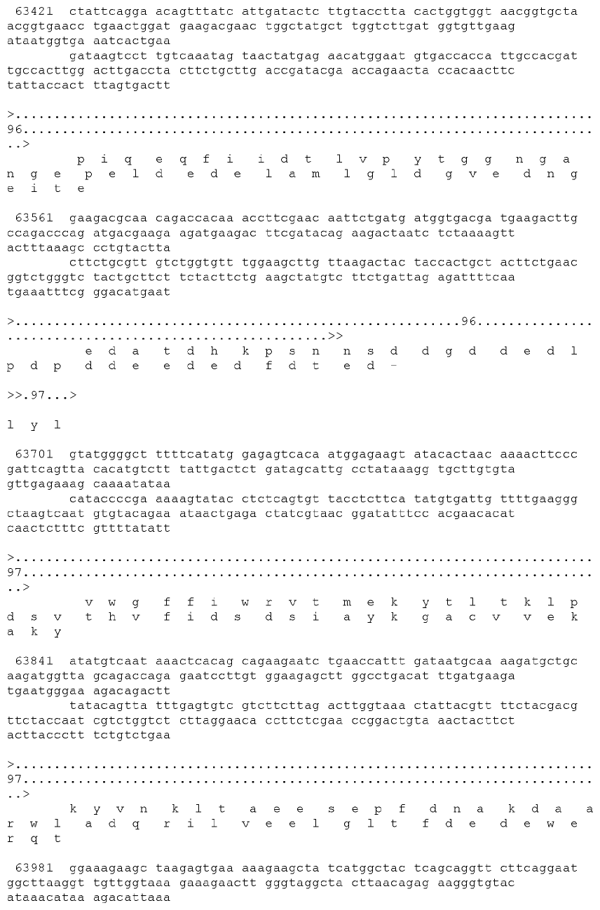

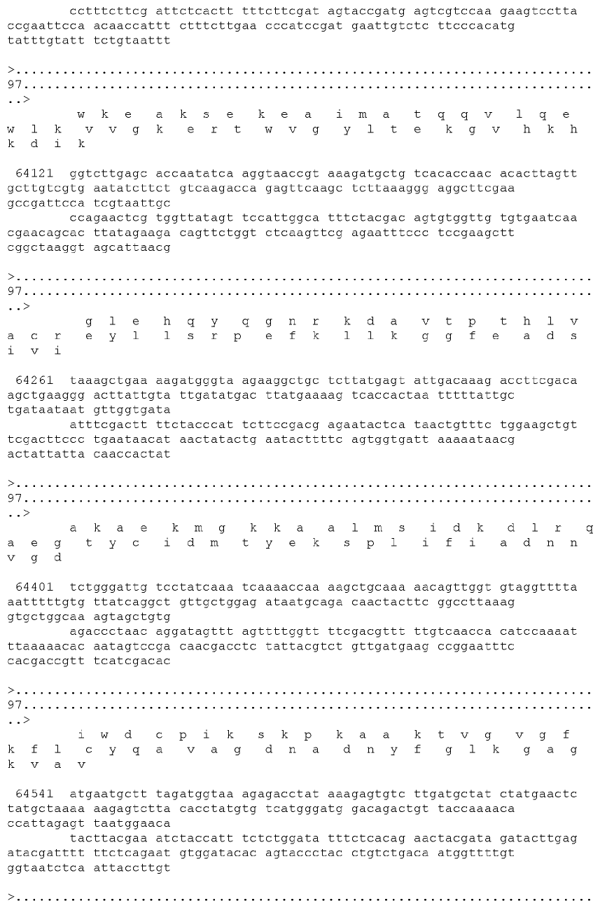

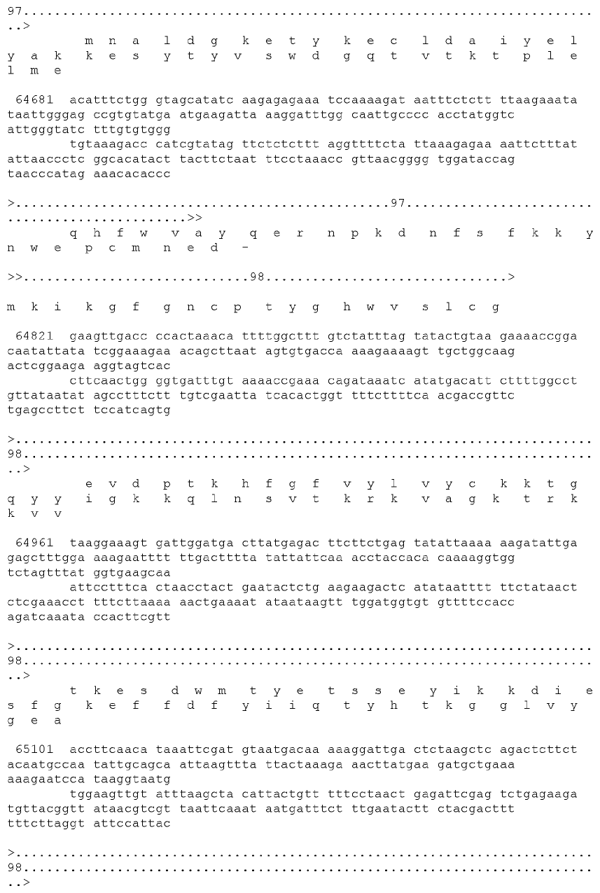

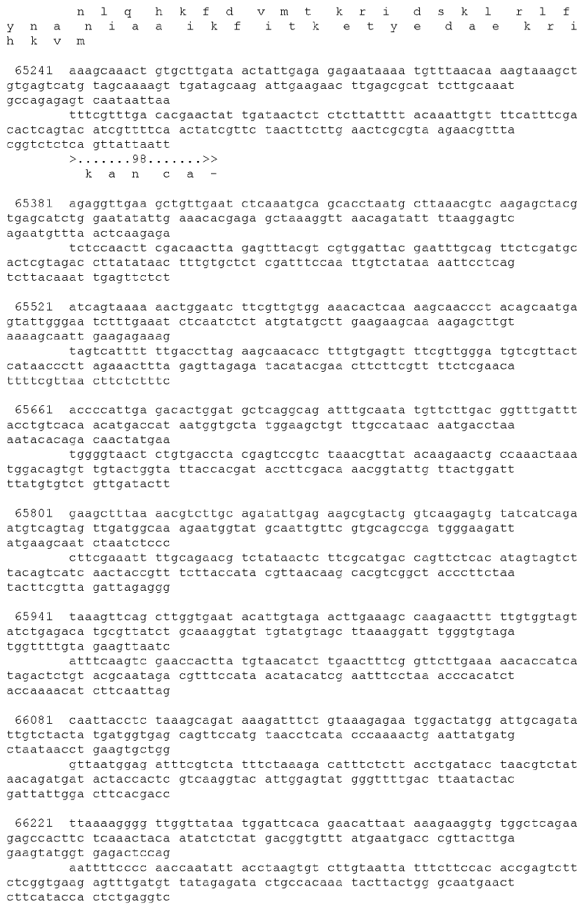

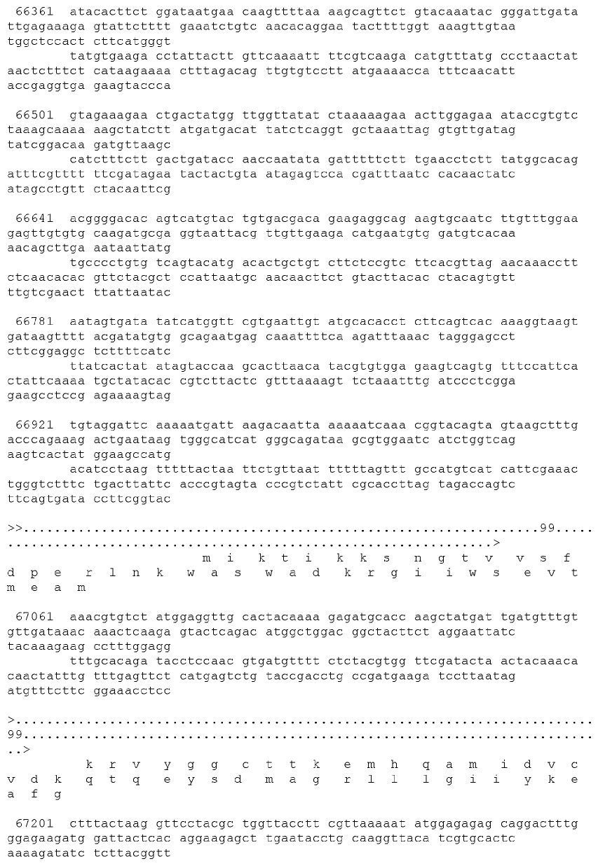

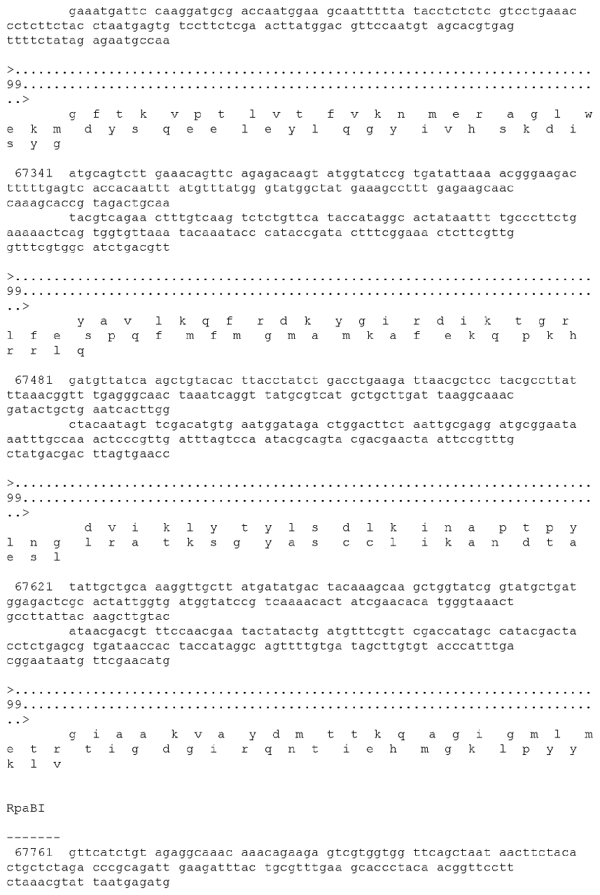

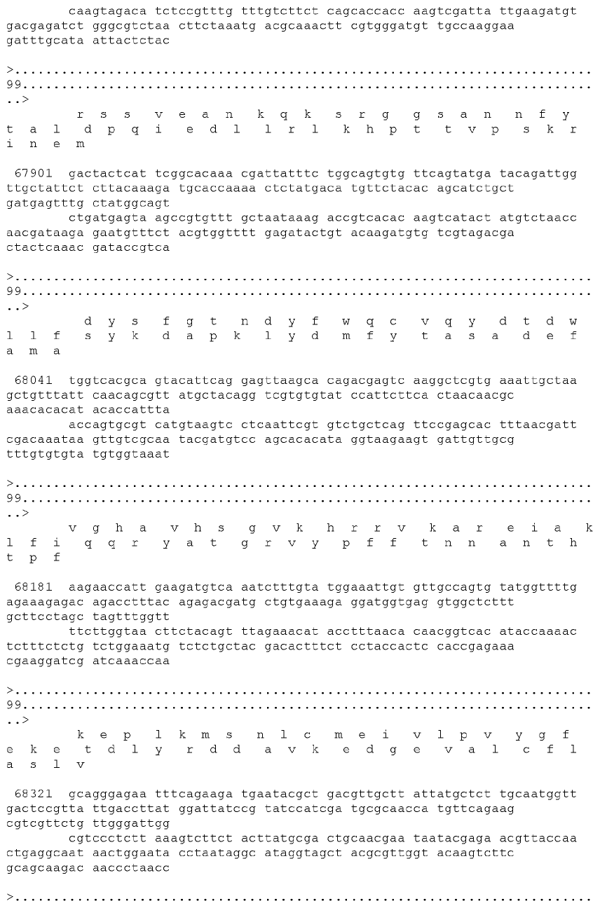

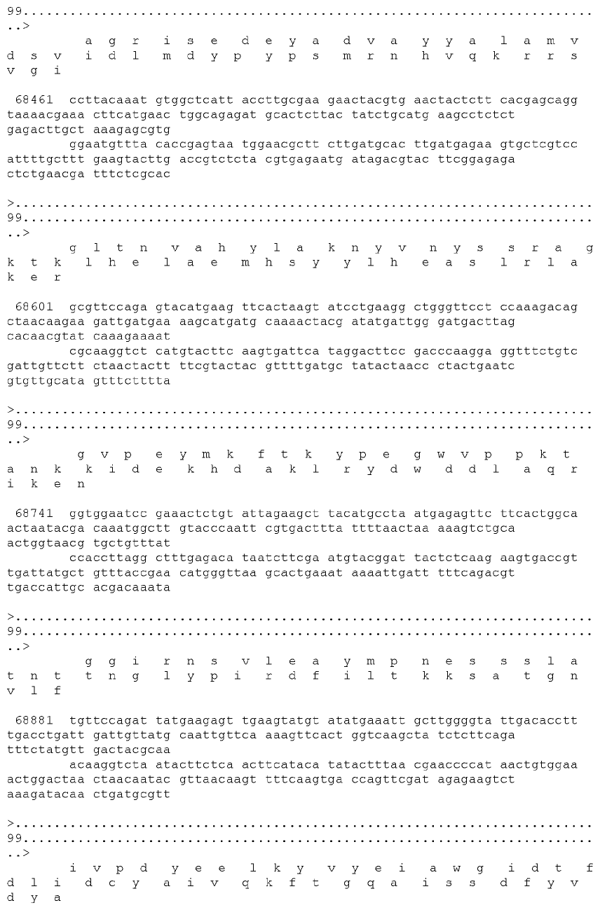

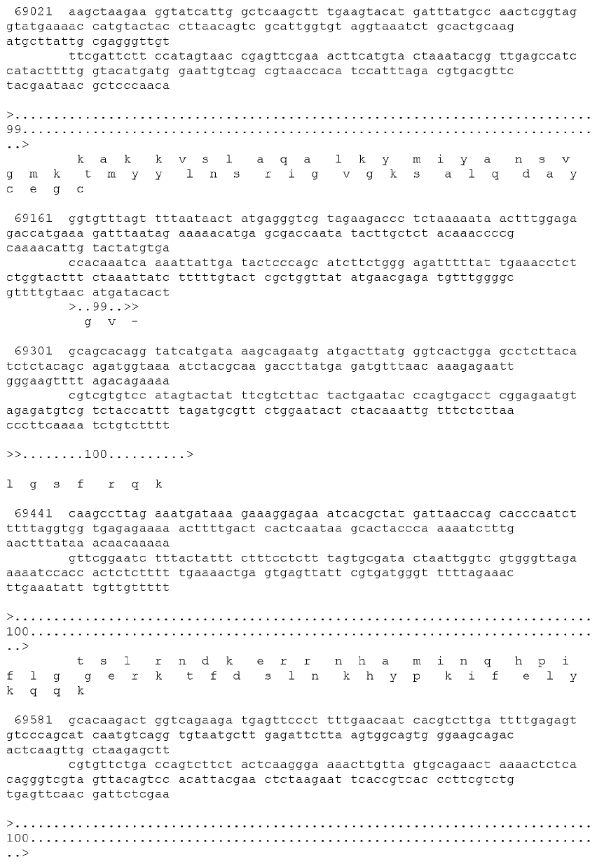

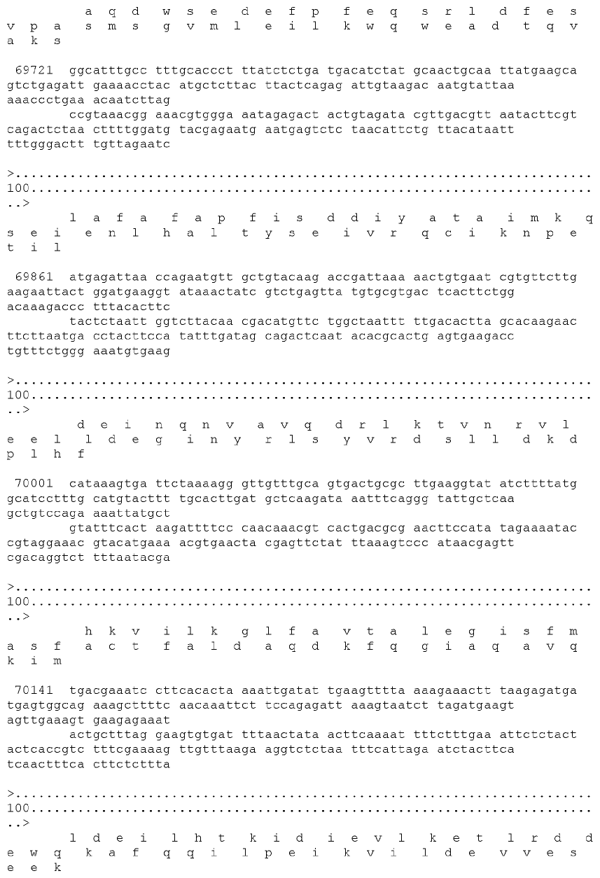

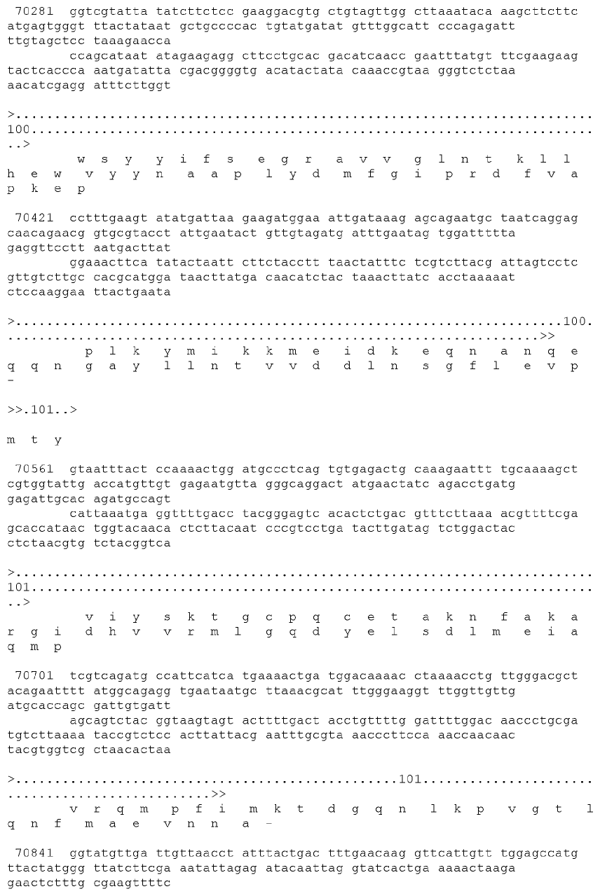

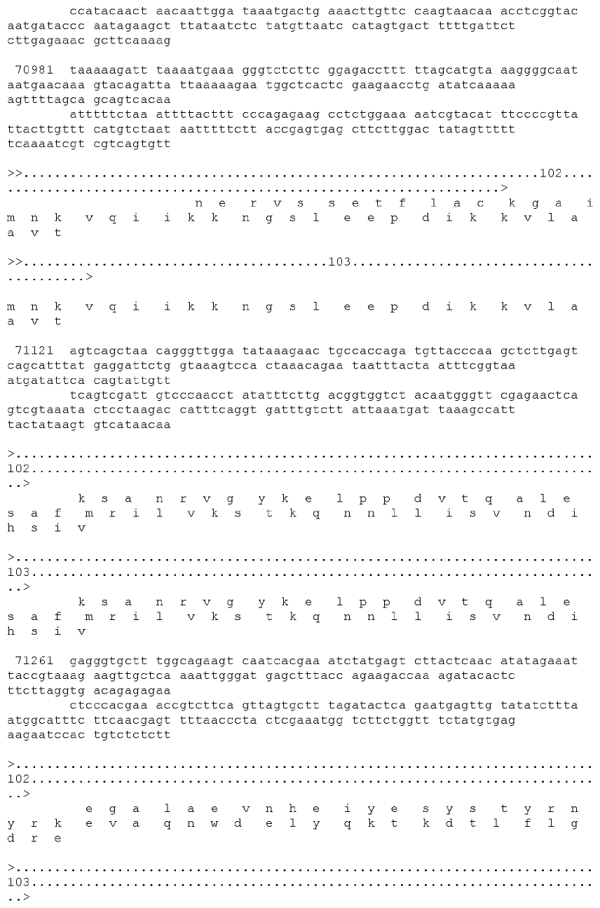

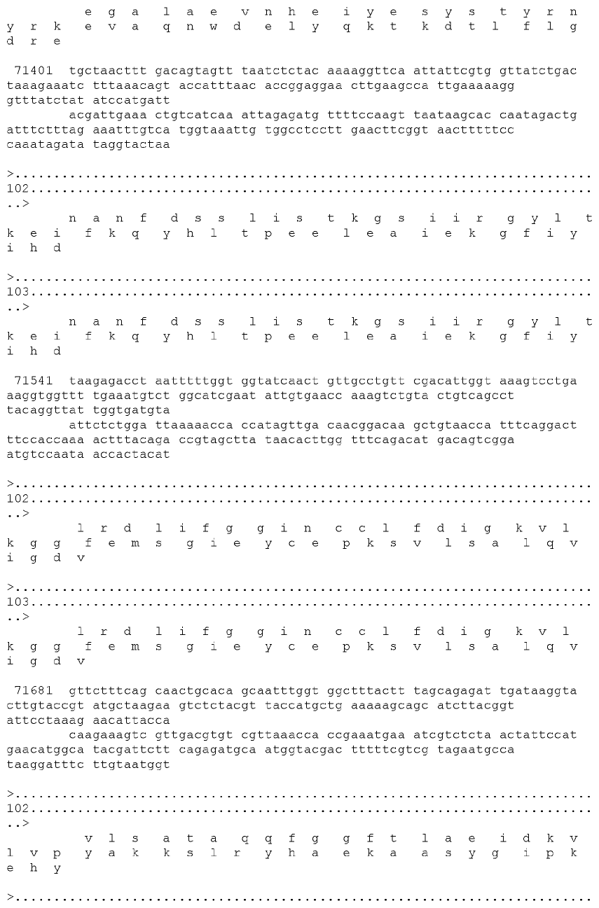

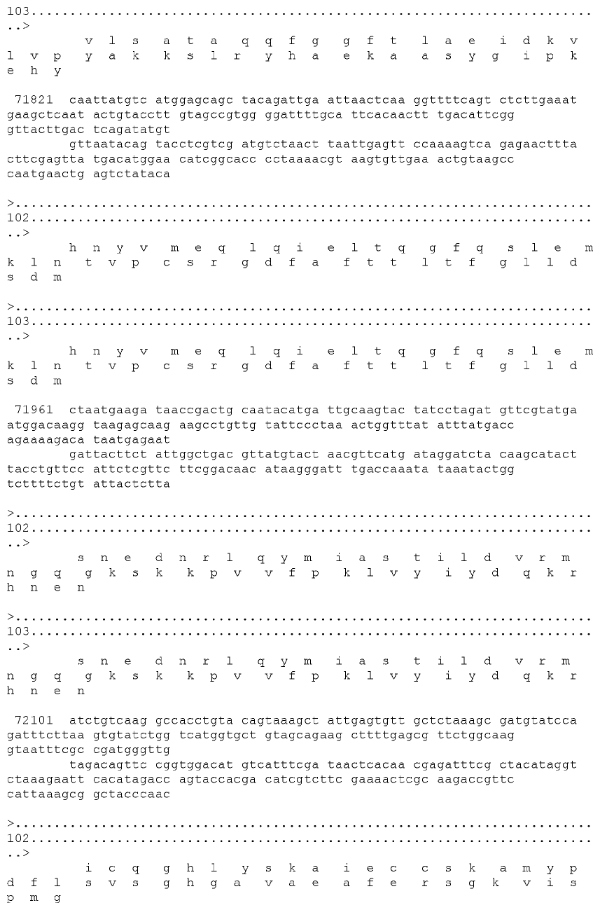

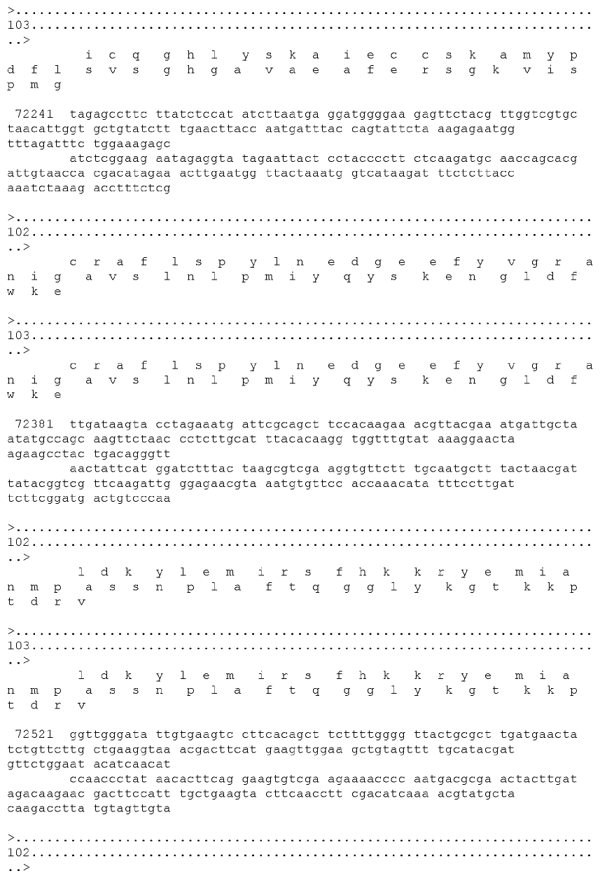

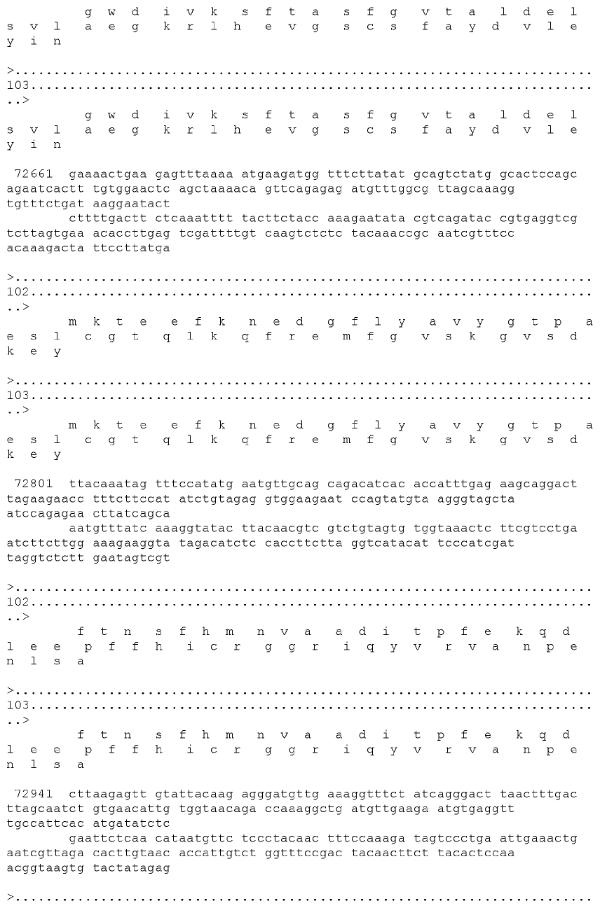

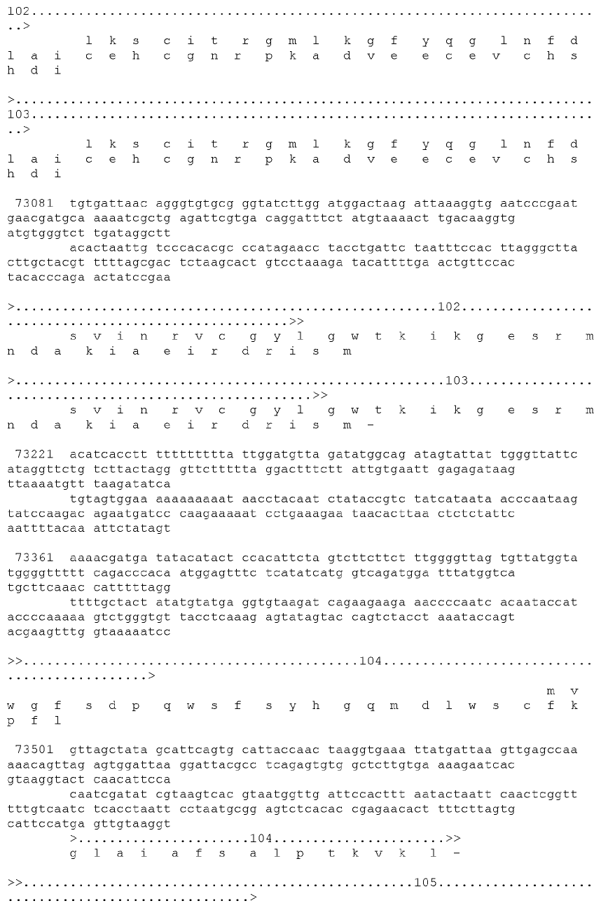

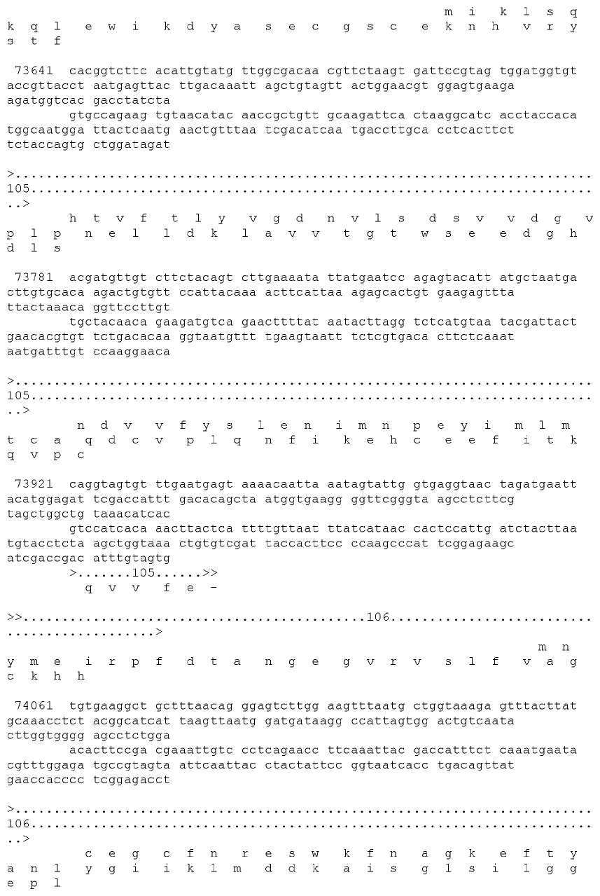

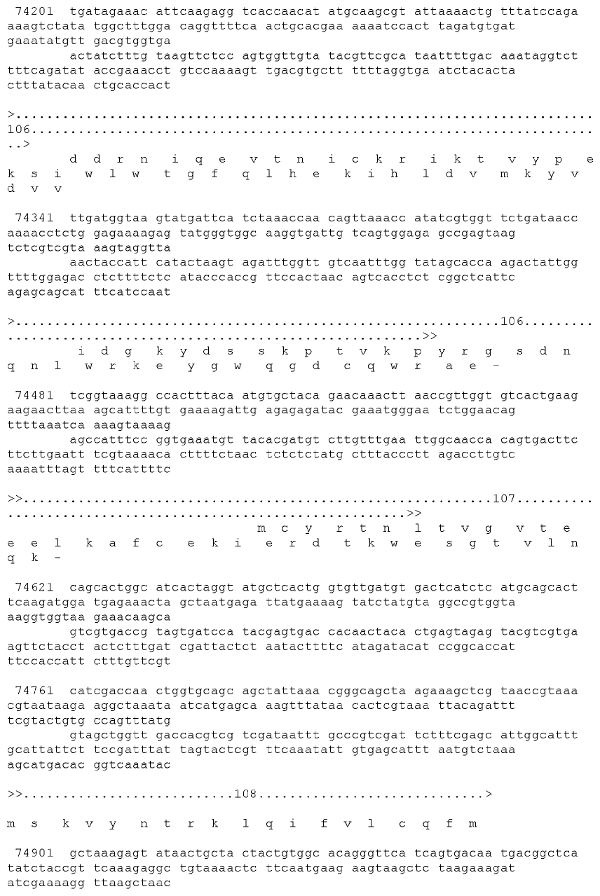

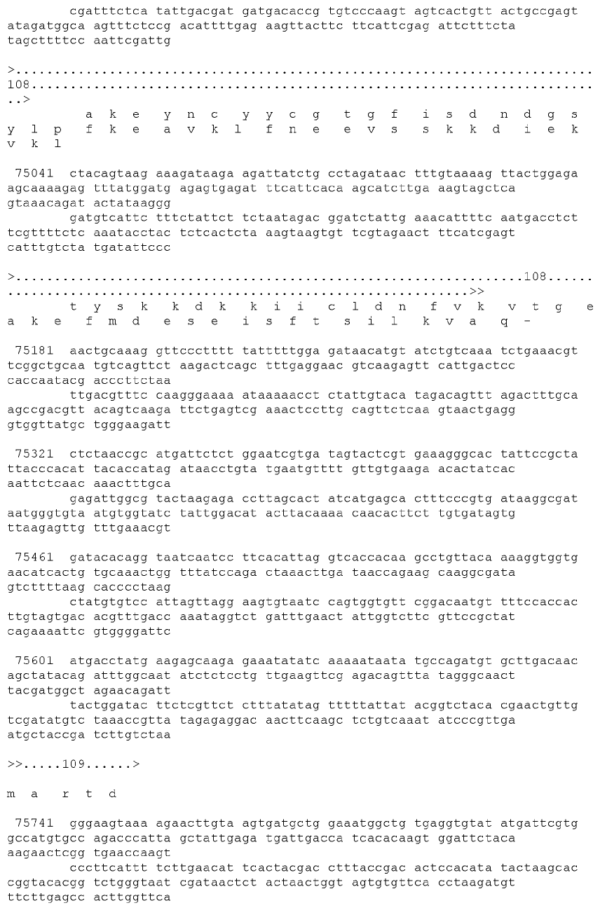

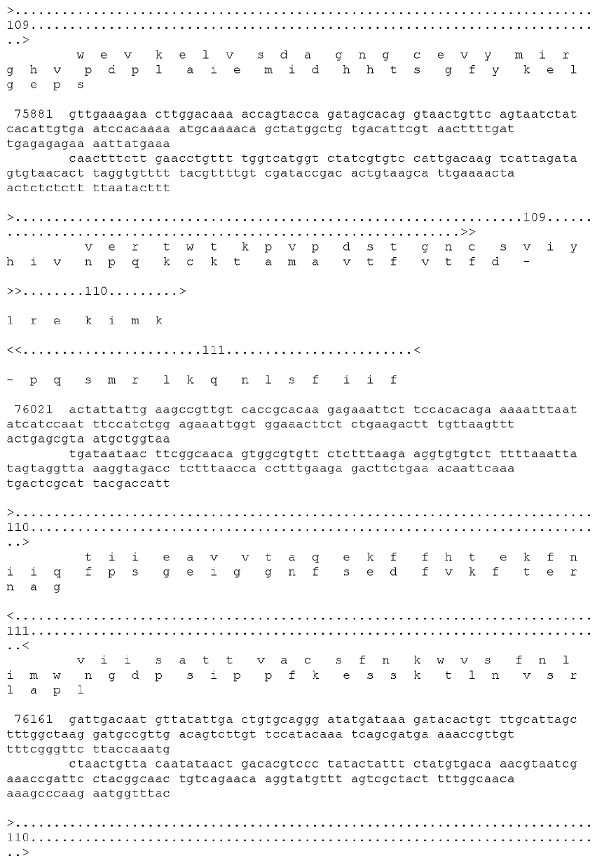

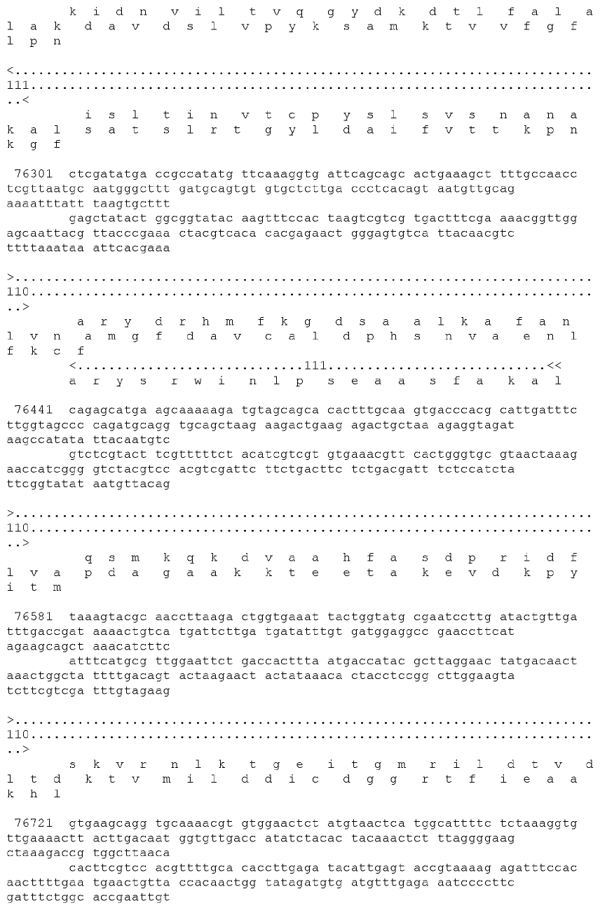

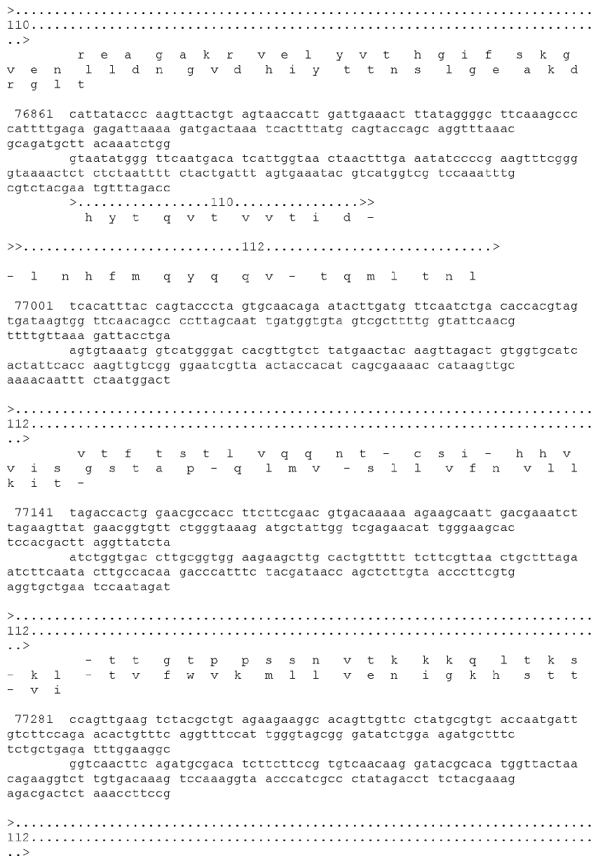

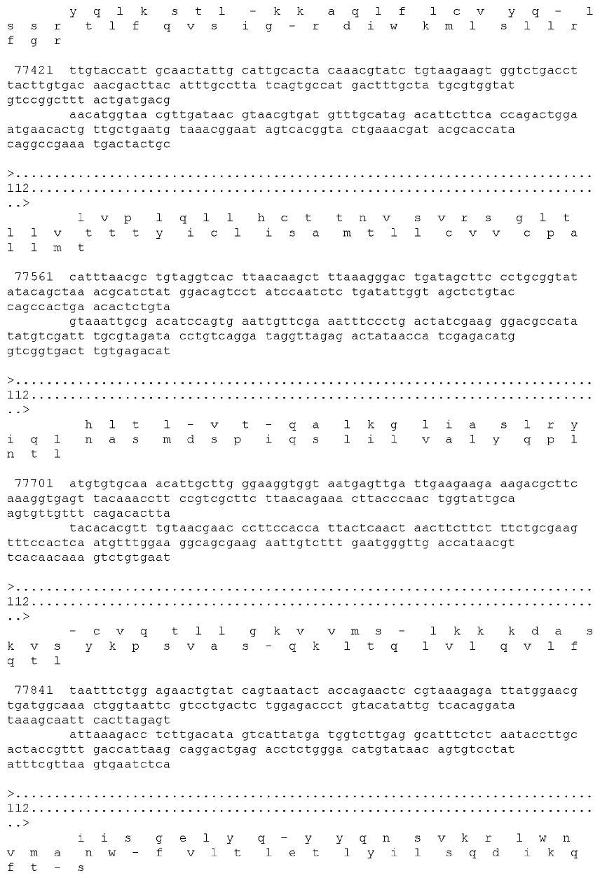

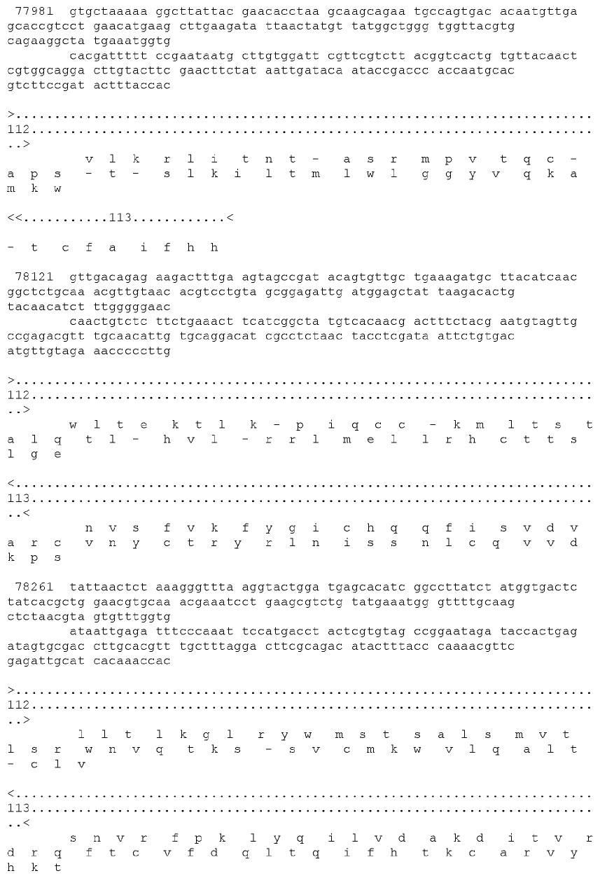

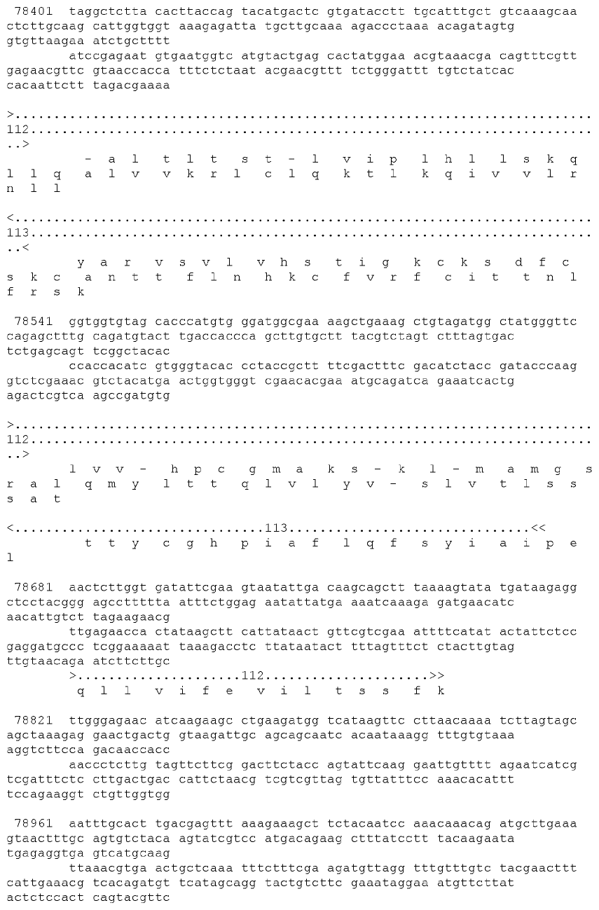

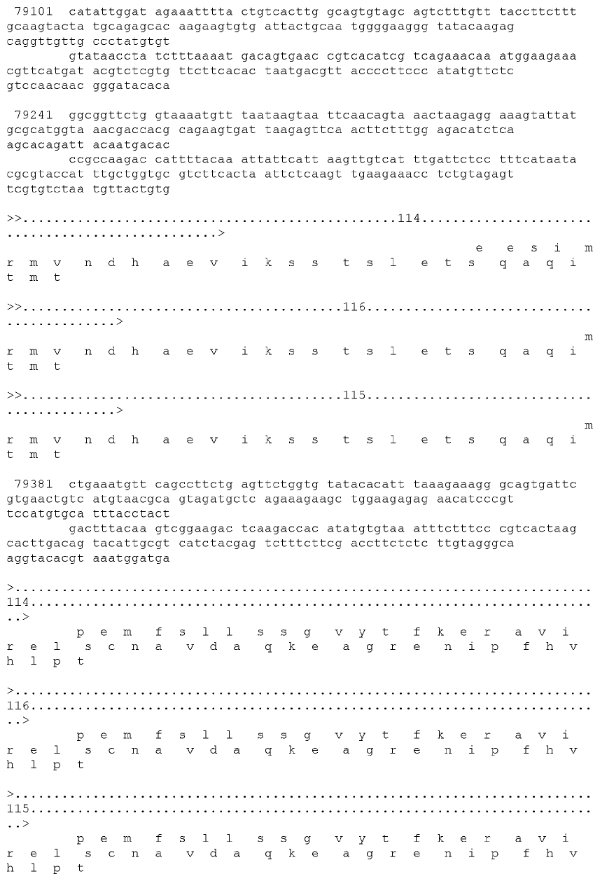

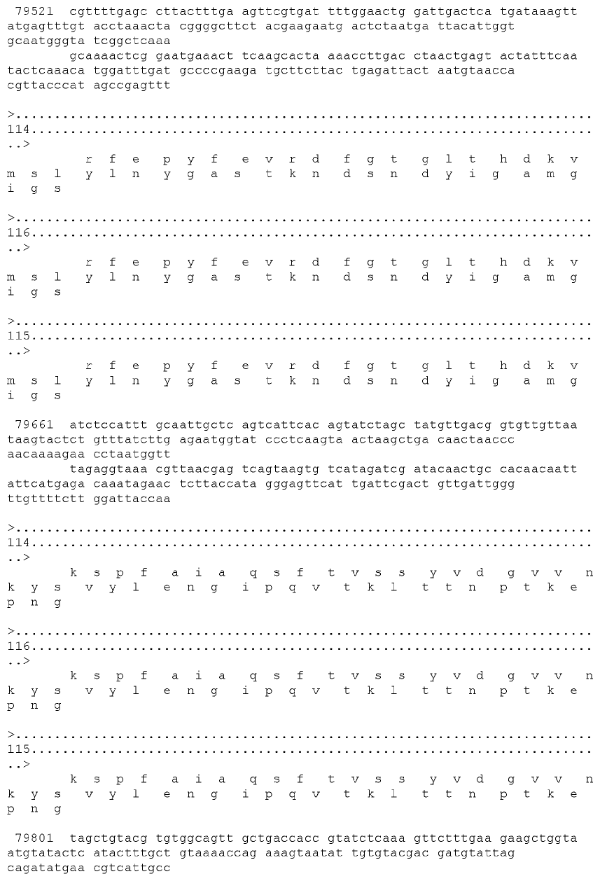

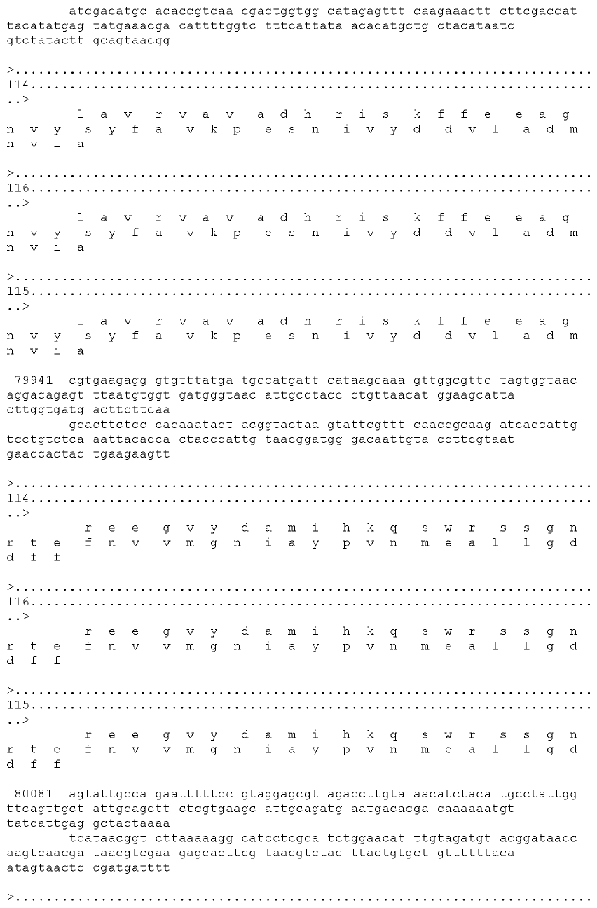

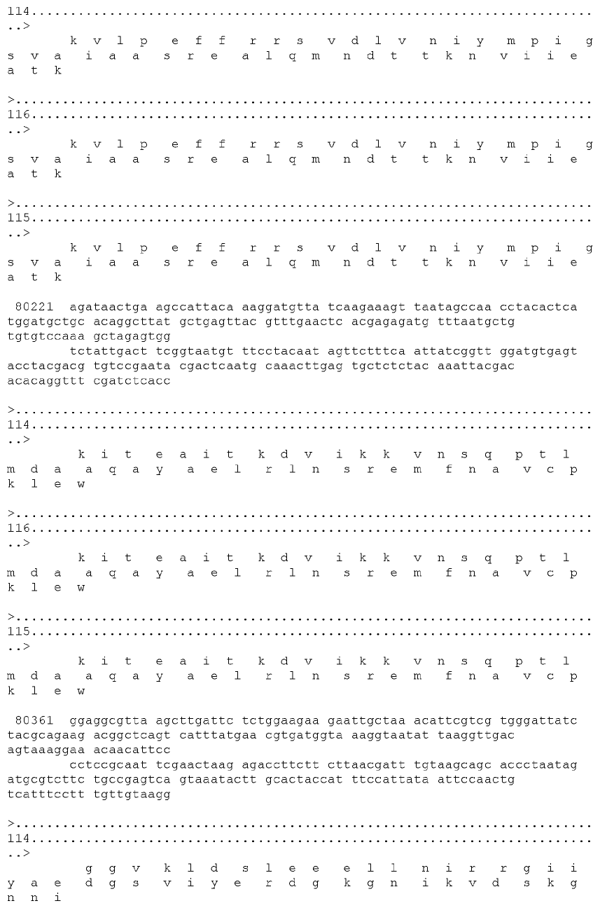

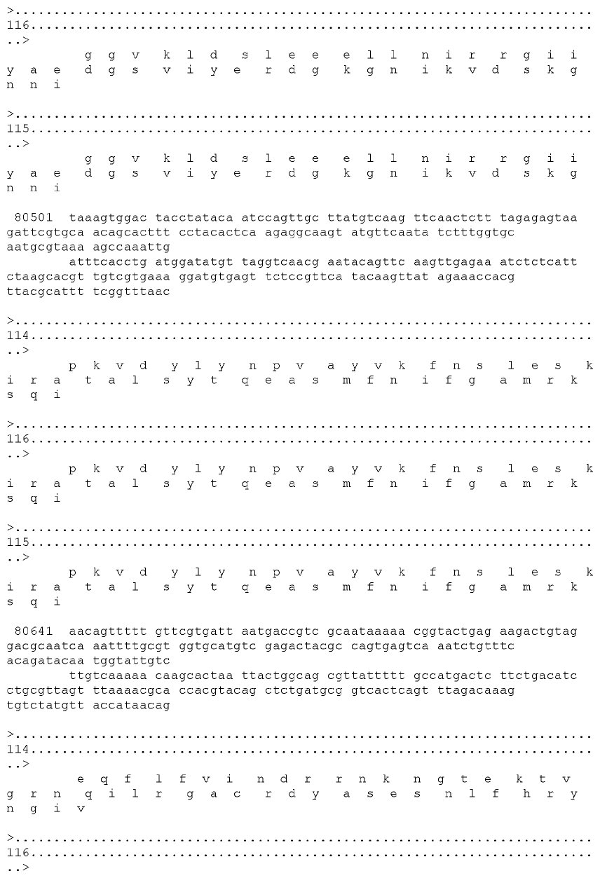

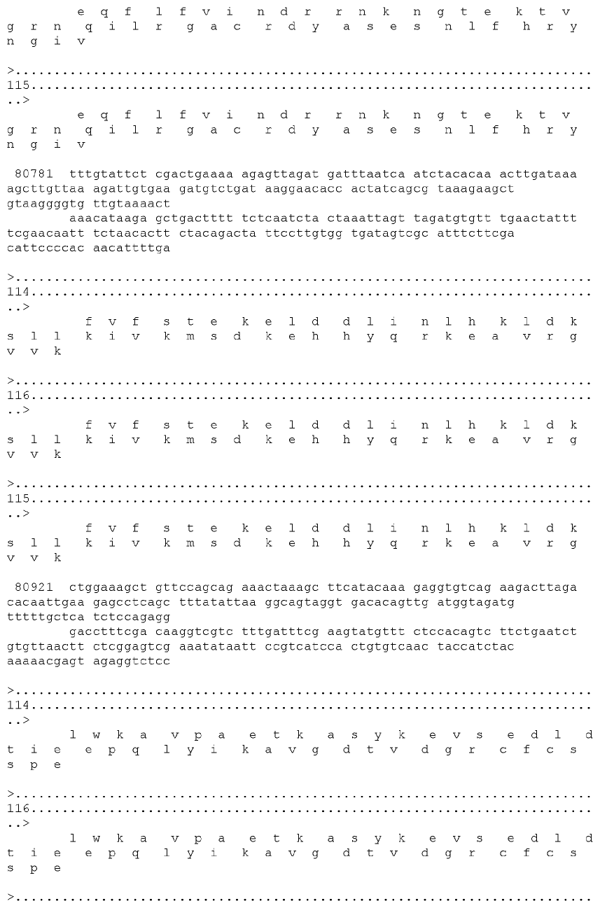

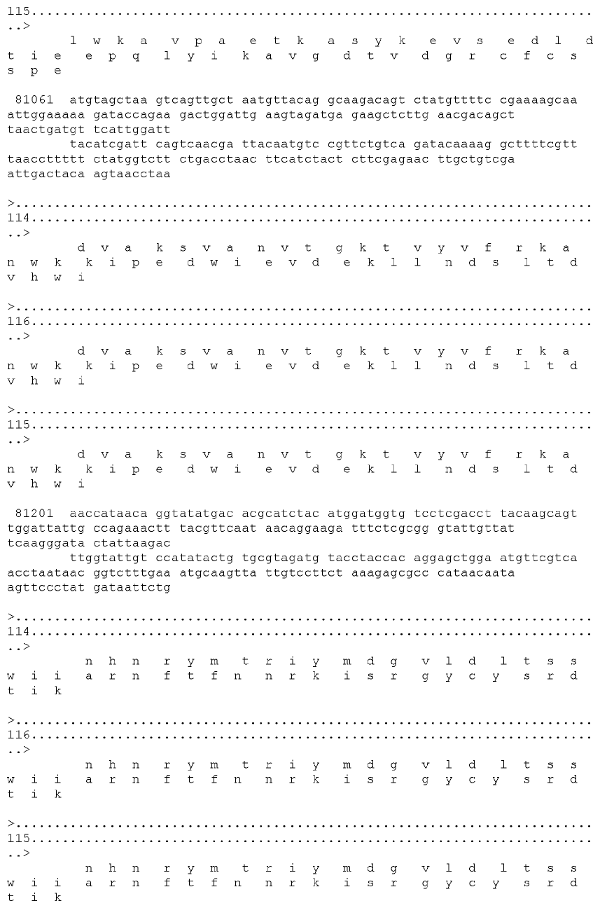

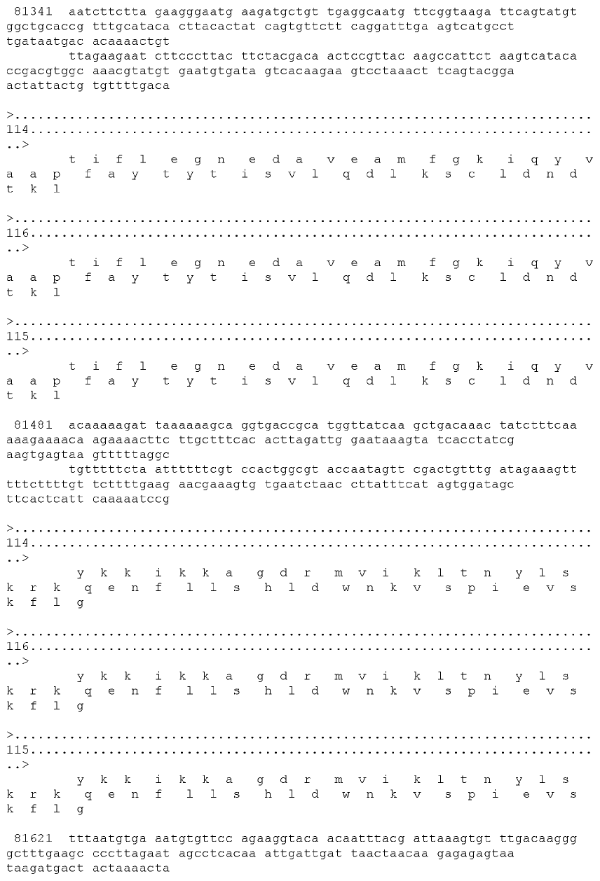

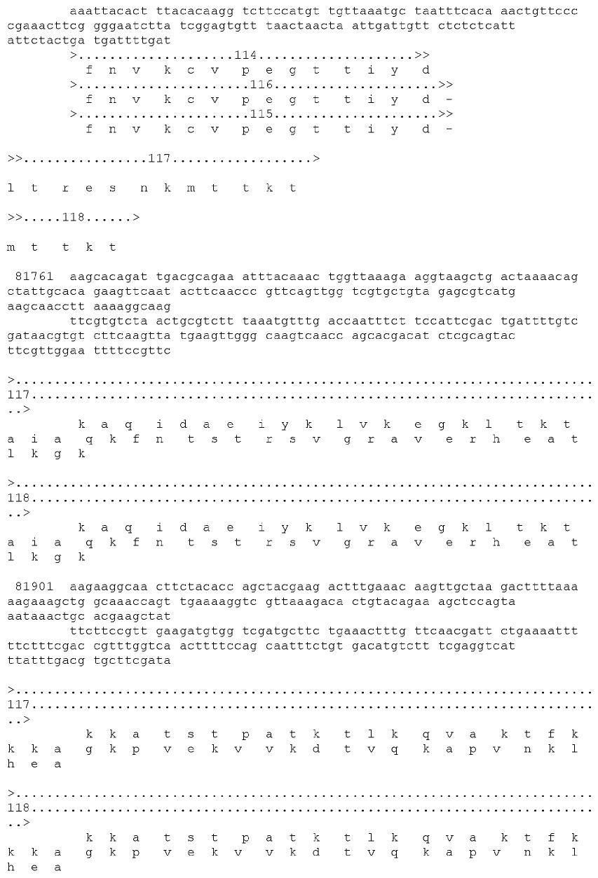

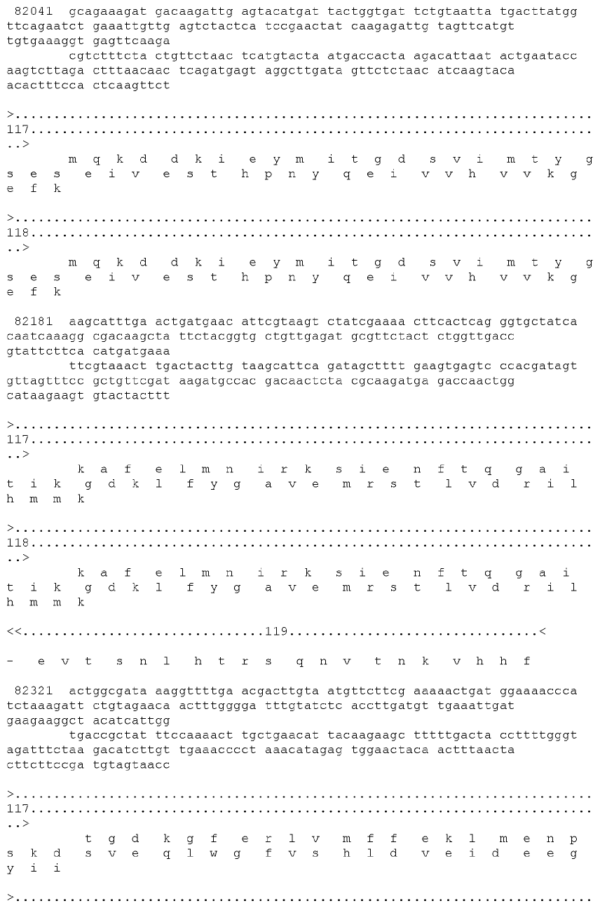

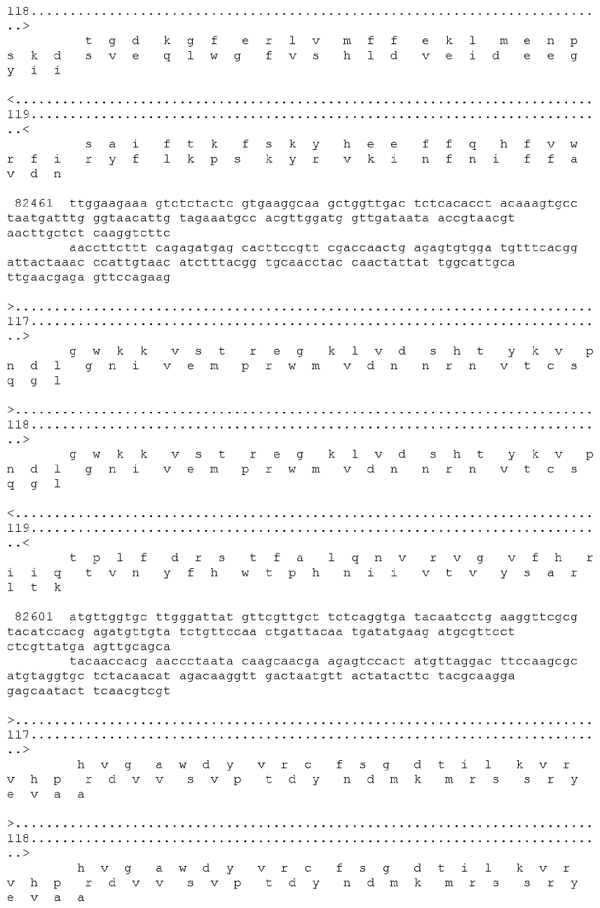

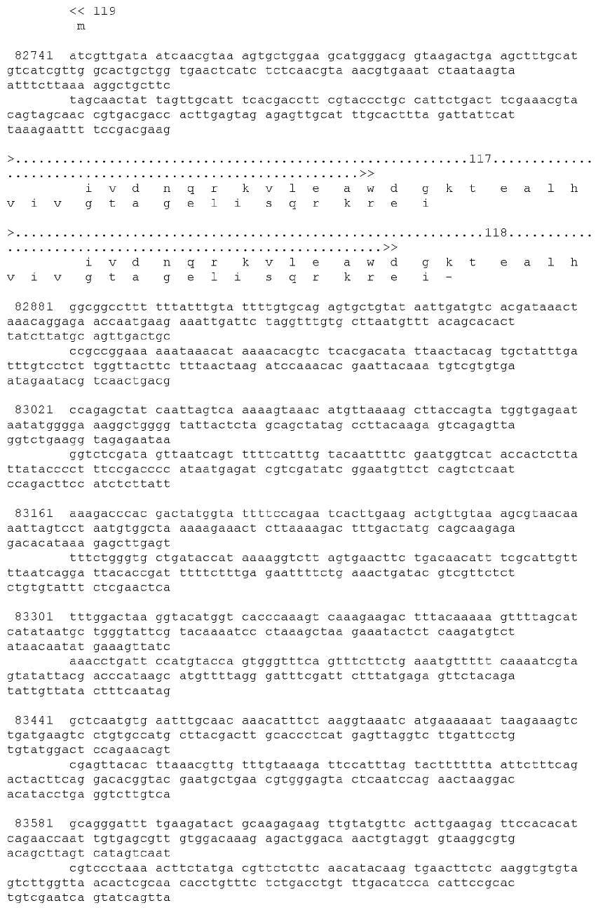

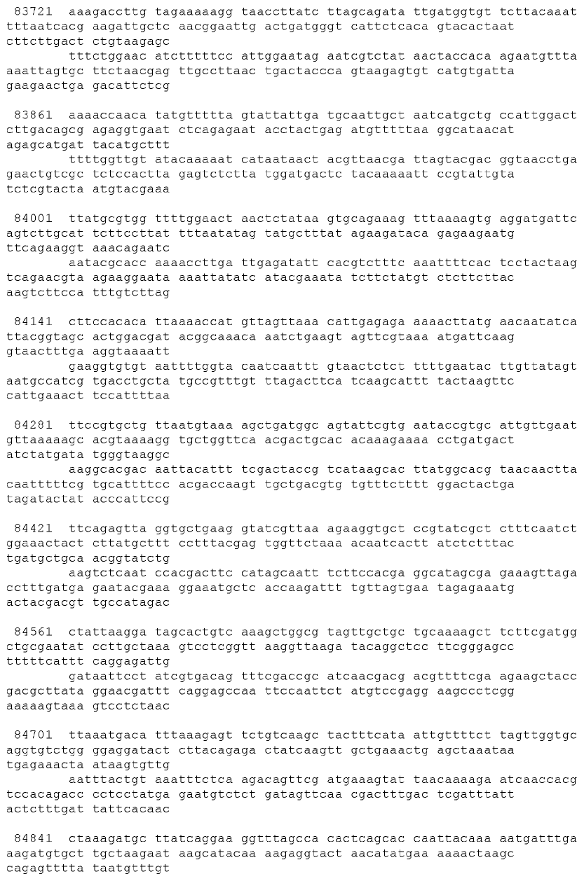

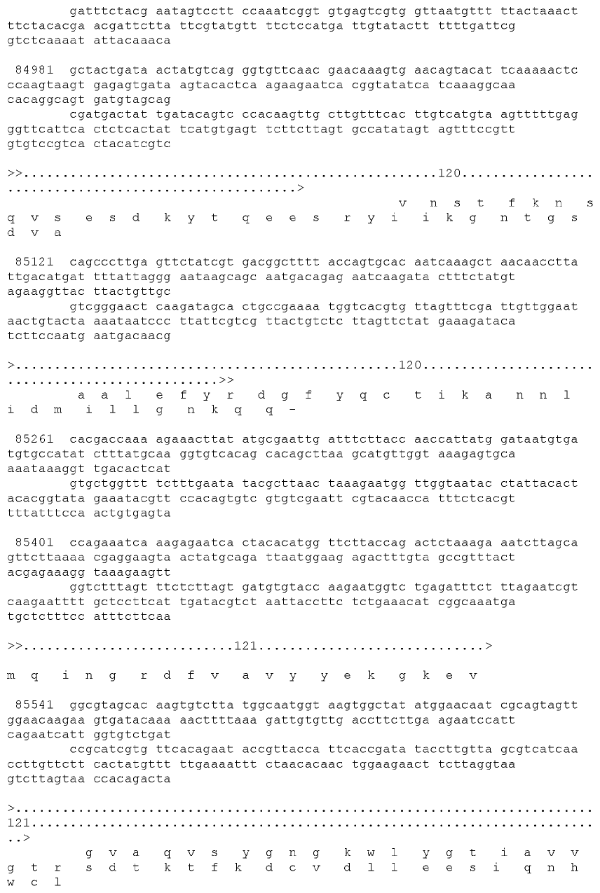

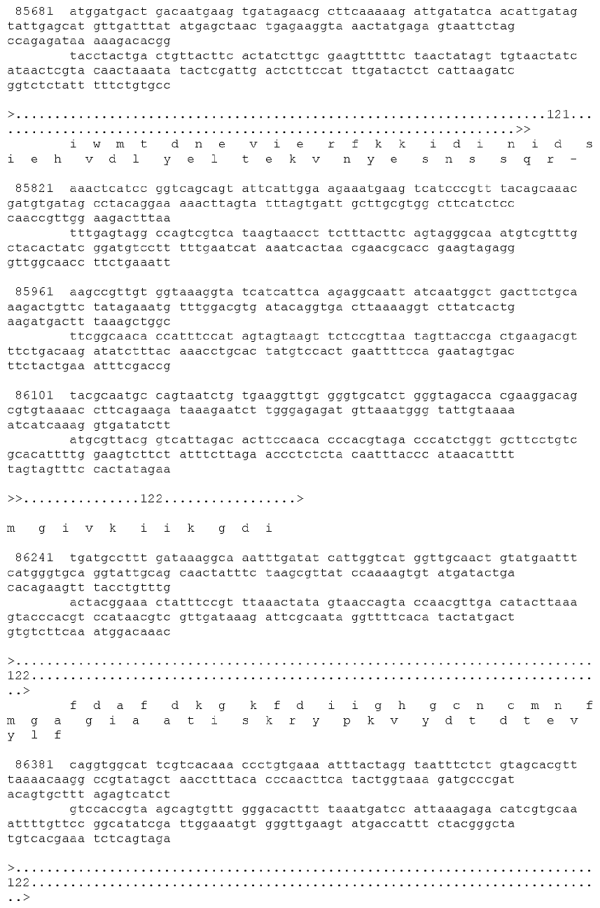

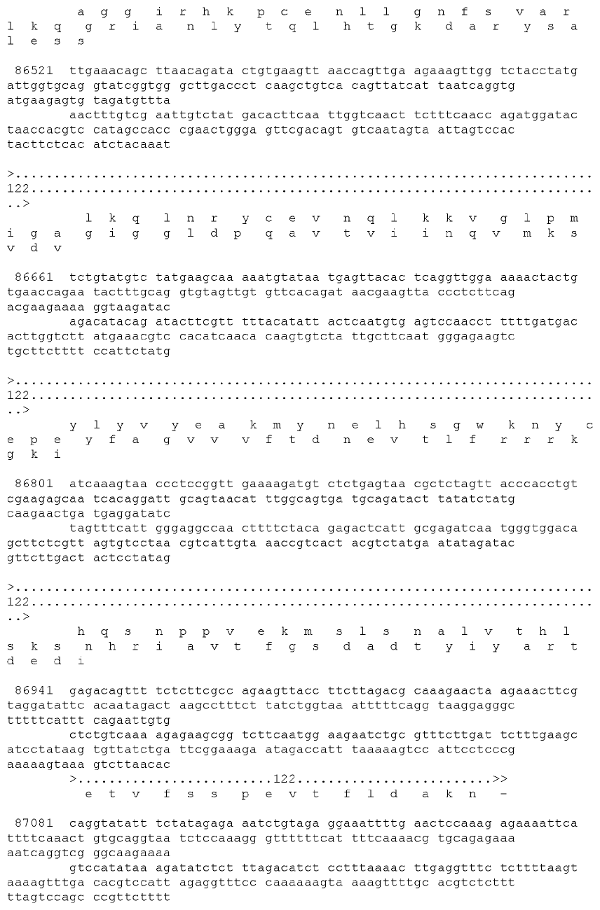

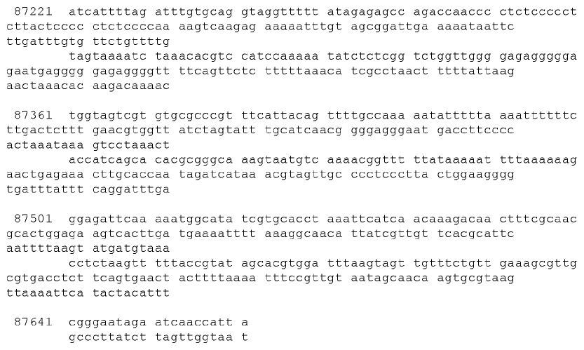

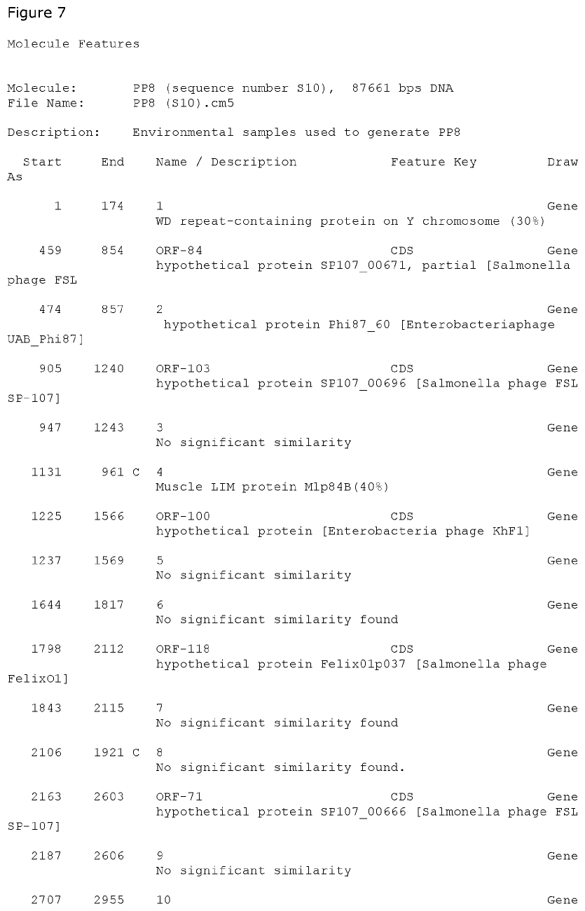

A graphical representation which depicts the location of the genes of the EV31/PP8 is shown in FIG. 5 and a detailed nucleotide sequence of the entire genome of showing sense strand (SEQ ID NO: 1), the antisense strand of the complementary sequence (SEQ ID NO:2), and the sequence of the proteins encoded therein (SEQ IDs NO: 3-124) along with the restriction endonuclease sites is provided in FIG. 6. FIG. 7 shows a detailed description of the EV31/PP8 molecule and proteins with annotations.

### Example 4—Clone Verification by Colony PCR

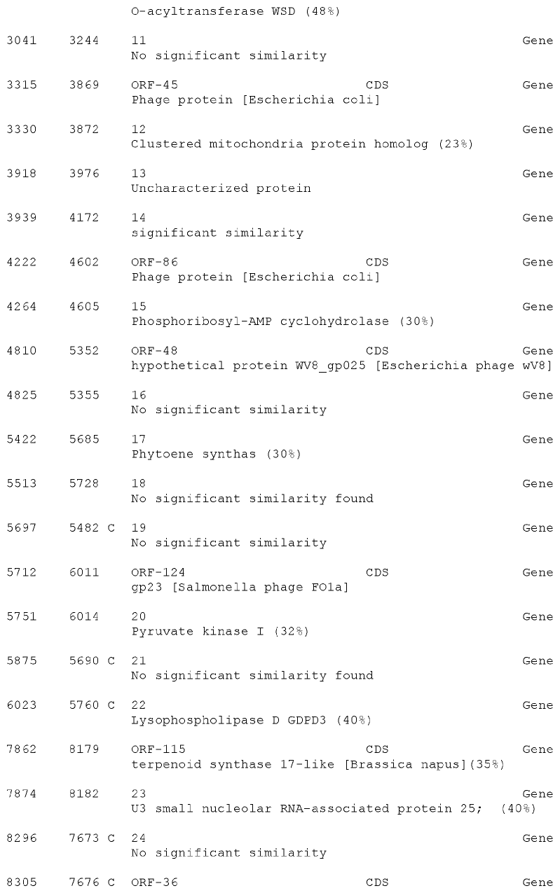

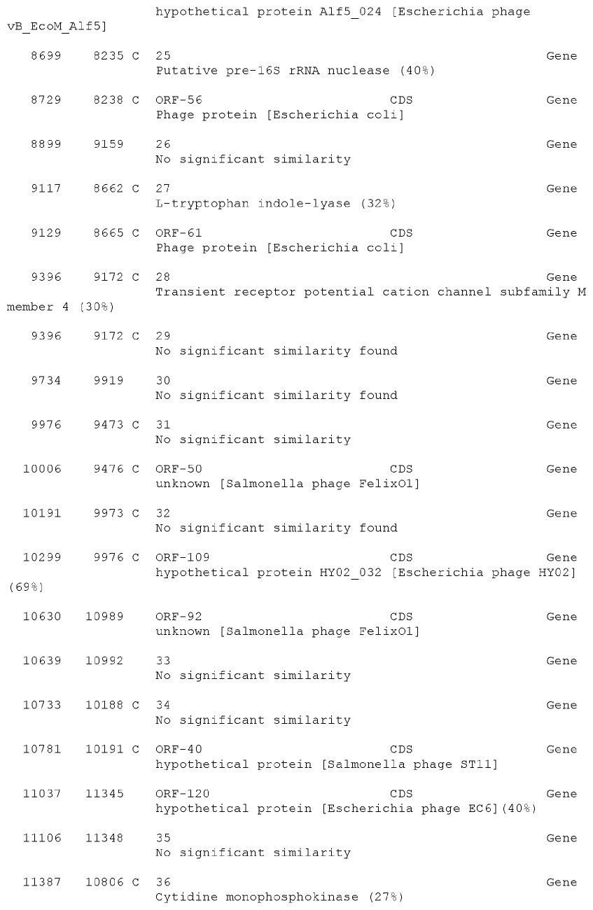

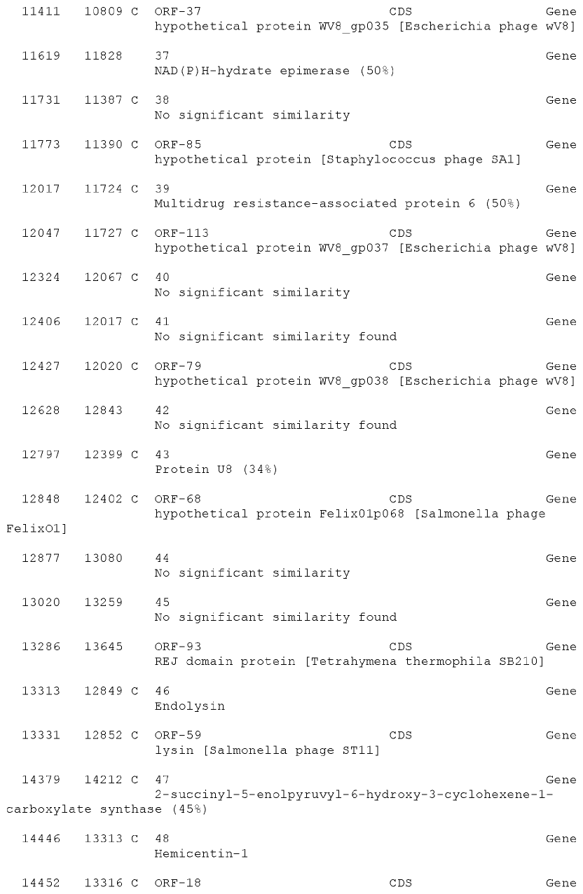

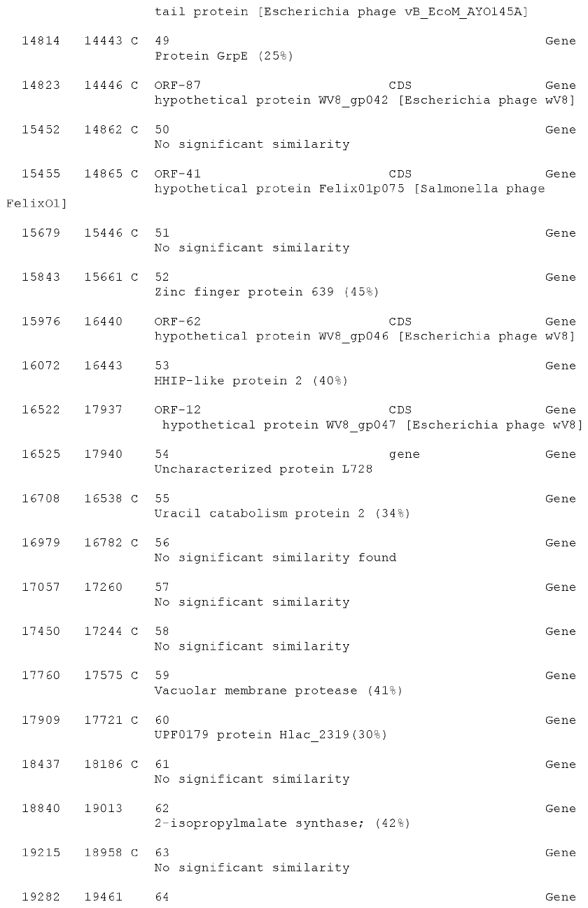

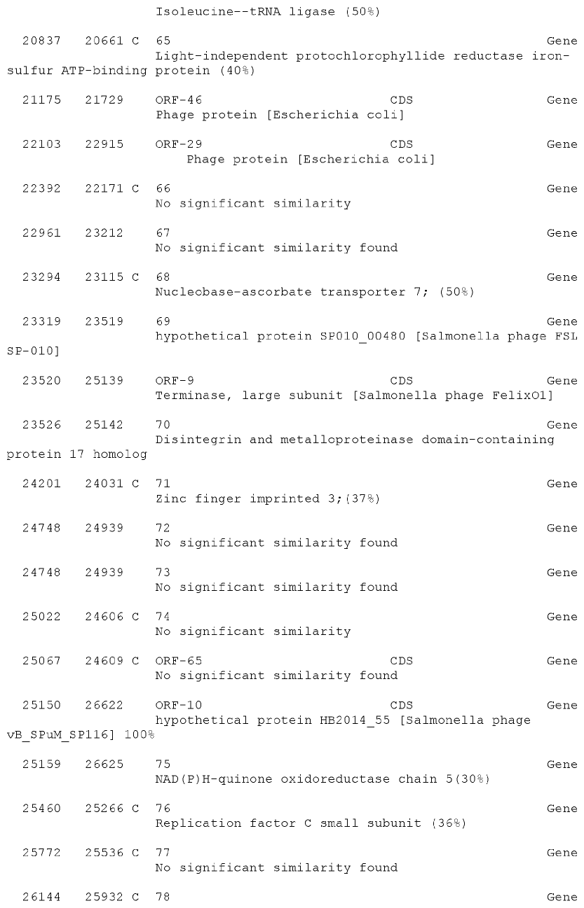

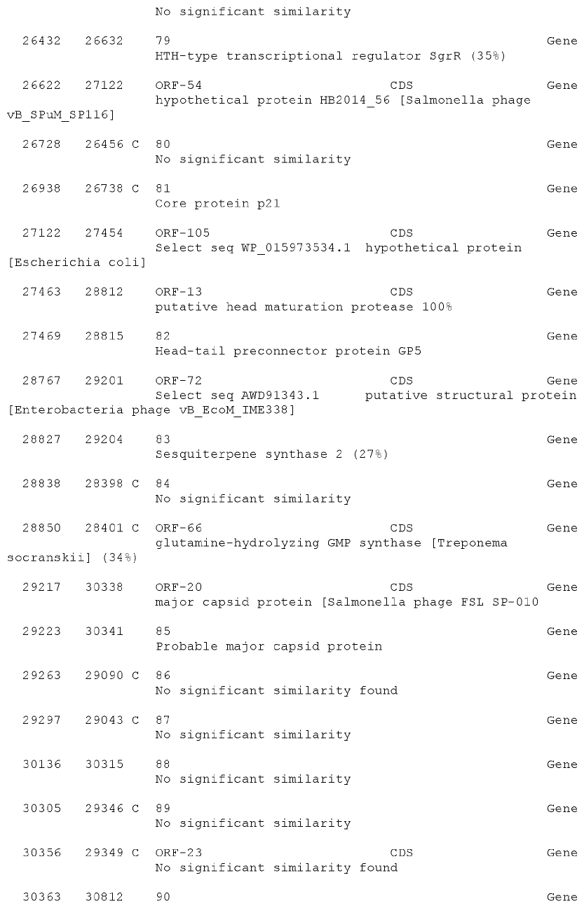

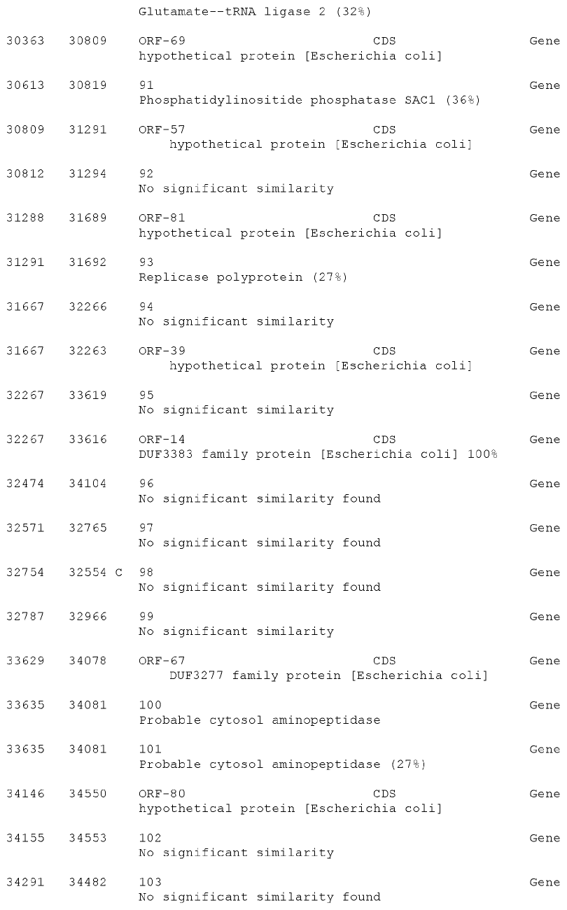

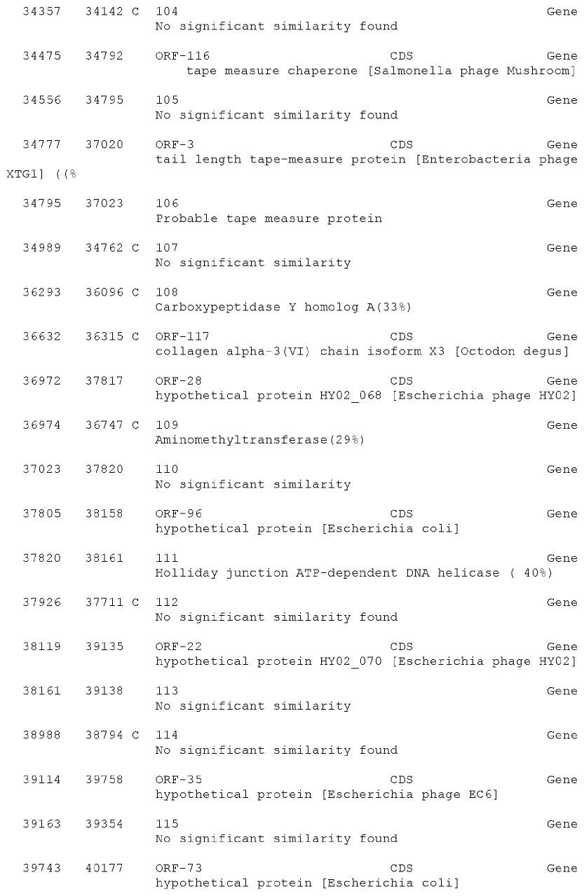

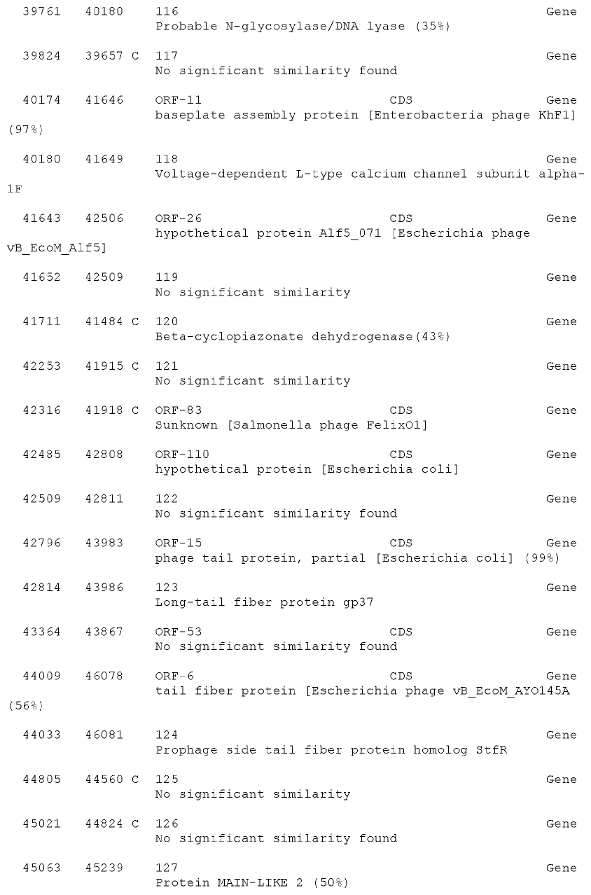

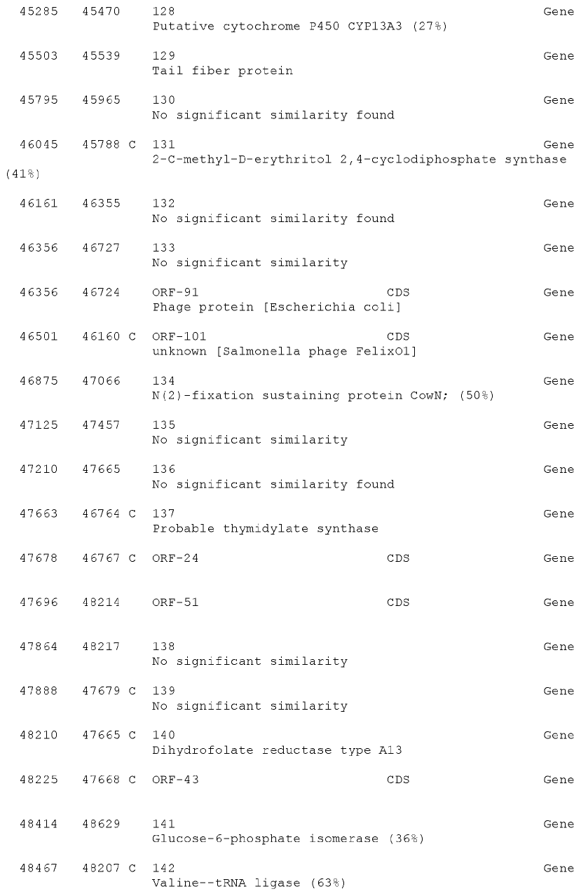

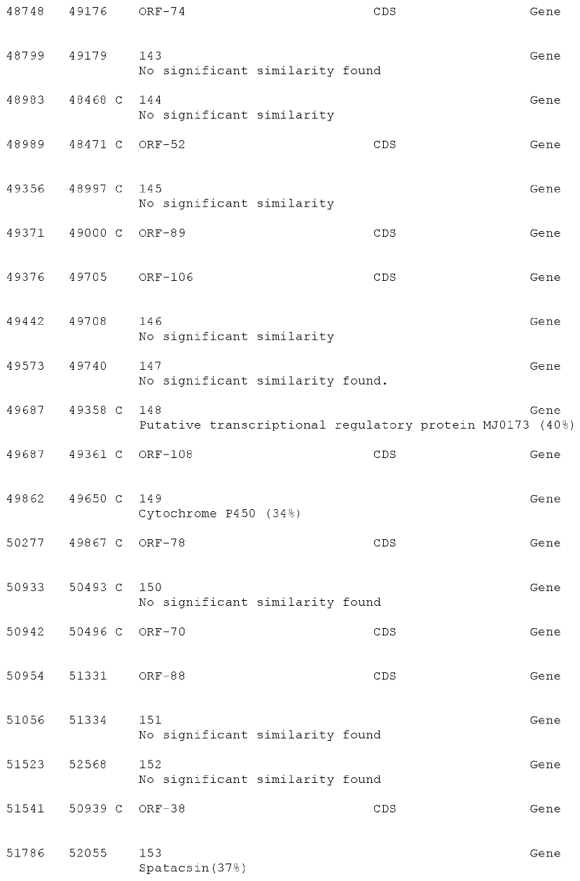

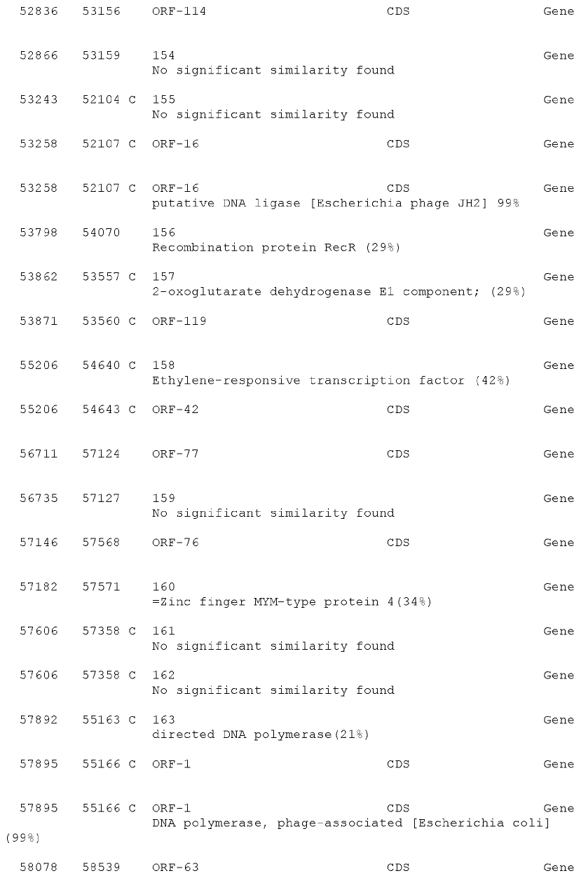

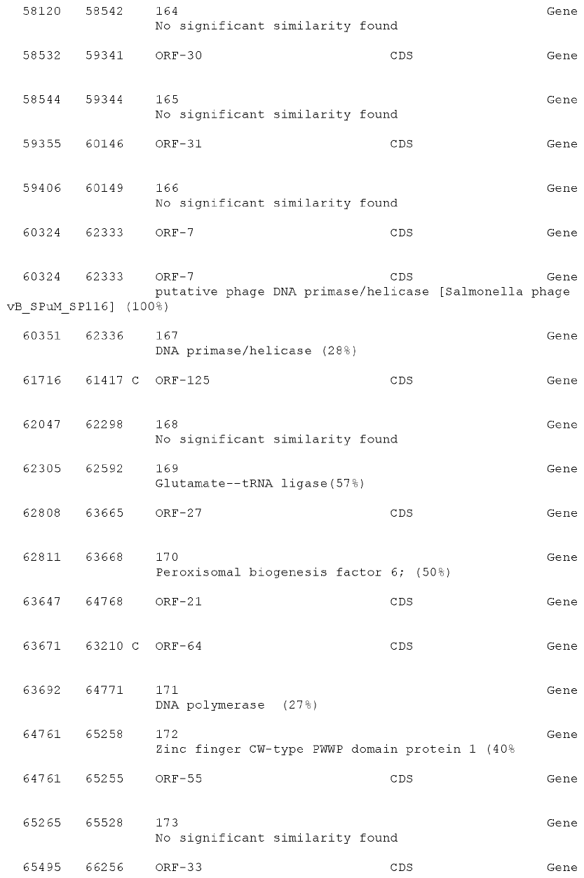

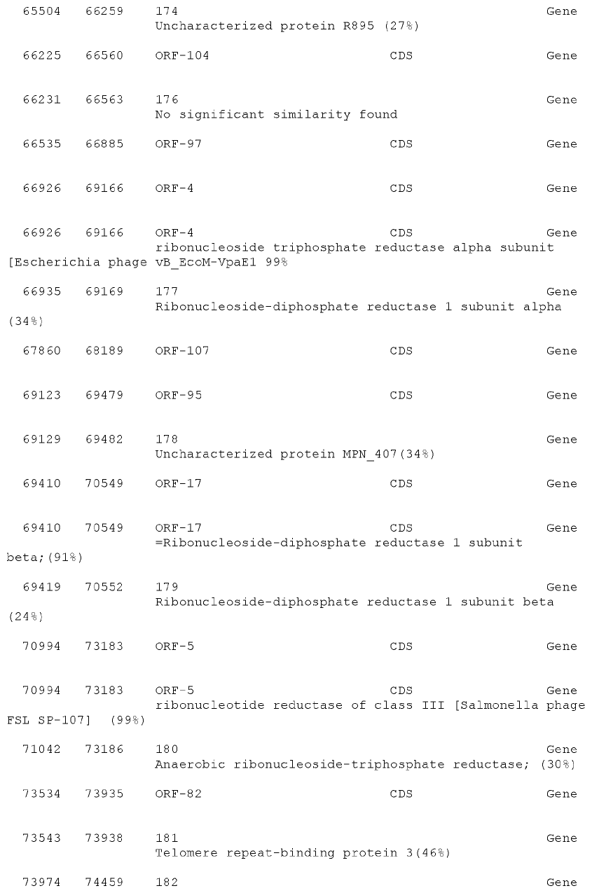

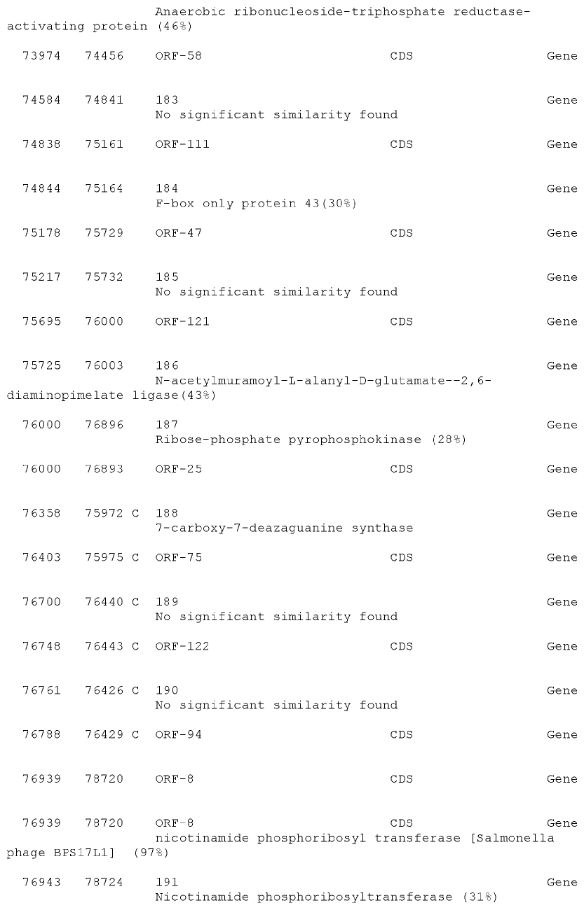

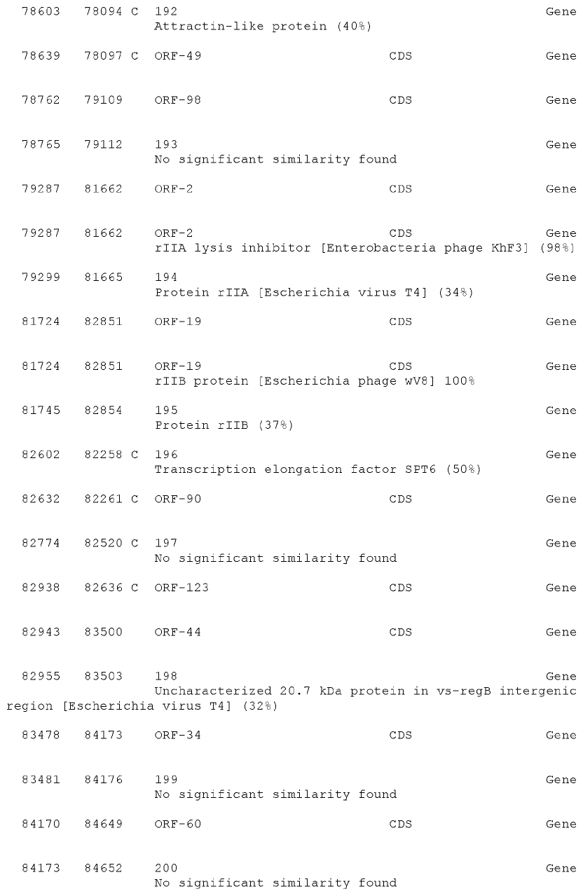

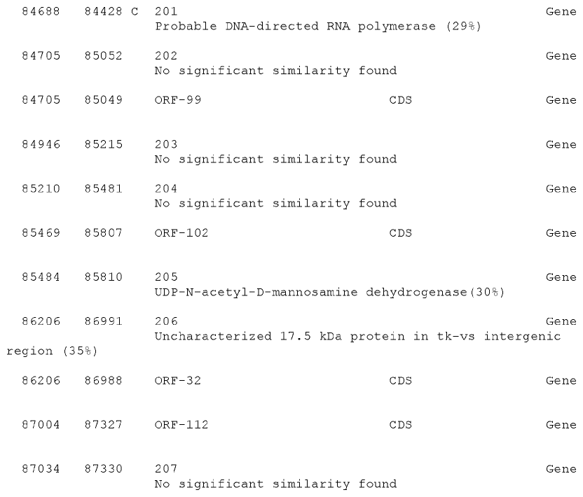

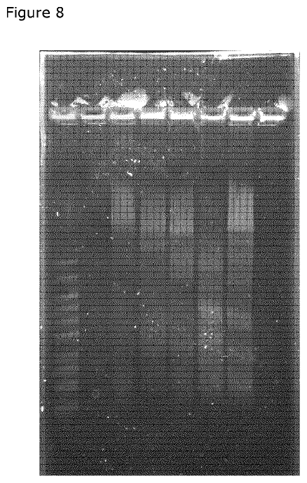

Screening for positive-transformants (plate growth colonies) was carried out as follows. Individual yeast colonies were placed in into 15 μl of lysis buffer for inoculation. In a separate tube, 5 μl of each mixture was transferred and stored at 4C, until ready for large scale grow up of positive colonies. The remaining 10 μl of cell suspension was boiled for 5 minutes at 95C, then immediately placed on ice, adding 40 μl of nuclease-free water and mix. 0.5 μl of lysate was added to each PCR reaction in a total volume of 50 μl and visualized by agarose gel electrophoresis. The resulting gel of the PP8 DNA digestion is shown in FIG. 8.

### Example 5—Development of Unique ORF's

Using the PP8 template, a mutant bacteriophage was generated. Native attachment proteins were removed by generating point mutations using homologous recombination.

Gene Disruption 14452-13316 (tail protein):
>PP8-F1F
SEQ ID No: 185
ACAAATAGTGAAGAGATAAACCAGGTTGAGCAAG
>PP8-tail-mut-R
SEQ ID No: 186
TTGACGTTGAATCTGGAGTCGATAGGTGCGACAGGTTACCAATGG
>PP8-tail-mut-F
SEQ ID No: 187
GTCGCACCTATCGACTCCAGATTCAACGTCAAGGTCTCACC
>PP8-F1R
SEQ ID No: 188
TTCCAAGACGGATTCGAACCGTCACTAGTACAAGG
Gene Disruption 14823-14446 (tail protein):
>PP8-F1F
SEQ ID No: 185
ACAAATAGTGAAGAGATAAACCAGGTTGAGCAAG
>PP8-hyp1-mut-R
SEQ ID No: 189
TTAATGATGTTATCTCGATAACGTCGACATGGAGACTCAGTAAATGG
>PP8-hyp1-mut-F
SEQ ID No: 190
TCTCCATGTCGACGTTATCGAGATAACATCATTAAGGTTGTACC
>PP8-F1R
SEQ ID No: 188
TTCCAAGACGGATTCGAACCGTCACTAGTACAAGG
Gene Disruption 16522-17937 (tail protein):
>PP8-F1F
SEQ ID No: 185
ACAAATAGTGAAGAGATAAACCAGGTTGAGCAAG
>PP8-hyp2-mut-R
SEQ ID No: 191
TTGAATAAACCGTTATCGCCTTCTTAAAGCAACCTGTATTGCGTTCTGC
>PP8-hyp2-mut-F
SEQ ID No: 192
TTGCTTTAAGAAGGCGATAACGGTTTATTCAACAAACCCTCATTTCATTG
>PP8-F1R
SEQ ID No: 188
TTCCAAGACGGATTCGAACCGTCACTAGTACAAGG
Gene Disruption 34777-37020 (tail protein):
>PP8-F3F
SEQ ID NO: 158
TTCTTAAGGAGGGTTATGAATGTGTTATACAGG
>PP8-tape-mut-R
SEQ ID No: 193
TCTGTGTAGTTCGGCCAACTGTAGTGTGCGAATGATGCAGCGAACATTC
>PP8-tape-mut-F
SEQ ID No: 194
TTCGCACACTACAGTTGGCCGAACTACACAGATACCATGAAGCAGTACTC
>PP8-F3R
SEQ ID NO: 167
GTGGTAAGGTAAGGTATGGAAGGATGGCAGTAG

The mutant bacteriophage can comprises four ORFs: ORF 1 is located at position 46090; ORF 2 is located at position 73195; ORF 3 is located at position 19991; ORF 4 is located at position 60431.

Using modification primers EV31/ORF1/F and EV31/ORF1/R for ORF1 (between nucleotide positions 46,090 and 46,091) and EV31/ORF2/F and ORF2/R for ORF 2 (between nucleotide positions 73,195 and 73,196) and cell free cloning we generated three EV31 mutant constructs. EV31 (ORF1), EV31 (ORF2), and EV31 (ORF1/2). In the construction of ORF2 we first removed the natural binding domain from EV31 and added a multiple restriction enzyme cassette. This cassette is then used to add new bacterial binding domains.

Both homologues recombination and insertion using restriction digests. For restriction digest, the enzyme TspRI allows insertion of a multiple cloning site (MCS). In one example, ORF3 is located at 19991 in ev31/pp8 sequence. In this example, the insertion of the MCS would be done by using TspRI. Once the MCS is inserted, the insertion an attachment gene of choice can done achieved by using restriction enzymes sites found in the MCS. Example of MCS for ORF3: GCCGGCAGTGGATCCCCGGGGAAGATATTC SEQ ID NO: 153. This MCS carries enzymes sites for Nael, TspRI, Xmnl, SmaI. The primers used for adding the MCS to site 19991 are: EV31 ORF3 primer f GCTACACTGCTGAGA SEQ ID NO: 154; EV31 ORF3 primer r TCTCAGCAGTGTAGC SEQ ID NO: 155.

The fourth ORF is located at 60431 in ev31/pp. In this example, the insertion of the MCS would be done by using TspRI. Once the MCS is inserted, the insertion an attachment gene of choice can done achieved by using restriction enzymes sites found in the MCS. The primers used for adding the MCS to site 60431 are: EV31 ORF4 primer f CATCAGATGCTGG SEQ ID NO: 156; EV31 ORF4 primer r CCAGCATCTGATG SEQ ID NO: 157.

### Example 6—Integration

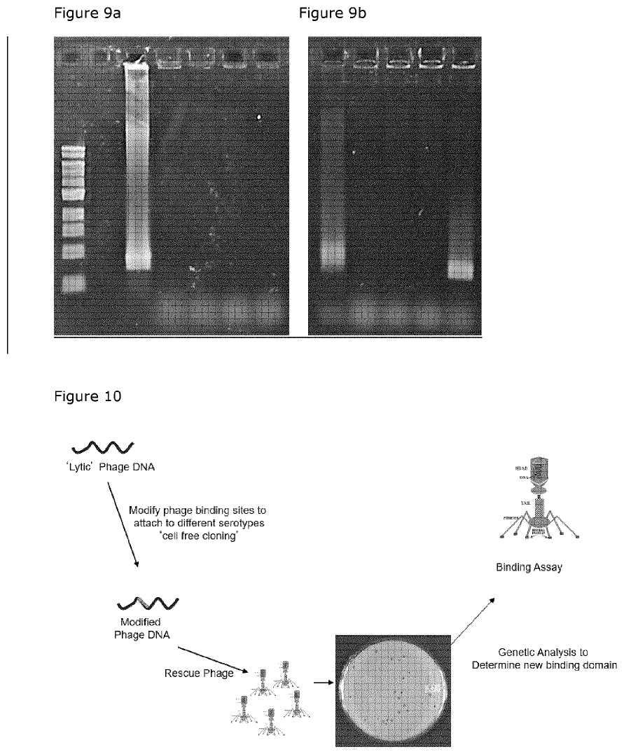

An analysis of the genome of the EV31/PP8 revealed a possible lysogenic gene located at 60351-62336. Mutation of the gene by generating an ORF at the site of 60431 (ORF4). Once the lysogenic gene were inactivated, we carried out integration studies to ensure integration did not occur. The results are shown in FIGS. 9a and 9b.

Under conditions that promote integration we confirmed that PP8 lacks the ability to integrate. The gel electrophoresis photograph identifies integration events demonstrated by a bacteriophage (bacteriophage induction control), determined by polymerase chain reaction (PCR) on whole bacterial cells. A respective primer set for each bacteriophage would give a positive PCR signal (right panel; lane 5) if the bacteriophage genetic material was integrated inside of the purified (bacteriophage particle-free) bacterial colonies. Contrarily, PP8 cannot integrate into the bacterial host cells, as indicated by the absence of a positive signal for the PP8 sequence in the photograph (left panel; lanes 5-7).

### Creating Conditions for Integration

Fresh overnight cultures of the bacterial host (Escherichia coli C) from glycerol stocks were prepared in Luria-Bertani (LB) broth. Once saturated, the cultures were diluted (1:100) in fresh LB broth, supplemented with 2 mM CaCl₂ and incubated until an OD₆₀₀ of 0.6. Mixtures of host (100 μL of E. coli C) and bacteriophage (100 μL at multiplicity of infection of 5) in 3 mL of molten, soft agar were overlaid onto previously, dried LB-agar plates. Following an overnight incubation, three colonies from each plate were picked, re-streaked onto fresh LB-agar plates and incubated overnight for three rounds. The purified colonies (free of contaminating bacteriophage particles) were inoculated into LB-2mM CaCl₂ broth and incubated overnight.

### Analyzing Potential Integration Events

Polymerase chain reaction (PCR) master mixes of GoTaq® DNA polymerase (Promega) were set up following the manufacture's recommendations along with the respective primers for each bacteriophage to be evaluated. Five μL from each overnight culture were spiked into their respective PCR reaction. Cycling conditions were altered to include whole-bacterial cell boiling in the initial denaturation period (95° C. for 10 minutes) and an annealing temperature of 59° C. Five μL of completed PCR reactions were subjected 1% agarose gel electrophoresis, stained and visualized under ultraviolet (UV) light. The results are shown in FIGS. 9a and 9b.

### Example 7—ORF1 Insertion SP5 Attachment Protein (between 46,090 and 46,091)

Using PP8 we developed of a MRSA specific PP8 binding phage by utilizing the PP8 template we removed native attachment genes and added attachment protein SP5 at the ORF 1 location (between 46,090 and 46,091) using homologous recombination. The primer sets used for this homologous recombination are:

1. Primer set for PP8 Fragment (bold) and
homologous recombination with SP5 gene (underline)
on 5′
>PP8-F3F
SEQ ID NO: 158
TAATACTCTACAGACACCACTAACTGATGCTGCTG
>PP8-SP5-R
SEQ ID NO: 159
CTCGTTTCAACATCTTTTATTTTGTACATACAAGGGATTAAGCAGTTCTT
ACCC
2. Amplification of SP5 (underline):
>SP5-F
SEQ ID NO: 161
ATGTACAAAATAAAAGATGTTGAAACGAG
>5P5-R
SEQ ID NO: 162
CACCCCTTAATTAAATAAAGTGTATTAGGGTC
3. Primer set for PP8 Fragment (bold) and
homologous recombination with SP5 gene (underline)
on 3′
>PP8-SP5-F
SEQ ID NO: 163
CACTTTATTTAATTAAGGGGTGATGACTGATTGTTAAGATGGTGTTAATA
TTC
>PP8-F3R
SEQ ID NO: 167
GTGGTAAGGTAAGGTATGGAAGGATGGCAGTAG

The sequence of the insertion (MRSA attachment protein SR5) is shown in SEQ ID NO: 168.

### Example 8—Phage Efficacy Against MRSA

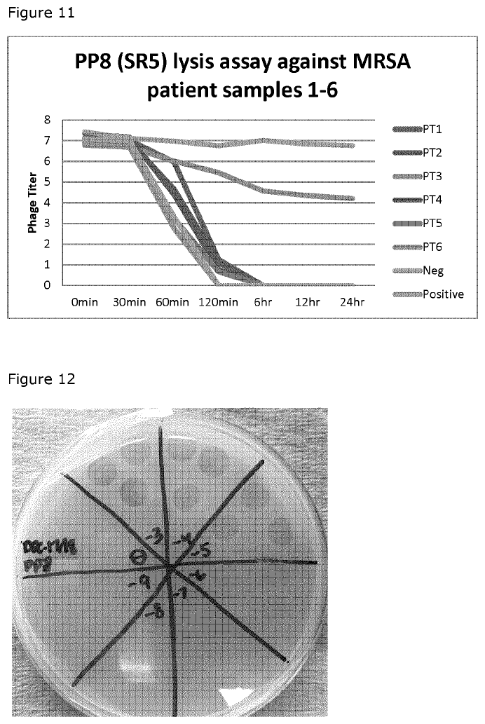

We tested our new PP8(SP5) phage against MRSA infected patient samples 1 through 6. These are MRSA positive patient samples from clinical isolation. An overview of the method is shown in FIG. 10 and the results are shown in FIGS. 11 and 12. Patient samples 1, 2, 4, 5, 6 were all lysed using PP8 (SR5). Patient sample 3 showed only a partial binding profile, which suggested that the binding may not have been specific enough to give a 100% lysis rate. Positive control was PP8 bacteriophage with in SA attachment site.

After sequence analysis of patient sample 3, through sequence analysis and blast searching for attachment sites, a new binding site was revealed.

### Example 9—ORF1 Insertion SP6 Attachment Protein (between 46,090 and 46,091)

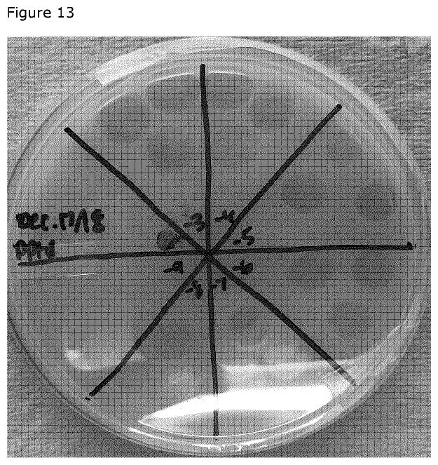

We also generated a PP8 SR6 mutant and tested this against Staphylococcus aureus. The result is shown in FIG. 13.

We then generated a new PP8 strain to attach to and lyse patient sample 3. We further generated a generate PP8 (SRS, SR6) mutant using homologous recombination by adding attachment protein SP6 to ORF 1 of the original PP8 template to generate PP8 (SRS, SR6). The primer sets used for this homologous recombination are:

1. Primer set for PP8 Fragment (bold) and
homologous recombination with SP6 gene (underline)
on 5′
>PP8-F3F
SEQ ID NO: 158
TAATACTCTACAGACACCACTAACTGATGCTGCTG
>PP8-SP6-R
SEQ ID NO: 160
CTCGTTTCAACATCTTTTATTTTGTACATACAAGGGATTAAGCAGTTCTT
ACCC
2. Amplification of 5P6 (underline):
>SP6-F
SEQ ID NO: 164
ATGTACAAAATAAAAGATGTTGAAACGAG
>SP6-R
SEQ ID NO: 165
TCACCCCTTAATTAAGTAAAGTGTATTAGGGTC
3. Primer set for PP8 Fragment (blue) and
homologous recombination with SP6 gene (underline)
on 3′
>PP8-SP6-F
SEQ ID NO: 166
AGACCCTAATACACTTTACTTAATTAAGGGGTGATGACTGATTGTTAAGA
TGGTG
>PP8-F3R
SEQ ID NO: 167
GTGGTAAGGTAAGGTATGGAAGGATGGCAGTAG

The sequence of the insertion (MRSA attachment protein SP6) is shown in SEQ ID NO: 169.

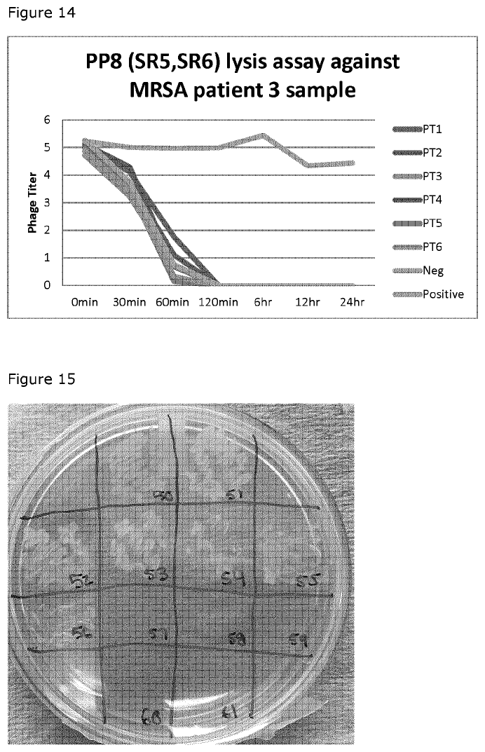

The resultant new strain of bacteriophage was called PP8(SP5, SP6). We used this new bacteriophage in conjunction with PP8(SP5) to determine if we could lyse patient samples 1 through 6 using these two new modified bacteriophages. As see in FIGS. 14 and 15, the new mutant bacteriophage lysed all six patient samples demonstrating that addition of a new attachment gene to our PP8 template allows for the specific targeting of a bacterium.

### Example 10—ORF2 Endolysis Gene Insertion (Inserted at 73195 in PP8)

1. Primer set for PP8 Fragment (bold) and
homologous recombination with foreign gene
(underline) on 5′
>PP8-F5F
SEQ ID NO: 170
AAGACTCGGAAGAAGGTAGTCACTAAGGAAAGTG
>PP8-endolysin-R
SEQ ID NO: 171
CCGTAAATCTTAGACCGTTGTCACTGAATCGCATGTCAAGTTTTACATAGA
AATCC
>endolysin-F
SEQ ID NO: 172
ATGCGATTCAGTGACAACGGTCTAAGATTTACGGCAGC
>endolysin-R
SEQ ID NO: 173
TTATGCTGCGTTACGCCCGATTTTCTCGGCAACGTCC
>PP8-endolysin-F
SEQ ID NO: 174
TTGCCGAGAAAATCGGGCGTAACGCAGCATAAAAGGTGATGTGGGTCTTGA
TAGG
>PP8-FSR
SEQ ID NO: 175
GCAACACTGTATCGGCTACTTCAAAGTCTTCTCTG

The insertion of the endolysis gene was carried out using normal molecular biology techniques. The sequence of the insertion is shown in SEQ ID NO: 176.

### Example 11—Insertion of Attachment Proteins in ORF 1 and ORF 2 Homologous Recombination in ORF1

1. Primer set for PP8 Fragment (bold) and
homologous recombination with foreign gene
(underline) on 5′
>PP8-F3F
SEQ ID NO: 158
TAATACTCTACAGACACCACTAACTGATGCTGCTG
>PP8-attachment_protein-R
SEQ ID NO: 177
CATATCCTGCGCCAGTCGCGACATACAAGGGATTAAGCAGTTCTTACCCA
AGC
2. Amplification of foreign gene (underline):
>attachment_protein-F
SEQ ID NO: 178
ATGTCGCGACTGGCGCAGGATATGAAAAAACTGG
>attachment_protein-R
SEQ ID NO: 179
TCAATCAGTATACCCGTATACCTGCTC
3. Primer set for PP8 Fragment (bold) and
homologous recombination with foreign gene
(underline) on 3′
>PP8-attachment_protein-F
SEQ ID NO: 180
TTGAGCAGGTATACGGGTATACTGATTGATGACTGATTGTTAAGATGGTG
>PP8-F3R
SEQ ID NO: 167
GTGGTAAGGTAAGGTATGGAAGGATGGCAGTAG

### Homologous Recombination in ORF2

1. Primer set for PP8 Fragment (bold) and
homologous recombination with foreign gene
(underline) on 5′
>PP8-F5F
SEQ ID NO: 170
AAGACTCGGAAGAAGGTAGTCACTAAGGAAAGTG
>PP8-attachment_protein-R
SEQ ID NO: 181
CATATCCTGCGCCAGTCGCGACATGTCAAGTTTTACATAGAAATCCTGTC
A
2. Amplification of foreign gene (underline):
>attachment_protein-F
SEQ ID NO: 182
ATGTCGCGACTGGCGCAGGATATGAAAAAACTGG
>attachment_protein-R
SEQ ID NO: 183
TCAATCAGTATACCCGTATACCTGCTC
3. Primer set for PP8 Fragment (bold) and
homologous recombination with foreign gene
(underline) on 3′
>PP8-attachment_protein-F
SEQ ID NO: 184
TTGAGCAGGTATACGGGTATACTGATTGAAAGGTGATGTGGGTCTTGATA
GG
>PP8-F5R
SEQ ID NO: 175
GCAACACTGTATCGGCTACTTCAAAGTCTTCTCTG

### Example 12—Bacteriophage Development to Target Escherichia coli, Salmonella enterica and Clostridium perfringens species

A sequence analysis of all pathogenic Escherichia coli, Salmonella enterica and Clostridium perfringens species currently causing mortality in Canadian poultry farms allowed evaluation and generation of a universal binding domain, which was used to genetically design phages to destroy these pathogenic bacteria. The process to achieve this was as follows:

1) Sequence analysis:

Samples of feces and other excrement were collected from Manitoba poultry farms for the identification of E. coli. and Salmonella enteric. Clostridium perfringens samples were supplied by an industry partner. All of these samples were used to isolate pathogenic bacteria as well as bacteriophage (to be used to build upon our bacteriophage library) present in the Canadian poultry population. Once pure cultures of pathogenic bacteria were attained, sequence analysis of the bacterium was carried out using an Illumine Miseq 2000. After analysis of these sequences, a ubiquitous attachment region for each bacterium was obtained. Using a Clone Manager genetic program, conserved attachment regions on the surface of various bacterial species were determined, and the generation of a genetic clone which can attach to the conserved bacterial binding domain was reverse engineered.

2) Insertion of conserved attachment region into template and propagation in yeast strain (CP109):

This ubiquitous attachment construct was sub-cloned into the disclosed bacteriophage template. Infectious bacteriophages were generated by transforming and propagating in yeast strain CP 109, which has the capability of holding multiple copies of the bacteriophage template. This was achieved using two methodologies:

a) in vivo by transformation and eventual induction of the bacteriophage template in yeast cells, or
b) in vitro by using cellular extracts of the yeast cells.

Regardless of the method used, the advantage of propagating using these methods lies in the avoidance of classical bacteriophage propagation in which potentially dangerous levels of bacterial endotoxins contaminate the preparations. These methods of phage production remove this hurdle, as yeast cells are used to grow the bacteriophage.

3) Determination of phage ability to target and infect multiple E.coli, Salmonella enterica and Clostridium perfringens pathogenic bacterial species:

Growth characteristics were carried out on the bacteriophage to ensure that the ubiquitous attachment protein was properly inserted. E.coli spp., Salmonella enterica and Clostridium perfringens were infected and phage growth analyzed. Lytic testing was carried out to ensure no integration took place. Cellular toxicity testing was carried out to validate the non-toxic extraction methods in yeast. The phages have been analyzed for binding ability and are ready for evaluation of phage treatment in broiler chickens.

### REFERENCES

Bonilla, N., Rojas M. I., Netto Flores Cruz, G., Hung, S. H., Rohwer, F., Barr, J. J. 2016. Phage on tap-a quick and efficient protocol for the preparation of bacteriophage laboratory stocks. PeerJ., 4, e2261.

Bourdin, G., Schmitt, B., Marvin Guy, L., Germond, J. E., Zuber, S., Michot, L., Reuteler, G., Brüssow, H. 2014. Amplification and purification of T4-like Escherichia coli phages for phage therapy: from laboratory to pilot scale. Appl Environ Microbiol. 80, 1469-1476.

Kropinski, A. M., Mazzocco, A., Waddell, T. E., Lingohr, E., Johnson, R. P. 2009. Enumeration of bacteriophages by double agar overlay plaque assay. Methods Mol Biol. 501, 69-76.

Pausz, C., Clasen, J. L., Suttle, C. A. 2009. Isolation independent methods of characterizing phage communities 1: strain typing using fingerprinting methods. Methods Mol Biol. 502, 255-278.

Sillankorva, S. 2018. Isolation of Bacteriophages for Clinically Relevant Bacteria. Methods Mol Biol. 1693, 23-30.

Van Twest, R., Kropinski, A. M. 2009. Bacteriophage enrichment from water and soil. Methods Mol Biol. 501, 15-21.

Gasset, M. (2010). Bacteriophage Holins and their Membrane Disrupting Ability, 123-148. https://doi.org/10.1002/9780470570548.ch6

Fischetti, V. A. (2008). Bacteriophage lysins as effective antibacterials. Current Opinion in Microbiology, 11(5), 393-400. https://doi.org/10.1016/j.mib.2008.09.012

Borysowski, J., Weber-Dabrowska, B., & Gorski, A. (2006) Bacteriophage Endolysins as a Novel Class of Antibacterial Agents. Experimental Biology and Medicine, 366-377. http://journals.sagepub.com/doi/10.1177/153537020623100402

1. (canceled)

2. A method of engineering bacteriophages comprising:
isolating a bacteriophage;
removing all attachment genes from a genome of said bacteriophage;
inserting a first unique open reading frame encoding one or more attachment genes and inserting a second unique open reading frame encoding one or more genes useful for overcoming bacterial defenses;
inserting a non-natural attachment gene into said first open reading frame, wherein said non-natural attachment gene is specific for attaching to a selected bacteria.

3. The method of claim 2 wherein one or more genes useful for overcoming bacterial defenses are endolysins, bio-film reducers, glycocalyx penetrators, or any combination thereof.

4-6. (canceled)

7. The method of any of claim 2, wherein said bacteriophage is lytic.

8. The method of claim 2 wherein said removing and said inserting utilizes cell free cloning of said bacteriophages.

9. (canceled)

18. A method of producing a mutant bacteriophage, the method comprising;
inactivating an attachment gene from a selected bacteriophage, the selected bacteriophage being isolated from bacteriophages from the environment;
inserting, into the selected bacteriophage, a first heterologous nucleic acid sequence comprising a first open reading frame encoding a first specific attachment gene, the first specific attachment gene being different than the inactivated attachment gene and being specific for a selected bacteria, to produce the mutant bacteriophage, and
inserting a second heterologous nucleic acid sequence comprising a second open reading frame encoding a gene useful for overcoming bacterial defenses.

19. (canceled)

20. The method of claim 18, wherein the gene useful for overcoming bacterial defenses comprises one or more of a biofilm degrading gene, a glycocalyx degrading gene, a gene encoding an antibacterial protein, or a gene for an enzyme that disrupts the bacterial wall, to produce the mutant bacteriophage.

21. The method of claim 20 wherein the gene for an enzyme that disrupts the bacterial wall is an endolysin.

22. The method of claim 21 wherein the endolysin comprises the nucleotide sequences of SEQ ID No: 138; SEQ ID No: 139; SEQ ID No: 140; SEQ ID No: 141; SEQ ID No: 142; or SEQ ID No: 143; or a fragment thereof.

23. The method of claim 20 wherein the gene for an enzyme that disrupts the bacterial wall comprises the nucleotide sequence of SEQ ID No: 144, or a fragment thereof.

24. The method of claim 20 wherein the biofilm degrading gene and glycocalyx degrading gene comprise one or more of Cathelicidin antimicrobial peptide LL-37; Histatin 3 (HTN3); Nisin; Dispersin B; Endo-1,4-β-glucanase (callulase); Aureolysin; NucB; Serine protease (SspA); LapG protease; Melittin; Endo-1,4-β-mannosidase (manA); or α-amylase, or a fragment thereof.

25. The method of claim 20 wherein the gene for an enzyme that disrupts the bacterial wall is a gene that targets linking chemistries in the bacterial cell wall.

26. The method of claim 25 wherein the gene that targets linking chemistries in the bacterial cell wall comprises nucleotide sequences of one or more of SEQ ID No: 145; SEQ ID No: 146; SEQ ID No: 146; SEQ ID No: 147; SEQ ID No: 149; SEQ ID No: 150; SEQ ID No: 151; SEQ ID No 152; or a fragment thereof.

27. The method of claim 18, further comprising screening for lysogenic genes, and inactivating said lysogenic genes.

28. The method of claim 18, wherein the selected bacteriophage is a bacteriophage with a low copy number of lysogenic genes.

29. The method of claim 18, wherein the step of inactivating, inactivates all attachment genes from the selected bacteriophage.

30. The method of claim 18, wherein the selected bacteriophage is one or more of a lytic bacteriophage, a bateriohage with a small genome size, or a bacteriophage with structural and functional genes to lyse gram negative and gram-positive bacteria, or any combination thereof.

31. The method of claim 18, wherein the first specific attachment gene is attachment gene SP5, attachment gene SP6, or fragment thereof.

32. The method of claim 18, wherein the first specific attachment gene comprises the nucleotide sequences of SEQ ID No: 125; SEQ ID No: 126; SEQ ID No: 127; SEQ ID No: 128; SEQ ID No: 129; SEQ ID No: 130; SEQ ID No: 131; SEQ ID No: 132; SEQ ID No: 133; SEQ ID No: 134; SEQ ID No: 135; SEQ ID No: 136; or SEQ ID No: 137; or fragment thereof.

33. The method of claim 18, wherein the first open reading frame further encodes a second specific attachment gene that is different than the first specific attachment gene.

34. A mutant bacteriophage produced according to the method of claim 18.

35. An anti-microbial composition for sanitizing or decontaminating a surface comprising the mutant bacteriophage of claim 18.

36. A method of decontaminating a surface suspected of containing an infectious bacteria, the method comprising applying a bacteriocidal effective amount of the composition of claim 35 to the surface.

37. (canceled)
